(19) United States
(12) Patent Application Publication
(10) Pub. No.: US 2025/0186379 A1
(43) Pub. Date: Jun. 12, 2025

(54) METHODS AND AGENTS FOR DECREASING INSULIN RESISTANCE

(71) Applicant: Whitehead Institute for Biomedical Research, Cambridge, MA (US)

(72) Inventors: Alessandra Dall'Agnese, Cambridge, MA (US); Jesse Platt, Cambridge, MA (US); Tong Ihn Lee, Somerville, MA (US); Richard A. Young, Boston, MA (US)

(21) Appl. No.: 18/294,966

(22) PCT Filed: Aug. 5, 2022
(86) PCT No.: PCT/US2022/039602

§ 371 Date: Feb. 2, 2024

Related U.S. Application Data
(60) Provisional application No. 63/230,031, filed on Aug. 5, 2021.

(51) Int. Cl. A61K 31/198; A61P 3/10; G01N 33/50

(52) U.S. Cl. CPC A61K 31/198; A61P 3/10; G01N 33/5023; G01N 33/5067

(57) ABSTRACT

Disclosed herein are methods and agents for decreasing insulin resistance and metformin resistance by modulating insulin receptor condensates. Also disclosed are methods of screening for agents to decrease insulin resistance.

### CROSS-REFERENCE TO RELATED APPLICATION(S)

This application claims the benefit of U.S. Provisional Application No. 63/230,031, filed Aug. 5, 2021. The entire teachings of the above application are incorporated herein by reference.

### GOVERNMENT SUPPORT

This invention was made with government support under R01 GM12351, T32 5T32DK007191-45 awarded by the National Institutes of Health; and PHY1743900 awarded by the National Science Foundation. The government has certain rights in the invention.

### BACKGROUND OF THE INVENTION

Insulin resistance is a common feature of type 2 diabetes (T2D), obesity, and metabolic syndrome. Insulin resistance is characterized by impaired sensitivity to insulin-mediated glucose uptake. Pancreatic beta cells initially compensate by releasing more insulin, resulting in hyperinsulinemia. Chronic hyperinsulinemia exacerbates insulin resistance and metabolic dysfunction and can lead to beta cell failure. The understanding of the mechanisms that give rise to insulin resistance is incomplete, but have been postulated to involve alterations in insulin signaling components as a consequence of chronic hyperinsulinemia, inflammation, oxidative stress, ER stress, fatty acid accumulation and mitochondrial dysfunction. The mysteries of insulin resistance extend to its first-line drug, metformin, which is prescribed to more than 150 million people. Metformin has been proposed to act via multiple mechanisms, including inhibition of respiratory complex I and activation of AMP-activated protein kinase (AMPK). How modulation of these metabolic functions might mitigate the effects of insulin resistance is not fully understood.

Insulin binding to the insulin receptor (IR), a receptor tyrosine kinase (RTK), activates PI3K-AKT and ERK signaling, internalization and recycling of IR and, for some IR molecules, transport into the nucleus where they bind and enhance expression of insulin-responsive genes.

### SUMMARY OF THE INVENTION

Applicant provides herein a condensate model for insulin receptor (IR) function in normal conditions and when dysregulated in chronic hyperinsulinemia-induced insulin resistance. It has been found that IR is incorporated into liquid-like condensates at the plasma membrane, in the cytoplasm and in the nucleus of liver cells, as well as evidence of insulin-dependent IR function in condensates. Insulin stimulation promotes further incorporation of IR into these dynamic condensates in insulin sensitive cells, which form and dissolve on short, sub-minute time-scales. In contrast, it has been surprisingly found that insulin stimulation does not promote further incorporation of IR into condensates in insulin resistant cells, where IR molecules within condensates exhibit less dynamic behavior. Metformin treatment of insulin resistant cells rescues IR condensate dynamics and insulin responsiveness. It has also been found that insulin resistant cells experience high levels of oxidative stress, which causes reduced condensate dynamics, and treatment of these cells with metformin reduces ROS levels and returns condensates to their normal dynamic behavior. On the basis of these surprising discoveries, Applicant provides herein methods and compositions for reducing insulin resistance as well as methods of screening for agents to reduce insulin resistance.

Some aspects of the present disclosure are directed to a method of decreasing insulin resistance in an insulin resistant cell, comprising contacting the cell with an agent that decreases a level of reactive oxygen species (ROS) in the cell, wherein the agent is not metformin. In some embodiments, contact of the cell with the agent and insulin increases incorporation of insulin receptor (IR) into cytoplasmic and/or nuclear condensates in the cell. In some embodiments, contact of the cell with the agent and insulin increases incorporation of insulin receptor (IR) into transcriptional condensates. In some embodiments, the transcriptional condensates comprise transcriptional condensates modulating the expression of one or more insulin responsive genes. In some embodiments, the cell is a mammalian (e.g., human) fat, liver, brain, kidney, muscle (e.g., skeletal muscle), or pancreatic islet cell (e.g., pancreatic b-cell). In some embodiments, the agent is contacted with the cell in vivo in a subject in need thereof.

Some aspects of the present disclosure are directed to a method of decreasing insulin resistance in an insulin resistant cell, comprising contacting the cell with an agent that incorporates into transcriptional condensates of the cell and increases expression of one or more insulin responsive genes in the presence of insulin. In some embodiments, the agent is a functional insulin receptor variant or derivative having increased affinity, as compared to wild-type insulin receptor, for the transcriptional condensates. In some embodiments, the agent is a functional insulin receptor variant or derivative having increased affinity, as compared to wild-type insulin receptor, for a transcriptional condensate in an elevated reactive oxygen species (ROS) environment. In some embodiments, the cell is a mammalian (e.g., human) fat, liver, brain, kidney, muscle (e.g., skeletal muscle) or pancreatic islet cell (e.g., pancreatic b-cell). In some embodiments, the agent is contacted with the cell in vivo in a subject in need thereof.

### Methods of Screening

Some aspects of the present disclosure are directed to a method of screening for a candidate agent to treat insulin resistance comprising contacting a test agent with a condensate and insulin receptor (IR) in an elevated reactive oxygen species (ROS) environment, wherein if the test agent increases flux of IR incorporation into the condensate as compared to a control then the test agent is identified as a candidate agent to treat insulin resistance.

Some aspects of the present disclosure are directed to a method of screening for a candidate agent to treat insulin resistance comprising contacting a test agent with a condensate and insulin receptor (IR), wherein the condensate exhibits reduced incorporation of IR in the presence of insulin as compared to a control, wherein if the test agent increases flux of IR incorporation into the condensate as compared to a control then the test agent is identified as a candidate agent to treat insulin resistance. In some embodiments, one or more components of the condensate has been exposed to elevated ROS levels.

Some aspects of the present disclosure are directed to a method of screening for a candidate agent to treat insulin resistance comprising contacting a test agent with a condensate, insulin receptor (IR), and insulin in an elevated reactive oxygen species (ROS) environment, wherein if the test agent increases IR incorporation into the condensate as compared to a control then the test agent is identified as a candidate agent to treat insulin resistance.

Some aspects of the present disclosure are directed to a method of screening for a candidate agent to treat insulin resistance comprising contacting a test agent with a condensate, insulin receptor (IR), and insulin, wherein the condensate exhibits reduced incorporation of IR in the presence of insulin as compared to a control, wherein if the test agent increases IR incorporation into the condensate as compared to a control then the test agent is identified as a candidate agent to treat insulin resistance. In some embodiments, one or more components of the condensate has been exposed to elevated ROS.

Some aspects of the present disclosure are directed to a method of screening for a candidate agent to treat insulin resistance comprising contacting a test agent with a mixture comprising a cell or in vitro transcription assay with condensate dependent expression of a reporter gene, insulin receptor (IR), and insulin, wherein the condensate exhibits reduced incorporation of IR in the presence of insulin as compared to a control, wherein if the test agent increases reporter gene expression as compared to a control then the test agent is identified as a candidate agent to treat insulin resistance. In some embodiments, the condensate is a synthetic in vitro condensate or an ex vivo condensate isolated from a cell. In some embodiments, the ex vivo condensate is isolated from an insulin resistant cell. In some embodiments, the insulin resistant cell is a mammalian (e.g., human) fat, liver, brain, kidney, muscle (e.g., skeletal muscle), or pancreatic islet cell (e.g., pancreatic b-cell).

Some aspects of the present disclosure are directed to a method of screening for a candidate agent to treat insulin resistance comprising (a) contacting a test agent with IR condensates and insulin, wherein the IR condensates exhibits increased average lifetime and/or increased percentage of long-lived IR condensates in the presence of insulin as compared to a control; and (b) measuring the average lifetime and/or percentage of long-lived IR condensates wherein if the test agent decreases average lifetime of the IR condensates and/or increases the percentage of long-lived IR condensates as compared to a control then the test agent is identified as a candidate agent to treat insulin resistance.

Some aspects of the present disclosure are directed to a method of screening for a candidate agent to modulate insulin receptor activity comprising (a) contacting a test agent with IR condensates and insulin: (b) measuring a property or behavior of said IR condensates as compared to a control, and (c) identifying the test agent as a candidate modulator of insulin receptor activity if the property or behavior of said IR condensates differ in the presence of the test agent as compared to the control, optionally wherein the property or behavior comprises average number of IR molecule(s) in IR condensates, average lifetime of IR condensates, and/or percentage of long-lived IR condensates.

In some embodiments, the condensate(s) is/are in a cell. In some embodiments, the cell is a mammalian (e.g., human) fat, liver, brain, kidney, muscle (e.g., skeletal muscle) or pancreatic islet cell (e.g., pancreatic b-cell). In some embodiments, the cell is an insulin resistant cell. In some embodiments, the cell is an insulin resistant and metformin resistant cell.

Some aspects of the present disclosure are directed to a method of characterizing an agent, comprising (i) contacting the agent with a cell comprising the insulin receptor, and (ii) measuring ability of the agent to modulate one or more of the following: (a) flux of IR incorporation into condensates within the cell in response to insulin; (b) average number of IR molecules in IR condensates in the cell; (c) average lifetime of IR condensates in the cell; (d) percentage of long-lived IR condensates in the cell.

In some embodiments, the cell is a mammalian (e.g., human) fat, liver, brain, kidney, muscle (e.g., skeletal muscle) or pancreatic islet cell (e.g., pancreatic b-cell). In some embodiments, the cell is an insulin resistant cell. In some embodiments, the cell is an insulin resistant and metformin resistant cell.

In some embodiments, the cell is contacted with insulin prior to step (i). In some embodiments, the cell is contacted with insulin prior to step (ii).

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, NJ, 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™ McKusick-Nathans Institute of Genetic Medicine, Johns Hopkins University (Baltimore, MD) and National Center for Biotechnology Information, National Library of Medicine (Bethesda, MD), as of May 1, 2010, World Wide Web URL: ncbi.nlm.nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1H demonstrate how insulin promotes IR incorporation into puncta in insulin sensitive HepG2 cells. FIG. 1A shows a schematic of cell treatments to study insulin signaling. This regimen puts the cells in culture conditions that are similar to normal physiological baseline and uses insulin stimuli that approximate normal physiological levels. FIG. 1B shows immunofluorescence (IF) images of IR using a validated antibody in cells treated acutely with 0 nM or 3 nM insulin for 5 min. IR fluorescence signal is shown in green. Dashed light blue lines represent nuclear outline determined by Hoechst stain (not shown). Orange, magenta and yellow boxes represent regions at the plasma membrane (PM), nucleus and cytoplasm (Cytop), respectively, that are magnified on the right. Scale bars are indicated in the images. FIG. 1C shows the quantification of IR signal at the PM, nucleus and cytoplasm in cells stimulated (3 nM) or not (0 nM) with insulin. FIG. 1D shows the live imaging time course of HepG2 cells expressing endogenous IR tagged with monomeric enhanced green fluorescent protein (IR-GFP) during insulin stimulation. The time of acquisition is shown in the above images. The dashed light blue lines represent the nuclear outline and the scale bar is indicated in the images. The above images are representative images of the three cells. The bottom left images contain magnified images of PM, nucleus and cytoplasm. The bottom right image is the quantification of IR puncta signal. FIG. 1E above image shows the immunoblot for IR and beta-actin control in cells treated acutely with 0 nM or 3 nM insulin. The bottom image shows quantification of relative IRb levels in HepG2 cells without (0 nM) or with (3 nM) acute insulin stimulation. FIG. 1F shows ChIP-seq tracks of IR, MED1 and RPB1 at the SMAD7 locus. FIG. 1G shows SMAD7 transcript levels determined by RNA-seq before and after insulin stimulation. FIG. 1H shows colocalization of IR and nascent RNA of SMAD7 determined by imaging IR-GFP and SMAD7 intronic RNA FISH in fixed cells treated with FF0 nM versus 3 nM insulin in the left image. The colocalization area (magenta box) is magnified at the bottom right corner of each image. The quantification of the percentage of RNA FISH puncta that colocalize with IR puncta and the IR signal at the RNA FISH puncta is in the right image.

FIGS. 2A-2E demonstrate how insulin receptor puncta exhibit features of liquid-like condensates. FIG. 2A shows a schematic of cell treatments (top image) and representative images of IR puncta undergoing deformation (left image), fission (center image) and fusion (right image). The observed (Obs) IR signal corresponds to the IR puncta signal measured, while the expected (Exp) IR intensity, corresponds to the IR puncta signal expected if the same puncta underwent deformation, fission or fusion. FIG. 2B shows the schematic of cell treatments (top image). A super-resolution image of endogenous IR labeled with Dendra2 in living HepG2 cells treated acutely with 0 nM or 3 nM insulin for 5 min is in the bottom left image. Representative images of IR clusters and corresponding time-correlated photoactivation localization microscopy (tcPALM) traces are in the bottom right image. FIG. 2C shows the frequency of IR cluster lifetime at the plasma membrane, cytoplasm and nucleus. The Average lifetime (τavg) is reported in the graphs. Light blue corresponds to HepG2 cells not acutely treated with insulin (0 nM insulin), while dark blue corresponds to HepG2 cells acutely treated with insulin for 5 min (3 nM insulin). FIG. 2D shows the number of transient clusters per cell at the plasma membrane, cytoplasm and nucleus based on tc-PALM. FIG. 2E shows the number of IR molecules per transient IR cluster based on tc-PALM. The average number is reported in parentheses on top of each histogram.

FIGS. 3A-3G demonstrate IR incorporation into condensates is attenuated in insulin resistant HepG2 cells. FIG. 3A shows the schematic of cell treatments to model insulin sensitivity and resistance. Cells were cultured in physiological or pathological concentrations of insulin to model differences between healthy individuals and patients with hyperinsulinemia. FIG. 3B shows the immunoblot and quantification to measure phosphorylated signaling proteins (pIRb, pAKT, pERK) relative to total insulin signaling proteins (IRb, AKT, ERK), showing decreased insulin signaling in cells treated with pathological levels of insulin (R) as compared to the cells treated with physiological levels of insulin (S). FIG. 3C shows the schematic of cell treatments (left image), the immunoblot for IR and beta-actin in cells cultured in physiological (S) or pathological (R) levels of insulin (center image), and the quantification of relative levels of IRb in HepG2 cells cultured in physiological (S) or pathological (R) levels of insulin (right image). FIG. 3D shows the schematic of cell treatments (top image) and IF for IR (green) in insulin resistant cells acutely treated with 0 nM or 3 nM insulin, showing attenuated incorporation of IR into condensates upon insulin stimulation in insulin resistant cells. FIG. 3E shows the quantification of IR signal in IR condensates at the plasma membrane (PM), nucleus, and cytoplasm of insulin resistant cells acutely treated with 0 nM or 3 nM insulin. FIG. 3F shows the colocalization of IR and nascent RNA of SMAD7 determined by imaging IR-GFP and SMAD7 intronic RNA FISH in fixed cells treated with 0 nM versus 3 nM insulin. The colocalization area (magenta box) is magnified at the bottom right corner of each image. The panel at the right image shows quantification of the percentage of RNA FISH puncta that colocalize with IR puncta. FIG. 3G shows SMAD7 transcript levels determined by RT-qPCR in cells treated with 0 nM or 3 nM insulin.

FIGS. 4A-4B show IR condensates have different biophysical properties in insulin sensitive and resistant HepG2 cells. FIG. 4A: Schematic of cell treatments (top). A superresolution image of endogenous IR labeled with Dendra2 in insulin sensitive and resistant living HepG2 cells (bottom left). Representative super resolved images of IR clusters and corresponding time-correlated photoactivation localization microscopy (tcPALM) traces (bottom right). FIG. 4B: Frequency of IR cluster lifetime at the PM, cytoplasm and nucleus. Average lifetime (τavg) is reported in the graphs. Light blue corresponds to insulin sensitive HepG2 cells, while red corresponds to insulin resistant HepG2 cells.

FIGS. 5A-5D show metformin rescues IR condensate behavior in insulin resistant HepG2 cells. FIG. 5A: Schematic of cell treatments (top). Imaging of IR-GFP in insulin sensitive and resistant cells treated with or without metformin (bottom). Metformin concentration is reported above the images. IR-GFP fluorescence signal is shown in green. Dashed light blue lines represent nuclear outline. Orange, magenta and yellow boxes represent regions at the plasma membrane (PM), nucleus and cytoplasm, respectively, that are magnified in B. Scale bars are indicated in the images. FIG. 5B: Magnification of IR-GFP condensates at the plasma membrane (PM), cytoplasm and nucleus (left). Quantification of IR-GFP signal at IR condensates at the plasma membrane, nucleus and cytoplasm (right). FIG. 5C: tc-PALM results on insulin sensitive (S), resistant (R) and metformin-treated resistant (RM) cells. The concentration of metformin used was 12.5 M and cells were imaged after insulin washout without being acutely stimulated with insulin. Frequency of transient IR cluster lifetime at the plasma membrane, cytoplasm and nucleus. Average lifetime (τavg) is reported in the graphs. Blue corresponds to insulin sensitive HepG2 cells, red corresponds to insulin resistant HepG2 cells and purple corresponds to insulin resistant HepG2 cells treated with metformin. FIG. 5D: Colocalization of IR and nascent RNA of SMAD7 determined by imaging IR-GFP and SMAD7 intronic RNA FISH in fixed insulin sensitive (S), insulin resistant (R) and metformin-treated insulin resistant (RM) cells all stimulated with 3 nM insulin for 5 min. Colocalization area (magenta box) is magnified at the bottom right corner of each image. Panel at right shows quantification of the percentage of RNA FISH puncta that colocalize with IR puncta.

FIGS. 6A-6E show high ROS levels in insulin resistant cells and suppression by metformin. FIG. 6A: IF for NRF2 (magenta) in insulin sensitive or resistant HepG2 cells, showing increased levels of this marker of oxidative stress in insulin resistant cells relative to insulin sensitive cells (left). Quantification of mean NRF2 signal intensity in nuclei of insulin sensitive (S) or resistant (R) cells (right). FIG. 6B: Images of cells treated with ROS-sensitive dye (middle). The brighter color (magenta) is associated with higher ROS levels. Relative ROS signal in insulin sensitive or resistant cells treated with or without 12.5 M metformin (right). FIG. 6C: Schematic of cell treatments (top). Imaging of IR-GFP in insulin sensitive cells (S), insulin sensitive cells treated with H₂O₂ (SH) or insulin resistant cells (R). Dashed light blue lines represent nuclear outline. Orange, magenta and yellow boxes represent regions at the plasma membrane (PM), nucleus and cytoplasm (cytop), respectively, that are magnified on the right. Scale bars are indicated in the images. Zoom of IR condensates at the PM, cytoplasm (Cytop) and nucleus. Quantification of IR signal in IR puncta at the PM, nucleus and cytoplasm in insulin sensitive (S), H2O2-treated sensitive (SH) and resistant (R) cells. FIG. 6D: tc-PALM results on insulin sensitive (blue), H2O2-treated sensitive (brown) and resistant (red) cells. The concentration of H₂O₂ used was 20 mM and cells were imaged after insulin washout without being acutely stimulated with insulin. Frequency of IR cluster lifetime at the PM, cytoplasm and nucleus. Average lifetime (τavg) is reported in the graphs. Blue corresponds to insulin sensitive HepG2 cells, brown corresponds to insulin sensitive cells treated with H₂O₂ and red corresponds to insulin resistant HepG2 cells. FIG. 6E: Colocalization of IR and nascent RNA of SMAD7 determined by imaging IR-GFP and SMAD7 intronic RNA FISH in fixed insulin sensitive (S), H2O2-treated sensitive (SH) and resistant (R) HepG2 cells all acutely stimulated with 3 nM insulin for 5 min. Colocalization area (magenta box) is magnified at the bottom right corner of each image. Panel at right shows quantification of the percentage of RNA FISH puncta that colocalize with IR puncta.

FIGS. 7A-7F show condensate rescue in insulin resistant human liver spheroids and liver tissue. FIG. 7A: Schematic of cell treatments for panels B-E. FIG. 7B: ELISA quantification of albumin production by human liver spheroids cultured with physiologic (blue) or pathologic (red) concentrations of insulin. FIG. 7C: ELISA quantification of insulin clearance by human liver spheroids cultured with physiologic (blue) or pathologic (red) concentrations of insulin. FIG. 7D: IF for IR in insulin sensitive human liver spheroids acutely treated with 0 nM or 3 nM insulin for 10 minutes. Image of an entire cell (left) and quantification of IR signal at IR condensates at the plasma membrane, cytoplasm and nucleus (right). FIG. 7E: IF for IR in insulin sensitive (left), insulin resistant (middle), and insulin resistant+12.5 M metformin (right) human liver spheroids. All samples were acutely treated with 3 nM insulin for 10 min. Quantification of IR signal in IR condensates at the plasma membrane (PM), cytoplasm and nucleus in insulin sensitive and resistant human liver spheroids. FIG. 7F: Graphic representation of human liver tissue experiment. IF for IR and CK18 in liver tissue from healthy donor (H), donor with T2D (T), and donor with T2D that had been treated with Metformin™. Dashed light blue lines represent nuclear outline determined by Hoechst stain (not shown). Orange, magenta and yellow boxes represent regions at the plasma membrane (PM), nucleus and cytoplasm, respectively, that are magnified in FIG. 14B. Scale bars are indicated in the images. Quantification of IR signal at IR condensates at the plasma membrane, cytoplasm and nucleus (left).

FIGS. 8A-8F show validation of insulin sensitive HepG2 cell model and reagents. FIG. 8A: Insulin signaling graphic. FIG. 8B: Cell model to study insulin signaling using physiological concentrations of insulin (top). Percentage viability of cells cultured in cell expansion media (Media+FBS) or in media containing physiological concentrations of insulin (Media−FBS) is reported in the graph (bottom). FIG. 8C: Experimental protocol and immunoblot to quantify phosphorylated insulin signaling proteins (pIRb, pAKT, pERK) over total insulin signaling proteins (IRb, AKT, ERK). FIGS. 8D and 8E: Validation of antibody against IR by immunoblot (FIG. 8D) and immunofluorescence (FIG. 8E). FIG. 8F: Quantification of number (N) of IR puncta at the plasma membrane (PM), nucleus and cytoplasm of HepG2 cells treated with 0 nM or 3 nM insulin for 5 min (relative to FIG. 1B).

FIGS. 9A-9D show HepG2 cells expressing fluorescently labeled endogenous IR express similar levels of IR as WT HepG2 cells and are insulin sensitive. FIG. 9A: Schematic of knock-in strategy. FIG. 9B: Schematic of cell treatments (top). Immunoblot for IR and beta-actin control in WT, IR-GFP and IR-Dendra2 cell lines. The shift in molecular weight is the expected size for the GFP or Dendra2 fusion with IR. FIG. 9C: Schematic of cell treatments (top) and assessment of insulin sensitivity of IR-GFP and IR-Dendra2 cell lines (bottom). Immunoblot and quantification of phosphorylated insulin signaling proteins relative to total insulin signaling proteins (pIR/IR, pAKT/AKT, pERK/ERK), demonstrating that endogenously tagged IR is functional and can activate insulin signaling upon insulin stimulation. FIG. 9D: Quantification of number (N) of IR puncta at the plasma membrane (PM), nucleus and cytoplasm of IR-GFP cells treated with 3 nM insulin for 0, 2.5 min, 5 min and 7.5 min (relative to FIG. 1D).

FIG. 10 shows the estimation of the number of IR molecules in HepG2 cells: Quantitative western blot with standard curve of purified IRbeta mCherry fusion protein (IRb-mCherry; first 6 lanes) and cell lysate containing a specific number of cells (last four lanes).

FIGS. 11A-11H show single-molecule statistics and validation of tcPALM analysis. FIG. 11A: Distribution of number of detections of each single molecule. Total number of 8335 single molecules are collected for plotting the histogram. FIG. 11B: Distribution of the lifetime span of single molecules with more than one detection. FIG. 11C: Distribution of the inter-detection period (dark-time) of single molecules with more than one detection. Total number of 867 multi-detection single molecules are collected for plotting the histograms in (FIG. 11B) and (FIG. 11C). FIG. 11D: Histogram of inter-detection period of identified transient clusters in live cells and pseudo-transient clusters in fixed cells selected with the same procedure as in live cells. The counts of each bin are normalized to the first bin, which mostly consists of counts of blinking events from single molecules (given that most single molecules have a lifetime span shorter than Is). FIG. 11E: Example of detection profiles including different types of temporal structures. The real time-correlated multimolecule bursts are highlighted with red lines in the cumulant plots. FIG. 11F: ODEs of the number of pre-converted (Moff) and post-converted (Mon) Dendra2 molecules in a ROI, followed by the analytical solution as a dual-exponential function. kₐ is the photo-activation rate, and k_{b} is the photo-bleaching rate, where applicant has assumed kₐ<k_{b} during the fitting. FIG. 11G: Distribution of fitted kₐ from many ROIs. FIG. 11H: Cumulative number of photo-converted (i.e. observed) molecules given various total number of pre-converted molecules (M) in the beginning.

FIGS. 12A-12B show metformin does not affect IR condensates in insulin sensitive cells and does not alter IR protein level in insulin resistant cells. FIG. 12A: Schematic of cell treatments (top). Imaging of IR-GFP in insulin sensitive cells treated with or without metformin (bottom). Metformin concentration is reported above the images. IR-GFP fluorescence signal is shown in green. Dashed light blue lines represent nuclear outline. Orange, magenta and yellow boxes represent regions at the plasma membrane (PM), nucleus and cytoplasm, respectively, that are magnified on the right (ZOOM). Scale bars are indicated in the images. FIG. 12B: Schematic of cell treatments (top). Immunoblot for IR and beta-actin in cells cultured in pathologic levels of insulin treated with (RM) or without (R) metformin (bottom left). Quantification of relative levels of IRb (bottom right).

FIGS. 13A-13B show Metformin does not affect IR condensates in insulin sensitive cells and does not alter IR protein level in insulin resistant cells. FIG. 13A: Schematic of cell treatments (top). Imaging of IR-GFP in insulin sensitive cells treated with or without metformin (bottom). Metformin concentration is reported above the images. IR-GFP fluorescence signal is shown in green. Dashed light blue lines represent nuclear outline. Orange, magenta and yellow boxes represent regions at the plasma membrane (PM), nucleus and cytoplasm, respectively, that are magnified on the right (ZOOM). Scale bars are indicated in the images. FIG. 13B: Schematic of cell treatments (top). Immunoblot for IR and beta-actin in cells cultured in pathologic levels of insulin treated with (RM) or without (R) metformin (bottom left). Quantification of relative levels of IRb (bottom right).

FIG. 14A-14D show human donor information and IR puncta in liver tissue. FIG. 14A: Table with baseline clinical information of human donors. FIG. 14B: Magnification of IR condensates at the plasma membrane (PM; orange), cytoplasm (yellow), nucleus (magenta) relative to FIG. 7F. FIG. 14C: IF for IR in liver tissue from duplicate healthy donor (H2), a duplicate donor with T2D (T2), and donor with T2D that had been treated with Metformin™. Dashed light blue lines represent nuclear outline determined by Hoechst stain (not shown). Scale bars are indicated in the images. FIG. 14D: Quantification of IR signal at IR condensates at the plasma membrane (PM), cytoplasm and nucleus of hepatocytes from liver tissue obtained from two healthy donors (H and H2), two donors with T2D (T and T2) and a donor with T2D that had been treated with Metformin™. Quantification for H, T and TM is the same as in FIG. 7F.

FIGS. 15-41 re-presents certain data from FIGS. 1-14 and provides additional data.

FIGS. 15A-15B demonstrates insulin receptor bodies in human liver cells. FIG. 15A shows representative immunofluorescence images for IR and CK18 in liver tissue from a healthy donor (Healthy), a donor with T2D (T2D), and a donor with T2D who had been treated with metformin (T2D+metformin). Dashed light blue lines represent the nuclear outline determined by Hoechst stain (not shown). Orange, magenta and yellow boxes represent regions at the plasma membrane, nucleus and cytoplasm, respectively, that are magnified on the right (ZOOM). Scale bars are indicated in the images. FIG. 15B shows quantification of IR signal in puncta at the plasma membrane, cytoplasm and nucleus for 7 healthy donors, 7 donors with T2D and 9 donors with T2D who had been treated with metformin.

FIGS. 16A-16E demonstrate insulin receptor bodies in HepG2 cells. FIG. 16A provides a schematic of cell treatments (top). This regimen puts the cells in culture conditions that are similar to those experienced by hepatocytes in situ and uses insulin stimuli that approximate normal physiological levels. Representative immunofluorescence images of IR using a validated antibody in cells stimulated with (3 nM) or without (0 nM) insulin for 5 min (bottom). IR fluorescence signal is shown in green. Dashed light blue lines represent nuclear outline determined by Hoechst stain (not shown). Orange, magenta and yellow boxes represent regions at the plasma membrane (PM), nucleus and cytoplasm (Cytop), respectively, that are magnified (ZOOM, middle). Scale bars are indicated in the images. FIG. 16B shows quantification of IR signal intensity in puncta at the plasma membrane (top), cytoplasm (middle) and nucleus (bottom) in cells stimulated (3 nM) or not (0 nM) with insulin. FIG. 16C provides a schematic of cell treatments to model insulin resistance (top). Cells were cultured in pathological concentrations of insulin to mimic conditions experienced by hepatocytes in situ of patients with hyperinsulinemia. Representative immunofluorescence images for IR (green) in insulin-resistant cells acutely stimulated with (3 nM) or without (0 nM) insulin, showing attenuated incorporation of IR into puncta upon insulin stimulation in insulin-resistant cells (bottom). Orange, magenta and yellow boxes represent regions at the plasma membrane (PM), nucleus and cytoplasm (Cytop), respectively, that are magnified (ZOOM, middle). Scale bars are indicated in the images. FIG. 16D provides a schematic of cell treatments to model insulin sensitivity, insulin resistance, and metformin treatment (top). Cells used were homozygous HepG2 cells expressing endogenous IR tagged with GFP (IR-GFP). Metformin concentration used is 12.5 μM. Representative images for IR-GFP in insulin-sensitive, insulin-resistant and metformin-treated insulin-resistant cells stimulated with insulin (3 nM) for 5 minutes (bottom). Orange, magenta and yellow boxes represent regions at the plasma membrane (PM), cytoplasm (Cytop) and nucleus, respectively, that are magnified (ZOOM). Scale bars are indicated in the images. FIG. 16E shows quantification of IR-GFP signal intensity in IR puncta at the plasma membrane, cytoplasm and nucleus of insulin-sensitive, insulin-resistant and metformin-treated insulin-resistant cells acutely stimulated with (3 nM) insulin.

FIGS. 17A-17I demonstrate insulin receptor puncta exhibit features of liquid-like condensates. FIG. 17A provides a schematic of cell treatments (top) and representative images of IR puncta undergoing deformation (bottom left), fission (bottom center) and fusion (bottom right). Quantification of total IR signal intensity over puncta pre- and post-deformation, fission or fusion. Images were taken 0.2 s or 0.5 s apart. FIG. 17B provides a schematic of cell treatments (top). Representative super-resolution tcPALM images of endogenous IR labeled with Dendra2 in living HepG2 cells stimulated acutely with (3 nM) or without (0 nM) insulin for 5 min (bottom left). Scale bars are indicated in the images. Representative tc-PALM traces (bottom right). FIG. 17C shows frequency of IR cluster lifetime at the plasma membrane, cytoplasm and nucleus. Average lifetime (τ_{avg}) of short-lived IR clusters+/−standard error of the mean (SEM) is reported in the graphs. Light blue corresponds to HepG2 cells not acutely stimulated with insulin (0 nM insulin), while dark blue corresponds to HepG2 cells acutely stimulated with insulin for 5 min (3 nM insulin). FIG. 17D shows number of IR clusters per cell at the plasma membrane, cytoplasm and nucleus based on tc-PALM. FIG. 17E shows number of IR-Dendra2 detections per IR cluster based on tc-PALM. Average number of IR detections per IR cluster is reported in parenthesis on top of each histogram. FIG. 17F provides representative images of endogenous IR tagged with GFP (IR-GFP) and phosphorylated IRS1 (pIRS1) in insulin-sensitive HepG2 cells stimulated acutely with (3 nM) or without (0 nM) insulin for 5 min (left). Two examples are represented per condition (top left and bottom left). Quantification of pIRS1 signal in IR condensates (right). FIG. 17G provides representative images of endogenous IR tagged with GFP (IR-GFP) in insulin-sensitive HepG2 cells stimulated acutely with 0 nM, 0.1 nM, 1 nM, 10 nM and 100 nM insulin for 5 min. FIG. 17H shows quantification of IR signal in condensates. FIG. 17I shows immunoblot and quantification of pIRS1 over total IRS1 in insulin-sensitive HepG2 cells stimulated acutely with 0 nM, 0.1 nM, 1 nM, 10 nM and 100 nM insulin for 5 min.

FIGS. 18A-18H demonstrate altered insulin receptor dynamics in insulin-resistant cells and rescue by metformin. FIG. 18A provides a schematic of cell treatments. FIG. 18B shows frequency of IR condensate lifetime at the plasma membrane, cytoplasm and nucleus in insulin-sensitive (light blue), insulin-resistant (red) and metformin-treated insulin-resistant (purple) cells. The concentration of metformin used was 12.5 μM and cells were imaged after insulin washout without being acutely stimulated with insulin. Average lifetime (τavg) of short-lived IR condensates+/−standard error of the mean (SEM) is reported in the graphs. FIG. 18C provides example images of endogenous IR tagged with GFP (IR-GFP, green) and phosphorylated IRS1 (pIRS1, magenta) in insulin-sensitive, insulin-resistant and metformin-treated insulin-resistant HepG2 cells stimulated acutely with 3 nM insulin for 5 min (left). Quantification of pIRS1 signal in IR condensates (right). FIG. 18D provides a schematic representation of IR-GFP-FKBP (IR-FKBP) construct and the effect of DMSO and AP1903 on IR-FKBP condensates. FIG. 18E provides representative images of IR-GFP-FKBP (IR-FKBP) in HepG2 cells treated with DMSO or AP1903 for 16 hours. FIG. 18F shows frequency of IR-Dendra2-FKBP condensate lifetime at the plasma membrane, cytoplasm and nucleus in HepG2 cells treated with DMSO or AP1903 for 16 hours. Average lifetime (τavg) of short-lived IR condensates+/−standard error of the mean (SEM) is reported in the graphs. FIG. 18G shows immunoblot and quantification for phosphorylated IR-GFP-FKBP (pIR-FKBP) and phosphorylated IRS1 (pIRS1) over total protein (IR-FKBP and IRS1). HepG2 cells expressing IR-GFP-FKBP (IR-FKBP) were treated with DMSO or AP1903 for 16 hours. FIG. 18H provides representative images of IR-GFP-FKBP (IR-FKBP) and phosphorylated IRS1 (pIRS1) in cells expressing IR-FKBP that were treated with DMSO or AP1903 for 16 hours.

FIGS. 19A-19F demonstrate high ROS levels promote IR condensate dysregulation. FIG. 19A provides representative immunofluorescence images for NRF2 (magenta) in insulin-sensitive or resistant HepG2 cells, showing increased levels of this marker of oxidative stress in insulin-resistant cells relative to insulin-sensitive cells (left). Dashed light blue lines represent nuclear outline. Quantification of mean NRF2 signal intensity in nuclei of insulin-sensitive (S) or resistant (R) cells (right). FIG. 19B provides representative images of cells treated with ROS-sensitive dye (left). The brighter color (magenta) is associated with higher ROS levels. Dashed light blue lines represent nuclear outline. Quantification of mean ROS signal in insulin-sensitive (S), insulin-resistant R) and metformin-treated insulin-resistant cells (RM) (right). Metformin concentration used was 12.5 μM. FIG. 19C provides a schematic of cell treatments (top). Representative images of IR-GFP in insulin-sensitive cells (S), insulin-resistant cells (R) or insulin-sensitive cells treated with H₂O₂(SH) (middle left). Dashed light blue lines represent nuclear outline. Orange, magenta and yellow boxes represent regions at the plasma membrane (PM), nucleus and cytoplasm (Cytop), respectively, that are magnified at the bottom (ZOOM). Scale bars are indicated in the images. Quantification of IR-GFP signal intensity in IR condensates at the plasma membrane (PM), cytoplasm and nucleus in insulin-sensitive (S), insulin-resistant (R) and H₂O₂-treated insulin-sensitive (SH) cells (right). FIG. 19D shows frequency of IR condensate lifetime at the plasma membrane, cytoplasm and nucleus in insulin-sensitive (light blue), insulin-resistant (red) and H₂O₂-treated insulin-sensitive (brown) cells. The concentration of H₂O₂ used was 20 mM. Average lifetime (T_{avg}) of short-lived IR condensates+/−standard error of the mean (SEM) is reported in the graphs. FIG. 19E provides schematic of cell treatments (top). Representative images of IR-GFP in insulin-sensitive cells (S, Sensitive), insulin-resistant cells (R, Resistant) or insulin-resistant cells treated with NAC (R NAC, Resistant NAC) (middle left). Dashed light blue lines represent nuclear outline. Orange, magenta and yellow boxes represent regions at the plasma membrane (PM), nucleus and cytoplasm (Cytop), respectively, that are magnified at the bottom (ZOOM). Scale bars are indicated in the images. Quantification of IR-GFP signal intensity in IR condensates at the plasma membrane (PM), cytoplasm and nucleus in insulin-sensitive (S), resistant (R) and NAC-treated insulin-resistant (R NAC) cells (middle right). FIG. 19F. shows frequency of IR condensate lifetime at the plasma membrane, cytoplasm and nucleus in insulin-sensitive (light blue), insulin-resistant (red) and NAC-treated insulin-resistant (purple) cells. The concentration of NAC used was 1 mM. Average lifetime (τ_{avg}) of short-lived IR condensates+/−standard error of the mean (SEM) is reported in the graphs.

FIGS. 20A-20C demonstrate a condensate model for insulin signaling and resistance. FIG. 20A shows an insulin receptor (green) is incorporated into condensates at the plasma membrane, at vesicle membranes, in the cytosol and in the nucleus, together with other insulin signaling proteins and, in the nucleus, with proteins involved in transcription (transcription factors, Mediator, RNA Polymerase II). FIG. 20B shows insulin stimulation promotes IR incorporation into condensates in insulin-sensitive cells and this effect is attenuated in insulin resistance. FIG. 20C shows that in insulin-resistant cells, IR condensates are longer lived and have less dynamic molecular exchange than those in insulin-sensitive cells, and this difference in IR condensate dynamics correlates with signal output.

FIGS. 21A-21H demonstrate human liver characterization and antibody validation. FIG. 21A provides representative hematoxylin and eosin (H&E) images of human livers from a healthy donor (Healthy), a donor with T2D (T2D) and a donor with T2D who had been treated with metformin (T2D Metformin). FIG. 21B shows quantification of relative glucose levels in livers from healthy donors (Healthy), donors with T2D (T2D) and donors with T2D who had been treated with metformin (T2D Metformin) as determined by metabolomics. FIG. 21C shows quantification of NAD/NADH ratio in livers from healthy donors (Healthy), donors with T2D (T2D) and donors with T2D who had been treated with metformin (T2D Metformin) as determined by metabolomics. FIGS. 21D-21E show validation of the antibody against IR by immunoblot (FIG. 21D) and immunofluorescence (FIG. 21E) and quantification. FIG. 21F shows automated quantification of IR signal in puncta in entire cells of healthy donors (Healthy), donors with T2D (T2D) and donors with T2D who had been treated with metformin (T2D Metformin). FIG. 21G shows quantification of IR signal in puncta at the plasma membrane, cytoplasm and nucleus in healthy donors (H1-H7, Healthy), donors with T2D (T1-T7, T2D) and donors with T2D who had been treated with metformin (TM1-TM9, T2D Metformin). Quantification for each individual donor is shown. FIG. 21H shows quantification of relative IR levels by immunoblot. IR level was normalized to CK18 and represented relative to sample Healthy 3 (H3).

FIGS. 22A-22I demonstrate validation of insulin-sensitive HepG2 cell model. FIG. 22A provides a schematic of cell treatment (top). Percent viability of cells cultured in cell expansion media (Media+FBS) or in media containing physiological concentrations of insulin (Media−FBS) is reported in the graph (bottom). FIG. 22B shows quantification of insulin clearance at 1, 4 or 24 hours in insulin-sensitive cells treated with 3 nM insulin. FIG. 22C provides an experimental protocol (top) and immunoblot with quantitation (bottom) to measure phosphorylated insulin signaling proteins (pIRb, pAKT, pERK) over total insulin signaling proteins (IRb, AKT, ERK). FIG. 22D shows gene ontology of the differentially expressed genes after 4 hours of 3 nM insulin stimulation. The y-axis corresponds to the KEGG pathways. The x-axis and the point size represent the “Gene Ratio” defined as the fraction of differentially expressed genes in each given ontology term (in this case KEGG pathway). The color corresponds to −log₁₀ (adjusted p-value). FIG. 22E shows relative expression of FASN and PCK1 in HepG2 cells acutely stimulated with (3 nM) or without (0 nM) insulin for 4 hours. FIG. 22F shows isotope tracing experiment showing relative palmitate labeling in HepG2 cells acutely stimulated with 0 nM or 1 nM insulin for 36 hours. FIG. 22G provides quantification of glucose production in cells stimulated with 0, 0.1, 1 or 10 nM insulin for 5 hours. FIG. 22H shows isotope tracing experiment showing relative glucose labeling in HepG2 cells acutely stimulated with 0, 1, 10 or 100 nM insulin for 24 hours. FIG. 22I provides immunoblot to quantify phosphorylated GSKα/β (pGSKα/β) over total GSKα/β protein in HepG2 cells acutely stimulated with 0 nM or 3 nM insulin for 5 minutes. This is the same experiment as in FIG. 26J.

FIG. 23 demonstrates automated quantification of IR signal intensity in puncta. FIG. 23 shows quantification of IR signal intensity in puncta in entire cells, relative to FIG. 16B.

FIGS. 24A-24B demonstrates quantification of the number of IR molecules in HepG2 cells. FIG. 24A shows quantitative western blot with standard curve of purified IRbeta mCherry fusion protein (IRb-mCherry; first 7 lanes) and cell lysate containing a specific number of cells (last four lanes). FIG. 24B provides an immunoblot for IRbeta (IRb) and beta-actin (bActin) in cells treated acutely with 0 nM or 3 nM insulin (left). Quantification of relative IRb levels in HepG2 cells without (0 nM) and with (3 nM) acute insulin stimulation (right). IRb level was normalized to beta-actin.

FIGS. 25A-25F demonstrate IR puncta in various cellular compartments in insulin-sensitive cells. FIG. 25A provides representative electron microscopy images for IR showing its presence near the plasma membrane, in the cytoplasm and in the nucleus. FIG. 25B provides representative immunofluorescence images for PI3K or AKT (magenta) together with IR (green) in insulin-sensitive HepG2 cells acutely stimulated with insulin for 5 minutes. Dashed light blue lines represent nuclear outline. Representative colocalization area (yellow box) is magnified at the bottom right corner of each image. FIG. 25C provides representative immunofluorescence images for clathrin or LAMP1 together with IR (green) in insulin-sensitive HepG2 cells acutely stimulated with insulin for 5 minutes. IR was detected either by immunofluorescence or by imaging endogenous IR-GFP. Dashed light blue lines represent nuclear outline. Representative colocalization area (yellow box) is magnified at the bottom right corner of each image. FIG. 25D provides representative immunofluorescence images for EEA1 (endosome marker) and IR, with a schematic representation of IR puncta associated with a portion of the vesicle membrane (left). Published electron microscopy image of IR and another receptor associated with a portion of the membrane of a vesicle, with a schematic representation of IR puncta associated with the vesicle (right). Reuse of the published image is granted under STM guidelines. FIG. 25E shows colocalization of IR and nascent RNA of FASN, SREBF1 and TIMM22 determined by imaging IR-GFP and FASN, SREBF1 and TIMM22 intronic RNA FISH in cells stimulated with 3 nM insulin. Colocalization area (magenta box) is magnified at the bottom right corner of each image. Scale bars are indicated in the images. FASN, SREBF1 and TIMM22 are known insulin-responsive genes. FIG. 25F shows ChIP-seq tracks of IR, MED1 and RPB1 at FASN, SREBF1 and TIMM22 loci.

FIGS. 26A-26N demonstrate validation of insulin-resistant HepG2 cell model. FIG. 26A provides a schematic of cell treatments. FIGS. 26B-26E shows immunoblot with quantitation to measure phosphorylated insulin signaling proteins (pIRb, pIRS1, pAKT, pERK) over total insulin signaling proteins (IRb, IRS1, AKT, ERK) in insulin-sensitive (S) and insulin-resistant (R) cells stimulated with 0 nM or 3 nM insulin for 5 minutes. FIG. 26F shows relative expression of FASN in insulin-resistant HepG2 cells acutely stimulated with 0 nM or 3 nM insulin for 4 hours. FIG. 26G shows isotope tracing experiment showing relative palmitate labeling in insulin-resistant HepG2 cells acutely stimulated with 0 nM or 1 nM insulin for 36 hours. FIG. 26H shows quantification of glucose production in insulin-resistant HepG2 cells stimulated with 0, 0.1, 1 or 10 nM insulin for 5 hours. FIG. 26I shows isotope tracing experiment showing relative glucose labeling in insulin-resistant HepG2 cells acutely stimulated with 0, 1, 10 or 100 nM insulin for 24 hours. FIG. 26J shows immunoblot to quantify phosphorylated GSKα/β (pGSKα/β) over total GSKα/β protein in insulin-sensitive (S) and insulin-resistant (R) HepG2 cells acutely stimulated with 0 nM or 3 nM insulin for 5 minutes. FIG. 26K shows immunoblot for IRbeta (IRb) and beta-actin (b-Actin) in insulin-sensitive and insulin-resistant cells unstimulated with insulin (left). Quantification of relative IRb levels (right). FIG. 26L shows enzyme-linked immunoassay (ELISA) for IRbeta (IRb) relative to total protein in insulin-sensitive (S) and insulin-resistant (R) cells unstimulated with insulin. FIG. 26M provides a schematic of proteolytic shaving experiment. Insulin-sensitive or resistant cells were either treated with TrypLE to digest the portions of proteins at the cell surface (Digested) or not (Undigested). Immunoblot with quantitation to measure IRalpha (IRa) and beta-actin (b-Actin) in digested and undigested insulin-sensitive (S) and insulin-resistant (R) cells. FIG. 26N shows proteomics quantification of insulin binding in insulin-sensitive (S) and insulin-resistant (R) cells treated with 3 nM insulin at 4° C. Peak area quantification is reported for two insulin peptides: GIVEQCCTSICSLYQLENYCN (SEQ ID NO: 11) (Insulin A-chain) and GFFYTPK (SEQ ID NO: 12) (insulin B-chain).

FIGS. 27A-27C demonstrate other models of insulin resistance. FIG. 27A provides a schematic of cell treatments (top). Imaging of IR-GFP in HepG2 cells treated with physiological concentrations of insulin (Sensitive, S) or pathological concentration of TNFα (TNFα) and acutely stimulated with 3 nM insulin for 5 minutes (middle). Quantification of IR signal intensity in IR condensates in entire cells (automated quantification), at the plasma membrane (PM), cytoplasm or nucleus of cells (bottom). FIG. 27B provides a schematic of cell treatments (top). Imaging of IR-GFP in HepG2 cells treated with physiological concentrations of insulin (Sensitive, S) or with high nutrients (either 1) pathological concentrations of glucose and fat and physiological concentration of insulin (high nutrients, GF) or 2) pathological concentrations of glucose, fat and insulin (high nutrients, GFI) and acutely stimulated with 3 nM insulin for 5 minutes (middle). Quantification of IR signal intensity in IR condensates in entire cells (automated quantification), at the plasma membrane (PM), cytoplasm or nucleus of cells (bottom). FIG. 27C shows ROS intensity in insulin-sensitive HepG2 cells, in cells treated with TNFα, high nutrients, or high nutrients and high insulin. Physiological concentration of insulin corresponds to 0.1 nM, pathological concentration of insulin corresponds to 3 nM, pathological concentration of TNFα corresponds to 100 pg/ml, pathological concentration of fat corresponds to 30 μM palmitic acid and 45 μM oleic acid, pathological concentration of glucose corresponds to 10 mM.

FIGS. 28A-28C demonstrate homozygous HepG2 cell lines expressing functional endogenous IR tagged with GFP or Dendra2. FIG. 28A provides a schematic of knock-in strategy. FIG. 28B provides a schematic of cell treatments (top). Immunoblot for IRbeta (IRb) and beta-actin (bActin) control in WT, IR-GFP and IR-Dendra2 cell lines (bottom left). The shift in molecular weight is the expected size for the GFP or Dendra2 fusion with IR. Quantitation of IRb levels (bottom right). FIG. 28C provides a schematic of cell treatments (top). Immunoblot with quantitation to measure phosphorylated insulin signaling proteins (pIRb and pAKT) over total insulin signaling proteins (IRb and AKT) in IR-GFP and IR-Dendra2 cells stimulated with 0 nM or 3 nM insulin for 5 minutes (bottom).

FIGS. 29A-29C demonstrate live-cell imaging of IR bodies in HepG2 cells. FIG. 29A shows live imaging time course of HepG2 cells expressing endogenous IR tagged with GFP during insulin stimulation. Time of acquisition is reported above images. Dashed light blue lines represent nuclear outline and scale bar are indicated in the images. Representative images of three cells (top). Orange, magenta and yellow boxes represent regions at the plasma membrane (PM), nucleus and cytoplasm, respectively, that are magnified at the bottom. FIG. 29B provides quantification of IR puncta signal at the plasma membrane (PM), nucleus and cytoplasm of IR-GFP cells stimulated with 3 nM insulin for 0, 2.5, 5 and 7.5 minutes. Data is represented as “relative to 0 minutes”. FIG. 29C provides quantification of number of IR puncta at the plasma membrane (PM), nucleus and cytoplasm of IR-GFP cells stimulated with 3 nM insulin for 0, 2.5, 5 and 7.5 minutes.

FIGS. 30A-30C demonstrate metformin effect on IR puncta. FIG. 30A provides a schematic of cell treatments (top). Imaging of IR-GFP in insulin-sensitive and insulin-resistant cells treated with or without metformin (bottom). Metformin concentration is reported above the images. IR-GFP fluorescence signal is shown in green. Dashed light blue lines represent nuclear outline. Orange, magenta and yellow boxes represent regions at the plasma membrane, nucleus and cytoplasm, respectively. Scale bars are indicated in the images. This is the same experiment as in FIG. 16F and thus the same images for insulin-sensitive cells, insulin-resistant cells and insulin-resistant cells treated with 12.5 μM metformin are reported in FIG. 16D. Quantification of IR signal in puncta and the number of IR puncta in insulin-sensitive or insulin-resistant cells treated with or without metformin. Quantification of IR signal in puncta in entire cells was automated. FIG. 30B shows imaging of IR-GFP in insulin-sensitive cells treated with or without 50 μM metformin and acutely stimulated with 3 nM insulin for 5 minutes. Dashed light blue lines represent nuclear outline. FIG. 30C shows immunoblot for IRbeta (IRb) and beta-actin (bActin) in cells cultured in pathologic levels of insulin treated with (RM) or without (R) 12.5 μM metformin (left). Quantification of relative levels of IRb (right).

FIGS. 31A-31E demonstrate IR puncta in human primary hepatocytes. FIG. 31A provides a schematic of cell treatments. FIG. 31B shows ELISA quantification of albumin production by human liver spheroids cultured with physiologic (blue) or pathologic (red) concentrations of insulin. FIG. 31C shows ELISA quantification of insulin clearance by human liver spheroids cultured with physiologic (blue) or pathologic (red) concentrations of insulin. FIG. 31D provides quantification of glucose production in human liver spheroids cultured with physiologic (blue) or pathologic (red) concentrations of insulin. FIG. 31E provides a schematic of cell treatments (top). Immunofluorescence for IR in insulin-sensitive, insulin-resistant and metformin-treated insulin-resistant human liver spheroids acutely treated with 0 nM or 3 nM insulin for 10 minutes (middle). Magnified images of IR punta at the plasma membrane, cytoplasm and nucleus (bottom left) and quantification of IR signal at IR condensates at the plasma membrane (PM), cytoplasm and nucleus (right).

FIGS. 32A-32C demonstrate IR puncta in human primary adipocytes. FIG. 32A provides a representative immunofluorescence image of perilipin (magenta) in human primary adipocyte. Nucleus is counterstained using Hoechst. FIG. 32B shows ELISA quantification of pAKT over AKT in human primary adipocytes treated with physiological (Sensitive) or pathological (Resistant) concentrations of insulin for 5 days and acutely stimulated (3 nM) or not (0 nM) with insulin for 15 minutes. FIG. 32C provides a schematic of cell treatments (top). Immunofluorescence for IR in insulin-sensitive (Sensitive), insulin-resistant (Resistant) and metformin-treated insulin-resistant (Resistant+Metformin) human primary adipocytes acutely treated with 0 nM or 3 nM insulin for 5 minutes (middle). Orange, yellow and magenta boxes represent regions at the plasma membrane, cytoplasm and nucleus, respectively, that are magnified at the bottom left. Quantification of IR signal at IR puncta at the plasma membrane, cytoplasm and nucleus (bottom right).

FIGS. 33A-33F demonstrate single-molecule statistics and validation of tc-PALM analysis. FIG. 33A shows distribution of the number of detections of single molecules in live cells. Total number of 80512 single molecules are collected for plotting the histogram. FIG. 33B shows distribution of the lifetime of single molecules. FIG. 33C shows distribution of the inter-detection period (dark-time) of single molecules with more than one detection. Total number of 6173 multi-detection single molecules are collected for plotting the histogram.

FIG. 33D provides a histogram of inter-detection period of identified transient clusters in live cells and pseudo-transient clusters in fixed cells selected with the same procedure as in live cells. The counts of each bin are normalized to the first bin, which mostly consists of counts of blinking events from single molecules (given that most single molecules have a lifetime span shorter than Is). FIG. 33E provides statistics of single molecules in live and fixed samples. FIG. 33F provides statistics of identified multimolecule bursts and outlier single molecules. Ideally, the true positive rate (TPR) can go beyond 90% based on the estimation of cut-offs (0.05 quantile) from real bursts.

FIG. 34 demonstrates IR-Dendra2 detections in condensates in entire cells. FIG. 34 shows quantification of the number of IR-Dendra2 detections per IR cluster in insulin-sensitive cells stimulated with (3 nM) and without (0 nM) insulin for 5 minutes. Average number of IR-Dendra2 detections per IR cluster is reported in parenthesis on top of each histogram.

FIGS. 35A-35B demonstrate a correlation between IR and pIRS1 signal intensity in condensates. FIG. 35A provides quantification of pIRS1 and IR signal in condensates. To obtain IR condensates with different levels of IR molecules, HepG2 cells expressing endogenous IR tagged with GFP were treated with siControl or siRNA for INSR for 18 hours or 24 hours. FIG. 35B provides quantification of pIRS1 signal inside and outside IR condensates.

FIGS. 36A-36B demonstrate increased IR condensate lifetime by inflammation and high nutrients. FIG. 36A provides a schematic of cell treatments (top). Tc-PALM quantification of IR condensate lifetime at the plasma membrane (PM), cytoplasm and nucleus in HepG2 cells expressing IR-Dendra2 treated with physiological concentrations of insulin (Sensitive, S) or pathological concentration of TNFα (TNFα). FIG. 36B provides a schematic of cell treatments (top). Tc-PALM quantification of IR condensate lifetime at the plasma membrane (PM), cytoplasm and nucleus in HepG2 cells expressing IR-Dendra2 treated with physiological concentrations of insulin (Sensitive, S) or with high nutrients either 1) pathological concentrations of glucose and fat and physiological concentration of insulin (high nutrients, GF) or 2) pathological concentrations of glucose, fat and insulin (high nutrients, GFI). Physiological concentration of insulin corresponds to 0.1 nM, pathological concentration of insulin corresponds to 3 nM, pathological concentration of TNFα corresponds to 100 pg/ml, pathological concentration of fat corresponds to 30 μM palmitic acid and 45 μM oleic acid, pathological concentration of glucose corresponds to 10 mM.

FIG. 37 demonstrates that metformin does not decrease IR condensate lifetime in insulin-sensitive cells. FIG. 37 shows Tc-PALM quantification of IR condensate lifetime at the plasma membrane (PM), cytoplasm and nucleus in insulin-sensitive HepG2 cells expressing IR-Dendra2 treated with and without 12.5 μM metformin for 1 day.

FIG. 38 demonstrates that metformin partially rescues phosphorylation of IRS1. FIG. 38 shows immunoblot and quantification of pIRS1 over total IRS1 in insulin-sensitive, insulin-resistant and metformin-treated insulin-resistant cells. Metformin concentrations used are reported in the imaged.

FIGS. 39A-39B demonstrate the effect of AP1903 in HepG2 cells. FIG. 39A provides immunoblot and quantification of the relative levels of expression of WT IR (IR WT) and IR-GFP-FKBP (IR-FKBP). FIG. 39B provides immunoblot and quantification of the relative levels of phosphorylated IRS1 and total IRS1 in untransfected, wildtype HepG2 cells treated with DMSO or AP1903.

FIGS. 40A-40B demonstrate oxidative stress effect on IR incorporation into condensates. FIG. 40A provides automated quantification of IR condensate signal intensity relative to FIG. 19C. FIG. 40B provides quantification of IR condensate signal relative to FIG. 19E.

FIG. 41 provides a table summarizing donor characteristics.

### DETAILED DESCRIPTION OF THE INVENTION

### Some Definitions

The term “expression” refers to the cellular processes involved in producing RNA and proteins and as appropriate, secreting proteins, including where applicable, but not limited to, transcription, translation, folding, modification and processing. Expression products include RNA transcribed from a gene and polypeptides obtained by translation of mRNA transcribed from a gene.

The terms “subject” and “individual” are used interchangeably herein, and refer to an animal, for example, a human from whom cells can be obtained and/or to whom treatment, including prophylactic treatment is provided. For treatment of conditions or disease states which are specific for a specific animal such as a human subject, the term subject refers to that specific animal. The terms “non-human animals” and “non-human mammals” as used herein interchangeably, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treating” and “treatment” refer to administering to a subject an effective amount of a composition so that the subject experiences a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

The terms “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two-standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, condensates refer to phase-separated multi-molecular assemblies. In some embodiments, condensates refer to in vitro condensates (sometimes referred to herein as “droplets”). In some embodiments, in vitro condensates are artificially created with one or more condensate components in a solution. As used herein, “transcriptional condensates” are phase-separated multi-molecular assemblies that occur at the sites of transcription and are high density cooperative assemblies of multiple components that can include transcription factors, co-factors (e.g., co-activator), chromatin regulators, DNA, non-coding RNA, nascent RNA, RNA polymerase II, kinases, proteasomes, topoisomerase, and/or enhancers.

### Methods of Decreasing Insulin Resistance

Some aspects of the present disclosure are directed to a method of decreasing insulin resistance in an insulin resistant cell, comprising contacting the cell with an agent that decreases a level of reactive oxygen species (ROS) in the cell, wherein the agent is not metformin.

As used herein, an “insulin resistant cell” is a cell that does not take up glucose in response to insulin or a cell with a decreased ability to take up glucose in response to insulin as compared to an appropriate control cell. In some embodiments, an insulin resistant cell uptakes at least about 10%, 20%, 30%, 40%, 50%, 60%, 70%, 80%, 90%, or substantially 100% less glucose than an appropriate control cell in the present of 0.1 nM of insulin.

The cell is not limited and may be any suitable cell. In some embodiments, the cell is a human cell; fetal cell; embryonic stem cell or embryonic stem cell-like cell, e.g., cell from the umbilical vein, e.g., endothelial cell from the umbilical vein; muscle, e.g., myotube, fetal muscle; blood cell, fetal blood cell, monocyte; B cell, e.g., Pro-B cell; brain, e.g., astrocyte cell, angular gyrus of the brain, anterior caudate of the brain, cingulate gyrus of the brain, hippocampus of the brain, inferior temporal lobe of the brain, middle frontal lobe of the brain, T cell, e.g., naïve T cell, memory T cell; CD4 positive cell; CD25 positive cell; CD45RA positive cell; CD45RO positive cell; IL-17 positive cell; a cell that is stimulated with PMA; Th cell; Thl7 cell; CD255 positive cell; CD127 positive cell; CD8 positive cell; CD34 positive cell; duodenum, e.g., smooth muscle tissue of the duodenum; skeletal muscle tissue; myoblast; stomach, e.g., smooth muscle tissue of the stomach, e.g., gastric cell; CD3 positive cell; CD14 positive cell; CD19 positive cell; CD20 positive cell; CD34 positive cell; CD56 positive cell; prostate cell; colon cell; crypt cell; intestine cell, e.g., large intestine cell; e.g., fetal intestine cell; bone cell, e.g., osteoblast; pancreas cell; adipose cell; adrenal gland cell; bladder cell; esophageal cell; heart cell, e.g., left ventricle, right ventricle, left atrium, right atrium, or aorta cell; lung cell; skin cell, e.g., fibroblast cell; ovary cell; breast cell; mammary epithelium cell; liver cell; DND41 cell; GM12878 cell; H1 cell; H2171 cell; HCC1954 cell; HCT-116 cell; HeLa cell; HepG2 cell; HMEC cell; HSMM tube cell; HUVEC cell; IMR90 cell; Jurkat cell; K562 cell; LNCaP cell; MCF-7 cell; MMlS cell; NHLF cell; NHDF-Ad cell; RPMI-8402 cell; U87 cell; VACO 9M cell; VACO 400 cell; or VACO 503 cell. In some embodiments, the cell is a mammalian (e.g., human) fat, liver, brain, kidney, muscle (e.g., skeletal muscle), or pancreatic islet cell (e.g., pancreatic b-cell).

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the agent is selected from the group consisting of a nucleic acid, a small molecule, a polypeptide, and a peptide.

In some embodiments, the agent is a small molecule. The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 Daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. In some embodiments, the small molecule comprises at least one, at least two, at least three, at least four, at least five, at least six, at least seven, at least eight, at least nine, at least ten or more aromatic side chains.

In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds. In some embodiments, the agent is sufficiently small to diffuse into a condensate. In some embodiments, the agent is less than about 4.4 kDa. In some embodiments, the agent has a partition coefficient for a condensate of at least 100, 150, 200, 300, 350, 400, 450, 500, 550, 600, 650, 700 or more. In some embodiments, the agent has a partition coefficient for a condensate of less than about 10, 20, 50, 100, 150, 200, 300, 350, 400, 450, 500, 550, or 600.

In some embodiments, the agent is a protein or polypeptide. The term “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, a small molecule (such as a fluorophore), etc. In some embodiments, the agent is a protein or polypeptide comprising at least 5, at least 10, at least 15, at least 20, at least 25, at least 30, or more aromatic amino acids.

In some embodiments, the agent consists of or comprises DNA or RNA or a derivative or analog thereof.

In some embodiments, the agent is a peptide mimetic. The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics.

In some embodiments, the agent (e.g., candidate agent, modified agent) consists of or comprises a peptide, polypeptide or protein and the number of aromatic rings is increased by substituting one or more non-aromatic amino acid residues with an aromatic amino acid residue (e.g., phenylalanine, tryptophan, tyrosine, and/or histidine). In some embodiments, the agent (e.g., candidate agent, modified agent) consists of or comprises a peptide, polypeptide or protein and the number of aromatic rings is increased by adding one or more aromatic amino acids. In some embodiments, the aromatic amino acid residue is not histidine. In some embodiments, the aromatic amino acid residue is phenylalanine. In some embodiments, the aromatic amino acid residue is a non-naturally occurring amino acid residue or a nonstandard amino acid residue (e.g., L-DOPA (1-3,4-dihydroxyphenylalanine)).

In some embodiments, the agent (e.g., candidate agent, modified agent) consists of or comprises a peptide, polypeptide or protein and the number of aromatic rings is decreased by replacing one or more aromatic amino acids with non-aromatic amino acids (e.g., alanine). In some embodiments, the number of aromatic rings is decreased by deleting or modifying one or more aromatic amino acids.

In some embodiment, the number of aromatic rings is decreased by deleting, modifying, and/or replacing two or more aromatic amino acids.

In some embodiments, the agent (e.g., candidate agent, modified agent) comprises an IDR. In some embodiments, a modified agent comprises an IDR. Regions of intrinsic disorder, also termed intrinsic (or intrinsically) disordered regions (IDR) or intrinsic (or intrinsically) disordered domains can be found in many protein condensate components. Each of these terms is used interchangeably throughout the disclosure. IDR lack stable secondary and tertiary structure. In some embodiments, an IDR may be identified by the methods disclosed in Ali, M., & Ivarsson, Y. (2018). High-throughput discovery of functional disordered regions. Molecular Systems Biology, 14(5), e8377. IDRs are known in the art and any suitable method may be used to identify an IDR.

In some embodiments, the agent is a derivative of metformin.

The agents may be administered in pharmaceutically acceptable solutions, which may routinely contain pharmaceutically acceptable concentrations of salt, buffering agents, preservatives, compatible carriers, adjuvants, and optionally other therapeutic ingredients.

The agents may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

In some embodiments, the level of ROS is decreased by at least about 10%, 20%, 30%, 40%, 50%, 60%, 70%, 80%, 90%, or substantially 100%. ROS are chemically reactive molecules containing oxygen. Exemplary ROS are peroxides (e.g., hydrogen peroxides), superoxide, hydroxyl radical, and singlet oxygen. Methods of measuring the level of ROS are not limited and may be any suitable method know in the art. In some embodiments, ROS levels are measured by Spin trapping, Chemiluminescent probes, Fluorescent probes, Detection of cellular and mitochondrial O2 using DHE and mitochondrion-targeted probe mitoSOX, Detection of total cellular and mitochondrial O2 by cyclic hydroxylamine spin probes, Detection of cytoplasmic and mitochondrial H2O2 by fluorescent protein-based redox probes, Detection of H2O2 and ONOO by boronate-based fluorescent probes, Immuno-spin trapping, or X- and L-Band ESR Spectroscopy. See, Dikalov S I, Harrison D G. Methods for detection of mitochondrial and cellular reactive oxygen species. Antioxid Redox Signal. 2014; 20(2):372-382. doi:10.1089/ars.2012.4886, incorporated herein by reference.

In some embodiments, contact of the cell with the agent and insulin increases incorporation of insulin receptor (IR) into cytoplasmic and/or nuclear condensates in the cell.

In some embodiments, contact of the cell with the agent and insulin increases incorporation of insulin receptor (IR) into transcriptional condensates. In some embodiments, the transcriptional condensates comprise transcriptional condensates modulating the expression of one or more insulin responsive genes.

In some embodiments, the agent is contacted with the cell in vivo in a subject in need thereof. In some embodiments, the subject exhibits insulin resistance. In some embodiments, the subject has an insulin resistance-associated disease. In some embodiments, the insulin resistance-associated disease is type-2 diabetes, metabolic syndrome, non-alcoholic fatty liver disease (NAFLD), polycystic ovarian syndrome (PCOS), or Alzheimer's Disease.

Some aspects of the present disclosure are directed to a method of decreasing insulin resistance in an insulin resistant cell, comprising contacting the cell with an agent that incorporates into transcriptional condensates of the cell and increases expression of one or more insulin responsive genes in the presence of insulin.

The cell is not limited and may be any cell disclosed herein. In some embodiments, the cell is a mammalian (e.g., human) fat, liver, brain, kidney, muscle (e.g., skeletal muscle) or pancreatic islet cell (e.g., pancreatic b-cell).

The agent is not limited and may be any agent described herein. In some embodiments, the agent is a functional insulin receptor variant or derivative having increased affinity, as compared to wild-type insulin receptor, for the transcriptional condensates. In some embodiments, the agent has at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more affinity for a transcriptional condensate (e.g., a transcriptional condensate associated with an insulin responsive gene) that wild-type insulin receptor.

In some embodiments, the agent is a functional insulin receptor variant or derivative having increased affinity (e.g., at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more), as compared to wild-type insulin receptor, for a transcriptional condensate (e.g., a transcriptional condensate associated with an insulin responsive gene) in an elevated reactive oxygen species (ROS) environment.

In some embodiments, the agent is contacted with the cell in vivo in a subject in need thereof. The subject is not limited and may be any subject described herein. In some embodiments, the subject exhibits insulin resistance. In some embodiments, the subject has an insulin resistance-associated disease. In some embodiments, the insulin resistance-associated disease is type-2 diabetes, metabolic syndrome, non-alcoholic fatty liver disease (NAFLD), polycystic ovarian syndrome (PCOS), or Alzheimer's Disease.

### Compositions

Some aspects of the present disclosure are directed to a composition (e.g., pharmaceutical composition) comprising one or more agents described herein for decreasing insulin resistance. In some embodiments, the composition further comprises metformin. In some embodiments, the composition further comprises insulin.

In addition to the active agent(s), the compositions typically comprise a pharmaceutically-acceptable carrier. The term “pharmaceutically-acceptable carrier”, as used herein, means one or more compatible solid or liquid vehicles, fillers, diluents, or encapsulating substances which are suitable for administration to a human or non-human animal. In preferred embodiments, a pharmaceutically-acceptable carrier is a non-toxic material that does not interfere with the effectiveness of the biological activity of the active ingredients. The term “compatible”, as used herein, means that the components of the pharmaceutical compositions are capable of being comingled with an agent, and with each other, in a manner such that there is no interaction which would substantially reduce the pharmaceutical efficacy of the pharmaceutical composition under ordinary use situations. Pharmaceutically-acceptable carriers should be of sufficiently high purity and sufficiently low toxicity to render them suitable for administration to the human or non-human animal being treated.

Some examples of substances which can serve as pharmaceutically-acceptable carriers are pyrogen-free water; isotonic saline; phosphate buffer solutions; sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. It will be appreciated that a pharmaceutical composition can contain multiple different pharmaceutically acceptable carriers.

A pharmaceutically-acceptable carrier employed in conjunction with the compounds described herein is used at a concentration or amount sufficient to provide a practical size to dosage relationship. The pharmaceutically-acceptable carriers, in total, may, for example, comprise from about 60% to about 99.99999% by weight of the pharmaceutical compositions, e.g., from about 80% to about 99.99%, e.g., from about 90% to about 99.95%, from about 95% to about 99.9%, or from about 98% to about 99%.

Pharmaceutically-acceptable carriers suitable for the preparation of unit dosage forms for oral administration and topical application are well-known in the art. Their selection will depend on secondary considerations like taste, cost, and/or shelf stability, which are not critical for the purposes of the subject invention, and can be made without difficulty by a person skilled in the art.

Pharmaceutically acceptable compositions can include diluents, fillers, salts, buffers, stabilizers, solubilizers and other materials which are well-known in the art. The choice of pharmaceutically-acceptable carrier to be used in conjunction with the compounds of the present invention is basically determined by the way the compound is to be administered. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof in certain embodiments. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. It will also be understood that a compound can be provided as a pharmaceutically acceptable pro-drug, or an active metabolite can be used. Furthermore, it will be appreciated that agents may be modified, e.g., with targeting moieties, moieties that increase their uptake, biological half-life (e.g., pegylation), etc.

The agents may be administered in pharmaceutically acceptable solutions, which may routinely contain pharmaceutically acceptable concentrations of salt, buffering agents, preservatives, compatible carriers, adjuvants, and optionally other therapeutic ingredients.

The agents may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

In some embodiments, agents may be administered directly to a tissue, e.g., a tissue in which the cancer cells are found or one in which a cancer is likely to arise. Direct tissue administration may be achieved by direct injection. The agents may be administered once, or alternatively they may be administered in a plurality of administrations. If administered multiple times, the agents may be administered via different routes. For example, the first (or the first few) administrations may be made directly into the affected tissue while later administrations may be systemic.

For oral administration, compositions can be formulated readily by combining the active agent(s) with pharmaceutically acceptable carriers well known in the art. Such carriers enable the agents to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In certain embodiments, the vehicle is a biocompatible microparticle or implant that is suitable for implantation into the mammalian recipient. Exemplary bioerodible implants that are useful in accordance with this method are described in PCT International Application Publication No. WO 95/24929, entitled “Polymeric Gene Delivery System”, which reports on a biodegradable polymeric matrix for containing a biological macromolecule. The polymeric matrix may be used to achieve sustained release of the agent in a subject. In some embodiments, an agent described herein may be encapsulated or dispersed within a biocompatible, preferably biodegradable polymeric matrix. The polymeric matrix may be in the form of a microparticle such as a microsphere (wherein the agent is dispersed throughout a solid polymeric matrix) or a microcapsule (wherein the agent is stored in the core of a polymeric shell). Other forms of polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents may be delivered using the bio-erodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations of the peptide, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation. Liposomes, for example, which may comprise phospholipids or other lipids, are nontoxic, physiologically acceptable carriers that may be used in some embodiments. Liposomes can be prepared according to methods known to those skilled in the art. In some embodiments, for example, liposomes may be prepared as described in U.S. Pat. No. 4,522,811. Liposomes, including targeted liposomes, pegylated liposomes, and polymerized liposomes, are known in the art (see, e.g., Hansen C B, et al., Biochim Biophys Acta. 1239(2):133-44, 1995; Torchilin V P, et al., Biochim Biophys Acta, 1511(2):397-411, 2001; Ishida T, et al., FEBS Lett. 460(1):129-33, 1999). In some embodiments, a lipid-containing particle may be prepared as described in any of the following PCT application publications, or references therein: WO/2011/127255; WO/2010/080724; WO/2010/021865; WO/2010/014895; WO2010147655.

In some embodiments, it may be advantageous to formulate oral or parenteral compositions in dosage unit form for ease of administration and uniformity of dosage. Unit dosage form as used herein refers to physically discrete units suited as unitary dosages for the subject to be treated; each unit containing a predetermined quantity of active compound calculated to produce the desired therapeutic effect in association with the required pharmaceutical carrier.

### Methods of Screening

Some aspects of the present disclosure are directed to a method of screening for a candidate agent to treat insulin resistance comprising contacting a test agent with a condensate and insulin receptor (IR) in an elevated reactive oxygen species (ROS) environment, wherein if the test agent increases flux of IR incorporation into the condensate as compared to a control (e.g., control condensate not contacted with the agent) then the test agent is identified as a candidate agent to treat insulin resistance.

In some embodiments, the method comprises measuring IR incorporation flux. Any suitable method may be used to measure IR incorporation flux. In some embodiments, IR incorporation flux is measured by time-correlated photoactivation localization microscopy. In some embodiments, the measured IR incorporation flux is compared to IR incorporation flux in a control condensate not contacted with the agent. In some embodiments, the IR comprises a detectable tag to facilitate detection/measurement.

The candidate agent may be any agent described herein. In some embodiments, the candidate agent is a small molecule. The condensate may be any condensate described herein. In some embodiments, the condensate is a transcriptional condensate. In some embodiments, the condensate is a transcriptional condensate associated with an insulin responsive gene. In some embodiments, the condensate is a synthetic in vitro condensate or an ex vivo condensate isolated from a cell. In some embodiments, the ex vivo condensate is isolated from an insulin resistant cell. In some embodiments, the insulin resistant cell is a mammalian (e.g., human) fat, liver, brain, kidney, muscle (e.g., skeletal muscle), or pancreatic islet cell (e.g., pancreatic b-cell). In some embodiments, the in vitro condensate comprises one or more condensate components from a transcriptional condensate (e.g., a transcriptional condensate associated with an insulin responsive gene). In some embodiments, the in vitro condensate comprises MED1.

Methods of making in vitro condensates as well as manipulating condensates can be found in WO 2019/183552 published Sep. 26, 2019, as well as in Hnisz et al., 2017. Cell 169, 13-23; Bradner et al., 2017. Cell 168, 629-643; Zamudio et al., 2019. Mol. Cell 76, 753-766.e6; and Boija et al., 2018. Cell 175, 1842-1855.e16, each incorporated by reference in its entirety.

Any suitable means of isolation of a condensate from a cell or composition is encompassed herein. In some embodiments, a condensate is chemically or immunologically precipitated. In some embodiments, a condensate is isolated by centrifugation (e.g., at about 5,000×g, 10,000×g, 15,000×g for about 5-15 minutes; about 10.000×g for about 10 min). A condensate may be isolated from a cell by lysis of the nucleus of a cell with a homogenizer (i.e., Dounce homogenizer) under suitable buffer conditions, followed by centrifugation and/or filtration to separate the condensate.

As used herein, the phrase “a condensate component” or the like refers to a peptide, protein, nucleic acid, signaling molecule, lipid, or the like that is part of a condensate or has the capability of being part of a condensate. In some embodiments, the component is within the condensate. In some embodiments, the component is necessary for condensate formation or stability. In some embodiments, the component is not necessary for condensate formation or stability. In some embodiments, the component is a protein or peptide and comprises one or more intrinsically ordered domains. In some embodiments, the component is a non-structural member of a condensate (e.g., not necessary for condensate integrity). In some embodiments, a condensate comprises, consists of, or consists essentially of 1, 2, 3, 4, 5, 6, 7, 8, 9, 10 or more components.

In some embodiments, the elevated ROS environment has a ROS concentration that is at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, or 20-fold greater than the ROS concentration in a non-elevated ROS environment. In some embodiments of the screening methods disclosed herein, the elevated ROS environment has a ROS concentration that is at least 1.2-fold greater than the ROS concentration in a non-elevated ROS environment.

In some embodiments, if the test agent increases the flux of IR incorporation by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, or 20-fold greater than in a suitable control condensate not treated with the test agent than the test agent is identified as a candidate agent to treat insulin resistance.

In some embodiments, the condensate is in a cell. The cell is not limited and may be any cell described herein. In some embodiments, the cell is mammalian (e.g., human) fat, liver, brain, kidney, muscle (e.g., skeletal muscle), or pancreatic islet cell (e.g., pancreatic b-cell). In some embodiments, the cell is in a test animal and the test agent is administered to the test animal (e.g., mouse, rat, rabbit, etc).

In some embodiments, the cells may be primary cells, may be isolated from or originate from a subject suffering from insulin resistance or an insulin resistance associated disorder, may be members of a cell line, may be cultured in 2D culture system e.g., on a surface of a well or plate, may be cultured in a three-dimensional culture system e.g., as spheroids or organoids, may be of a cell type that normally expresses the insulin receptor and responds to insulin by exhibiting one or more physiological changes in response to insulin.

In some embodiments, multiple candidate agents are screened and one or more agents having a comparable or greater effect than metformin are identified.

Some aspects of the present disclosure are directed to a method of screening for a candidate agent to treat insulin resistance comprising contacting a test agent with a condensate and insulin receptor (IR), wherein the condensate exhibits reduced incorporation of IR in the presence of insulin as compared to a control, wherein if the test agent increases flux of IR incorporation into the condensate as compared to a control then the test agent is identified as a candidate agent to treat insulin resistance.

In some embodiments, the method comprises measuring IR incorporation. Any suitable method may be used to measure IR incorporation. In some embodiments, IR incorporation is measured by time-correlated photoactivation localization microscopy. In some embodiments, the measured IR incorporation is compared to IR incorporation in a control condensate not contacted with the agent. In some embodiments, the IR comprises a detectable tag to facilitate detection/measurement.

In some embodiments, the condensate exhibits at least about 10%, 20%, 30%, 40%, 50%, 60%, 70%, 80%, 90%, or substantially 100% less IR incorporation in the presence of insulin than a suitable control condensate (e.g., a condensate not associated with insulin resistance). In some embodiments, if the test agent increases the flux of IR incorporation by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, or 20-fold greater than in a suitable control condensate not treated with the test agent than the test agent is identified as a candidate agent to treat insulin resistance.

The candidate agent may be any agent described herein. In some embodiments, the candidate agent is a small molecule. The condensate may be any condensate described herein. In some embodiments, the condensate is a transcriptional condensate. In some embodiments, the condensate is a transcriptional condensate associated with an insulin responsive gene. In some embodiments, the condensate is a synthetic in vitro condensate or an ex vivo condensate isolated from a cell. In some embodiments, the ex vivo condensate is isolated from an insulin resistant cell. In some embodiments, the insulin resistant cell is a mammalian (e.g., human) fat, liver, brain, kidney, muscle (e.g., skeletal muscle), or pancreatic islet cell (e.g., pancreatic b-cell). In some embodiments, the in vitro condensate comprises one or more condensate components from a transcriptional condensate (e.g., a transcriptional condensate associated with an insulin responsive gene). In some embodiments, the in vitro condensate comprises MED1.

In some embodiments, one or more components of the condensate has been exposed to elevated ROS levels. In some embodiments, the elevated ROS level is a ROS level that is at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, or 20-fold greater than the ROS level in a non-elevated ROS environment.

In some embodiments, the condensate is in a cell. The cell is not limited and may be any cell described herein. In some embodiments, the cell is mammalian (e.g., human) fat, liver, brain, kidney, muscle (e.g., skeletal muscle), or pancreatic islet cell (e.g., pancreatic b-cell). In some embodiments, the cell is in a test animal and the test agent is administered to the test animal (e.g., mouse, rat, rabbit, etc.).

In some embodiments, the cells may be primary cells, may be isolated from or originate from a subject suffering from insulin resistance or an insulin resistance associated disorder, may be members of a cell line, may be cultured in 2D culture system e.g., on a surface of a well or plate, may be cultured in a three-dimensional culture system e.g., as spheroids or organoids, may be of a cell type that normally expresses the insulin receptor and responds to insulin by exhibiting one or more physiological changes in response to insulin.

In some embodiments, multiple candidate agents are screened and one or more agents having a comparable or greater effect than metformin are identified.

Some aspects of the present disclosure are directed to a method of screening for a candidate agent to treat insulin resistance comprising contacting a test agent with a condensate, insulin receptor (IR), and insulin in an elevated reactive oxygen species (ROS) environment, wherein if the test agent increases IR incorporation into the condensate as compared to a control then the test agent is identified as a candidate agent to treat insulin resistance.

In some embodiments, the method comprises measuring IR incorporation. Any suitable method may be used to measure IR incorporation. In some embodiments, IR incorporation is measured by time-correlated photoactivation localization microscopy. In some embodiments, the measured IR incorporation is compared to IR incorporation in a control condensate not contacted with the agent. In some embodiments, the IR comprises a detectable tag to facilitate detection/measurement.

In some embodiments, the test agent, condensate and IR are contacted with about a 0.1 nM concentration of insulin.

In some embodiments, if the condensate exhibits at least about 10%, 20%, 30%, 40%, 50%, 60%, 70%, 80%, 90%, or substantially 100% more IR incorporation in the presence of agent than a suitable control condensate (e.g., a condensate not contacted with the agent) than the test agent is identified as a candidate agent to treat insulin resistance.

In some embodiments, the elevated ROS environment has a ROS concentration that is at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, or 20-fold greater than the ROS concentration in a non-elevated ROS environment.

The candidate agent may be any agent described herein. In some embodiments, the candidate agent is a small molecule. The condensate may be any condensate described herein. In some embodiments, the condensate is a transcriptional condensate. In some embodiments, the condensate is a transcriptional condensate associated with an insulin responsive gene. In some embodiments, the condensate is a synthetic in vitro condensate or an ex vivo condensate isolated from a cell. In some embodiments, the ex vivo condensate is isolated from an insulin resistant cell. In some embodiments, the insulin resistant cell is a mammalian (e.g., human) fat, liver, brain, kidney, muscle (e.g., skeletal muscle), or pancreatic islet cell (e.g., pancreatic b-cell). In some embodiments, the in vitro condensate comprises one or more condensate components from a transcriptional condensate (e.g., a transcriptional condensate associated with an insulin responsive gene). In some embodiments, the in vitro condensate comprises MED1.

In some embodiments, the condensate is in a cell. The cell is not limited and may be any cell described herein. In some embodiments, the cell is mammalian (e.g., human) fat, liver, brain, kidney, muscle (e.g., skeletal muscle), or pancreatic islet cell (e.g., pancreatic b-cell). In some embodiments, the cell is in a test animal and the test agent is administered to the test animal (e.g., mouse, rat, rabbit, etc.).

In some embodiments, the cells may be primary cells, may be isolated from or originate from a subject suffering from insulin resistance or an insulin resistance associated disorder, may be members of a cell line, may be cultured in 2D culture system e.g., on a surface of a well or plate, may be cultured in a three-dimensional culture system e.g., as spheroids or organoids, may be of a cell type that normally expresses the insulin receptor and responds to insulin by exhibiting one or more physiological changes in response to insulin.

In some embodiments, multiple candidate agents are screened and one or more agents having a comparable or greater effect than metformin are identified.

Some aspects of the present disclosure are directed to a method of screening for a candidate agent to treat insulin resistance comprising contacting a test agent with a condensate, insulin receptor (IR), and insulin (e.g., at a concentration of 0.1 nM), wherein the condensate exhibits reduced incorporation of IR in the presence of insulin as compared to a control, wherein if the test agent increases IR incorporation into the condensate as compared to a control then the test agent is identified as a candidate agent to treat insulin resistance.

In some embodiments, the method comprises measuring IR incorporation. Any suitable method may be used to measure IR incorporation. In some embodiments, IR incorporation is measured by time-correlated photoactivation localization microscopy. In some embodiments, the measured IR incorporation is compared to IR incorporation in a control condensate not contacted with the agent. In some embodiments, the IR comprises a detectable tag to facilitate detection/measurement.

In some embodiments, the condensate exhibits at least about 10%, 20%, 30%, 40%, 50%, 60%, 70%, 80%, 90%, or substantially 100% less IR incorporation in the presence of insulin than a suitable control condensate (e.g., a condensate not associated with insulin resistance). In some embodiments, if the test agent increases the flux of IR incorporation by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, or 20-fold greater than in a suitable control condensate not treated with the test agent than the test agent is identified as a candidate agent to treat insulin resistance.

The candidate agent may be any agent described herein. In some embodiments, the candidate agent is a small molecule. The condensate may be any condensate described herein. In some embodiments, the condensate is a transcriptional condensate. In some embodiments, the condensate is a transcriptional condensate associated with an insulin responsive gene. In some embodiments, the condensate is a synthetic in vitro condensate or an ex vivo condensate isolated from a cell. In some embodiments, the ex vivo condensate is isolated from an insulin resistant cell. In some embodiments, the insulin resistant cell is a mammalian (e.g., human) fat, liver, brain, kidney, muscle (e.g., skeletal muscle), or pancreatic islet cell (e.g., pancreatic b-cell). In some embodiments, the in vitro condensate comprises one or more condensate components from a transcriptional condensate (e.g., a transcriptional condensate associated with an insulin responsive gene). In some embodiments, the in vitro condensate comprises MED1.

In some embodiments, one or more components of the condensate has been exposed to elevated ROS levels. In some embodiments, the elevated ROS level is a ROS level that is at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, or 20-fold greater than the ROS level in a non-elevated ROS environment.

In some embodiments, the condensate is in a cell. The cell is not limited and may be any cell described herein. In some embodiments, the cell is mammalian (e.g., human) fat, liver, brain, kidney, muscle (e.g., skeletal muscle), or pancreatic islet cell (e.g., pancreatic b-cell). In some embodiments, the cell is in a test animal and the test agent is administered to the test animal (e.g., mouse, rat, rabbit, etc.).

In some embodiments, the cells may be primary cells, may be isolated from or originate from a subject suffering from insulin resistance or an insulin resistance associated disorder, may be members of a cell line, may be cultured in 2D culture system e.g., on a surface of a well or plate, may be cultured in a three-dimensional culture system e.g., as spheroids or organoids, may be of a cell type that normally expresses the insulin receptor and responds to insulin by exhibiting one or more physiological changes in response to insulin.

In some embodiments, multiple candidate agents are screened and one or more agents having a comparable or greater effect than metformin are identified.

Some aspects of the present disclosure are directed to a method of screening for a candidate agent to treat insulin resistance comprising contacting a test agent with a mixture comprising a cell or in vitro transcription assay with condensate dependent expression of a reporter gene, insulin receptor (IR), and insulin, wherein the condensate exhibits reduced incorporation of IR in the presence of insulin as compared to a control, wherein if the test agent increases reporter gene expression as compared to a control then the test agent is identified as a candidate agent to treat insulin resistance.

In some embodiments, the method comprises measuring IR incorporation. Any suitable method may be used to measure IR incorporation. In some embodiments, IR incorporation is measured by time-correlated photoactivation localization microscopy. In some embodiments, the measured IR incorporation is compared to IR incorporation in a control condensate not contacted with the agent. In some embodiments, the IR comprises a detectable tag to facilitate detection/measurement.

In some embodiments, the condensate exhibits at least about 10%, 20%, 30%, 40%, 50%, 60%, 70%, 80%, 90%, or substantially 100% less IR incorporation in the presence of insulin than a suitable control condensate (e.g., a condensate not associated with insulin resistance).

In some embodiments, if the test agent increases reporter gene expression by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, or 20-fold greater than in a suitable control condensate not treated with the test agent than the test agent is identified as a candidate agent to treat insulin resistance.

The candidate agent may be any agent described herein. In some embodiments, the candidate agent is a small molecule. The condensate may be any condensate described herein. In some embodiments, the condensate is a transcriptional condensate. In some embodiments, the condensate is a transcriptional condensate associated with an insulin responsive gene. In some embodiments, the condensate is a synthetic in vitro condensate or an ex vivo condensate isolated from a cell. In some embodiments, the ex vivo condensate is isolated from an insulin resistant cell. In some embodiments, the insulin resistant cell is a mammalian (e.g., human) fat, liver, brain, kidney, muscle (e.g., skeletal muscle), or pancreatic islet cell (e.g., pancreatic b-cell). In some embodiments, the in vitro condensate comprises one or more condensate components from a transcriptional condensate (e.g., a transcriptional condensate associated with an insulin responsive gene). In some embodiments, the in vitro condensate comprises MED1.

In some embodiments, the condensate is in a cell. The cell is not limited and may be any cell described herein. In some embodiments, the cell is mammalian (e.g., human) fat, liver, brain, kidney, muscle (e.g., skeletal muscle), or pancreatic islet cell (e.g., pancreatic b-cell). In some embodiments, the cell is in a test animal and the test agent is administered to the test animal (e.g., mouse, rat, rabbit, etc.).

In some embodiments, the cells may be primary cells, may be isolated from or originate from a subject suffering from insulin resistance or an insulin resistance associated disorder, may be members of a cell line, may be cultured in 2D culture system e.g., on a surface of a well or plate, may be cultured in a three-dimensional culture system e.g., as spheroids or organoids, may be of a cell type that normally expresses the insulin receptor and responds to insulin by exhibiting one or more physiological changes in response to insulin.

In some embodiments, multiple candidate agents are screened and one or more agents having a comparable or greater effect than metformin are identified.

Some aspects of the present disclosure are directed to a method of screening for a candidate agent to treat insulin resistance comprising (a) contacting a test agent with IR condensates and insulin, wherein the IR condensates exhibits increased average lifetime and/or increased percentage of long-lived IR condensates in the presence of insulin as compared to a control; and (b) measuring the average lifetime and/or percentage of long-lived IR condensates wherein if the test agent decreases average lifetime of the IR condensates and/or increases the percentage of long-lived IR condensates as compared to a control then the test agent is identified as a candidate agent to treat insulin resistance.

Any suitable method may be used to measure the average lifetime and/or percentage of long-lived IR condensates. In some embodiments, the average lifetime and/or percentage of long-lived IR condensates is measured by time-correlated photoactivation localization microscopy. In some embodiments, the measured the average lifetime and/or percentage of long-lived IR condensates is compared to the average lifetime and/or percentage of long-lived IR condensates in a control condensate not contacted with the agent. In some embodiments, the IR comprises a detectable tag to facilitate detection/measurement.

In some embodiments, the condensate exhibits at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, or 20-fold increased average lifetime and/or increased percentage of long-lived IR condensates in the presence of insulin than a suitable control condensate (e.g., a condensate not associated with insulin resistance).

In some embodiments, if the test agent decreases average lifetime and/or the percentage of long-lived IR condensates by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, or 20-fold than in a suitable control condensate not treated with the test agent than the test agent is identified as a candidate agent to treat insulin resistance.

In some embodiments, an IR condensate is considered long-lived if it is present for about 100-150 seconds. In some embodiments, an IR condensate is considered long-lived if it is present for about 120 seconds.

The candidate agent may be any agent described herein. In some embodiments, the candidate agent is a small molecule. The condensate may be any condensate described herein. In some embodiments, the condensate is a transcriptional condensate. In some embodiments, the condensate is a transcriptional condensate associated with an insulin responsive gene. In some embodiments, the condensate is a synthetic in vitro condensate or an ex vivo condensate isolated from a cell. In some embodiments, the ex vivo condensate is isolated from an insulin resistant cell. In some embodiments, the insulin resistant cell is a mammalian (e.g., human) fat, liver, brain, kidney, muscle (e.g., skeletal muscle), or pancreatic islet cell (e.g., pancreatic b-cell). In some embodiments, the in vitro condensate comprises one or more condensate components from a transcriptional condensate (e.g., a transcriptional condensate associated with an insulin responsive gene). In some embodiments, the in vitro condensate comprises MED1.

In some embodiments, the condensate is in a cell. The cell is not limited and may be any cell described herein. In some embodiments, the cell is mammalian (e.g., human) fat, liver, brain, kidney, muscle (e.g., skeletal muscle), or pancreatic islet cell (e.g., pancreatic b-cell). In some embodiments, the cell is in a test animal and the test agent is administered to the test animal (e.g., mouse, rat, rabbit, etc.).

In some embodiments, the cells may be primary cells, may be isolated from or originate from a subject suffering from insulin resistance or an insulin resistance associated disorder, may be members of a cell line, may be cultured in 2D culture system e.g., on a surface of a well or plate, may be cultured in a three-dimensional culture system e.g., as spheroids or organoids, may be of a cell type that normally expresses the insulin receptor and responds to insulin by exhibiting one or more physiological changes in response to insulin.

In some embodiments, multiple candidate agents are screened and one or more agents having a comparable or greater effect than metformin are identified.

Some aspects of the present disclosure are directed to a method of screening for a candidate agent to modulate insulin receptor activity comprising (a) contacting a test agent with IR condensates and insulin: (b) measuring a property or behavior of said IR condensates as compared to a control, and (c) identifying the test agent as a candidate modulator of insulin receptor activity if the property or behavior of said IR condensates differ in the presence of the test agent as compared to the control. The property or behavior is not limited and may be any suitable property or behavior (e.g., formation, stability, or morphology of the condensate). In some embodiments, the property or behavior comprises average number of IR molecule(s) in IR condensates, average lifetime of IR condensates, and/or percentage of long-lived IR condensates. In some embodiments, the IR comprises a detectable tag to facilitate detection/measurement.

In some embodiments, the method comprises measuring the average number of IR molecule(s) in IR condensates. Any suitable method may be used. In some embodiments, the average number of IR molecule(s) in IR condensates is measured by time-correlated photoactivation localization microscopy. In some embodiments, the measured average number of IR molecule(s) in IR condensates is compared to the average number of IR molecule(s) in a control condensate not contacted with the agent.

In some embodiments, the method comprises measuring the average lifetime of IR condensates. Any suitable method may be used. In some embodiments, the average lifetime of IR condensates is measured by time-correlated photoactivation localization microscopy. In some embodiments, the measured average lifetime of IR condensates is compared to the average lifetime of IR condensates in a control condensate not contacted with the agent.

In some embodiments, the method comprises measuring the percentage of long-lived IR condensates. Any suitable method may be used. In some embodiments, the percentage of long-lived IR condensates is measured by time-correlated photoactivation localization microscopy. In some embodiments, the measured percentage of long-lived IR condensates is compared to the percentage of long-lived IR condensates in a control condensate not contacted with the agent. In some embodiments, a long-lived IR condensate is present for about 120 seconds or more.

In some embodiments, the condensate(s) is/are in a cell. In some embodiments, the cell is a mammalian (e.g., human) fat, liver, brain, kidney, muscle (e.g., skeletal muscle) or pancreatic islet cell (e.g., pancreatic b-cell). In some embodiments, the cell is an insulin resistant cell. In some embodiments, the cell is an insulin resistant and metformin resistant cell.

Some aspects of the present disclosure are directed to a method of characterizing an agent, comprising (i) contacting the agent with a cell comprising the insulin receptor, and (ii) measuring ability of the agent to modulate one or more of the following: (a) flux of IR incorporation into condensates within the cell in response to insulin; (b) average number of IR molecules in IR condensates in the cell; (c) average lifetime of IR condensates in the cell; (d) percentage of long-lived IR condensates in the cell.

In some embodiments, the method comprises measuring the flux of IR incorporation into condensates within the cell in response to insulin. Any suitable method may be used. In some embodiments, the flux of IR incorporation into condensates within the cell in response to insulin is measured by time-correlated photoactivation localization microscopy. In some embodiments, the measured flux of IR incorporation into condensates within the cell in response to insulin is compared to the flux in a control condensate in a cell not contacted with the agent.

In some embodiments, the method comprises measuring the average number of IR molecules in IR condensates in the cell. Any suitable method may be used. In some embodiments, the average number of IR molecules in IR condensates in the cell is measured by time-correlated photoactivation localization microscopy. In some embodiments, the measured average number of IR molecules in IR condensates in the cell is compared to the average number of IR molecules in IR condensates in a control cell not contacted with the agent.

In some embodiments, the method comprises measuring the average lifetime of IR condensates in the cell. Any suitable method may be used. In some embodiments, the average lifetime of IR condensates in the cell is measured by time-correlated photoactivation localization microscopy. In some embodiments, the measured average lifetime of IR condensates in the cell is compared to the average lifetime of IR condensates in a control cell not contacted with the agent.

In some embodiments, the method comprises measuring the percentage of long-lived IR condensates in the cell. Any suitable method may be used. In some embodiments, the percentage of long-lived IR condensates in the cell is measured by time-correlated photoactivation localization microscopy. In some embodiments, the measured percentage of long-lived IR condensates in the cell is compared to the percentage of long-lived IR condensates in a control cell not contacted with the agent. In some embodiments, a long-lived IR condensate is present for about 120 seconds or more.

In some embodiments, the cell is a mammalian (e.g., human) fat, liver, brain, kidney, muscle (e.g., skeletal muscle) or pancreatic islet cell (e.g., pancreatic b-cell). In some embodiments, the cell is an insulin resistant cell. In some embodiments, the cell is an insulin resistant and metformin resistant cell.

In some embodiments, the cell is contacted with insulin prior to step (i). In some embodiments, the cell is contacted with insulin prior to step (ii). In some embodiments, the cell is contacted with about 0.1 nM of insulin.

In some embodiments of the screening methods disclosed herein, the condensate has a detectable tag. The detectable tag can be used to determine if contact with the test agent modulates formation, stability, or morphology of the condensate. In some embodiments, a cell is a genetically engineered to express the detectable tag. In some embodiments, the detectable tag is incorporated into the condensate (e.g., a component of the condensate, the regRNA). The term “detectable tag” or “detectable label” as used herein includes, but is not limited to, detectable labels, such as fluorophores, radioisotopes, colorimetric substrates, or enzymes; heterologous epitopes for which specific antibodies are commercially available, e.g., FLAG-tag; heterologous amino acid sequences that are ligands for commercially available binding proteins, e.g., Strep-tag, biotin; fluorescence quenchers typically used in conjunction with a fluorescent tag on the other polypeptide; nucleic acid intercalating agents, and complementary bioluminescent or fluorescent polypeptide fragments. A tag that is a detectable label or a complementary bioluminescent or fluorescent polypeptide fragment may be measured directly (e.g., by measuring fluorescence or radioactivity of, or incubating with an appropriate substrate or enzyme to produce a spectrophotometrically detectable color change for the associated polypeptides as compared to the unassociated polypeptides). A tag that is a heterologous epitope or ligand is typically detected with a second component that binds thereto, e.g., an antibody or binding protein, wherein the second component is associated with a detectable label.

In some specific embodiments of the screening methods disclosed herein, the condensate comprises a mediator component or fragment thereof comprising an IDR. In some embodiments, the mediator component or fragment thereof comprising an IDR further comprises a label.

In some embodiments of the screening methods disclosed herein, “assessing” comprises measuring a physical property as compared to a control or reference. For example, assessing if the condensate is dissolved or the stability of a condensate is modulated may comprise measuring the period of time a condensate exists as compared to a control condensate not subject to a test condition or agent. assessing if the shape or size of a condensate is modulated can comprise comparing the shape of a condensate as compared to a control condensate not subject to a test condition or agent. In some embodiments, one or more properties of a condensate may be “assessed” to be modulated if they are changed by a statistically significant amount (e.g., at least 10%, at least 20%, at least 30%, at least 50%, at least 75%, or more).

In some embodiments of the screening methods disclosed herein, the step of determining if contact with the test agent modulates (flux of) IR incorporation into the condensate, average lifetime and/or percentage of long-lived IR condensates, average number of IR molecule(s) in IR condensates, size, dissolution, formation, stability, or morphology of the condensate is performed using microscopy, which is not limited. In some embodiments, the microscopy is deconvolution microscopy, structured illumination microscopy, time-correlated photoactivation localization microscopy, or interference microscopy. In some embodiments, the step of determining if contact with the test agent modulates (flux of) IR incorporation into the condensate, average lifetime and/or percentage of long-lived IR condensates, average number of IR molecule(s) in IR condensates, formation, stability, or morphology of the condensate is performed using DNA-FISH, RNA-FISH, or a combination thereof.

In some embodiments of the screening methods disclosed herein, the cell or condensate does not express a reporter gene prior to contact with a test agent and expresses a reporter gene after contact with an agent that enhances condensate formation, stability, size, function, or morphology. In some embodiments, the cell does express a reporter gene prior to contact with a test agent and stops or reduces expression of the reporter gene after contact with an agent that dissolves condensates, reduces condensate stability, or prevents/suppresses condensate formation.

In some embodiments of the screening methods disclosed herein, a high throughput screen (HTS) is performed. A high throughput screen can utilize cell-free or cell-based assays (e.g., a condensate containing cell as described herein, an in vitro condensate, an isolated in vitro condensate). High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hüser.

The term “hit” generally refers to an agent that achieves an effect of interest in a screen or assay, e.g., an agent that has at least a predetermined level of modulating effect on cell survival, cell proliferation, gene expression, protein activity, or other parameter of interest being measured in the screen or assay. Test agents that are identified as hits in a screen may be selected for further testing, development, or modification. In some embodiments a test agent is retested using the same assay or different assays. For example, a candidate anticancer agent may be tested against multiple different cancer cell lines or in an in vivo tumor model to determine its effect on cancer cell survival or proliferation, tumor growth, etc. Additional amounts of the test agent may be synthesized or otherwise obtained, if desired. Physical testing or computational approaches can be used to determine or predict one or more physicochemical, pharmacokinetic and/or pharmacodynamic properties of compounds identified in a screen. For example, solubility, absorption, distribution, metabolism, and excretion (ADME) parameters can be experimentally determined or predicted. Such information can be used, e.g., to select hits for further testing, development, or modification. For example, small molecules having characteristics typical of “drug-like” molecules can be selected and/or small molecules having one or more unfavorable characteristics can be avoided or modified to reduce or eliminated such unfavorable characteristic(s).

In some embodiments structures of hit compounds are examined to identify a pharmacophore, which can be used to design additional compounds. An additional compound may, for example, have one or more altered, e.g., improved, physicochemical, pharmacokinetic (e.g., absorption, distribution, metabolism and/or excretion) and/or pharmacodynamic properties as compared with an initial hit or may have approximately the same properties but a different structure. An improved property is generally a property that renders a compound more readily usable or more useful for one or more intended uses. Improvement can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties. In some embodiments a molecular target of a hit compound is identified or known. In some embodiments, additional compounds that act on the same molecular target may be identified empirically (e.g., through screening a compound library) or designed.

Data or results from testing an agent or performing a screen may be stored or electronically transmitted. Such information may be stored on a tangible medium, which may be a computer-readable medium, paper, etc. In some embodiments a method of identifying or testing an agent comprises storing and/or electronically transmitting information indicating that a test agent has one or more propert(ies) of interest or indicating that a test agent is a “hit” in a particular screen, or indicating the particular result achieved using a test agent. A list of hits from a screen may be generated and stored or transmitted. Hits may be ranked or divided into two or more groups based on activity, structural similarity, or other characteristics

Once a candidate agent is identified, additional agents, e.g., analogs, may be generated based on it. An additional agent, may, for example, have increased cancer cell uptake, increased potency, increased stability, greater solubility, or any improved property. In some embodiments a labeled form of the agent is generated. The labeled agent may be used, e.g., to directly measure binding of an agent to a molecular target in a cell. In some embodiments, a molecular target of an agent identified as described herein may be identified. An agent may be used as an affinity reagent to isolate a molecular target. An assay to identify the molecular target, e.g., using methods such as mass spectrometry, may be performed. Once a molecular target is identified, one or more additional screens maybe performed to identify agents that act specifically on that target.

The test agent for the screening methods disclosed herein are not limited and may be a type of agent described herein (e.g., a protein, nucleic acid, small molecule, etc.)

Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A compound library may comprise natural products and/or compounds generated using non-directed or directed synthetic organic chemistry. In some embodiments a library is a small molecule library, peptide library, peptoid library, cDNA library, oligonucleotide library, or display library (e.g., a phage display library). In some embodiments a library comprises agents of two or more of the foregoing types. In some embodiments oligonucleotides in an oligonucleotide library comprise siRNAs, shRNAs, antisense oligonucleotides, aptamers, or random oligonucleotides.

A library may comprise, e.g., between 100 and 500,000 compounds, or more. In some embodiments a library comprises at least 10,000, at least 50,000, at least 100,000, or at least 250,000 compounds. In some embodiments compounds of a compound library are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments compounds that have been identified as “hits” or “leads” in a drug discovery program and/or analogs thereof. In some embodiments a library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments a test agent is not an agent that is found in a cell culture medium known or used in the art, e.g., for culturing vertebrate, e.g., mammalian cells, e.g., an agent provided for purposes of culturing the cells. In some embodiments, if the agent is one that is found in a cell culture medium known or used in the art, the agent may be used at a different, e.g., higher, concentration when used as a test agent in a method or composition described herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

It is reported herein that IR is incorporated into liquid-like condensates at the plasma membrane, in the cytoplasm and in the nucleus of liver cells. Insulin stimulation promotes further incorporation of IR into these dynamic condensates in insulin sensitive cells but not in insulin resistant cells, where IR molecules within condensates exhibit less dynamic behavior. Metformin treatment of insulin resistant cells rescues IR condensate dynamics and insulin responsiveness. Insulin resistant cells are subjected to high levels of oxidative stress, which was found to cause reduced condensate dynamics, and treatment of these cells with metformin reduces levels of reactive oxygen species (ROS) and returns IR condensates to their normal dynamic behavior.

### Results

Insulin Promotes IR Incorporation into Puncta in HepG2 Cells

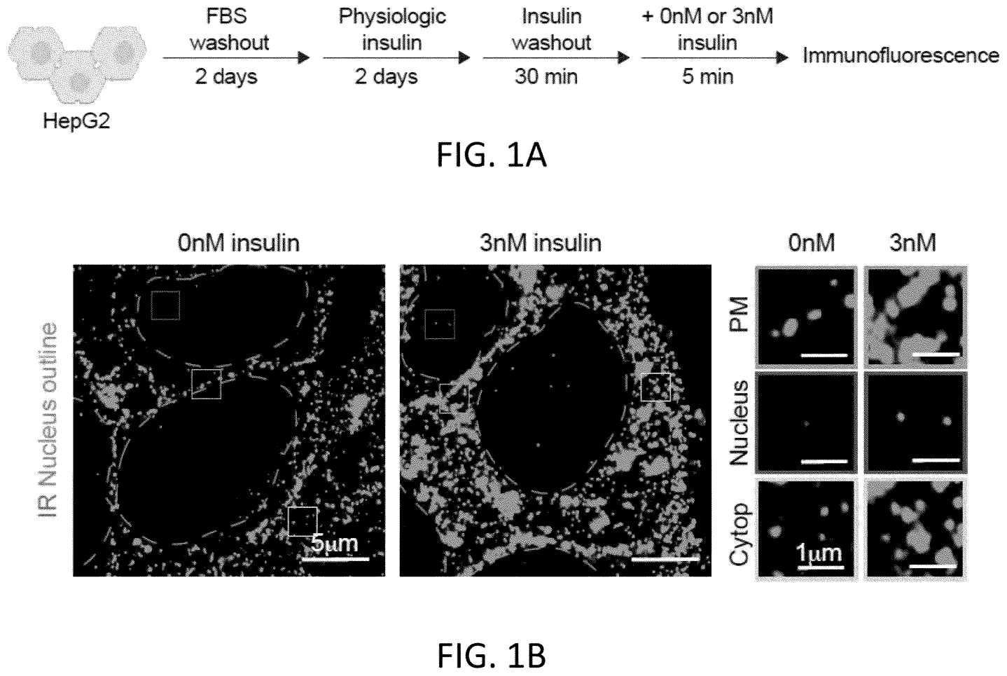

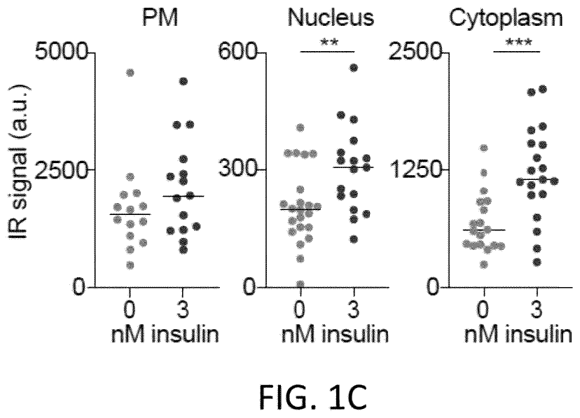

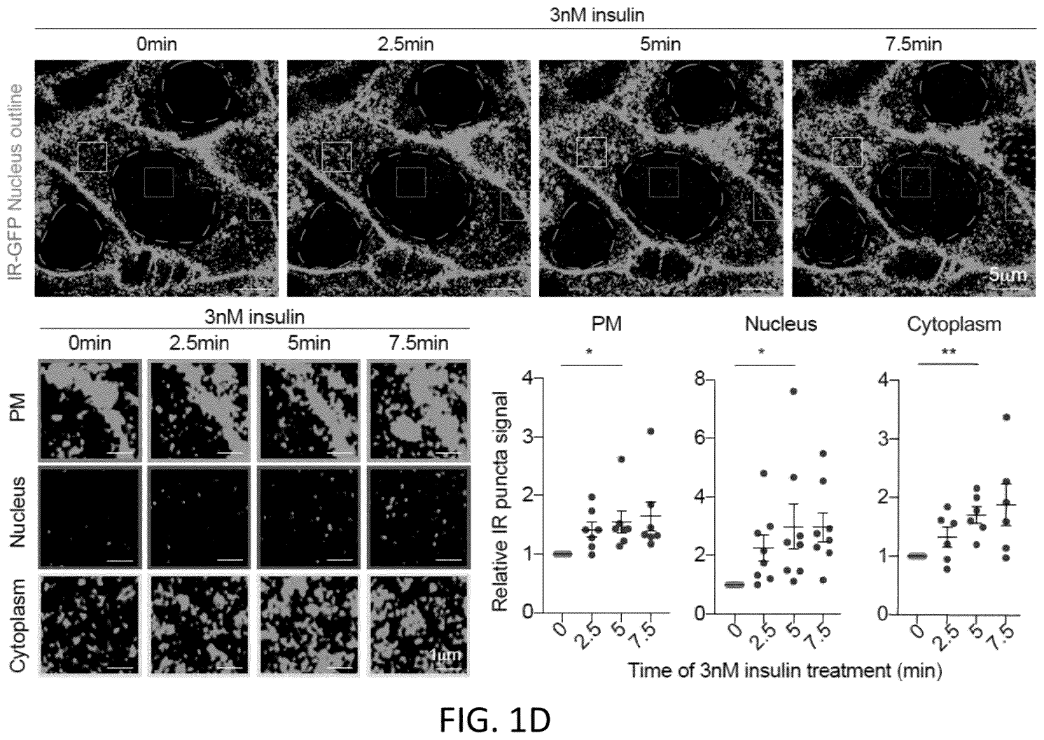

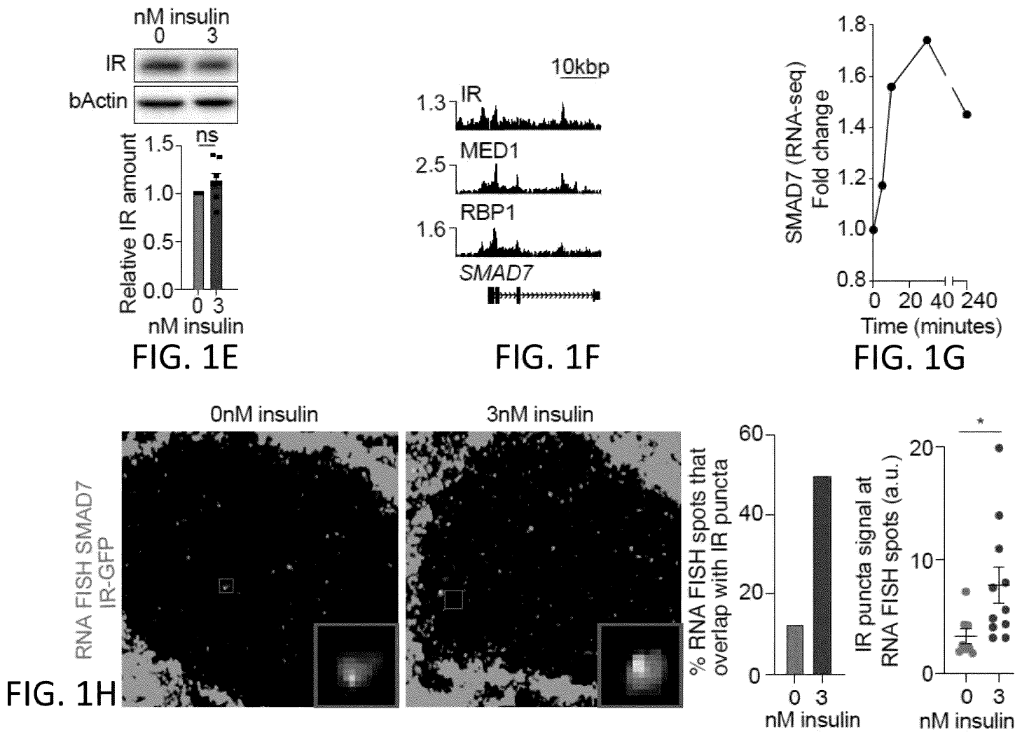

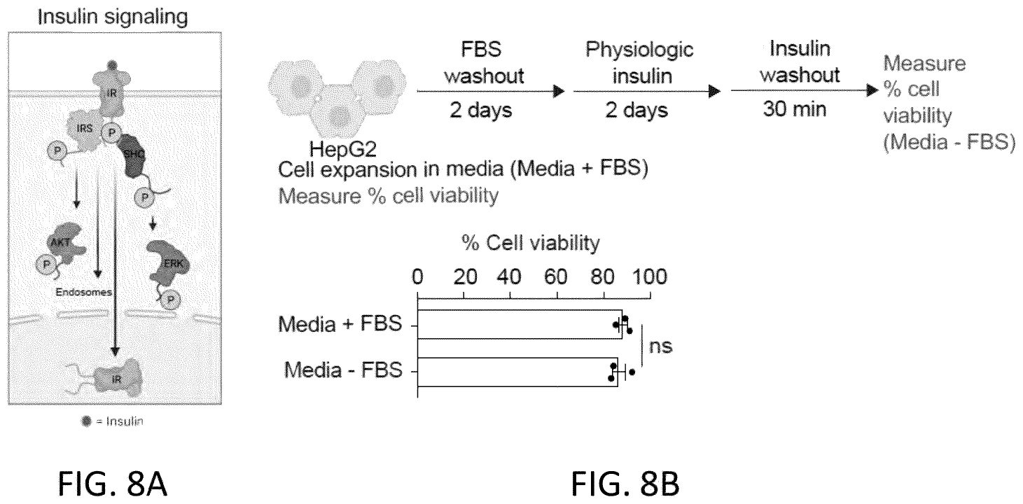

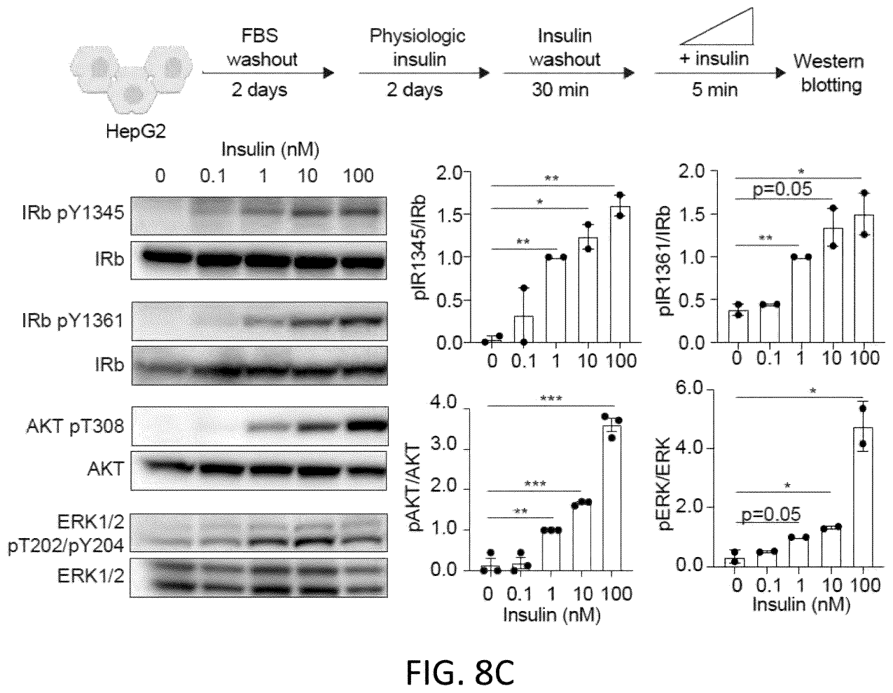

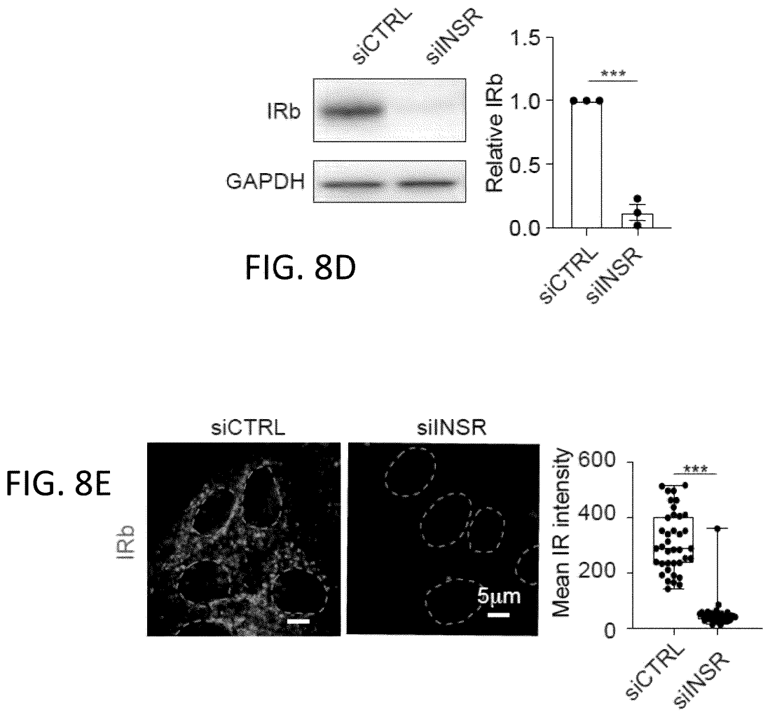

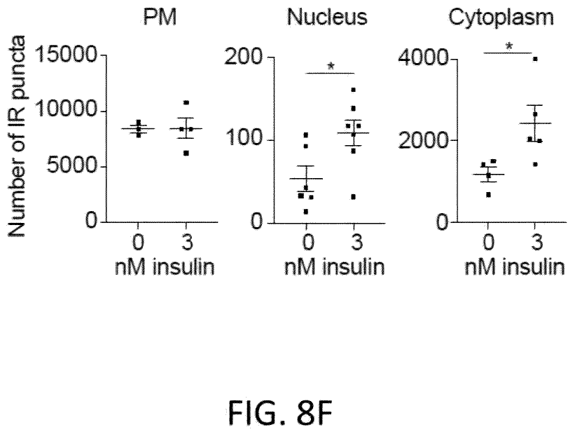

The insulin receptor can be found in the plasma membrane, in the cytoplasm and in the nucleus (FIG. 8A) (30-38). To confirm and expand on previous observations that IR occurs in puncta in cells (30, 37, 38), applicant studied the location of IR in insulin sensitive HepG2 cells (FIG. 1, 8). HepG2 cells were used as an initial model system due to their demonstrated utility in the study of insulin signaling and resistance, and because they are amenable to genetic modification (25, 32, 50, 51). Cells were cultured in media containing physiological concentrations of glucose and insulin (FIG. 1A), where cell viability was maintained and physiological levels of insulin were observed to stimulate conventional measures of insulin responsiveness, including IR phosphorylation and AKT and ERK pathway activation (FIG. 8B, C). IR was monitored using immunofluorescence (IF) microscopy with an IR-specific antibody (FIG. 8D, E), and IR was found to be incorporated into puncta at the plasma membrane, cytoplasm and nucleus of HepG2 cells in the absence of insulin stimulation (FIG. 1B). Stimulation of the HepG2 cells with insulin caused an increase in the number of IR puncta detected in the cytoplasm and nucleus (FIG. 8F) and in the signal intensity in these puncta (FIG. 1B, C).

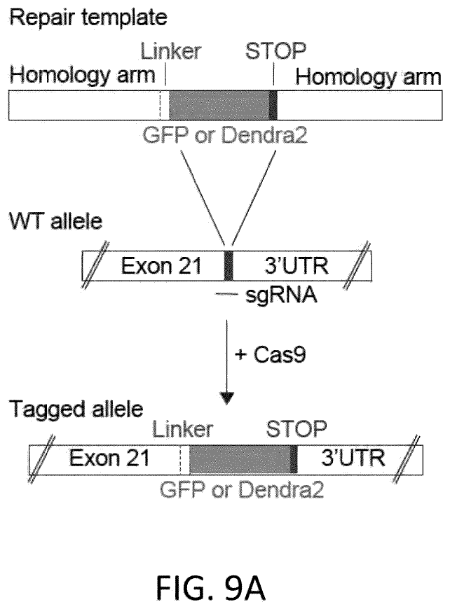

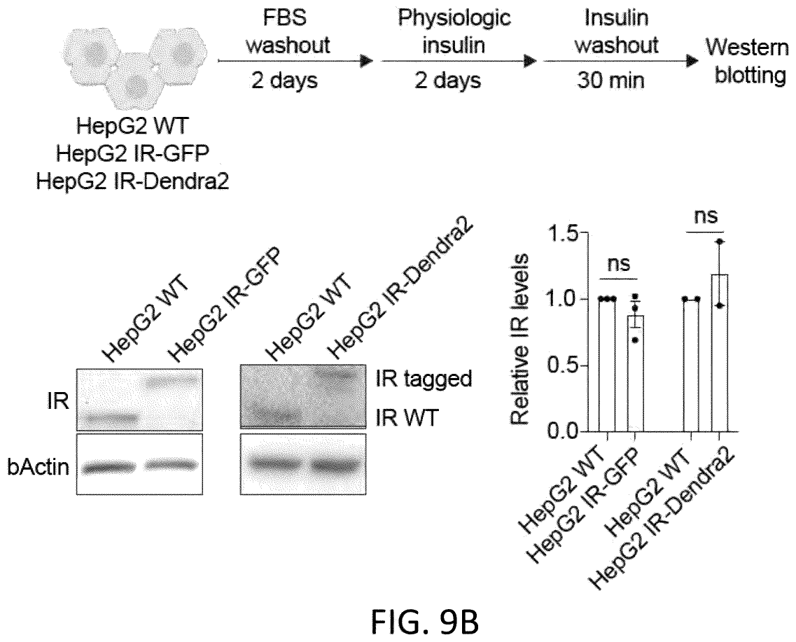

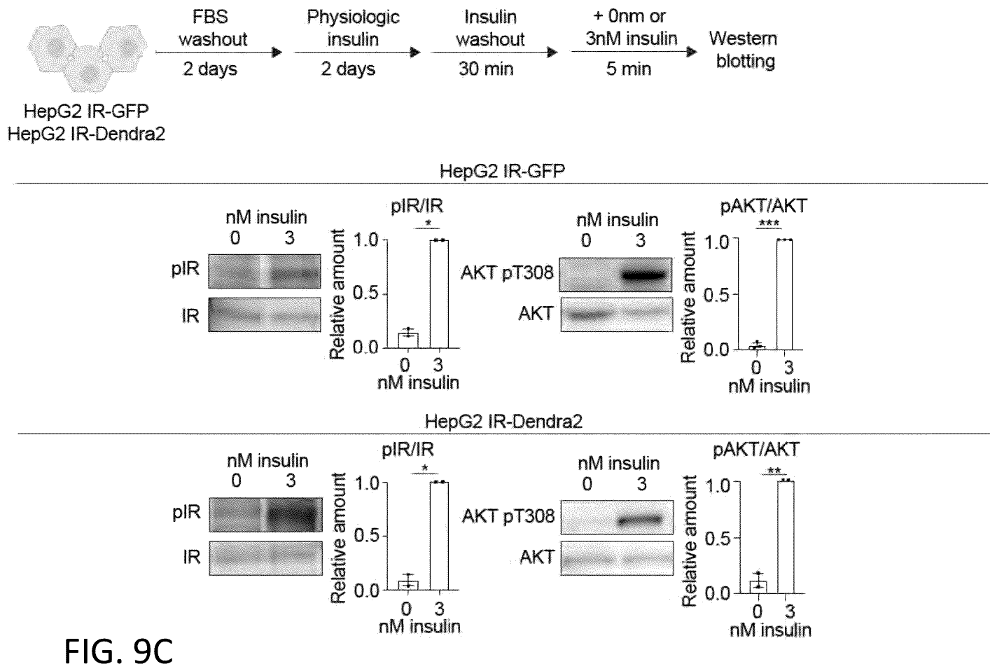

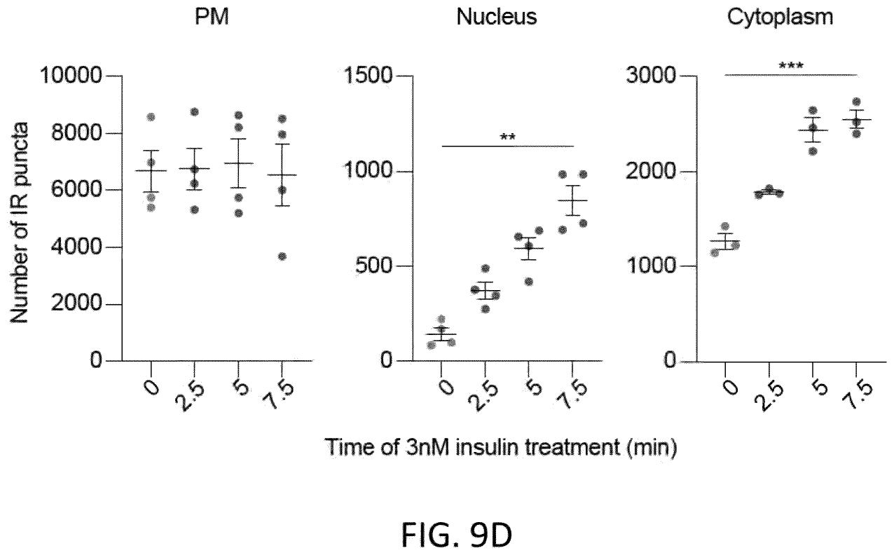

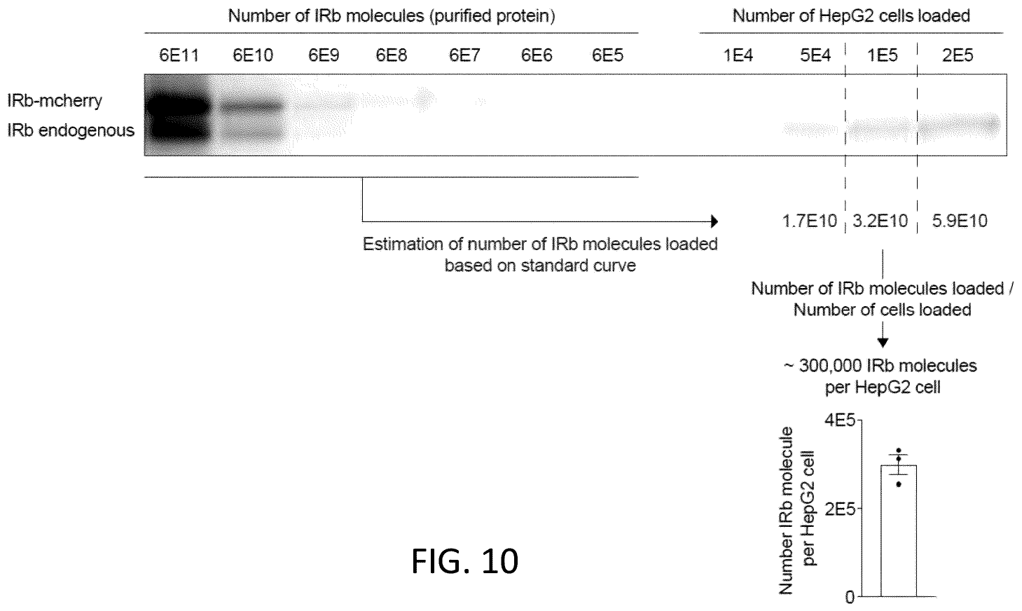

It was next sought to examine whether the insulin-stimulated increase of IR into punctate bodies, initially observed by IF in fixed cells, was also observed with live cell imaging of a fluorescently tagged IR protein. HepG2 cells were engineered to express endogenous IR as a fusion protein with monomeric enhanced green fluorescent protein (IR-GFP) (FIG. 9A). IR-GFP was expressed at the same levels as WT IR and was functional, as cells expressing this fusion protein maintained insulin sensitivity (FIG. 9B, C). Live cell imaging of IR-GFP revealed the same pattern of punctate signal that was observed for WT IR by IF in fixed cells, with increased numbers of cytoplasmic and nuclear puncta (FIG. 9D), and elevated IR-GFP signal in these puncta (FIG. 1D), during a time course following insulin stimulation. Western Blot analysis indicates HepG2 cells contain ˜300,000 IR molecules (FIG. 10A), consistent with estimates for human hepatocytes (52) and show that the unstimulated and insulin-stimulated cells contain similar levels of IR (FIG. 1E). Given the similar levels of IR in unstimulated and insulin-stimulated cells, it was inferred from the imaging results that the increased signal in puncta reflects increased incorporation of IR molecules into these bodies from the surrounding intracellular environment, and not changes in the overall level of the protein. Further study of these punctate bodies, described below, revealed that they have hallmark features of biomolecular condensates, so applicant will henceforth refer to them as condensates.

Evidence that the IR molecules in nuclear condensates are functionally active was sought by studying whether IR is incorporated into transcriptional condensates at genes whose expression is responsive to insulin stimulation. Insulin stimulation has been reported to increase IR occupancy at insulin-responsive genes (32), so it was investigated whether insulin stimulation promotes IR incorporation into transcriptional condensates at SMAD7, which is bound by IR together with proteins that are components of transcriptional condensates (MED1 and RPB1) (45, 53, 54) (FIG. 1F), and is upregulated upon insulin stimulation (FIG. 1G). Imaging of IR-GFP and SMAD7 intronic RNA FISH probes in cells showed that insulin stimulation leads to increased colocalization of IR-containing condensates with SMAD7 nascent RNA (FIG. 1H). These results indicate that insulin stimulation causes an increase in IR levels in transcriptional condensates at a gene whose expression is insulin responsive.

Insulin Receptor Molecules are Incorporated into Liquid-Like Condensates

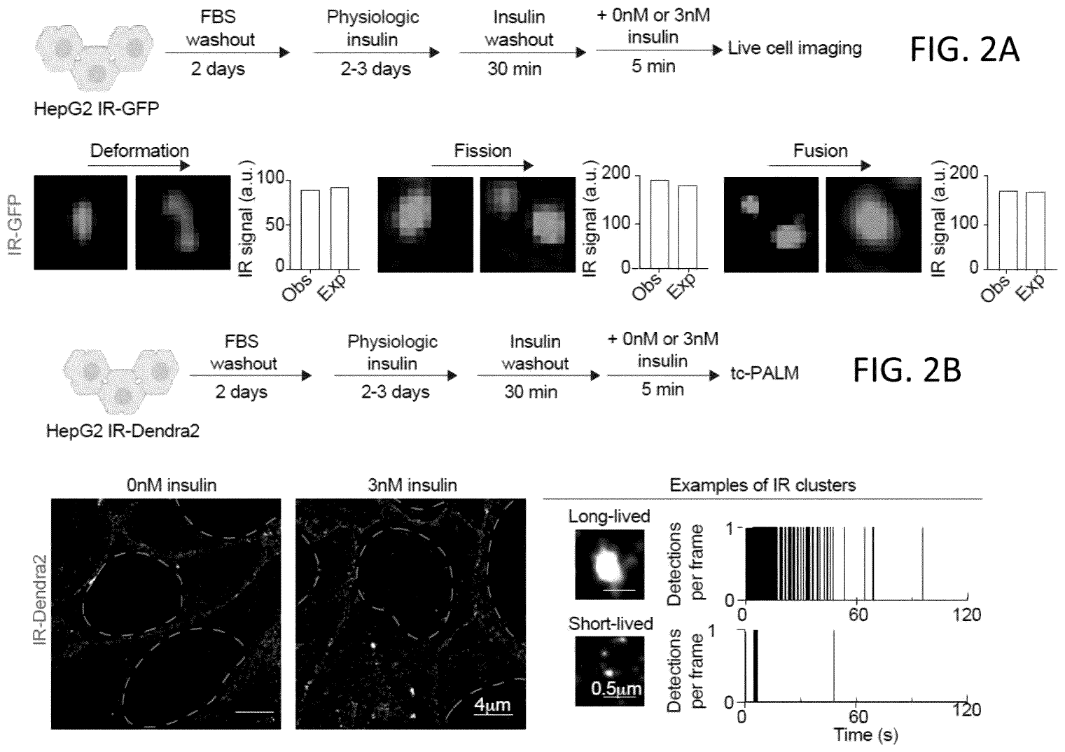

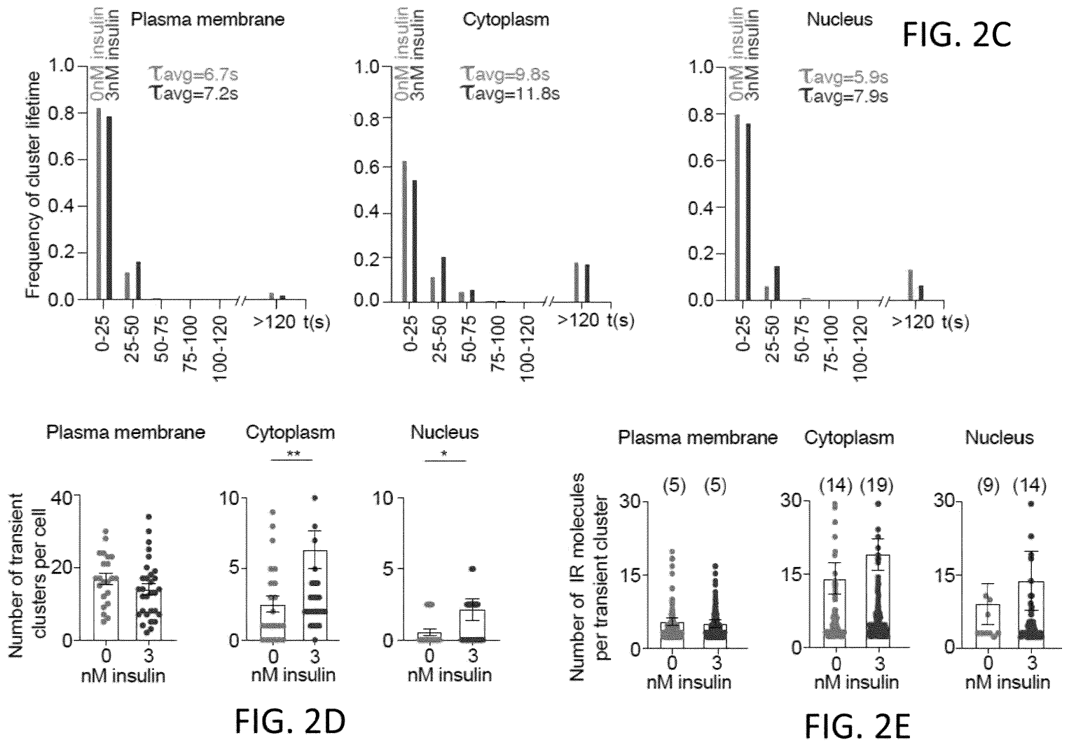

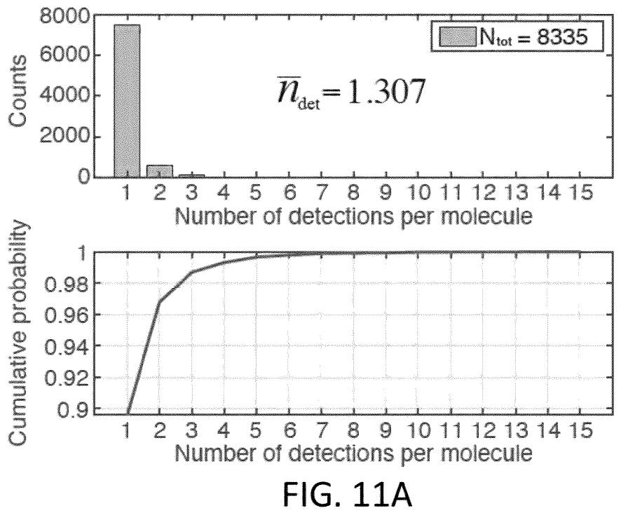

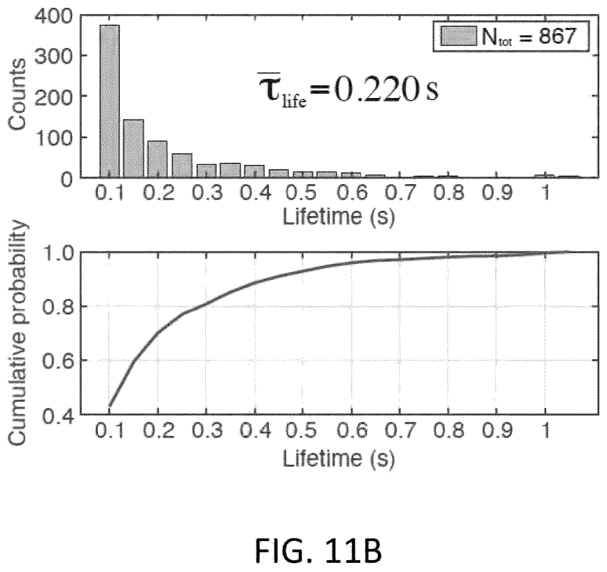

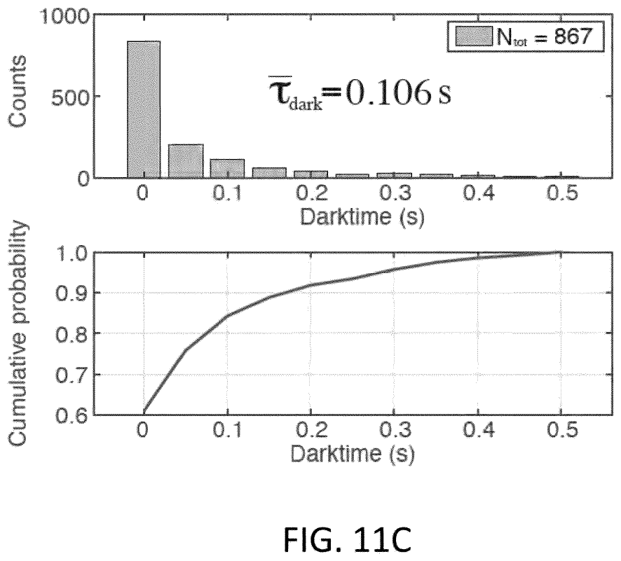

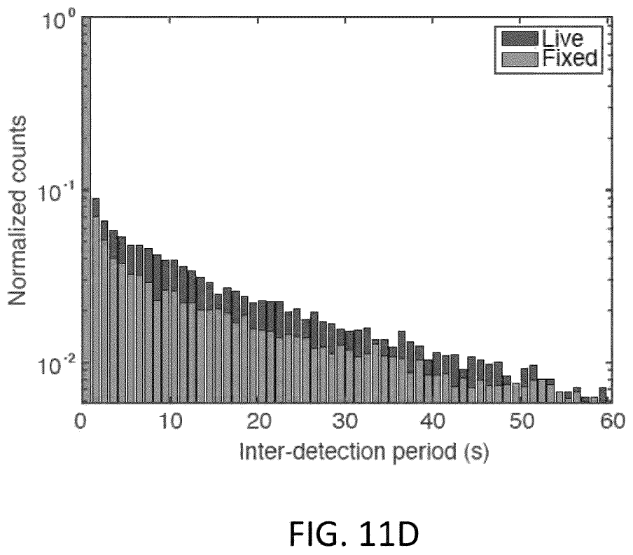

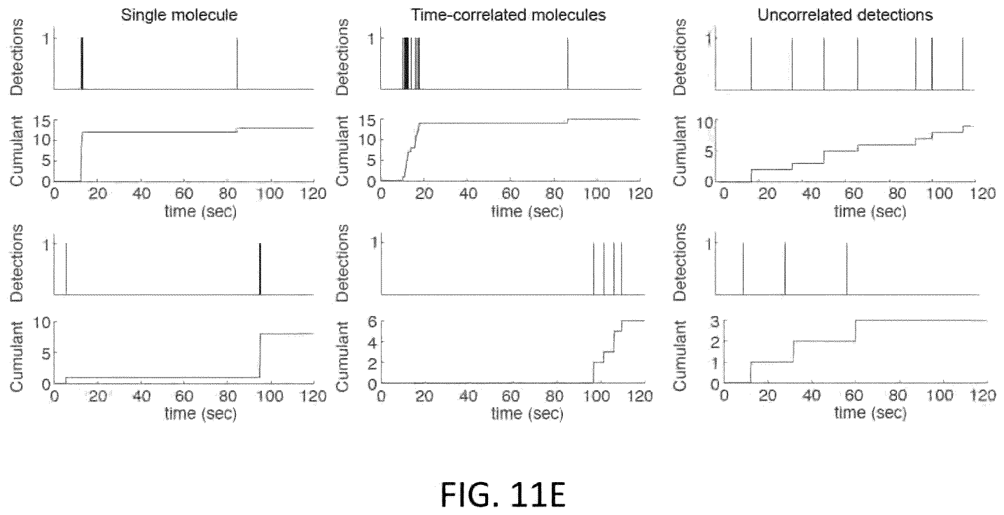

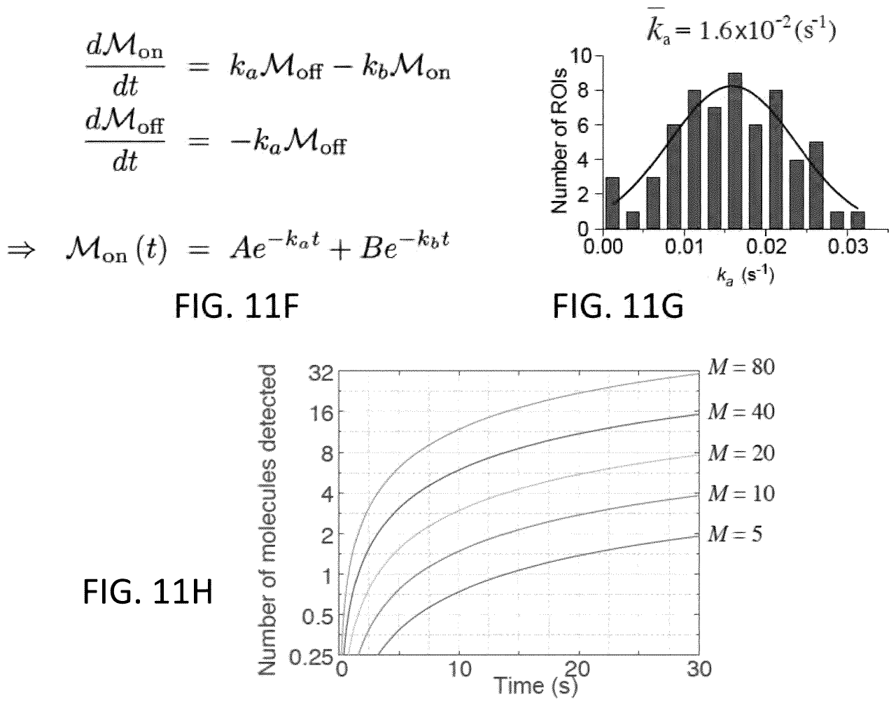

Confocal microscopy of HepG2 IR-GFP cells revealed IR-GFP puncta in the plasma membrane and cytoplasm undergoing deformation, fission and fusion (FIG. 2A), which are hallmarks of liquid-like condensates. To gain additional insights into the behavior of IR molecules in these condensates, a HepG2 cell line was engineered to express endogenous IR as a fusion protein with Dendra2 (IR-Dendra2) (FIG. 9A), which allows for time correlated Photoactivatable Light Microscopy (tc-PALM). Several control analyses of the single molecule photochemistry have been performed to validate the statistics of the identified clusters (FIG. 11, see Material and Methods). IR-Dendra2 was expressed at the same levels as WT IR and was functional, as cells expressing this fusion protein maintain insulin sensitivity (FIG. 9B, C) 1R-Dendra2 cells were subjected to tc-PALM and the results revealed that IR forms clusters at the plasma membrane, cytoplasm and nucleus that exhibit various lifetimes, consistent with dynamic formation and dissolution (FIG. 2B-E). In cells exposed to 0 nm or 3 nm insulin, the majority of IR clusters (˜85%) were short-lived (lifetime <120 s) and had an average lifetime of 6-12 s (FIG. 2C), comparable to those measured for transient transcriptional condensates in various cell types (53-55). A smaller fraction of clusters (˜15%) were present for considerably longer lifetimes (>120 s) (FIG. 2B, C). Insulin stimulation produced an increase in the number of IR clusters in the cytoplasm and nucleus of these cells (FIG. 2D) and an increase in the number of IR molecules in these clusters (FIG. 2E). In the cytoplasm, the average number of IR molecules per cluster was estimated to be 14 (2-264) in unstimulated cells, and 19 (2-295) in insulin stimulated cells (FIG. 2E). In the nucleus, the average number of IR molecules per cluster was estimated to be 9 (2-46) in unstimulated cells, and 14 (2-283) in insulin stimulated cells (FIG. 2E). These results show that multiple molecules of IR are incorporated into dynamic clusters, consistent with the behavior expected for biomolecular condensates, and that insulin stimulation leads to an increase in the number of IR-containing clusters and in the number of IR molecules per cluster in the cytoplasm and nucleus.

IR Incorporation into Condensates is Attenuated in Insulin Resistant HepG2 Cells

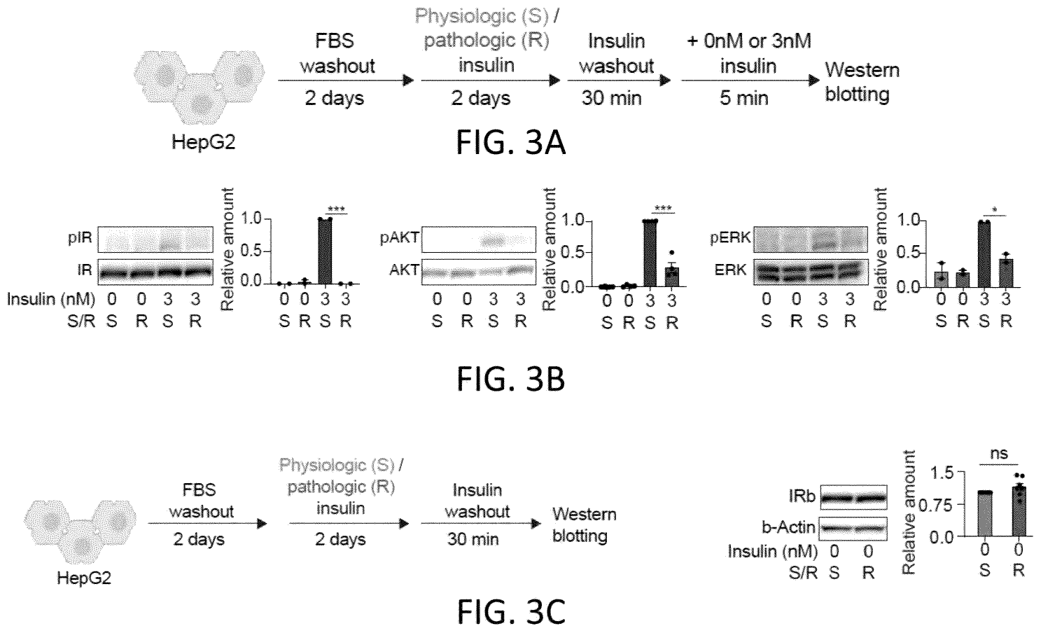

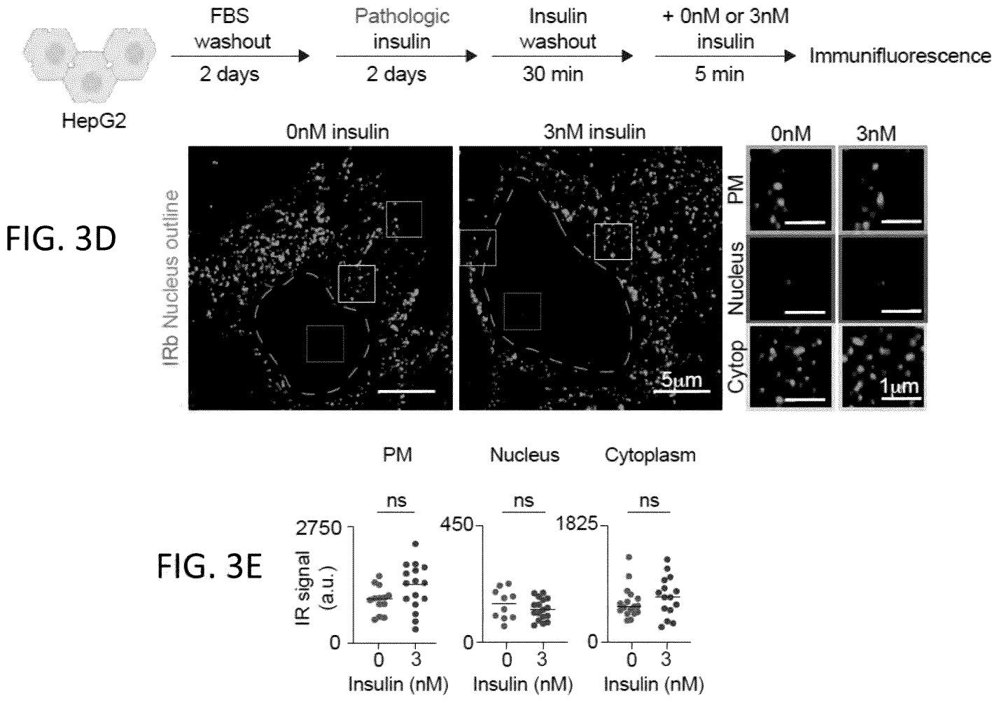

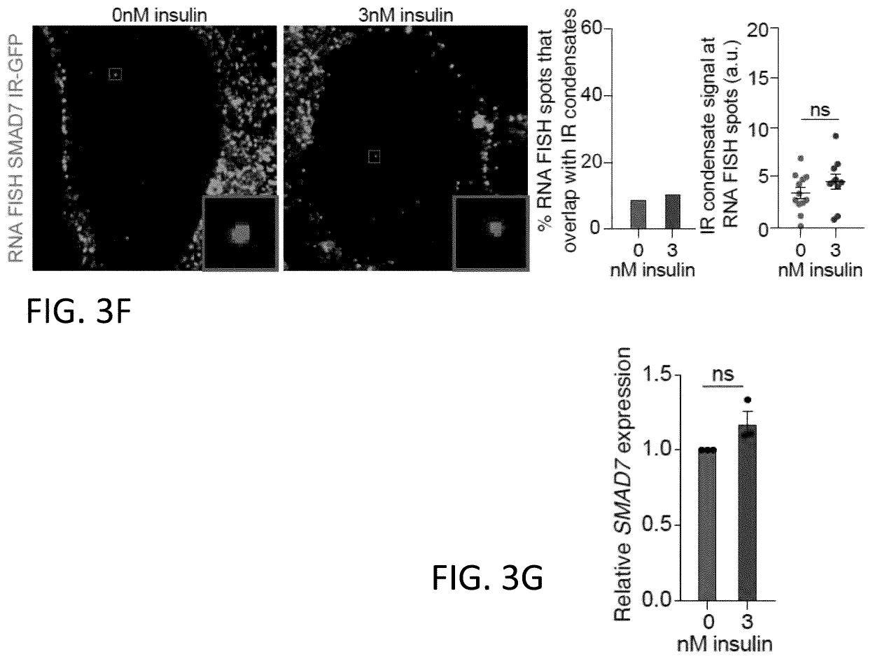

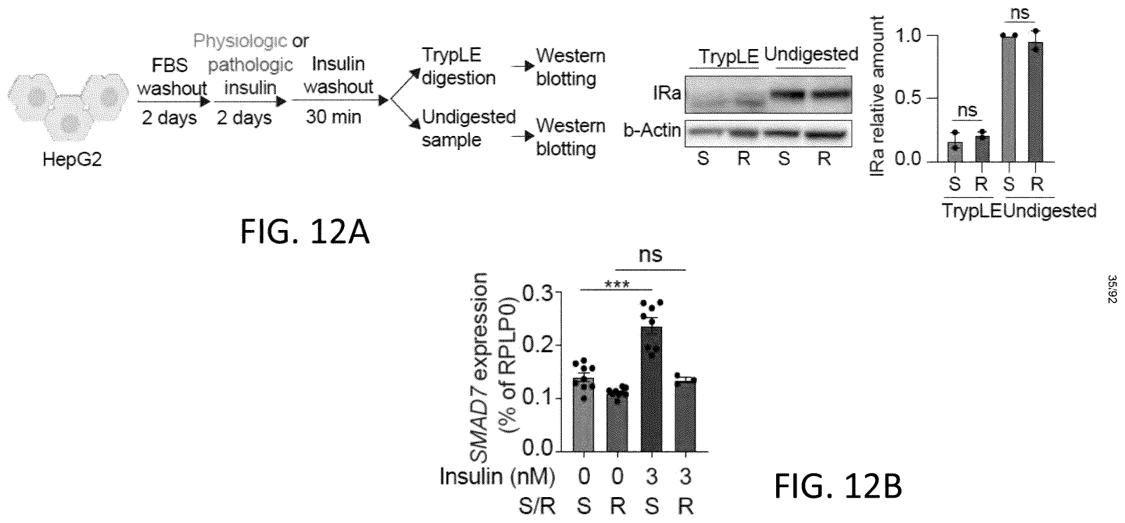

To investigate whether condensates associated with IR are altered in insulin resistance, HepG2 cells were exposed to either physiologic levels (0.1 nM) or pathologic levels (3 nM) of insulin for two days (FIG. 3A). Cells exposed to pathologic levels of insulin showed hallmarks of insulin resistance, including reduced phosphorylation of IR, AKT and ERK after insulin stimulation (FIG. 3B). Insulin sensitive and resistant cells contained similar amounts of IR (FIG. 3C; FIG. 12A), so the attenuated signaling was not due to a substantial change in IR levels.

IF imaging of IR in the insulin resistant cells revealed that it is incorporated into condensates at the plasma membrane, cytoplasm and nucleus in a manner similar to that observed for insulin sensitive cells (compare FIGS. 1B and 3D). However, in these insulin resistant cells, acute treatment with insulin (3 nM) did not promote incorporation of additional IR into condensates (FIG. 3D-E), in contrast to the effects observed in insulin sensitive cells (FIG. 1). Furthermore, insulin stimulation of these resistant cells did not increase the levels of IR in transcriptional condensates at the insulin responsive gene SMAD7 (FIG. 3F) nor did it cause upregulation of SMAD7 gene expression (FIGS. 3G and 12B). These results show that IR is incorporated into plasma membrane, cytoplasmic and nuclear condensates in both insulin sensitive and resistant HepG2 cells, but the ability of insulin stimulation to enhance IR incorporation into condensates is attenuated in the resistant cells.

### Altered IR Dynamics in Insulin Resistant Cells

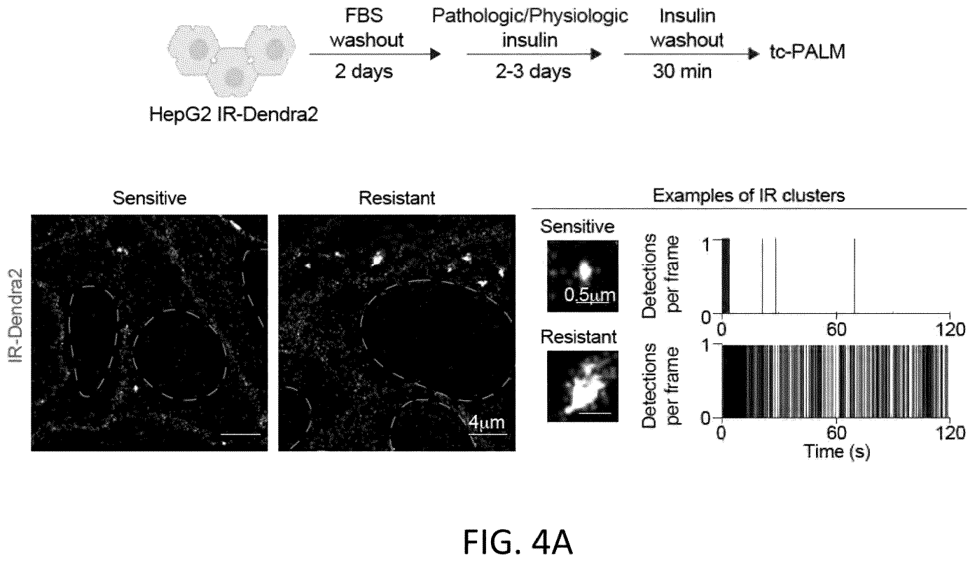

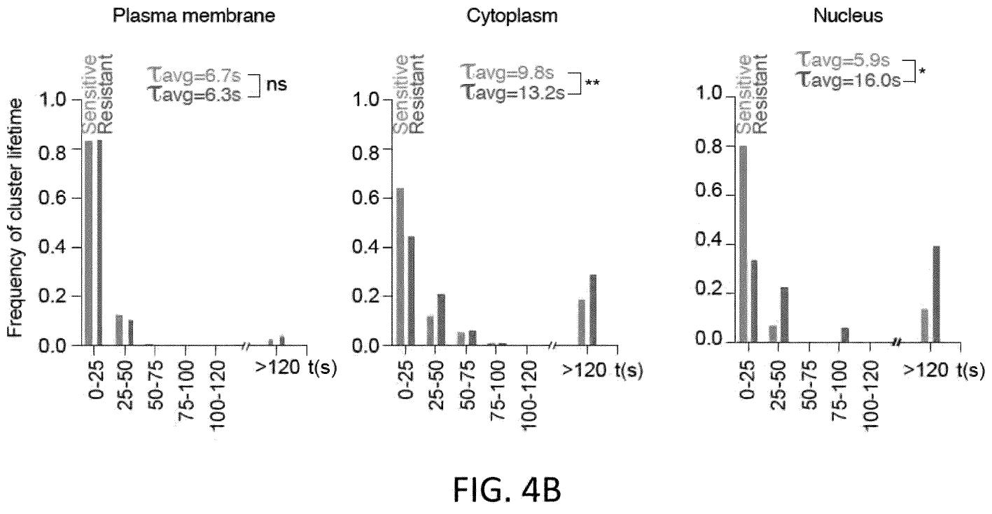

It was investigated whether the dynamics of IR condensates is altered in insulin resistant cells (FIG. 4). HepG2 cells expressing IR-Dendra2 were exposed to physiological levels (0.1 nM) of insulin to maintain insulin sensitivity, or pathologic levels (3 nM) of insulin to promote insulin resistance (FIG. 4A). tc-PALM was used to measure IR cluster dynamics in the insulin sensitive and resistant cells in the absence of insulin stimulation. The results showed that IR molecules remained in clusters for longer lifetimes in the cytoplasm and nucleus in insulin resistant cells relative to insulin sensitive cells (FIG. 4B). The average lifetime of short-lived IR clusters in sensitive versus insulin resistant cells increased from 9.8 s to 13.2 s in the cytoplasm and from 5.9 s to 16 s in the nucleus, and the percentage of long-lived IR clusters increased in the cytoplasm (1.4-fold) and nucleus (3.6-fold) (FIG. 4B). These results suggest that the insulin resistant state is associated with reduced IR condensate dynamics, reflected in the longer lifetime of these condensates, which may account for the attenuated responses observed during insulin stimulation.

### Metformin Rescues IR Condensate Behavior in Insulin Resistant HepG2 Cells

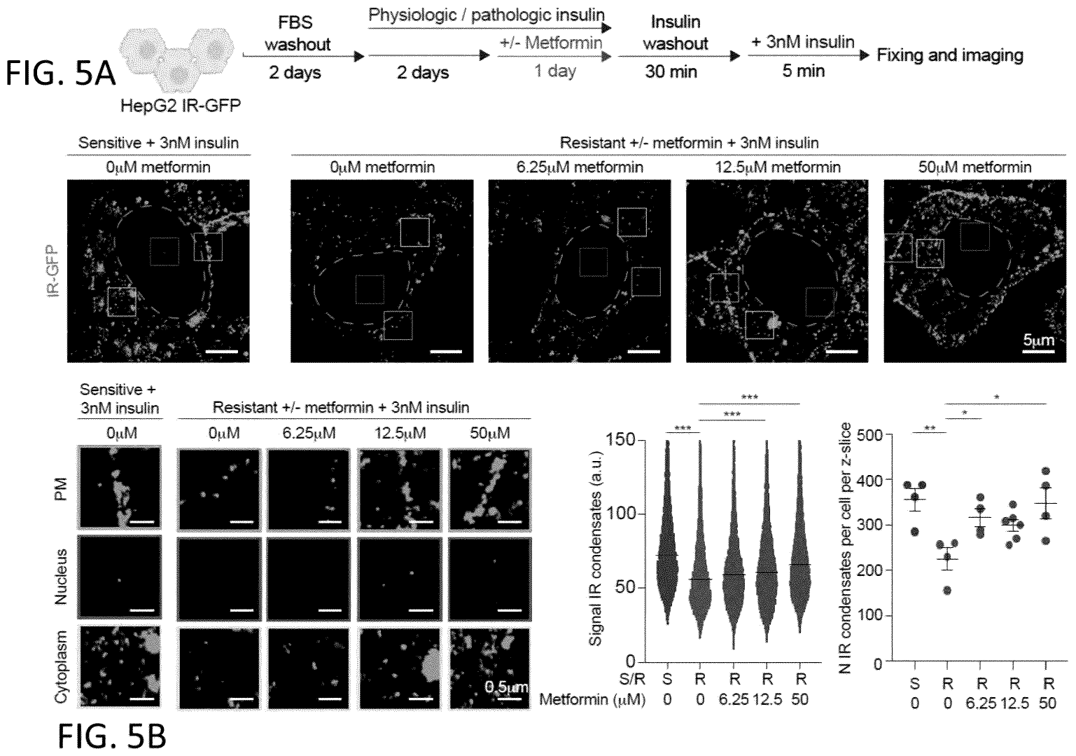

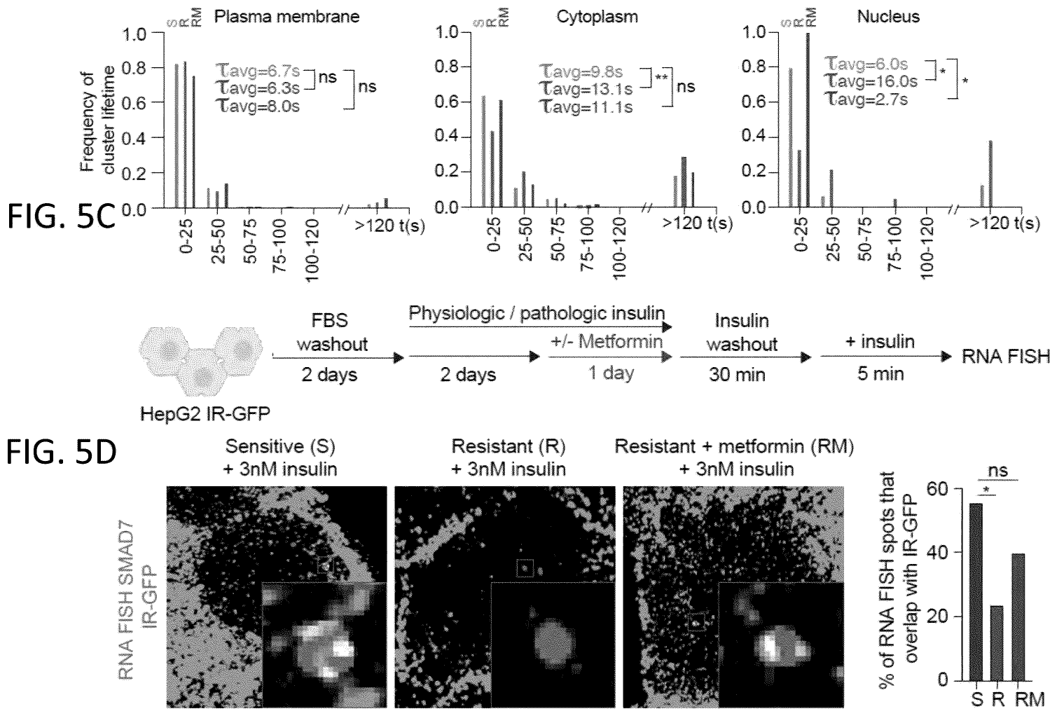

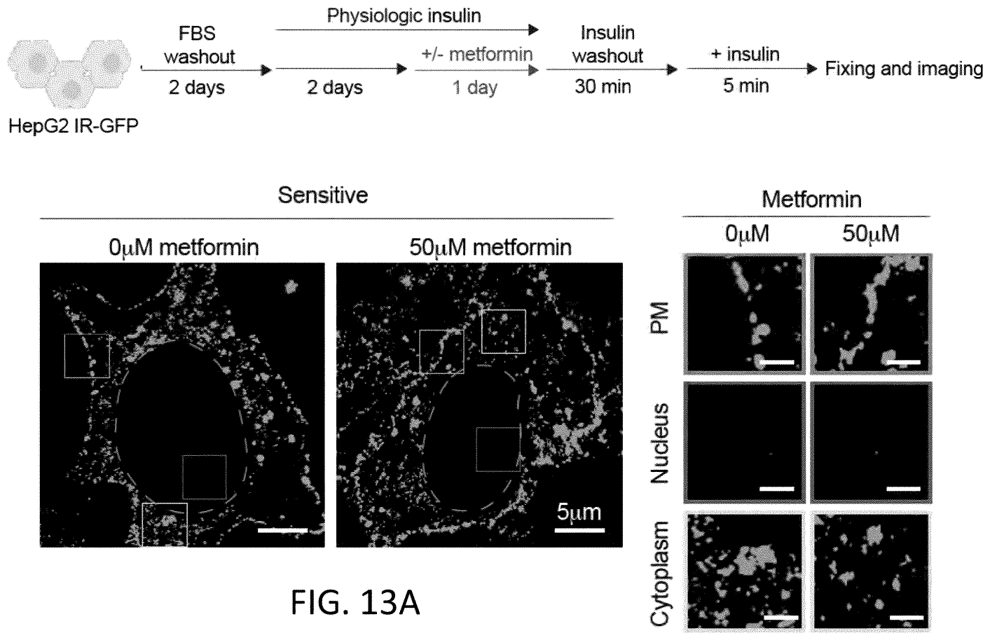

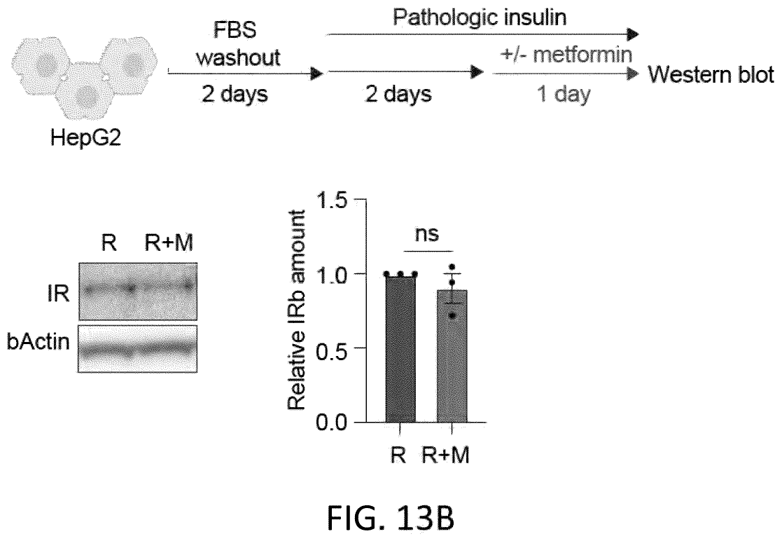

If condensate dysregulation contributes to insulin resistance, then one might expect that treatment of insulin resistant cells with metformin, the front-line drug for the treatment of insulin resistance in type II diabetes (T2D), would reverse the condensate dysregulation observed in insulin resistant HepG2 cells. Treatment of insulin sensitive HepG2 cells with 50 nM metformin had little or no effect on IR condensates (FIG. 13A). In contrast, treatment of insulin resistant HepG2 cells with metformin led to a dose-dependent increase in the number of IR containing condensates and the intensity of IR signal in these condensates (FIG. 5A,B). The rescue of IR condensates was not due to changes in IR levels (FIG. 13B). Furthermore, IR condensate rescue was significant at 12.5 uM metformin, which approximates the concentration of metformin in the plasma of T2D patients.

The effect of metformin treatment on IR condensate dynamics was next examined by performing tc-PALM in the insulin sensitive, resistant and metformin-treated resistant cells in their baseline state, in the absence of insulin stimulation. Metformin treatment of resistant cells had little effect on IR cluster lifetimes in the plasma membrane, but reduced cluster lifetimes in the cytoplasm and nucleus to times that were similar to those in insulin sensitive cells (FIG. 5C). For example, while ˜40% of cytoplasmic IR clusters in insulin resistant cells had a lifetime of 0-25 s, ˜60% of IR clusters in the cytoplasm of insulin sensitive and metformin-treated resistant cells had a lifetime of 0-25 s (FIG. 5C). Similarly, the frequency of nuclear IR clusters with 0-25 s lifetimes, which was reduced in the resistant cells relative to sensitive cells, was increased by the metformin treatment (FIG. 5C). Thus, metformin treatment rescues the less dynamic properties of IR-containing condensates in insulin resistant cells.

The effect of metformin treatment on IR-associated transcriptional condensates was then examined. In insulin resistant cells, insulin stimulation did not cause increased levels of IR in transcriptional condensates at the insulin responsive gene SMAD7, nor did it upregulate SMAD7 gene expression (FIG. 3F-G). However, metformin treatment of these insulin resistant cells increased IR levels in SMAD7 transcriptional condensates (FIG. 5D) and elevated SMAD7 gene expression (FIG. 5E), thus partially rescuing these defects in insulin resistant cells.

### High ROS Levels in Insulin Resistant Cells are Suppressed by Metformin

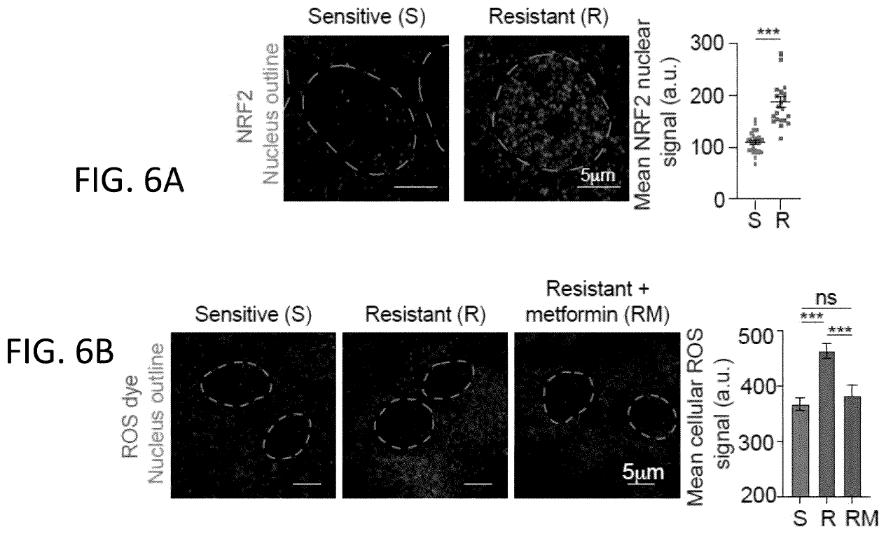

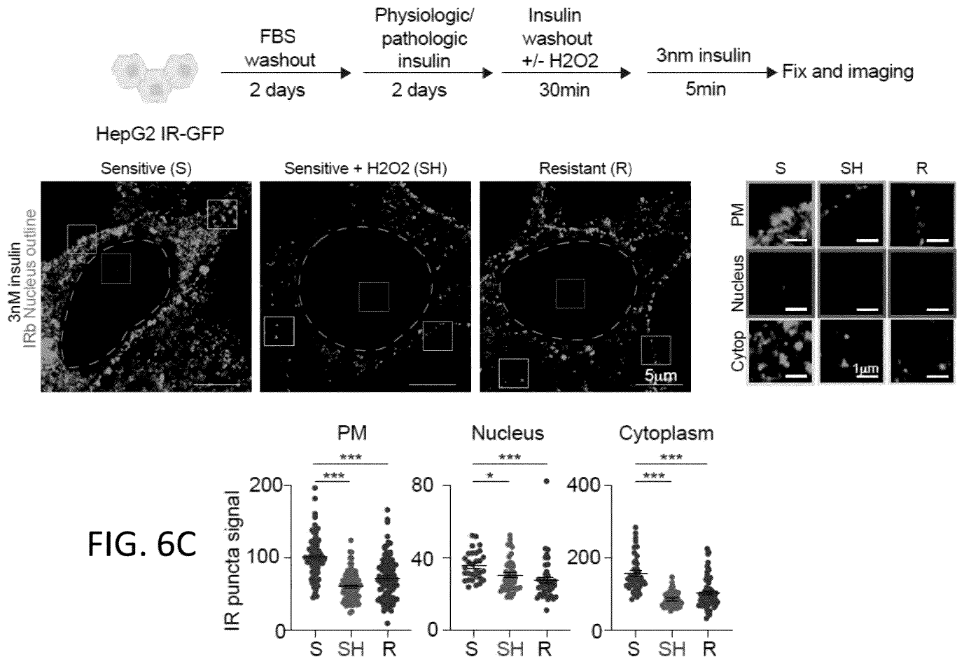

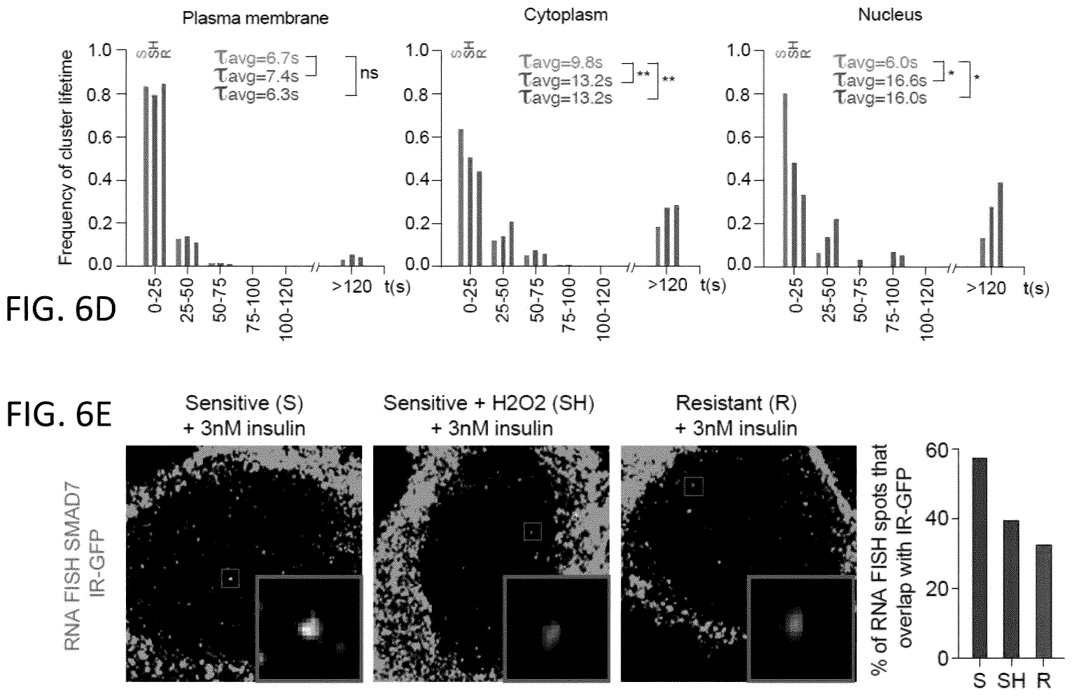

To test the hypothesis that high levels of reactive oxygen species (ROS) contribute to dysregulated IR condensates in insulin resistant cells, it was first determined if insulin resistant cells are subjected to higher levels of oxidative stress than insulin sensitive cells. Imaging of NRF2, a marker of oxidative stress, revealed that insulin resistant cells experienced higher levels of oxidative stress than insulin sensitive cells (FIG. 6A). Quantification of ROS using a ROS-sensitive dye revealed that ROS levels were higher in insulin resistant cells and, furthermore, that metformin treatment of these cells reduced ROS levels to those found in insulin sensitive cells (FIG. 6B). If oxidative stress causes condensate dysregulation in insulin sensitive cells, then treatment of insulin sensitive cells with the oxidizing agent H₂O₂ might be expected to phenocopy the effects seen with insulin resistance. Indeed, treatment of insulin sensitive cells with H₂O₂ for 30 min caused a reduction in the incorporation of IR into condensates with insulin stimulation, phenocopying the condensate behavior in insulin resistant cells (FIG. 6C). H₂O₂ treatment caused reduced IR condensate dynamics in insulin sensitive cells (FIG. 6D), as observed in insulin resistant cells (compare FIGS. 5C and 6D). Furthermore, H₂O₂ treatment caused a reduction in IR incorporation in transcriptional condensates (FIG. 6E). These results suggest that chronic hyperinsulinemia can lead to excess levels of ROS in insulin resistant HepG2 cells, that high levels of H₂O₂ alter IR incorporation into condensates, and that metformin can at least partially rescue dysfunctional IR-condensates behaviors as a consequence of reduced ROS.

### Condensate Rescue in Insulin Resistant Human Liver Spheroids and Liver Tissue

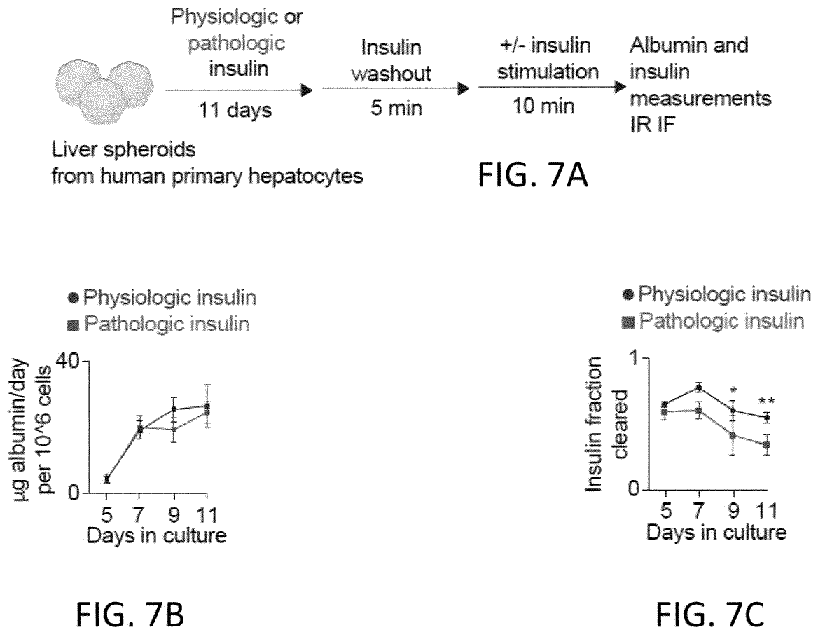

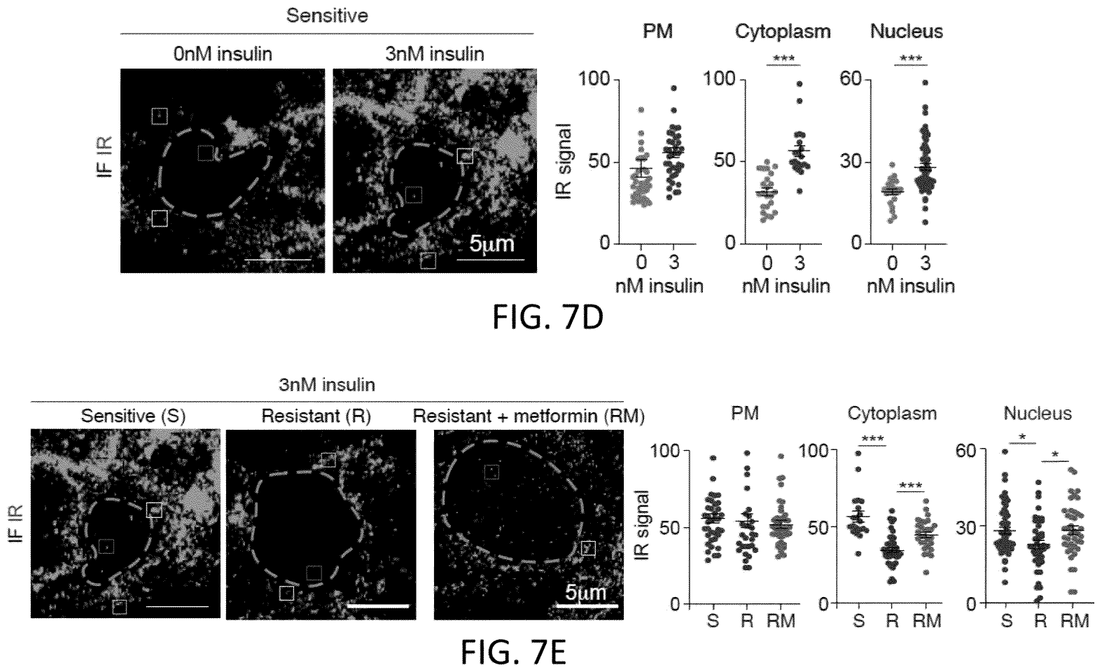

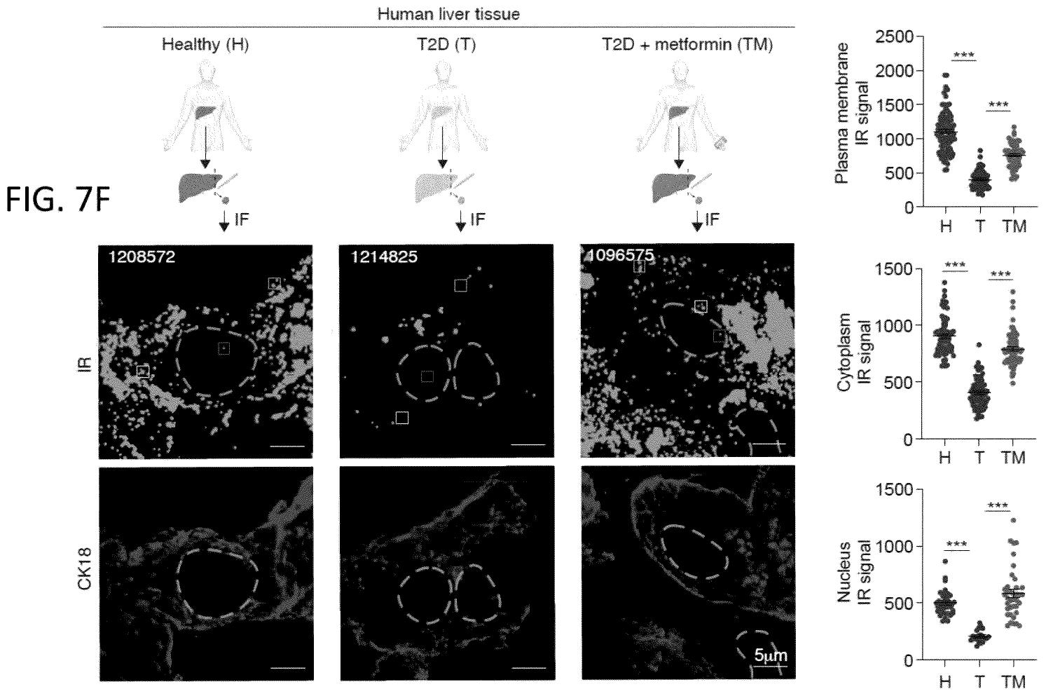

Applicant has observed IR-associated condensates in HepG2 cells, that these condensates are altered in insulin resistant cells, and that treatment of the resistant cells with metformin can rescue IR-associated condensate behavior. Applicant turned to primary human hepatocytes, the “gold standard” for evaluating hepatic insulin signaling, to investigate whether these effects are also observed in primary cells (FIG. 7). Primary human hepatocytes can form three-dimensional spheroids and can be cultured for days with physiological or pathological concentrations of insulin while maintaining their cell identity and function (IG. 7A-C). Primary human hepatocyte spheroids are insulin sensitive if cultured with physiological concentrations of insulin, and insulin resistant if subjected to insulin levels characteristic of chronic hyperinsulinemia (FIG. 7C). IF imaging of insulin sensitive human liver spheroids revealed that IR is found in condensates at the plasma membrane, cytoplasm and nucleus, and that insulin stimulation leads to an increase in IR signal intensity in cytoplasmic and nuclear condensates (FIG. 7D). In insulin resistant spheroids, by contrast, IR incorporation into cytoplasmic and nuclear condensates was diminished (FIG. 7E), but metformin treatment partially rescued this attenuation of IR condensate signal (FIG. 7E). Applicant concludes that insulin resistant liver spheroids show an attenuated IR-condensate response to insulin that is rescued by metformin treatment.

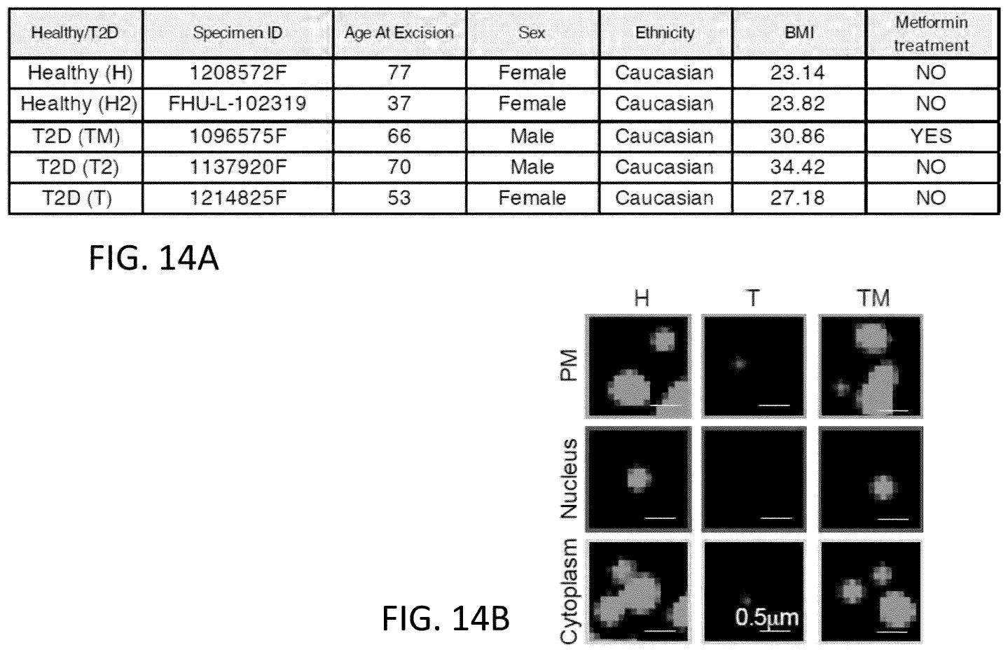

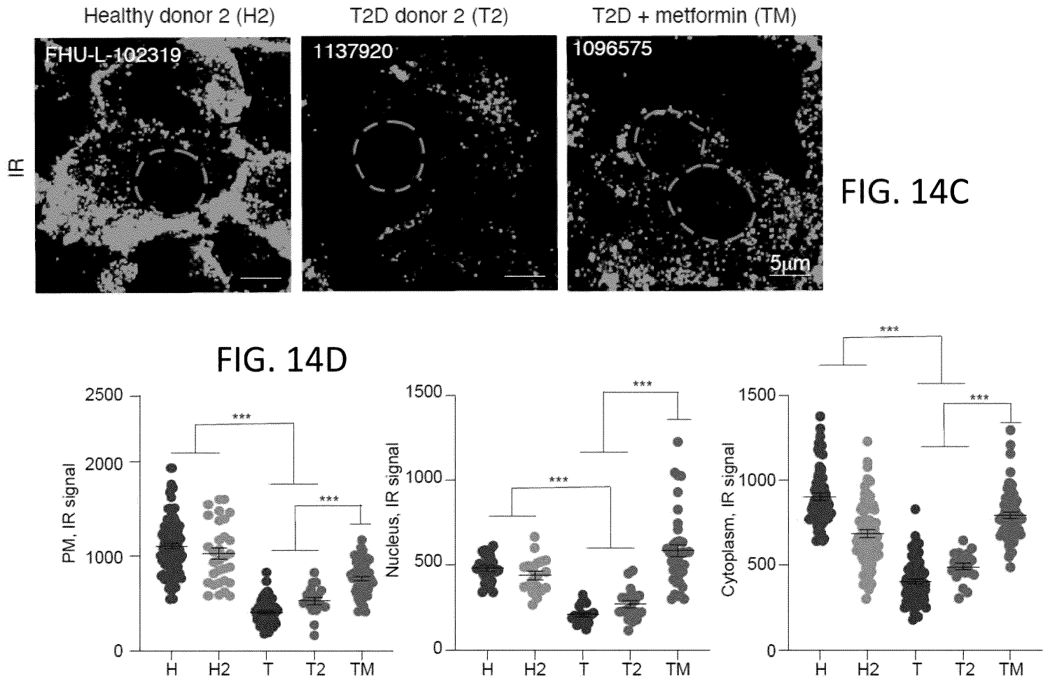

It was reasoned that if the IR condensate phenotypes observed in cells are relevant to insulin sensitivity and resistance in human tissues, IR containing condensates should be observed in human liver tissue, exhibit dysfunction in liver tissue from donors with T2D, and that this might be rescued by metformin treatment. Human liver tissue was obtained from two healthy donors, two donors with T2D and one donor with T2D under treatment with metformin (FIG. 14A). IF for CK18 was used to assess tissue quality, cell morphology and as a marker for hepatocytes. Imaging of these tissues revealed that IR occurs in condensates in the plasma membrane, the cytoplasm and the nucleus of hepatocytes from healthy donors (FIG. 7F, 14B-D). This IR condensate signal was substantially reduced in condensates in tissue from donors with T2D (FIG. 7F, 14B-D), as observed for both insulin resistant HepG2 cells and human hepatocyte spheroids (FIGS. 1B,C, 3D, E and 7D,E). Remarkably, liver tissue from the donor with T2D who was treated with metformin had increased IR signals in condensates that approximated those seen in tissue from healthy donors (FIG. 7F, 14B-D). These results indicate that hepatocytes within human liver tissue experience similar condensate phenotypes observed with insulin sensitivity and resistance in the HepG2 and primary hepatocyte spheroid model systems.

### DISCUSSION

A condensate model for insulin receptor (IR) function in normal conditions and when dysregulated in chronic hyperinsulinemia-induced insulin resistance was considered. The results reveal that IR is incorporated into dynamic condensates at the plasma membrane, in the cytoplasm and in the nucleus and acute insulin stimulation promotes additional incorporation of IR into cytoplasmic and nuclear condensates. Chronic hyperinsulinemia alters the dynamics of these condensates. The altered IR-condensate dynamics observed in chronic hyperinsulinemia are rescued by metformin, the first-line drug for T2D. Elevated ROS levels can account for the altered condensate dynamics in chronic hyperinsulinemia and metformin reduces ROS levels in insulin resistant cells.

Recent studies have shown that the components of diverse signaling pathways, including those involving receptor tyrosine kinases, T cell receptor, ß-catenin/WNT and others, involve the assembly of protein molecules into liquid-like biomolecular condensates at the plasma membrane, in the cytoplasm and nucleus (39-42, 70). The evidence herein indicates that this is also the case for the insulin receptor. The evidence herein indicates that chronic hyperinsulinemia also produces altered IR condensate dynamics, and that this alteration is associated with reduced IR-mediated transcriptional activation at insulin-responsive genes.

It was found that dysregulated ROS levels are associated with altered IR condensate dynamics, which may provide a mechanism to explain the findings herein. Increased ROS can cause protein oxidation (71), which can alter the dynamic behaviors of proteins characteristic of liquid-like condensates (62). ROS-induced alteration of proteins in condensates may be a common mechanism in the pathogenesis of insulin resistance-associated diseases, including T2D, metabolic syndrome, non-alcoholic fatty liver disease (NAFLD), polycystic ovarian syndrome (PCOS), and Alzheimer's Disease (72-75). Indeed, metformin has been shown to decrease ROS production and improve patient outcomes in T2D and other diseases characterized by high ROS levels (66-69, 76-79). The mitochondrial respiratory chain complex is the primary target of metformin, but it may be the reduction in ROS levels and the consequent benefit to condensate dynamics that is key to enhancing the dynamics of IR condensates.

A condensate model for insulin receptor (IR) dysfunction function has implications for development of novel therapeutics for T2D and other diseases that involve condensate dysregulation. For example, the condensate assays described here might be leveraged to develop novel therapeutics that improve clinical outcomes for patients who cannot tolerate metformin or become resistant to the drug with prolonged use. Such therapeutics might also provide benefits to patients with other diseases where condensate dysregulation is also thought to play a role (80-82).

### Materials and Methods

### Human Liver Donor Samples

Samples of human livers were purchased from BioIVT. Sample ID numbers and donor information are reported in FIG. 14A. Frozen samples were embedded in OCT compound (Tissue-Tek, 4583), re-frozen on dry ice and stored at −80° C. Embedded samples were sectioned using the cryostat at the W. M. Keck Microscopy Facility, MIT. Sectioning was performed at −21° C. to generate 10 um-thick slices that were then placed on a Superfrost Plus VWR Micro Slides (VWR, 48311-703) and stored at −20° C.

### Cell Culture

HepG2 cells (ATCC HB-8065™) were cultured in EMEM (ATCC 30-2003) supplemented with 10% FBS (Sigma Aldrich, F4135) at 37° C. with 5% CO2 in a humidified incubator. For passaging, cells were washed in PBS (Life Technologies, AM9625) and TrypLE Express Enzyme (Life Technologies, 12604021) was used to detach cells from plates and dissociate cell clumps. To ensure proper cell dissociation, cells were incubated with TrypLE at 37° C. with 5% CO2 in a humidified incubator for 5 min, they were then mechanically dissociated pipetting them up and down using a 5 mL serological pipette attached to an unfiltered 200 ul pipette tip 8 times. Cells were incubated 5 more minutes at 37° C. with 5% CO2 in a humidified incubator, and were then further dissociated mechanically as previously described. TrypLE was quenched with EMEM 10% and cells were plated in new tissue culture-graded plates.

For human liver spheroid experiments, primary human hepatocytes from a 50-year-old male donor (BioIVT; lot #SMC) were used. Cells were thawed in Cryopreserved Hepatocyte Recovery Media (CHRM, ThermoFisher), spun down at 100 g for 8 min, and resuspended in seeding medium (William's E with 5.5 mM glucose, 2 mM GlutaMax, 15 mM HEPES, 5% FBS, 1% Pen/Strep, 100 nM hydrocortisone, and insulin 200 pM or 800 pM corresponding to the proper experimental group). Spheroids were formed using custom alginate microwells. In brief, 120,000 cells were seeded per well and spun at 50 g for 2 min to seed microwells, and cultured in a volume of 300 uL seeding medium. After 24 hours, cells were switched to maintenance media for the remainder of the experiment. This maintenance media was composed of William's E plus 6.25 μg/ml transferrin, 6.25 ng/ml selenium, 0.125% fatty acid-free BSA, 20 μM linoleic acid, 5.5 mM glucose, 2 mM GlutaMax, 15 mM HEPES, 0.5% Pen/Strep, and 100 nM hydrocortisone. Insulin was supplemented with concentrations adjusted to mimic healthy and disease-inducing states, either 200 pM for physiological or 800 pM for pathologic insulin levels. Media was exchanged every 48 hours throughout the experiment.

HEK293T cells (ATCC, CRL-3216) were used for the production of purified IRb protein. HEK293T cells were cultured in DMEM (GIBCO, 11995-073) supplemented with 10% FBS (Sigma Aldrich, F4135), 2 mM L-glutamine (Gibco, 25030) and 100 U/mL penicillin-streptomycin (Gibco, 15140), at 37° C. with 5% CO2 in a humidified incubator.

### Endogenously-Tagged Cell Line Generation

CRISPR/Cas9 system was used to generate genetically modified HepG2 cell lines. Target sequences were cloned into a plasmid containing sgRNA backbone, a codon-optimized version of Cas9 and mCherry. For each target, two Cas9 plasmids were generated. For the generation of the IR-mEGFP and IR-Dendra2 endogenously tagged lines, homology directed repair templates were cloned into pUC19 using NEBuilder HiFi DNA Assembly Master Mix (NEB, E2621S). The homology repair template consisted of mEGFP or Dendra2 cDNA sequence flanked on either side by 800 bp homology arms amplified from HepG2 genomic DNA using PCR (FIG. 9A). The following sgRNA sequences with PAM sequence in parentheses were used for CRISPR/Cas9 targeting:

sgRNA_IR_C-term_1:
(SEQ ID NO: 1)
CACGGTAGGCACTGTTAGGA(AGG)
sgRNA_IR_C-term_2:
(SEQ ID NO: 2)
TAGGCACTGTTAGGAAGGAT(TGG)

To generate genetically modified cell lines, 2×106 cells were transfected with 500 ng of Cas9 plasmid 1, 500 ng of Cas9 plasmid 2, and 1,000 ng of non-linearized homology repair template using Lipofectamine 3000 (Invitrogen, L3000). Cells were sorted 48 hours after transfection for the presence of the mCherry fluorescent protein encoded on the Cas9 plasmid to enrich for transfected cells. This population of cells were allowed to expand for 1.5 to 2 weeks before sorting a second time for the presence of mEGFP or Dendra2 and single cells were plated into individual wells of a 96-well plate. The single cells were cultured in conditioned EMEM media for 1-1.5 months. 20-30 colonies were screened for successful targeting using PCR genotyping to confirm insertion. PCR genotyping was performed using Phusion polymerase (Thermo Scientific, F531S). Using the following primers, PCR products were amplified according to manufacturer specifications:

IR_fwd:
(SEQ ID NO: 3)
GGAGAATGTGCCCCTGGAC
IR_rev:
(SEQ ID NO: 4)
TTGGTAACCAAACGAGTCCACCT

To make conditioned media, HepG2 cells were cultured in fresh EMEM media (EMEM (ATCC 30-2003) supplemented with 10% FBS (Sigma Aldrich, F4135) for 3 days and saved the media (old EMEM media). The composition of conditioned EMEM media is as follows: 50% fresh EMEM media and 50% old EMEM media. The conditioned media was filter sterilized prior to use.

### Cell Treatments

For insulin sensitivity and resistance experiments in HepG2 cells, cells were washed with EMEM once and cultured in EMEM for two days. Cells were then treated for two days with either physiological (0.1 nM) or pathological (3 nM) levels of insulin (Sigma Aldrich, I9278-5ML) in EMEM supplemented with 1.25% fatty acid-free bovine serum albumin (BSA, Sigma Aldrich, A8806-5G). To wash out insulin, cells were washed in EMEM seven times, which includes three quick washes, three 5 min washes and a long 20 minute-wash in EMEM at 37° C. In order to investigate insulin response, cells were acutely treated for 5 min with insulin diluted in EMEM supplemented with 1.25% fatty acid-free BSA at 37° C. with 5% CO2 in a humidified incubator. The concentration of insulin used is reported in the figures.

For insulin sensitivity and resistance experiments in human liver spheroids, cells were cultured in maintenance media composed of William's E plus 6.25 μg/ml transferrin, 6.25 ng/mL selenium, 0.125% fatty acid-free BSA, 20 uM linoleic acid, 5.5 mM glucose, 2 mM GlutaMax, 15 mM HEPES, 0.5% Pen/Strep, 100 nM hydrocortisone and either 0.2 nM (to maintain insulin sensitivity) or 0.8 nM (to induce insulin resistance) insulin for 11 days. Media was exchanged every 48 hours throughout the experiment. On day 11, spheroids were rinsed with insulin-free William's E and then stimulated with either 0 or 3 nM insulin for 10 min at 37° C. with 5% CO2 in a humidified incubator.

Metformin (SIGMA, D150959-5G) was resuspended in sterile water to a concentration of 1M and diluted to the reported concentrations in the figures in cell media.

For oxidative stress, insulin sensitive cells were treated with 20 mM H₂O₂ (SIGMA) for 30 min.

### Measure of Percentage Cell Viability

Cells were detached from plates and dissociated from clumps using TrypLE. TrypLE was quenched with EMEM supplemented with 10% FBS. Dead cells were stained with trypan blue and the percentage of cell viability was then measured using the Countess II FL (Applied Biosystems, A27977).

siRNA Experiments

HepG2 cells were reverse transfected using Lipofectamine™ RNAiMAX Transfection reagent following the manufacturer's instructions. Cells were dissociated using TrypLE as previously described, cells were then seeded in 6-multiwells in 1 ml EMEM supplemented with 10% FBS and the transfection reagent. Cells were cultured with the transfection reagents for 2-3 days prior to collection for Western blot and immunofluorescence.

siRNA used were INSR siRNA pool (Dharmacon Inc, L-003014-00-0005) and ON-TARGETplus Non-targeting Control Pool (Horizon Discovery, D-001810-01-05).

### Western Blot

Cells were washed with ice-cold PBS and lysed in Cell Lytic M (Sigma-Aldrich C2978) supplemented with protease and phosphatase inhibitors (Sigma Aldrich, 11873580001 and 4906837001) directly on the wells. Lysates were placed into a 1.5 ml tube and mixed at 4° C. for 20 min and then centrifuged at 13,000 rpm for 15 min. Protein concentration was determined using a BCA Protein Assay Kit (Life Technologies, 23250) according to the manufacturer's instructions. Equal amounts of protein (5-50 μg per sample) were separated on 10% or 12% Bis-Tris gels in 5% XT MOPS running buffer (Bio-Rad Laboratories, 1610788) at 100V until dye front reached the end of the gel. Protein was then transferred to a 0.45 μM PVDF membrane (Millipore, IPVH00010) in ice cold transfer buffer (25 mM Tris, 192 mM glycine, 20% methanol) at 300 mA for 1 hour or 250 mA for 2 hours at 4° C. After the transfer, membranes were blocked in 5% non-fat milk (LabScientific, M0842) dissolved in TBST (2% Tris HCl pH 8.0, 1.3% 5M NaCl, 0.05% Tween 20) or 5% BSA (VWR, 102643-516) in 1×TBST for 15 min to 1 hour at room temperature shaking. Membranes were then incubated overnight at 4° C. in 1:1000 primary antibody in 5% non-fat milk in TBST or 5% BSA in TBST. BSA was used for immunoblotting phosphorylated proteins, otherwise milk was used. Membranes were then washed three times for 5 min in TBST shaking at room temperature prior to incubation in 1:10,000 secondary antibody in 5% non-fat milk in TBST for 1 hour at room temperature. This was followed by three 10-minute washes in TBST. Membranes were developed with ECL substrate (Thermo Scientific, 34080) and imaged using a CCD camera. Immunoblot quantification was performed using the “analyze gel” tool on Fiji/ImageJ v2.1.0/153c.

The following primary antibodies were used for WB: anti-phosphorylated insulin receptor (Abcam, ab60946 and Cell Signaling 3026), anti-insulin receptor beta (Cell Signaling, 23413; Bethyl, A303-712A or Cell Signaling, 3025), anti-insulin receptor alpha (Cell Signaling, 74118), anti-phosphorylated AKT (Cell Signaling, 4056), anti-AKT (Cell Signaling, 9272), anti-phosphorylated ERK (Cell Signaling, 4377), anti-ERK (Cell Signaling, 9102), anti-beta Actin (Sigma Aldrich, A5441). The following secondary antibodies were used: donkey anti-rabbit IgG (Cytiva Life Sciences, NA934-1ML) and sheep anti-mouse IgG (Sigma Aldrich, NXA931V).

For quantitative western blot analysis, equal numbers of cells were cultured on a 6-well plate. To estimate the number of cells per well, cells in two wells were dissociated with TrypLE (Life Technologies, 12604021) and counted using the Countess II (Applied Biosystems, A27977). Cells from another well were lysed on the plate as described above. A dilution series of purified IRb-mcherry and HepG2 cellular lysate was separated on 10% Bis-Tris gels in 5% XT MOPS running buffer. Immunoblotting was performed as above. Bands were quantitated using Fiji/ImageJ v2.1.0/153c, from which applicant calculated the estimated number of molecules of IRb per HepG2 cell.

### Immunofluorescence

HepG2 cells, human liver spheroids and human tissue liver sections were fixed in 4% PFA (VWR, BT140770-10X10) in PBS for 10 min at room temperature. Cells were washed in PBS three times for 5 min and then blocked with 4% IgG-free BSA (VWR, 102643-516) for 15-60 min at room temperature. Cells were then incubated with 1:500 primary antibody in 4% IgG-free BSA at 4° C. overnight. The next day, cells were washed three times with PBS and incubated with 1:500 secondary antibodies in 4% IgG-free BSA at room temperature for 1 hr covered in foil. Cells were washed three times with PBS for 5 min. DNA was stained using 1:5000 Hoechst in PBS for 5 min at RT. Cells were washed three times with PBS for 5 min, stored at 4° C. until imaging. For tissue sections, samples were mounted using Vectashield mounting media (Vector Laboratories, Inc, H-1000). LSM880 microscope with Airyscan detector (ZEISS) was used for image acquisition. Images were then processed using Fiji/ImageJ v2.1.0/153c.

Primary antibodies used were anti-insulin receptor beta (Cell Signaling, 23413), anti-NRF2 antibody (Abcam, ab62352) and anti-cytokeratin 18 (CK18) (Abcam, ab668). Secondary antibodies used were Alexa Fluor 488 goat anti-rabbit IgG (Thermo Fischer Scientific, A11008), Alexa Fluor 647 goat anti-rabbit IgG (Thermo Fischer Scientific, A21244), Alexa Fluor 568 goat anti-mouse IgG (Thermo Fischer Scientific, A11031). Images were acquired at LSM880 Microscope with Airyscan detector with 63× objective using Zen Black software (ZEISS) at the W. M. Keck Microscopy Facility, MIT. Images were then processed using Fiji/ImageJ v2.1.0/153c.

### Live-Cell Imaging

Cells expressing endogenous IR tagged with GFP were grown on 35 mm glass bottom dishes (MatTek Corporation, P35G-1.5-20-C). Cells were imaged at 37° C. using the SM880 Microscope with Airyscan detector with 63× objective and Zen Black software (ZEISS) at the W.M. Keck Microscopy Facility, MIT. Images were then processed using Fiji/ImageJ v2.1.0/153c.

### ROS Staining and Live-Cell Imaging

Cells expressing endogenous IR tagged with GFP were treated with ROS dye (Abcam, ab186029) following the manufacturer's instructions. After maintaining insulin sensitivity or inducing insulin resistance, cell media was removed and 1000×ROS Deep Red Stock Solution was diluted to 1× in PBS with calcium and magnesium and was added to the cells. Cells were incubated at 37° C. with 5% CO2 in a humidified incubator for 30 min. Cells were imaged at 37° C. using the LSM880 Microscope with Airyscan detector with 63×objective and Zen Black software (ZEISS) at the W.M. Keck Microscopy Facility, MIT. Images were then processed using Fiji/ImageJ v2.1.0/153c.

### RNA FISH

Pipettes and bench were treated with RNaseZap (Life Technologies, AM9780). Cells were fixed with 4% PFA in PBS for 10 min at RT. Cells were washed three times with PBS for 5 min. Cells were permeabilized with 0.5% TritonX100 in 1×RNasefree PBS (Invitrogen, AM9625) for 10 min at room temperature. Cells were washed three times with RNasefree PBS for 5 min. Cells were washed once with 20% Stellaris RNA FISH Wash Buffer A (Biosearch Technologies, Inc., SMF-WA1-60), 10% Deionized Formamide (EMD Millipore, S4117) in RNase-free water (Life Technologies, AM9932) for 5 min at room temperature. Cells were then hybridized with 90% Stellaris RNA FISH Hybridization Buffer (Biosearch Technologies, SMF-HB1-10), 10% Deionized Formamide, 12.5 μM Stellaris RNA FISH probes designed to hybridize introns of the SMAD7 transcript. Hybridization was performed O/N at 37° C. Cells were then washed twice with Wash Buffer A for 30 min at 37° C. and once with Stellaris RNA FISH Wash Buffer B (Biosearch Technologies, SMF-WB1-20) for 5 min at room temperature. Images were acquired at LSM880 Microscope with Airyscan detector with 63× objective using Zen Black software (ZEISS) at the W.M. Keck Microscopy Facility, MIT.

Stellaris® FISH Probes, Custom Assay with
TAMRA Dye (LGC Bioserch, SMF-1001-5).
Sequence:
(SEQ ID NO: 5)
acacttcacacaccaaagca gacatgtgctgcaaaatcct
gggaacaattacagctccta accagtgagtttctctaagt
caacatgaaatgcgtcccat atgccaaatgcccaaaactc
gcaggtaccccacaaaaaag caagctcagttcctgaagag
aacaataccctcagtctcag aacatcctgagcgtgttatt
ctttcaaaagctgccacttc gagctcaaactttgaagcct
ctcgctacaaagaggacagc gccaataaagttcccttttg
ttacaaaactctctgccctg gaaaaggaggaggtttgggc
aggaaaacagcacacagggc ctgcaatgctggaattccaa
aaaaatgtcccattttccca acaatctcctggaagcaagt
ggattgtctatctgtacgtc ctttcaaaagacacccatcc
atgacacatccatgtccaag tgatagcttccaacacatgc
caaaatgaaagcccccagac ggaggctagaagatgagtct
gatactgactcaaggacgct tatttttgaaggctctcctc
ggggtgcattttcagattat gcaatgtttcctaagctact
cccagttcatgatcacaatt tcagtgaaaccatgagctgt
tcactccacaatggcattag catgctacagtgtcaccaaa
tacaagtctacgcagctaca caactgctttgagagtgctg
ttttagtgtgcgagcaagtc aagtgtctaccatcatcagg
cttgctcaggtaacacacag aaaccagagggaactttcct
gaacttgggcaaggacatca tacagtatcctttcacgagg
tgaaaatagcccggagagga tcttggtgtgttaaccacta
ccaacaggtttaggtagagt ttctctctctagcagatact
gagttggcttcgtaagaagc gtgaaagttatcaccctgtt

### Imaging Analyses

Fiji/ImageJ v2.1.0/153c was used to quantify IR fluorescence intensity per cell for the IR antibody validation experiment. With the polygon selection tool, a polygon was drawn around a cell outline. The average fluorescence intensity in the polygon (=in the cell) was determined using the measure tool on Fiji/ImageJ v2.1.0/153c. Background was then subtracted by a threshold determined by averaging the background intensity in a rectangular region of interest outside of the cells.

To quantify IR fluorescent signal in puncta, Fiji/ImageJ v2.1.0/153c was used. A circle or an oval was drawn around IR puncta using the oval selection tool and the average fluorescence intensity in the circle/oval (=in the puncta) was determined using the measure tool on Fiji. Background was then subtracted as previously described. To quantify IR fluorescent signal at puncta in various cellular compartments, applicant identified the location of the plasma membrane, cytoplasm, nucleus and RNA FISH spots as follows. The plasma membrane location was identified based on IR immunofluorescence signal, IR-GFP fluorescent signal or CK18 immunofluorescence signal or cell edge. The nucleus was determined by the Hoechst stain for immunofluorescence and tc-PALM experiments or by IR-GFP fluorescence when HepG2 cells expressing IR-GFP were imaged. Given the overlap between Hoechst fluorescence and GFP fluorescence, artefactual green fluorescence coming from the Hoechst confounded IR puncta identification in the nucleus. Since the differences in IR puncta intensity and concentration between cytoplasm and nucleus in HepG2 cells was clear, nuclear outline was easily drawn based on IR-GFP fluorescent signal. Cytoplasm was considered the region between the plasma membrane and the nucleus. RNA FISH spots were identified based on RNA FISH probe fluorescence. Within the FISH spot, IR intensity was measured using the measure tool.

Identification of overlap between RNA FISH spots and IR puncta. An IR punctum overlapped with an RNA FISH spot if the IR signal intensity at the RNA FISH spot was at least 3 times higher than the nuclear background (average GFP signal in a rectangle of the nucleus not containing IR puncta).

To estimate the number of IR puncta at the plasma membrane, cytoplasm, nucleus or in entire cells, IR puncta were initially counted in a cell slice using Fiji. The number obtained from the cell slice was then multiplied based on the estimated size of the plasma membrane, cytoplasm, nucleus or entire cell, which were obtained considering the length and width of the cell under investigation and the estimated height (˜5 um).

To quantify the properties of IR puncta upon metformin treatment, Airyscan images from all conditions were maximally-projected in the z-plane and background subtracted by a threshold determined by averaging the background intensity in a rectangular region of interest outside of the cells. For segmenting IR puncta, the images were first subtracted by a median-filtered image (10 px) and then subjected to a Laplace of Gaussian filter (sigma=1). Filtered images were then thresholded on signal intensity (intensity >mean image intensity+2*standard deviation of image intensity). Thresholded binary images were then subjected to a morphological opening operation with a 3×3 filled structuring element to remove small objects. The mean intensity of the background-subtracted raw image was then measured for each segmented puncta (c-in), and background intensity (c-out) was calculated from the mean intensity of an inverted mask of the called puncta. The partition ratio of each segmented puncta was calculated as (c-in/c-out), and the size of each puncta was measured as the pixel area of the segmented puncta.

Quantification of the ROS dye fluorescence intensity per cell was performed using Fiji. Using the polygon selection tool on Fiji/ImageJ v2.1.0/153c, a polygon was drawn around a cell outline, which was identified by looking at the IR-GFP channel. The average ROS dye fluorescence intensity in the polygon (=in the cell) was determined using the measure tool on Fiji/ImageJ v2.1.0/153c. This was repeated for 107 insulin-sensitive cells, 70 insulin-resistant cells and 134 insulin-resistant cells treated with metformin.

Fusion, fission, or deformation events were identified in time-lapse imaging of the endogenously-tagged IR-GFP HepG2 line. To confirm bona fide fusion or fission events, applicant compared the measured fluorescence intensity to an expected fluorescent intensity of such events calculated as follows.

Iexp=Io*Ao/A1

Iexp is the expected fluorescent signal if the punctum underwent deformation, fusion and fission. Io and Ao are the fluorescent signal intensity (I) and area (A) of the punctum before the potential deformation fusion and fission event. A1 is the area of the punctum after the potential deformation fusion and fission event. If Iexp is similar to the observed intensity of the punctum (Iobs) after the potential deformation, fusion and fission, then it was hypothesized that the punctum underwent deformation, fusion and fission. For fission events, A1 is the sum of the area of the two puncta and the observed intensity (Iobs) is the average intensity of both puncta.

### Chromatin Immunoprecipitation-Sequencing (ChIP-Seq)

ChIP-seq experiments were performed by the Center for Functional Cancer Epigenetics (CFCE) at the Dana-Farber Cancer Institute. For ChIP-seq analysis, cells were cross-linked with 2 mM DSG for 45 min at room temperature followed by fixation for 10 min with 1% formaldehyde at room temperature on a shaker at 850 rpm. Crosslinked nuclei were quenched with 0.125 M glycine for 5 min at room temperature and washed with PBS (containing protease inhibitor and HDAC inhibitor Sodium Butyrate (NaBut). After fixation, pellets were resuspended in 200 ul of 1% SDS (50 mM Tris-HCl pH 8, 10 mM EDTA) and sonicated in 1 ml AFA fiber millitubes for 25 min using a Covaris E220 instrument (setting: 140 peak incident power, 5% duty factor and 200 cycles per burst) 600 seconds per sample. Chromatin was diluted 5 times with ChIP Dilution buffer (1% Triton X-100, 2 mM EDTA pH 8, 150 mM NaCl, 20 mM Tris-HCl pH 8) and was immunoprecipitated with 10 μg of primary antibody against IR (Bethyl A303-712A) and Dynabeads® Protein A/G. ChIP-seq libraries were constructed using NEBNext Ultra™ II kit from New England Biolabs. 75-bp paired-end reads were sequenced on a Nextseq instrument. 75-bp single-end reads were sequenced on an Illumina NextSeq instrument.
MED1 ChIP-seq was taken from GEO: GSM2040029 RPB1 ChIP-seq was taken from GEO: GSM2864931 (14)

### ChIP-Seq Analysis

ChIP-seq bioinformatics analysis for insulin receptor was performed on the Whitehead High-Performance Computing Facility using the nf-core ChIP-seq pipeline v1.2.1 (81) with Nextflow v20.04.1. Quality control of .fastq files was performed with FastQC v0.11.9. Trim Galore! v0.6.4_dev was used to trim low quality reads. Alignment was performed against the hg19 genome assembly using BWA v0.7.17-r1188 (82). Peak calling was performed using MACS2 v2.2.7.1 (83). Preseq v2.0.3 (84) and MultiQC v1.9 were used for quality control. Browser tracks were prepared to represent reads per million per basepair (rpm/bp).

### RT-qPCR and RNA-Sequencing

RNA was extracted using TRIzol™ reagent following the manufacturer's instructions (Thermo Fisher Scientific, 15596026). cDNA synthesis was performed using qScript cDNA Supermix according to the manufacturer's instructions, using 1000 ng RNA as input (qScript cDNA SuperMix, QuantaBio—95048-500). qPCR was performed on a Thermo Fisher Scientific QuantStudio 6 machine using Fast SYBR™ Green Master Mix (Thermo Fisher Scientific 4385618). Expression data is presented after calculating the relative expression compared with the housekeeping gene RPLP0, using the equation Relative Quantification (RQ)=100/(2\hat{ }(Target Gene Ct−RPLP0Ct). When data is reported relative to a sample condition, the condition of reference was set as 1 and the data of the other conditions were reported as a ratio (condition/condition of reference).

RNA sequencing was performed by the Whitehead Institute Genome Technology Core. Libraries were prepared using the KAPA HyperPrep stranded RNA kit following manufacturer's instructions. Samples were sequenced on a HiSeq2500 in High-Output mode generating 50 bases, single-end reads.

RT-qPCR primers
MF_RPLP0_qF
(SEQ ID NO: 6)
gcagcatctacaaccctgaag
MF_RPLP0_qR
(SEQ ID NO: 7)
gcagacagacactggcaaca
MF_SMAD7_qF
(SEQ ID NO: 8)
gggctttcagattcccaact
MF_SMAD7_qR
(SEQ ID NO: 9)
ctcgtcttctcctcccagtat

### RNA-Sequencing Analysis

RNA-sequencing (RNA-seq) bioinformatics analysis was performed on the Whitehead High-Performance Computing Facility using the nf-core RNA-seq pipeline v1.4.2 (81) with Nextflow v20.04.1. Quality control of .fastq files was performed with FastQC v0.11.8. The reads were single-end and the strandedness was set to reverse. Low quality sequences were trimmed using Trim Galore!v0.6.4. Alignment was performed against the hg19 genome assembly using STAR v2.6.1d (85) and duplicates were marked using Picard MarkDuplicates v2.21.1. Quantification of transcripts using featureCounts v1.6.4 (86). Differential expression analysis was performed using edgeR v3.26.5 (87). deepTools v3.3.1 (88), dupRadar v1.14.0 (89), Qualimap v.2.2.2-dev (90), and MultiQC v1.7 were used for quality control. ERCC92 was used as RNA spike-in.

### Live-Cell Super-Resolution Imaging (Tc-PALM)

Widefield Live-cell super-resolution imaging was performed in a photo-activation localization microscopy (PALM) approach using a Nikon Eclipse Ti microscope with a 100× oil immersion objective. The 405 nm and 561 nm laser beams were combined in an external platform with customized power densities to image Dendra2-tagged molecules as previously reported (56). Cells were maintained at 37° C. in a temperature-controlled platform during imaging, and the CO2 level was maintained at 5% by Leibovitz's L-15 Medium with no phenol red (ThermoFisher #21083027). In each imaging cycle, a 2400-frame video stream including a (256 pixel)2 region of interest (ROI) was recorded in 20 Hz acquisition rate with the EM-gain setting as 1000 on an Andor iXon Ultra 897 EMCCD. Each pixel conjugates with a (160 nm)2 area on the sample side. After PALM imaging, the Hoechst-stained nuclei of the same ROI were imaged with stronger 405 nm excitation through DAPI filter. For the insulin-untreated condition, cells were firstly imaged in 1.5 ml insulin-free L-15 Medium for 15-20 min. For the insulin-treated condition, 1.5 ml fresh-made, prewarmed L-15 medium containing 2× insulin was directly added to the same dish while it was still on the platform, followed by a 5-minute wait, then proceeding to imaging.

tc-PALM Analysis

Detection localization. For each frame of a raw image, Gaussian particles were identified by pixelwise test of hypotheses, whose peak positions were individually fitted at subpixel resolution by maximum-likelihood regression with Gauss-Newton method (91). An additional deflation loop was performed to avoid missing dimmer particles when they were overshadowed by neighboring brighter ones. This multi-particle detection localization procedure has been integrated in a published, open-source MATLAB software called MTT (91).

Spatial clustering. DBSCAN and “manual selection” hybridized approach was applied to group spatially clustered detections via the qSR software (92). Firstly, DBSCAN was performed to generalize a proposal map of spatially clustered detections. Given that IR condensates can be tiny and transient, a “loose” parameter setting was used when performing the DBSCAN (length-scale=120 nm, N_min=4). This parameter combination was determined by comparing the rendered super-resolved reconstructions with the color-coded cluster maps until the clustering results visually make sense for most ROIs. Secondly, individual clusters were manually selected based on the clustering proposal map from last step. Some custom MATLAB code was used to reconstruct the IR distribution of each ROI superposed with the corresponding nuclei image, which was further cross-compared with the corresponding cluster map to determine which region each cluster belongs to (i.e., plasma-membrane, cytoplasm, or nucleus).

Temporal clustering. For each spatial cluster, time-correlated PALM (tc-PALM) analysis was performed along the time axis to extract the truly colocalized, time-correlated multimolecule bursting events (11, 13-14). The lifetime of a burst is simply defined as the timespan from the first to the last detections. More details about the quantitative validations and statistics of tc-PALM analysis can be found in NOTES and FIG. 11.

### Albumin Quantification

To assess hepatocyte spheroid function, media was collected during every media exchange and albumin secretion was assayed via ELISA (Bethyl Laboratories, E80-129) following the manufacturer's instructions.

### Insulin Clearance Quantification

Insulin clearance was evaluated by collecting media after 48 hours in culture and quantified via ELISA (R&D, DY8056) following manufacturer's instructions. Clearance fraction was calculated by dividing the measured insulin concentration in cultured media by the measured insulin concentration in the cell-free control wells.

### Protein Purification

Human cDNA encoding the beta subunit of the insulin receptor (IRb; residues 763-1382) was cloned into a mammalian expression vector. The base vector was engineered to include sequences encoding an N-terminal FLAG tag followed by mCherry and a 14 amino acid linker sequence “GAPGSAGSAAGGSG. (SEQ ID NO: 10)” cDNA sequences were inserted in-frame following the linker sequence using NEBuilder HiFi DNA Assembly Master Mix (NEB, E2621S). The expression construct was subjected to Sanger sequencing to confirm the sequence.

For protein expression, IRb-mcherry plasmid was transfected into HEK293T cells (ATCC, CRL-3216) using Polyethylenimine. Cells were cultured for 72 hours, scraped off the plate, and washed with ice-cold PBS (Life Technologies, AM9625). Cells were centrifuged at 500 g for 5 min and the cell pellet was stored at −80° C.

The cell pellet was resuspended in 35 ml Lysis Buffer (20 mM HEPES pH7.4, 150 mM NaCl, 1 mM EDTA, 0.5% NP40, with fresh inhibitors and 1 mM DTT). Cell lysate was rocked for 30 min at 4° C. and spun down at 12,000×g 15 min. 35 ml of supernatant was removed to a fresh tube and centrifuged again if cloudy. 300 ul of washed Anti-Flag M2 magnetic beads (Sigma-Aldrich M8823) was added to the lysate, which was then rotated overnight at 4° C. The next day beads were pelleted at 500 rpm for 5 min, washed with 35 ml BD Buffer (10 mM HEPES, 450 mM NaCl, 5% glycerol with fresh inhibitors), transferred to Eppendorf tube and washed 3 to 5 times with BD Buffer using a magnetic rack. Elution was performed overnight with 500 ul Dialysis Buffer (50 mM HEPES, 150 mM NaCl, 5 mM MgCl2, 5% glycerol) plus 50 ul Flag peptide (5 mg/ml stock solution). The next day the sample was eluted with the magnetic rack and washed with 250 ul Dialysis buffer with no peptide. The sample was dialyzed with 500 ml buffer, which was changed 1 to 2 times at 4° C.

### Statistical Analysis

Statistical analysis was performed using Prism (GraphPad, La Jolla, CA). tc-PALM data was analyzed using one-sided t-test, the rest of the data was analyzed using t-test or t-test with Welch's correction. Data is represented as individual values and mean or as individual values and mean±SEM. In all figures, * p<0.05, ** p<0.01, ***p<0.001.

### Notes

### Photochemistry of Single Dendra2 Molecules

Given that there could be an ambiguous mapping from the number of detections to the number of Dendra2 molecules, several control analyses of the single molecule photochemistry have to be done to validate the statistics of the real clusters (which ideally consist of colocalized, time-correlated, multimolecule bursts). The CRISPR/Cas9 edited IR-Dendra2 cells were either fixed or keeping alive and imaged in L-15 medium with the exact laser setups. After the same ROI was imaged for a long time, most Dendra2 molecules were photo-converted and bleached, whereupon rest of the intact single molecules were sparsely photo-converted and recorded, and the consequent colocalized detections from the same molecule can be well spatiotemporally isolated and grouped. 94% of the single molecules only generate one detection, which results in the average number of detections per molecule being close to one (ndet≈1.077). The average lifetime of single molecules is 0.059 s, and only 1% of them has a lifetime longer than 0.25 s. Among those multiple-detection molecules, 65% of them result in the same emitting event occupying two adjacent frames, and the real average dark-time between blinking events is around 0.2 s.

### Validation of the Existence of Dynamic Clustering in Live Cells

Applicant identified pseudo-transient clusters in fixed cells with the exact procedures and criteria as for searching transient clusters in live cells. For spatially clustered structures, significantly larger dark times in live cells, compared to fixed cells under identical condition, is a sign of the bursting dynamics in live cells (54). This is exactly what was observed, and such larger dark times of clusters in live cells cannot be explained by longer intrinsic inter-detection period of Dendra2 single molecules in live-cell samples. Furthermore, Applicant normalized the number of tc-PALM identified bursts by the total number of detections of the same ROI, thus are able to estimate the number of identified bursts per 10,000 detections as 67.02. Meanwhile, among the tc-PALM identified bursts, Applicant obtained the number of detections and lifetime of the 0.05 quantile at the lower-bound side as 4 and 0.85 s, respectively. If Applicant use these two numbers as the cut-off for the set of Dendra2 single molecules applicant measured in live samples, only 4.67 molecules among 10,000 detections can pass the threshold. This indicates that the true positive rate (TPR) can easily go beyond 90%: 67.02÷(67.02+4.67)=93.5%; even in the worst case (all the bursts below the 0.05 quantile were single molecules), the corresponding TPR is 93.5%×95%≈89%. Even for the outlier single molecules that pass the cut-off, their statistics (including duration time, inter-detection period, and number of detections) are still quite different from the of tc-PALM identified bursts. In another extreme test, Applicant applied several additional high cut-offs to the tc-PALM identified bursts (in some cases, the TPR was pushed to 98%), whereupon applicant is still able to recapitulate all the significant trends of lifetime-shifting in cytoplasm and nuclei after different perturbations (data not shown). This observation is reasonable: given that IRs are much less abundant in the cytoplasm and nuclei, un-clustered background of randomly bound IRs can be safely ignored; therefore, any time-correlated, multi-detection events inside in the cytoplasm or nuclei are very likely to result from real clusters, which are insensitive to the FPR cut-off. Gathering all these evidences together, applicant is able to validate the existence of multi-molecule dynamical clustering of IRs in live cells, which yields transient bursting dynamics with distinct properties than single molecules and are robustly, physiologically responsive.

### REFERENCES

1. J. Boucher, A. Kleinridders, C. R. Kahn, Insulin receptor signaling in normal and insulin-resistant states. Cold Spring Harb Perspect Biol 6, (2014).
2. M. P. Czech, Insulin action and resistance in obesity and type 2 diabetes. Nat Med 23, 804-814 (2017).
3. R. H.F., Insulin resistance and bronze diabetes. The New England Journal of Medicine 201, 201-205 (1929).
4. R. A. DeFronzo et al., Type 2 diabetes mellitus. Nat Rev Dis Primers 1, 15019 (2015).
5. N. Rachdaoui, Insulin: The Friend and the Foe in the Development of Type 2 Diabetes Mellitus. Int J Mol Sci 21, (2020).
6. G. I. Shulman, Cellular mechanisms of insulin resistance. J Clin Invest 106, 171-176 (2000).
7. G. I. Shulman, Cellular mechanisms of insulin resistance in humans. Am J Cardiol 84, 3J-10J (1999).
8. S. B. Biddinger, C. R. Kahn, From mice to men: insights into the insulin resistance syndromes. Annu Rev Physiol 68, 123-158 (2006).
9. R. R. Henry, B. Gumbiner, T. Flynn, A. W. Thorburn, Metabolic effects of hyperglycemia and hyperinsulinemia on fate of intracellular glucose in NIDDM. Diabetes 39, 149-156 (1990).
10. M. H. Shanik et al., Insulin resistance and hyperinsulinemia: is hyperinsulinemia the cart or the horse?Diabetes Care 31 Suppl 2, S262-268 (2008).
11. A. Tzatsos, K. V. Kandror, Nutrients suppress phosphatidylinositol 3-kinase/Akt signaling via raptor-dependent mTOR-mediated insulin receptor substrate 1 phosphorylation. Mol Cell Biol 26, 63-76 (2006).
12. J. Hirosumi et al., A central role for JNK in obesity and insulin resistance. Nature 420, 333-336 (2002).
13. Z. Gao et al., Inhibition of insulin sensitivity by free fatty acids requires activation of multiple serine kinases in 3T3-L1 adipocytes. Mol Endocrinol 18, 2024-2034 (2004).
14. M. T. Nguyen et al., JNK and tumor necrosis factor-alpha mediate free fatty acid-induced insulin resistance in 3T3-L1 adipocytes. J Biol Chem 280, 35361-35371 (2005).
15. G. Perseghin, K. Petersen, G. I. Shulman, Cellular mechanism of insulin resistance: potential links with inflammation. Int J Obes Relat Metab Disord 27 Suppl 3, S6-11(2003).
16. G. S. Hotamisligil, B. M. Spiegelman, Tumor necrosis factor alpha: a key component of the obesity-diabetes link. Diabetes 43, 1271-1278 (1994).
17. B. B. Lowell, G. I. Shulman, Mitochondrial dysfunction and type 2 diabetes. Science 307, 384-387 (2005).
18. S. Hurrle, W. H. Hsu, The etiology of oxidative stress in insulin resistance. Biomed J 40, 257-262 (2017).
19. O. K. Kim, W. Jun, J. Lee, Mechanism of ER Stress and Inflammation for Hepatic Insulin Resistance in Obesity. Ann Nutr Metab 67, 218-227 (2015).
20. V. E. Parker, R. K. Semple, Genetics in endocrinology: genetic forms of severe insulin resistance: what endocrinologists should know. Eur J Endocrinol 169, R71-80 (2013).
21. S. I. Taylor et al., Mutations in insulin-receptor gene in insulin-resistant patients. Diabetes Care 13, 257-279 (1990).
22. D. Accili et al., Mutations in the insulin receptor gene in patients with genetic syndromes of insulin resistance and acanthosis nigricans. J Invest Dermatol 98, 77S-81S (1992).
23. R. M. Furnica, L. Istasse, D. Maiter, A severe but reversible reduction in insulin sensitivity is observed in patients with insulinoma. Ann Endocrinol (Paris) 79, 30-36 (2018).
24. D. L. Esposito et al., A novel T608R missense mutation in insulin receptor substrate-1 identified in a subject with type 2 diabetes impairs metabolic insulin signaling. J Clin Endocrinol Metab 88, 1468-1475 (2003).
25. M. Honma et al., Selective insulin resistance with differential expressions of IRS-1 and IRS-2 in human NAFLD livers. Int J Obes (Lond) 42, 1544-1555 (2018).
26. V. Aguirre et al., Phosphorylation of Ser307 in insulin receptor substrate-1 blocks interactions with the insulin receptor and inhibits insulin action. J Biol Chem 277, 1531-1537 (2002).
27. L. Y. Qiao, J. L. Goldberg, J. C. Russell, X. J. Sun, Identification of enhanced serine kinase activity in insulin resistance. J Biol Chem 274, 10625-10632 (1999).
28. O. J. Shah, Z. Wang, T. Hunter, Inappropriate activation of the TSC/Rheb/mTOR/S6K cassette induces IRS1/2 depletion, insulin resistance, and cell survival deficiencies. Curr Biol 14, 1650-1656 (2004).
29. G. Rena, D. G. Hardie, E. R. Pearson, The mechanisms of action of metformin. Diabetologia 60, 1577-1585 (2017).
30. M. J. Amaya et al., The insulin receptor translocates to the nucleus to regulate cell proliferation in liver. Hepatology 59, 274-283 (2014).
31. I. D. Goldfine, G. J. Smith, Binding of insulin to isolated nuclei. Proc Natl Acad Sci USA 73, 1427-1431 (1976).
32. M. L. Hancock et al., Insulin Receptor Associates with Promoters Genome-wide and Regulates Gene Expression. Cell 177, 722-736 e722 (2019).
33. J. D. Nelson, R. C. LeBoeuf, K. Bomsztyk, Direct recruitment of insulin receptor and ERK signaling cascade to insulin-inducible gene loci. Diabetes 60, 127-137 (2011).
34. D. A. Podlecki et al., Nuclear translocation of the insulin receptor. A possible mediator of insulin's long term effects. J Biol Chem 262, 3362-3368 (1987).
35. J. L. Carpentier, Insulin receptor internalization: molecular mechanisms and physiopathological implications. Diabetologia 37 Suppl 2, S117-124 (1994).
36. S. Fagerholm, U. Ortegren, M. Karlsson, I. Ruishalme, P. Stralfors, Rapid insulin-dependent endocytosis of the insulin receptor by caveolae in primary adipocytes. PLoS One 4, e5985 (2009).
37. T. Boothe et al., Inter-domain tagging implicates caveolin-1 in insulin receptor trafficking and Erk signaling bias in pancreatic beta-cells. Mol Metab 5, 366-378 (2016).
38. J. Gustavsson et al., Localization of the insulin receptor in caveolae of adipocyte plasma membrane. FASEB J 13, 1961-1971 (1999).
39. X. Su et al., Phase separation of signaling molecules promotes T cell receptor signal transduction. Science 352, 595-599 (2016).
40. A. V. Zamudio et al., Mediator Condensates Localize Signaling Factors to Key Cell Identity Genes. Mol Cell 76, 753-766 e756 (2019).
41. L. B. Case, J. A. Ditlev, M. K. Rosen, Regulation of Transmembrane Signaling by Phase Separation. Annu Rev Biophys 48, 465-494 (2019).
42. C. Lin et al., Receptor tyrosine kinases regulate signal transduction through a liquid-liquid phase separated state. bioRxiv, (2019).
43. S. J. Nair et al., Phase separation of ligand-activated enhancers licenses cooperative chromosomal enhancer assembly. Nat Struct Mol Biol 26, 193-203 (2019).
44. A. Patel et al., ATP as a biological hydrotrope. Science 356, 753-756 (2017).
45. B. R. Sabari et al., Coactivator condensation at super-enhancers links phase separation and gene control. Science 361, (2018).
46. C. H. Foyer, Redox control of flowering. Nat Chem Biol 17, 504-505 (2021).
47. M. A. Abdul-Ghani, R. A. DeFronzo, Pathogenesis of insulin resistance in skeletal muscle. J Biomed Biotechnol 2010, 476279 (2010).
48. Y. Wang et al., Metformin Improves Mitochondrial Respiratory Activity through Activation of AMPK. Cell Rep 29, 1511-1523 e1515 (2019).
49. N. Apostolova et al., Mechanisms of action of metformin in type 2 diabetes: Effects on mitochondria and leukocyte-endothelium interactions. Redox Biol 34, 101517 (2020).
50. S. Sefried, H. U. Haring, C. Weigert, S. S. Eckstein, Suitability of hepatocyte cell lines HepG2, AML12 and THLE-2 for investigation of insulin signalling and hepatokine gene expression. Open Biol 8, (2018).
51. S. Yadollah, N. Kazemipour, S. Bakhtiyari, S. Nazifi, Palmitate-induced insulin resistance is attenuated by Pioglitazone and EGCG through reducing the gluconeogenic key enzymes expression in HepG2 cells. J Med Life 10, 244-249 (2017).
52. M. Watanabe, H. Hayasaki, T. Tamayama, M. Shimada, Histologic distribution of insulin and glucagon receptors. Braz J Med Biol Res 31, 243-256 (1998).
53. Cisse, I I et al., Real-time dynamics of RNA polymerase II clustering in live human cells. Science 341, 664-667 (2013).
54. W. K. Cho et al., Mediator and RNA polymerase II clusters associate in transcription-dependent condensates. Science 361, 412-415 (2018).
55. W. K. Cho et al., RNA Polymerase II cluster dynamics predict mRNA output in living cells. Elife 5, (2016).
56. S. P. Mukherjee, R. H. Lane, W. S. Lynn, Endogenous hydrogen peroxide and peroxidative metabolism in adipocytes in response to insulin and sulfhydryl reagents. Biochem Pharmacol 27, 2589-2594 (1978).
57. J. M. May, C. de Haen, Insulin-stimulated intracellular hydrogen peroxide production in rat epididymal fat cells. J Biol Chem 254, 2214-2220 (1979).
58. H. I. Krieger-Brauer, H. Kather, Human fat cells possess a plasma membrane-bound H2O2-generating system that is activated by insulin via a mechanism bypassing the receptor kinase. J Clin Invest 89, 1006-1013 (1992).
59. K. Mahadev et al., The NAD(P)H oxidase homolog Nox4 modulates insulin-stimulated generation of H₂O₂ and plays an integral role in insulin signal transduction. Mol Cell Biol 24, 1844-1854 (2004).
60. H. I. Krieger-Brauer, P. K. Medda, H. Kather, Insulin-induced activation of NADPH-dependent H₂O₂ generation in human adipocyte plasma membranes is mediated by Galphai2. J Biol Chem 272, 10135-10143 (1997).
61. S. Iwakami et al., Concentration-dependent dual effects of hydrogen peroxide on insulin signal transduction in H4IIEC hepatocytes. PLoS One 6, e27401 (2011).
62. X. Huang et al., ROS regulated reversible protein phase separation synchronizes plant flowering. Nat Chem Biol 17, 549-557 (2021).
63. J. Guillen-Boixet et al., RNA-Induced Conformational Switching and Clustering of G3BP Drive Stress Granule Assembly by Condensation. Cell 181, 346-361 e317 (2020).
64. J. B. Rayman, K. A. Karl, E. R. Kandel, TIA-1 Self-Multimerization, Phase Separation, and Recruitment into Stress Granules Are Dynamically Regulated by Zn(2). Cell Rep 22, 59-71 (2018).
65. M. Y. El-Mir et al., Dimethylbiguanide inhibits cell respiration via an indirect effect targeted on the respiratory chain complex I. J Biol Chem 275, 223-228 (2000).
66. X. Hou et al., Metformin reduces intracellular reactive oxygen species levels by upregulating expression of the antioxidant thioredoxin via the AMPK-FOXO3 pathway. Biochem Biophys Res Commun 396, 199-205 (2010).
67. B. Onken, M. Driscoll, Metformin induces a dietary restriction-like state and the oxidative stress response to extend C. elegans Healthspan via AMPK, LKB1, and SKN-1. PLoS One 5, e8758 (2010).
68. G. Ashabi, L. Khalaj, F. Khodagholi, M. Goudarzvand, A. Sarkaki, Pre-treatment with metformin activates Nrf2 antioxidant pathways and inhibits inflammatory responses through induction of AMPK after transient global cerebral ischemia. Metab Brain Dis 30, 747-754 (2015).
69. S. Prasad et al., Role of Nrf2 and protective effects of Metformin against tobacco smoke-induced cerebrovascular toxicity. Redox Biol 12, 58-69 (2017).
70. M. Zeng et al., Phase Transition in Postsynaptic Densities Underlies Formation of Synaptic Complexes and Synaptic Plasticity. Cell 166, 1163-1175 eli 12 (2016).
71. B. S. Berlett, E. R. Stadtman, Protein oxidation in aging, disease, and oxidative stress. J Biol Chem 272, 20313-20316 (1997).
72. W. Kidson, Polycystic ovary syndrome: a new direction in treatment. Med J Aust 169, 537-540 (1998).
73. A. Armandi, C. Rosso, G. P. Caviglia, E. Bugianesi, Insulin Resistance across the Spectrum of Nonalcoholic Fatty Liver Disease. Metabolites 11, (2021).
74. G. S. Watson, S. Craft, The role of insulin resistance in the pathogenesis of Alzheimer's disease: implications for treatment. CNS Drugs 17, 27-45 (2003).
75. I. A. Pomytkin, H₂O₂ Signalling Pathway: A Possible Bridge between Insulin Receptor and Mitochondria. Curr Neuropharmacol 10, 311-320 (2012).
76. M. Mahrouf et al., Metformin reduces angiotensin-mediated intracellular production of reactive oxygen species in endothelial cells through the inhibition of protein kinase C. Biochem Pharmacol 72, 176-183 (2006).
77. N. Ouslimani et al., Metformin decreases intracellular production of reactive oxygen species in aortic endothelial cells. Metabolism 54, 829-834 (2005).
78. S. Soberanes et al., Metformin Targets Mitochondrial Electron Transport to Reduce Air-Pollution-Induced Thrombosis. Cell Metab 29, 503 (2019).
79. H. Nasri, M. Rafieian-Kopaei, Metformin: Current knowledge. J Res Med Sci 19, 658-664 (2014).
80. Y. Shin, C. P. Brangwynne, Liquid phase condensation in cell physiology and disease. Science 357, (2017).
81. S. Alberti, D. Dormann, Liquid-Liquid Phase Separation in Disease. Annu Rev Genet 53, 171-194 (2019).
82. S. Alberti, A. A. Hyman, Biomolecular condensates at the nexus of cellular stress, protein aggregation disease and ageing. Nat Rev Mol Cell Biol 22, 196-213 (2021).
83. M. Berenguer, L. Martinez, S. Giorgetti-Peraldi, Y. Le Marchand-Brustel, R. Govers, A serum factor induces insulin-independent translocation of GLUT4 to the cell surface which is maintained in insulin resistance. PLoS One 5, e15560 (2010).
84. S. H. Song et al., Direct measurement of pulsatile insulin secretion from the portal vein in human subjects. J Clin Endocrinol Metab 85, 4491-4499 (2000).
85. M. Storch, M. Rössle, L. Kerp, Pulsatile Insulinsekretion in die Pfortader bei Leberzirrhose. Med Wochenschr 118, 134-138 (1993).
86. N. Porksen et al., Human insulin release processes measured by intraportal sampling. Am J Physiol Endocrinol Metab 282, E695-702 (2002).

### Example 2

This Example both re-presents certain data from Example 1 and provides additional data.

Insulin receptor (IR) signaling is central to normal metabolic control and is dysregulated in metabolic diseases such as type 2 diabetes. It is reported here that IR is incorporated into liquid-like condensates at the plasma membrane, in the cytoplasm and in the nucleus of human hepatocytes and adipocytes. Insulin stimulation promotes further incorporation of IR into these dynamic condensates in insulin-sensitive cells but not in insulin-resistant cells, where both IR accumulation and dynamic behavior are reduced. Treatment of insulin-resistant cells with metformin, a first-line drug used to treat type 2 diabetes, can rescue IR accumulation and the dynamic behavior of these condensates. This rescue is associated with metformin's role in reducing reactive oxygen species that interfere with normal condensate dynamics. These results indicate that changes in the physico-mechanical features of IR condensates contribute to insulin resistance and have implications for improved therapeutic approaches.

### Results

### Insulin Receptor Bodies in Human Liver Cells

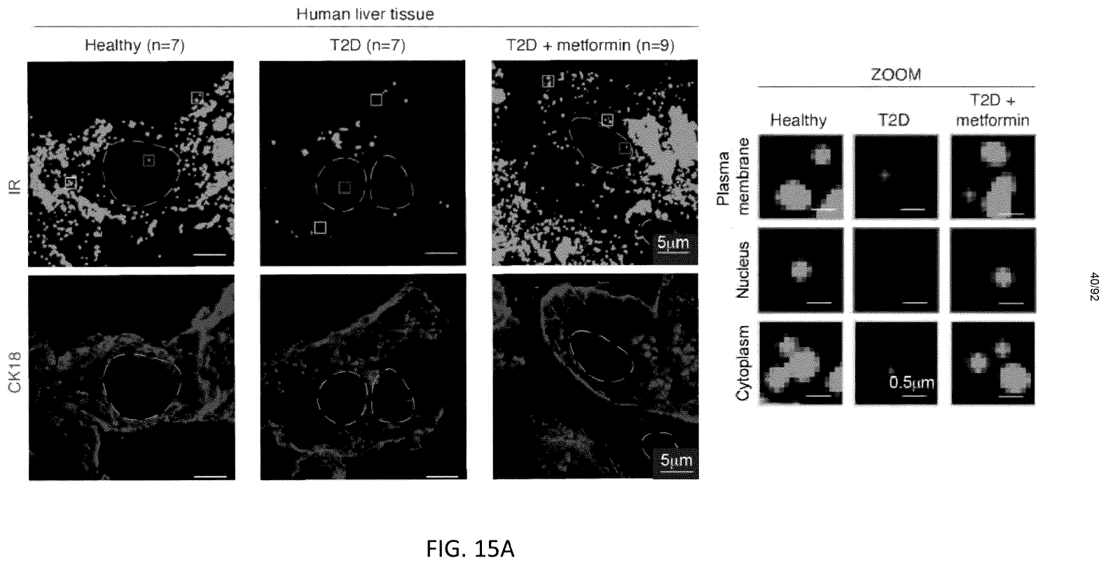

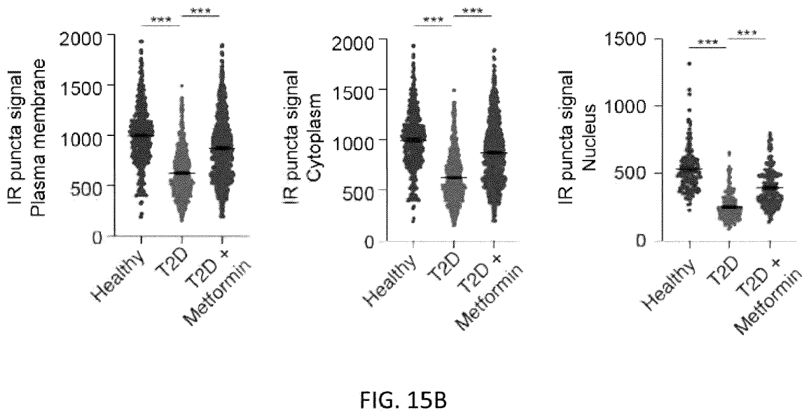

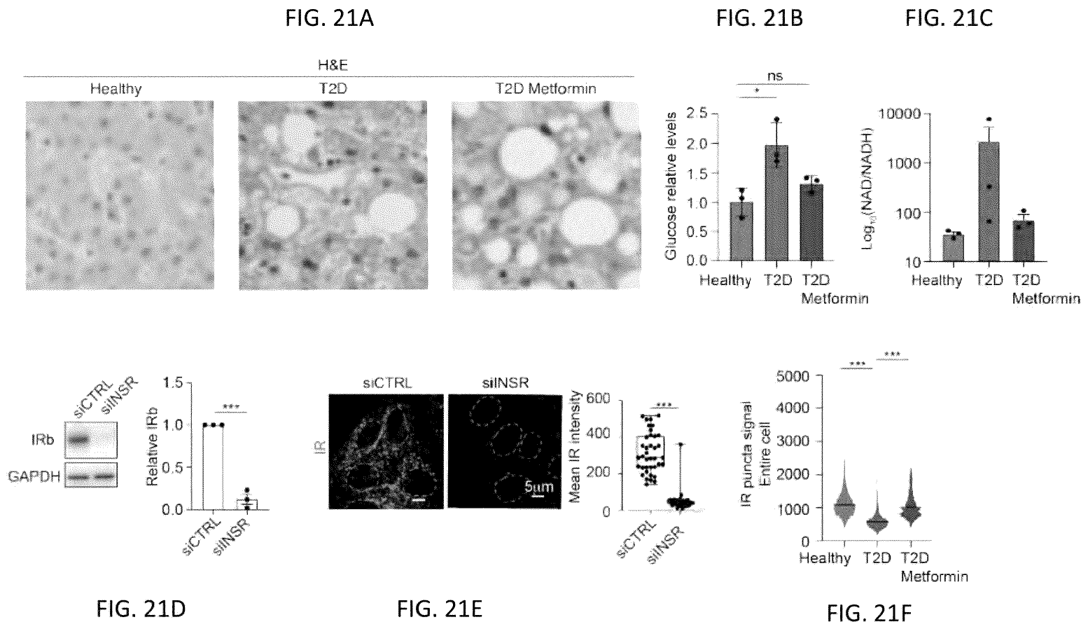

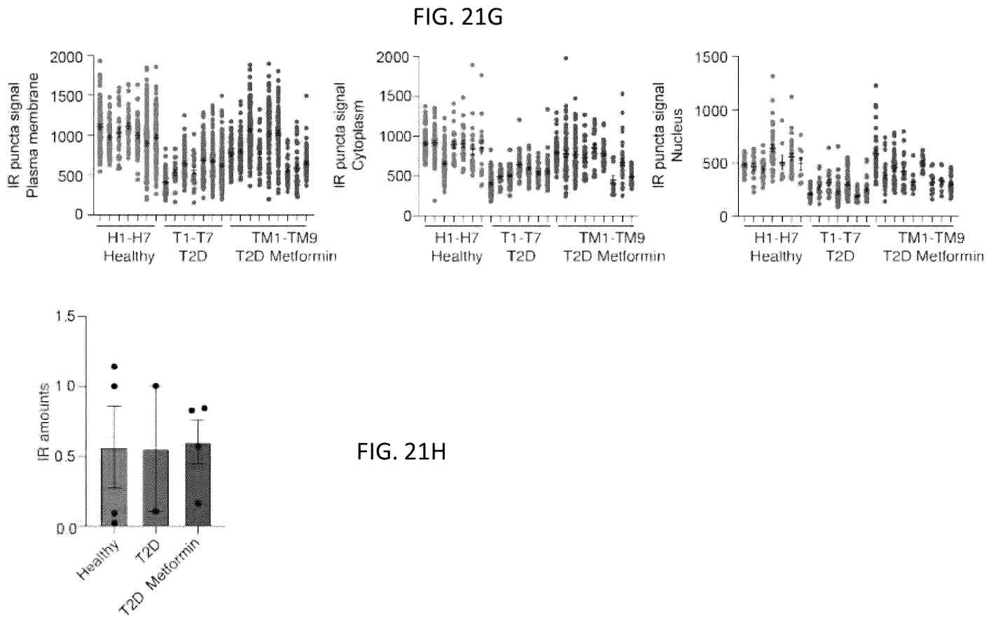

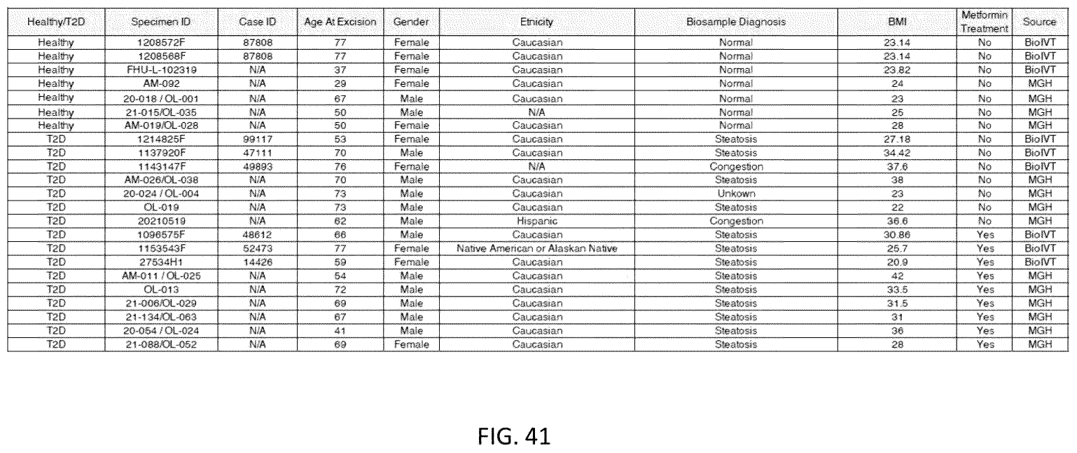

Biomolecular condensates can be visualized as punctate bodies in cells and IR has previously been observed in punctate bodies in diverse cultured cells^{16,39,40}. Applicant investigated whether IR puncta occur in healthy human liver tissue and whether such puncta differ in T2D patients treated with and without metformin, the front-line drug for T2D. 23 human liver tissue samples were examined, comprising seven from healthy donors, seven from donors with T2D and nine from donors with T2D under treatment with metformin (FIG. 15, FIG. 41). These liver tissues exhibited histologic and metabolic features, as well as redox states, expected for healthy donors, donors with T2D and donors with T2D under metformin treatment^{18,41-45} (FIGS. 21A-21C, FIG. 41). Immunofluorescence for CK18 was used to assess tissue quality, cell morphology and as a marker for hepatocytes (FIG. 15A). Imaging of these tissues with a validated antibody for IR (FIGS. 21D-21E) revealed that IR occurs in punctate bodies in hepatocytes from healthy donors, but these signals were significantly reduced in tissues from T2D donors that were not treated with metformin (FIGS. 15A-15B, FIGS. 21F-21G). It was notable that hepatocytes from metformin-treated donors with T2D had IR punctate signals similar to those observed in healthy tissues. These differences were evident in puncta formed in the plasma membrane, the cytoplasm and the nucleus (FIG. 15, FIG. 21G). The total levels of IR protein spanned a similar range in donor tissues from healthy and T2D donors (FIG. 21H), suggesting that the reduced punctate signal in the tissue of T2D donors lacking metformin treatment is not simply due to a difference in the overall level of IR protein. These results suggest that the incorporation of IR into puncta in human hepatocytes is attenuated in T2D and is rescued to some extent by metformin treatment.

### Insulin Receptor Bodies in HepG2 Cells

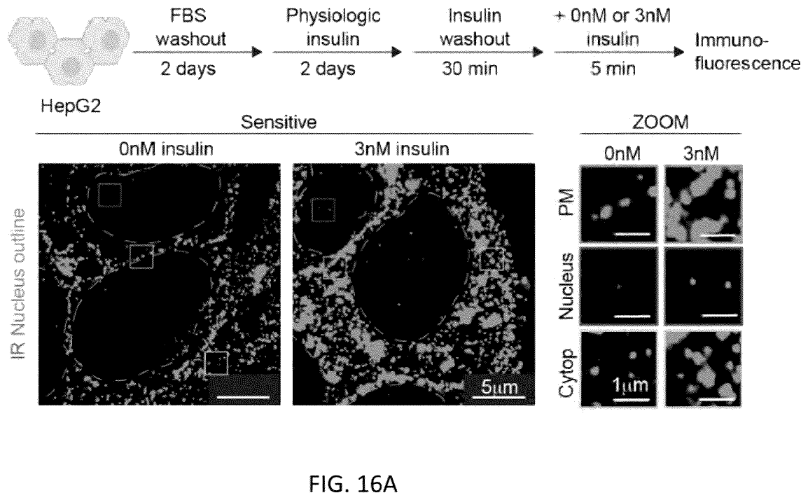

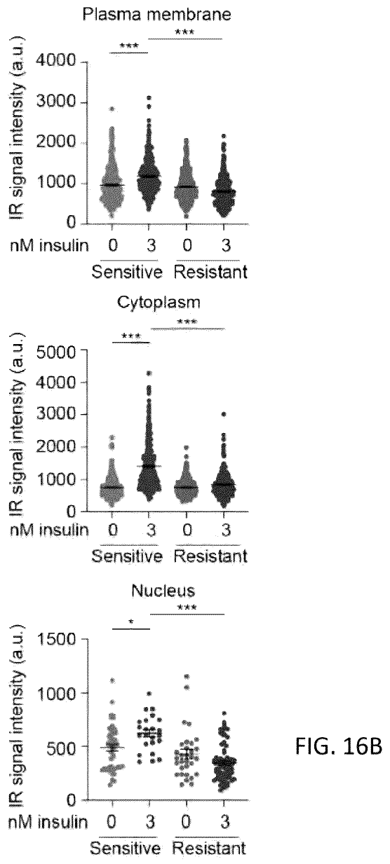

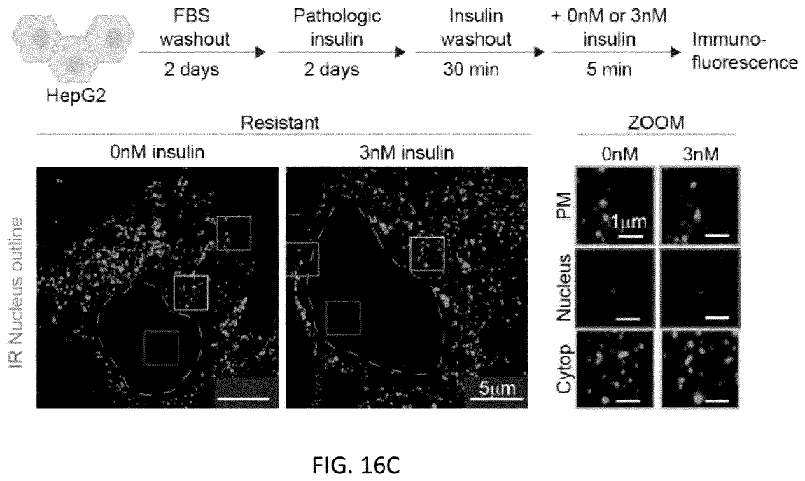

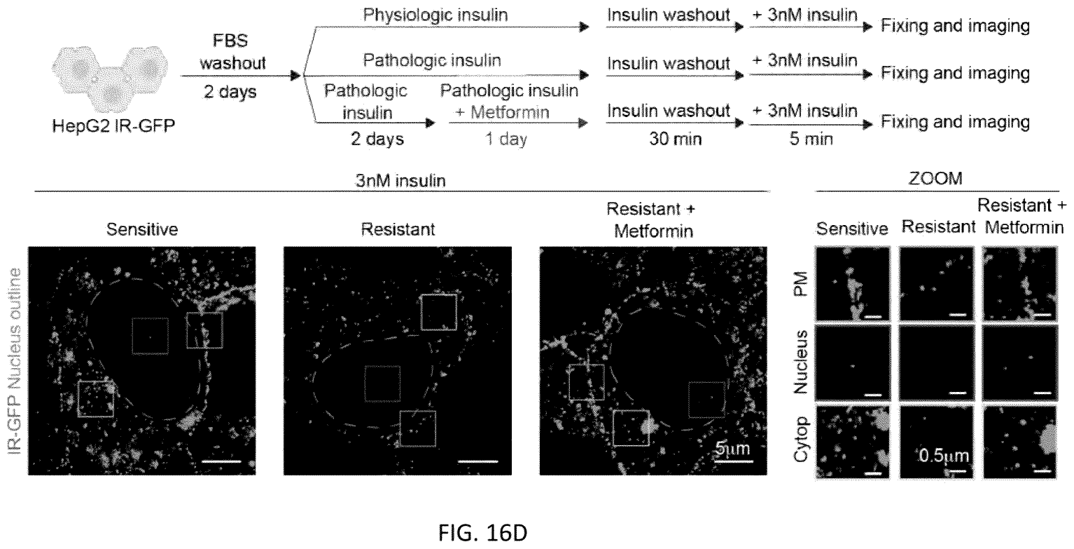

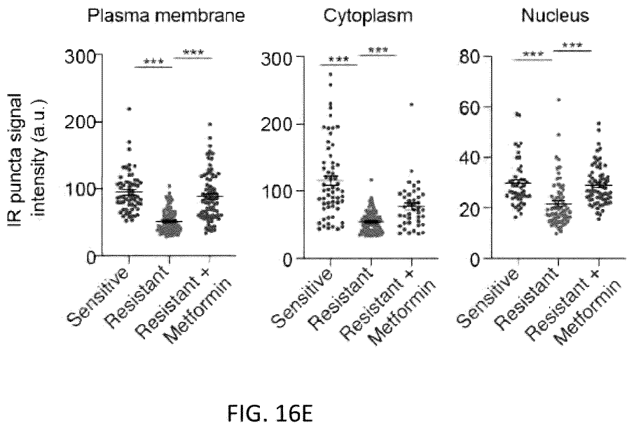

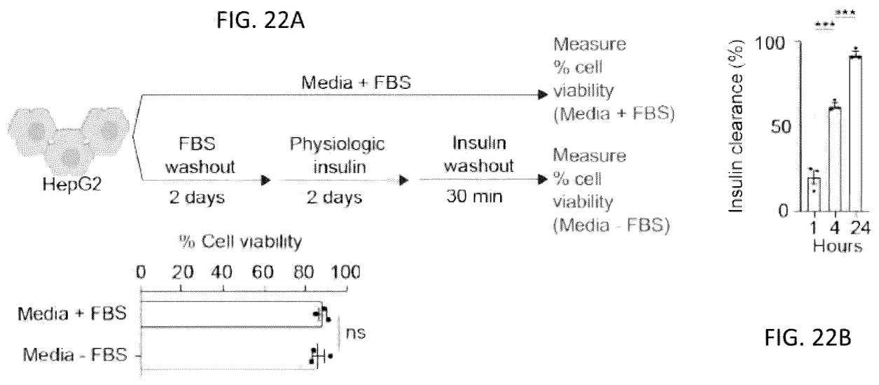

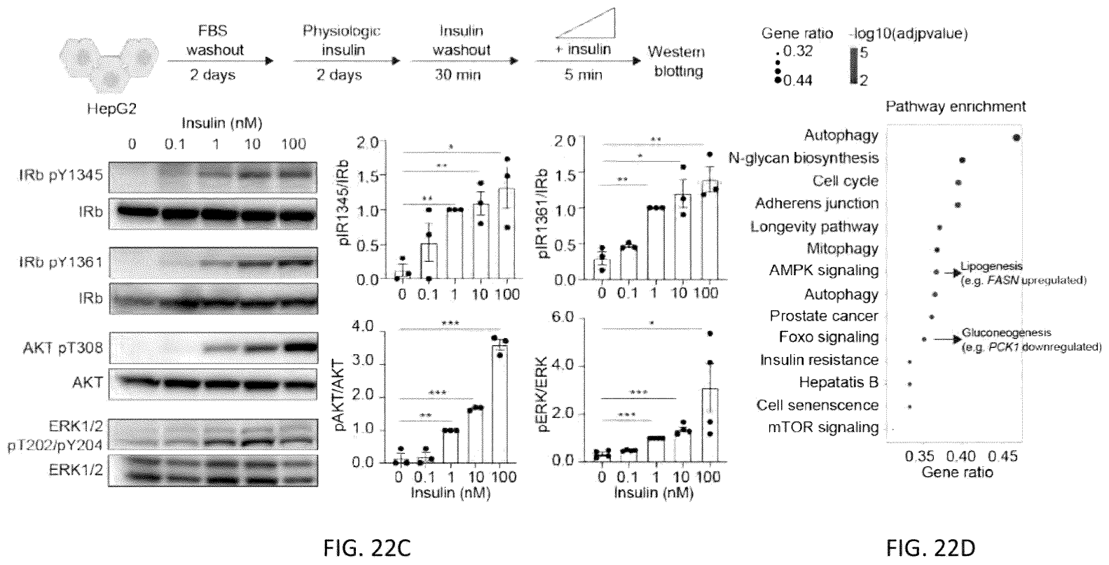

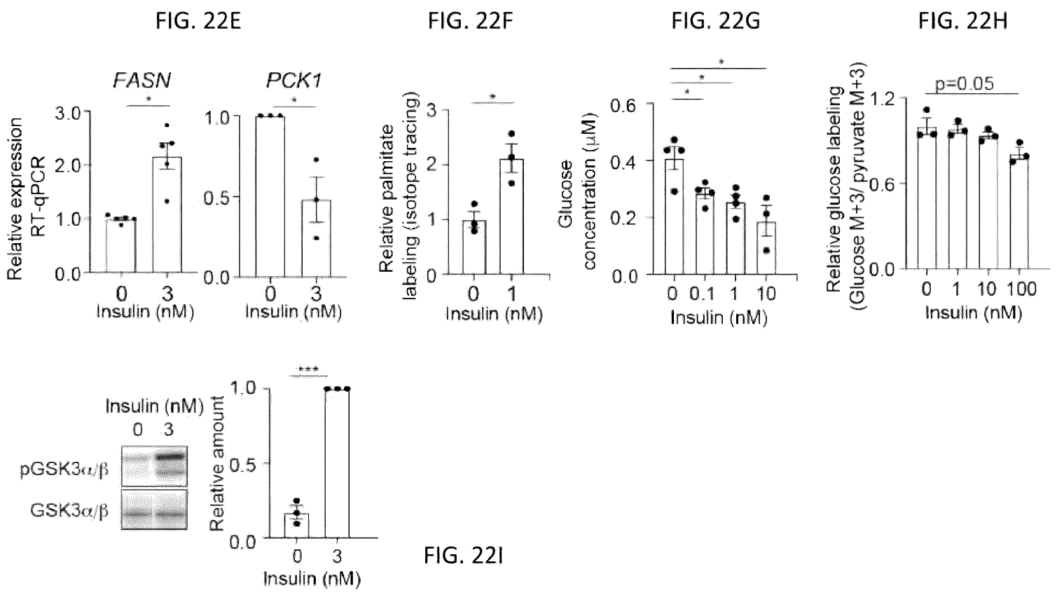

To further investigate the features of IR puncta in hepatocytes, applicant turned to HepG2 cells because of their demonstrated utility in the study of insulin signaling and resistance, and because they are amenable to genetic modification^{11,46,47}. Cells were cultured in media containing physiological concentrations of glucose (5 mM) and insulin (0.1 nM)⁴⁸⁻⁵⁰ (FIG. 16A). Cell culture conditions were selected to mimic those experienced by hepatocytes in situ and cell viability and the ability of cells to clear insulin remained high under these conditions (FIGS. 22A-22B). To confirm that these cells were insulin-sensitive, conventional assays of insulin sensitivity were performed. Acute insulin stimulation induced IR phosphorylation (FIG. 22C), AKT and ERK pathway activation (FIG. 22C), upregulation of lipogenic genes and downregulation of gluconeogenesis genes (FIGS. 22D-22E), increased lipogenesis (FIG. 22F), decreased glucose production (FIGS. 22G-22H), and increased GSK3 phosphorylation (FIG. 22I). Thus, the HepG2 cells cultured in this fashion exhibit the conventional features associated with insulin sensitivity.

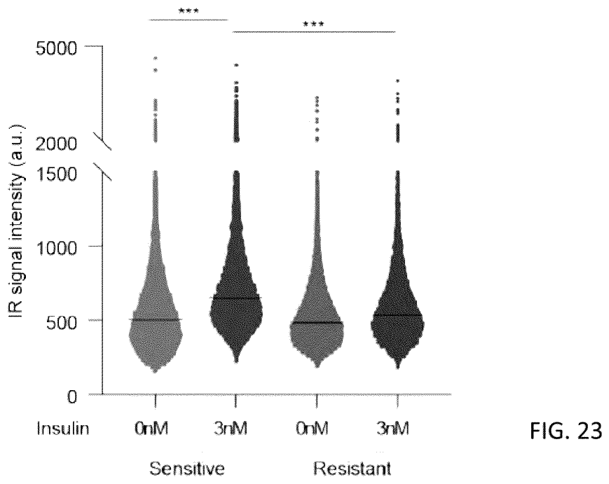

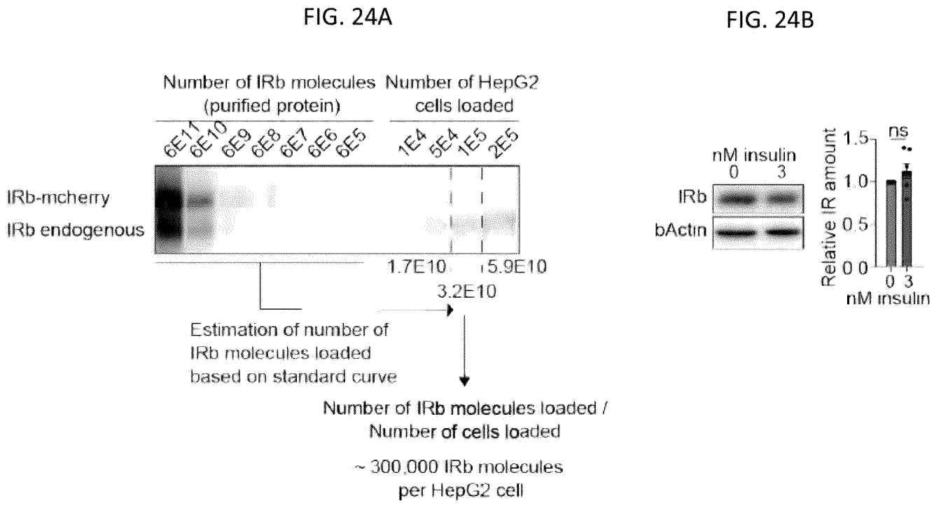

IR localization was monitored in HepG2 cells by immunofluorescence super-resolution microscopy and was found to be incorporated into puncta at the plasma membrane, cytoplasm and nucleus of HepG2 cells in the absence of insulin stimulation, and this signal was elevated with insulin stimulation (FIGS. 16A-16B, FIG. 23). Western blot analysis indicated HepG2 cells contain ˜300,000 IR molecules/cell (FIG. 24A), consistent with estimates for human hepatocytes⁵¹, and showed that the unstimulated and insulin-stimulated cells contain similar levels of IR (FIG. 24B). Given the similar levels of IR in unstimulated and insulin-stimulated cells, applicant inferred from the imaging results that the increased IR signal in puncta reflects increased incorporation of IR molecules into these bodies from the surrounding intracellular environment, and not changes in the overall level of the protein.

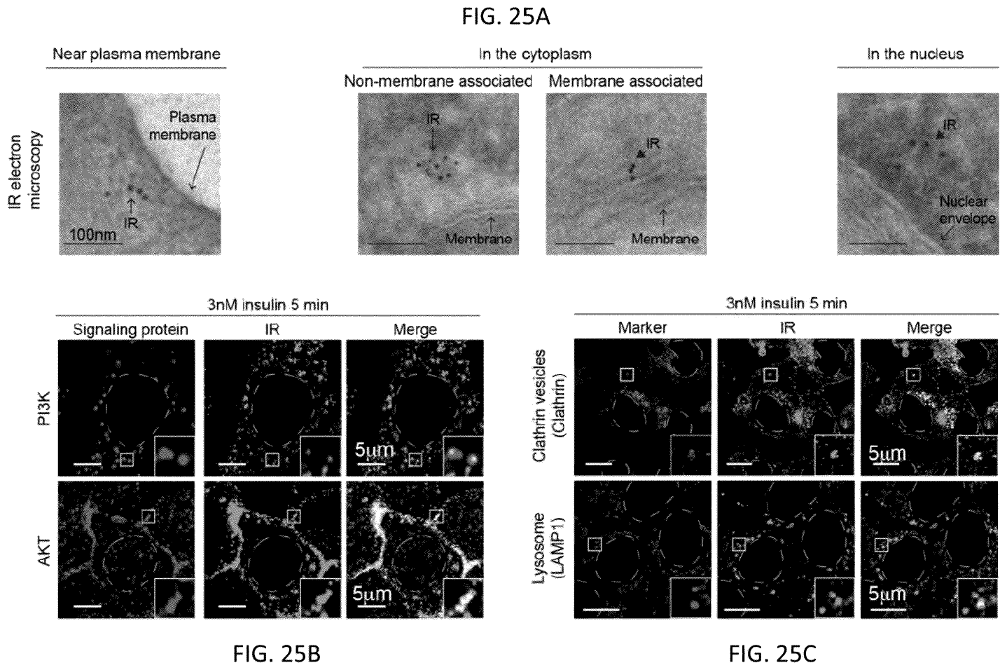

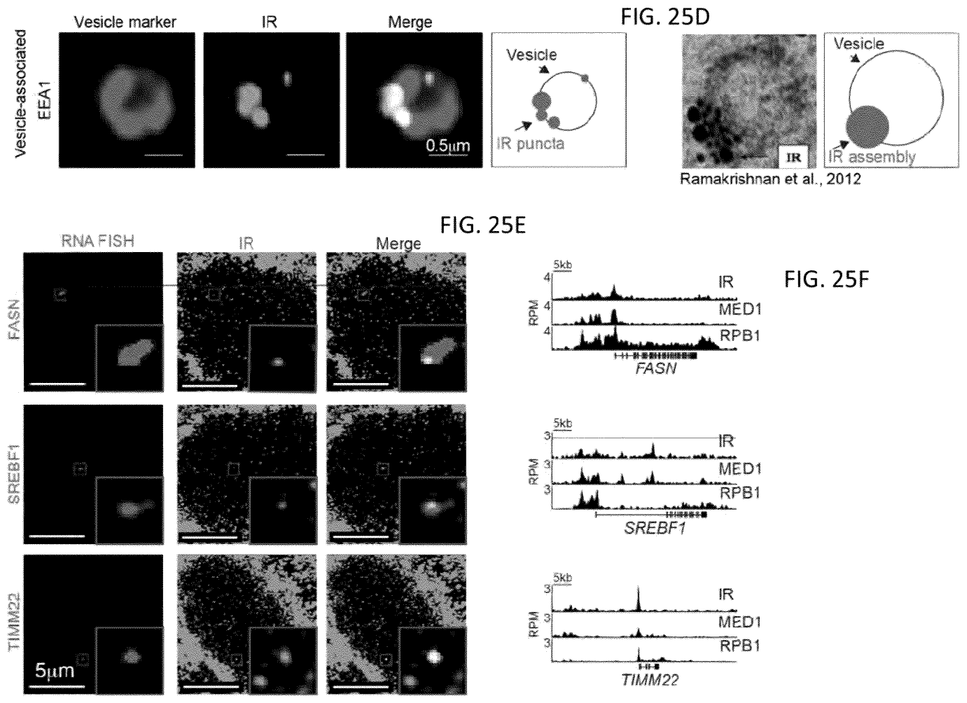

Active IR has been reported to localize at plasma membrane microdomains or signalosomes with other signaling proteins, enter the cytoplasm via endocytosis and become associated with lysosomes or be recycled to the plasma membrane, and enter the nucleus and bind to insulin responsive genes^{10-16,39,40}. The observations with IR bodies in the plasma membrane, cytoplasm and nucleus of HepG2 cells are consistent with these prior reports (FIG. 16A; FIG. 25). Electron microscopy with IR-specific antibodies confirmed that IR can be found near the plasma membrane, in the cytoplasm (some associated with membranes and some not) and in the nucleus (FIG. 25A). Super-resolution microscopy confirmed that IR puncta can colocalize with a portion of the insulin signaling proteins AKT and PI3K (FIG. 25B), that IR puncta can colocalize with clathrin vesicles and lysosomes (FIG. 25C), and that IR puncta can be found at the periphery of endosome vesicles (FIG. 25D). These results suggest that IR puncta are not simply concentrations of IR constrained as a consequence of being fully enveloped by membranes, but instead can sometimes be partially associated with membranes, consistent with previously published results for IR⁵² and other protein condensates associated with plasma membranes and endosomes such as those formed by other signaling factors and neuronal post-synaptic densities^{27,30,53-56}. In the nucleus, IR puncta were found colocalized with markers of transcriptional condensates (MED1 and RNA Polymerase II) at the insulin responsive genes FASN, SREBF1 and TIMM22 (FIG. 25E), and this was confirmed by ChIP-seq analysis of these proteins at these genes (FIG. 25F).

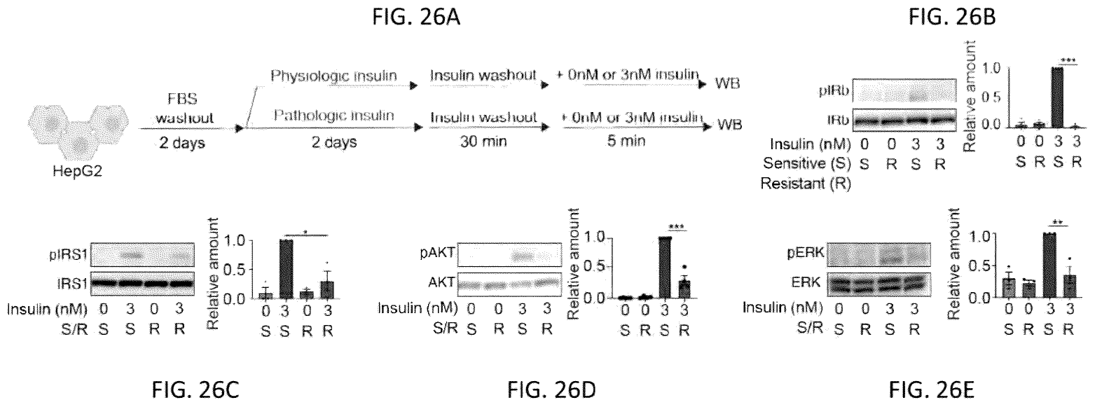

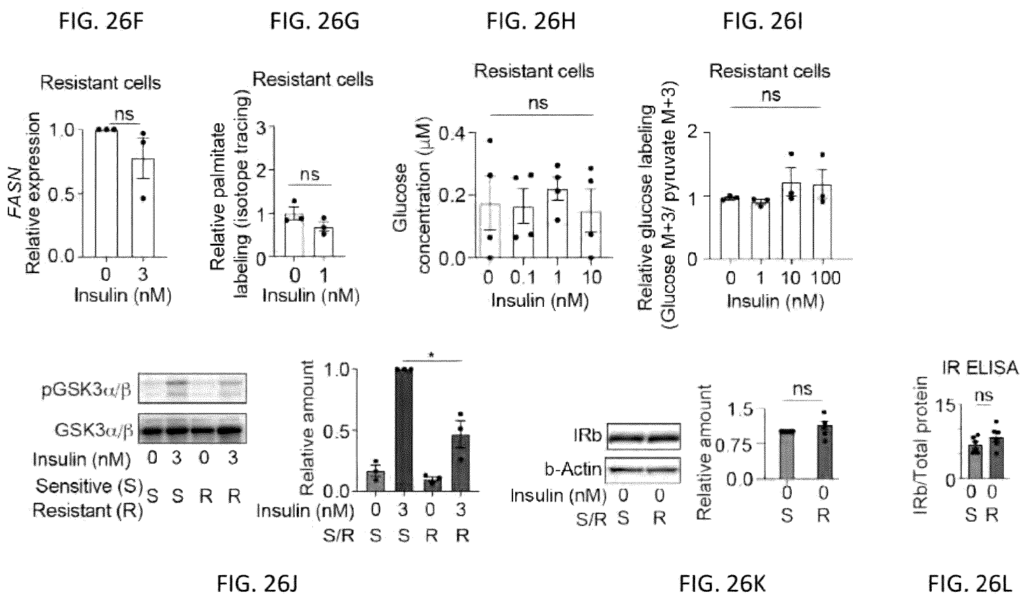

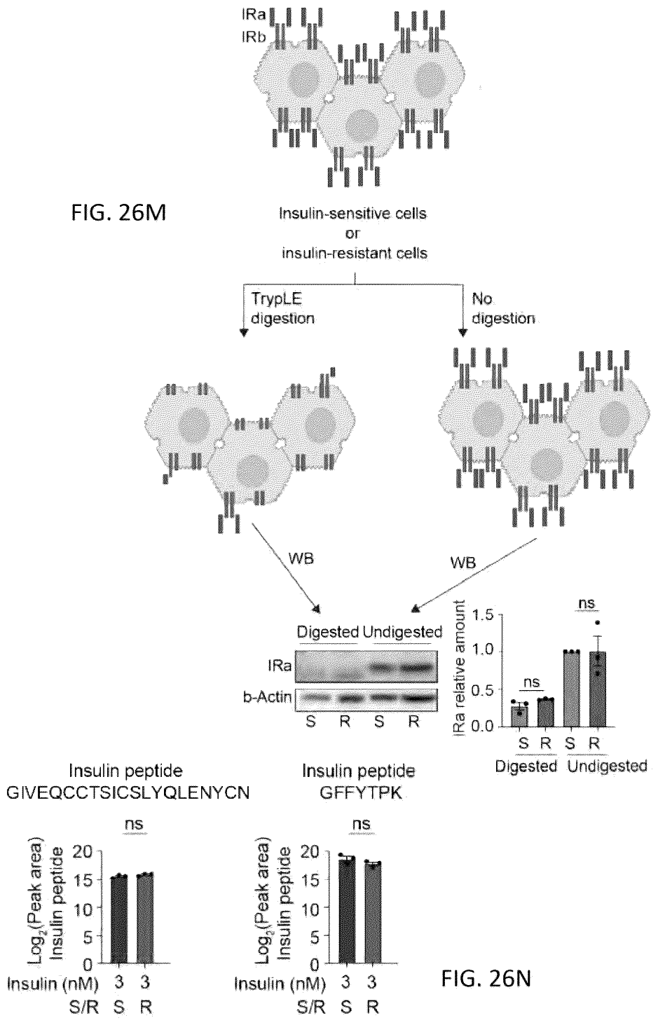

To investigate whether IR puncta are altered in insulin resistance, applicant compared the insulin-sensitive HepG2 cells to cells in which an insulin-resistant state was induced by hyperinsulinemia. HepG2 cells were exposed to either physiologic levels (0.1 nM) or pathologic levels (3 nM) of insulin^{48-50,57} for two days (FIG. 26A). Cells exposed to pathologic levels of insulin showed hallmarks of insulin resistance including after insulin stimulation: reduced phosphorylation of IR, IRS1, AKT and ERK (FIGS. 26B-26E), unchanged expression of the lipogenic gene FASN (FIG. 26F), unchanged lipogenesis and glucose production (FIGS. 26G-26I), and decreased phosphorylation of GSK3 (FIG. 26J). Insulin-sensitive and resistant cells contained similar amounts of IR in whole cell extracts and at the cell surface (FIGS. 26K-26M). Insulin binding was also similar between insulin-sensitive and resistant cells (FIG. 26N). These results suggest that the attenuated IR signaling was not due to a substantial change in IR levels in cells and at the plasma membrane, nor due to changes in the ability of insulin receptor to bind insulin.

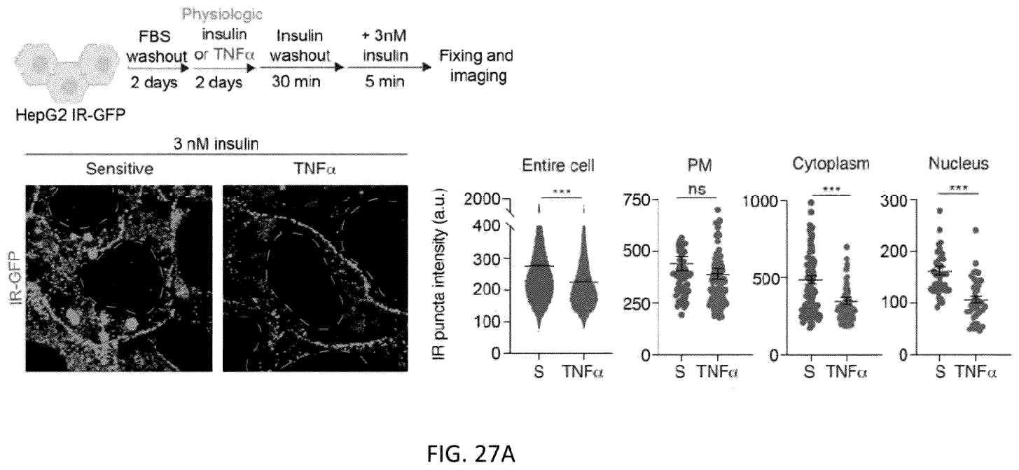

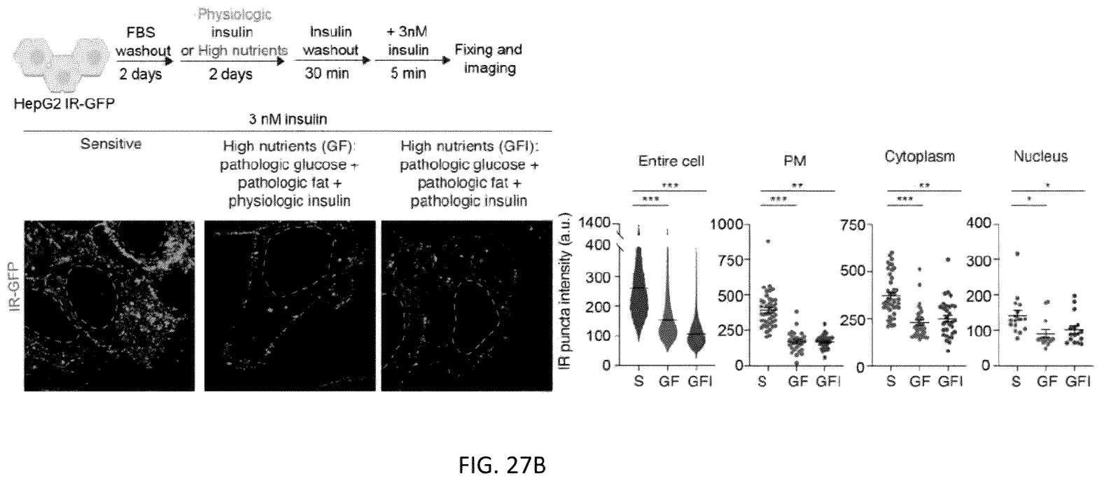

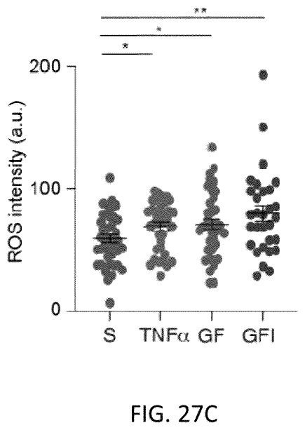

Immunofluorescence imaging of IR in insulin-resistant cells revealed that it is incorporated into puncta at the plasma membrane, cytoplasm and nucleus in a manner similar to that observed for insulin-sensitive cells (compare FIG. 16C and FIG. 16A, 0 nM insulin). However, in these insulin-resistant cells, acute treatment with insulin (3 nM) did not promote incorporation of additional IR into puncta (FIGS. 16B-16C), in contrast to the effects observed in insulin-sensitive cells (FIGS. 16A-16B). If the observed IR puncta defects are common features of insulin-resistant cells, then cells treated with other conditions expected to induce insulin resistance, such as chronic inflammation and high nutrient levels^{17,18}, should exhibit IR puncta defects that phenocopy those caused by hyperinsulinemia. Treatment of cells with pathological concentrations of TNFα or with high nutrients also caused a decrease in insulin-stimulated IR incorporation into puncta similar to that observed for hyperinsulinemia (FIG. 27). These results suggest that IR puncta dysfunction, defined here with respect to accumulation of molecules in puncta, may be a common feature of insulin resistance induced by diverse factors.

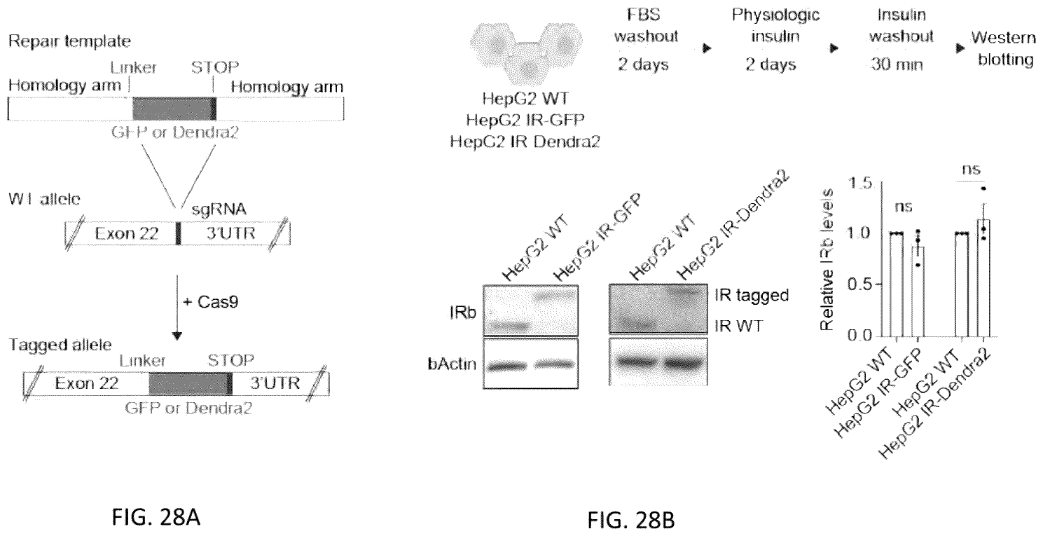

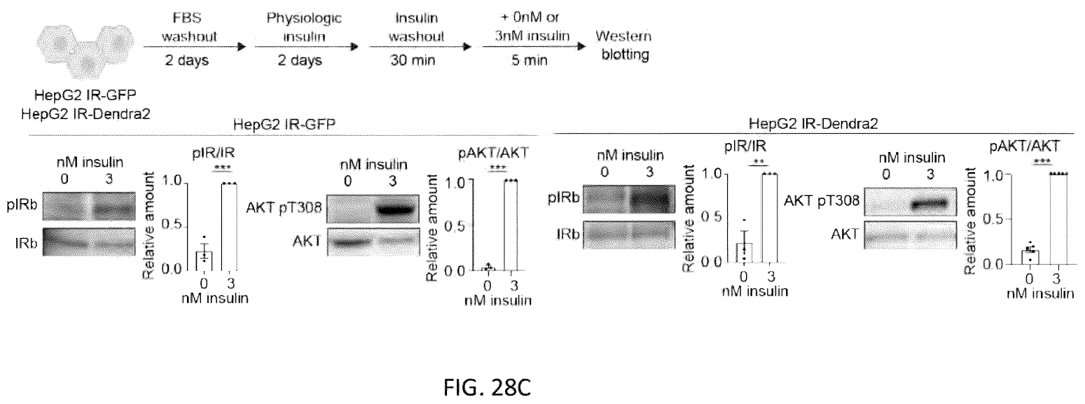

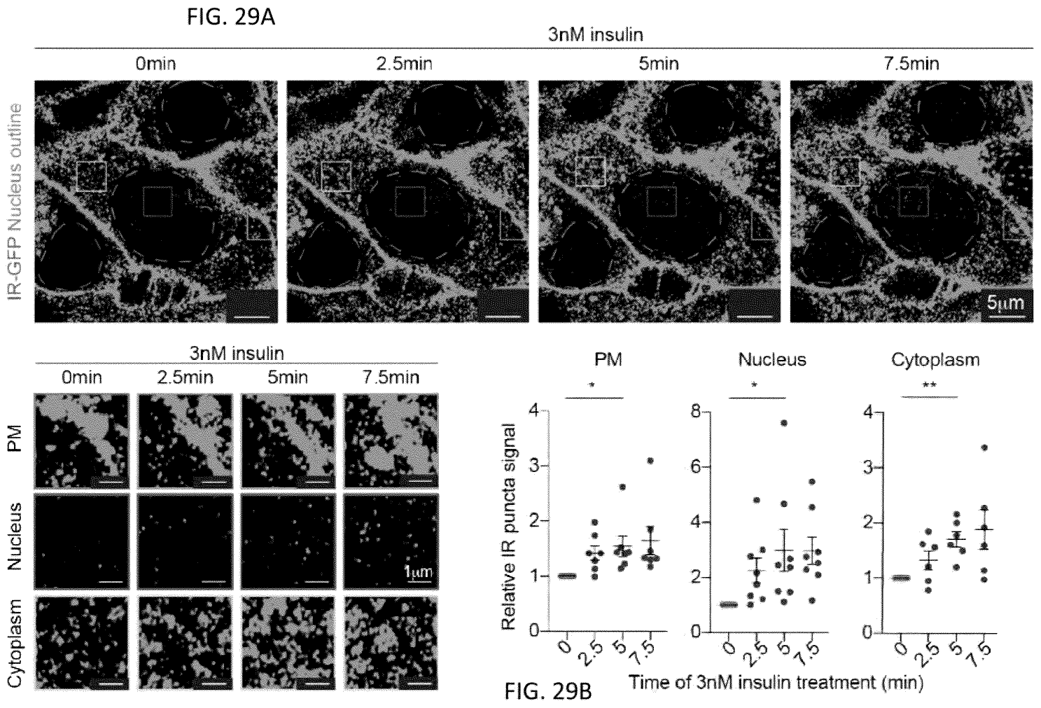

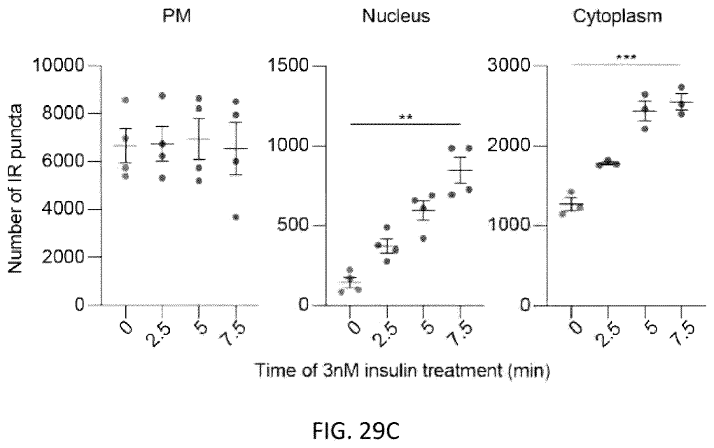

To confirm these observations and enable imaging of IR in live cells, HepG2 cells were engineered to express endogenous IR as a fusion protein with monomeric enhanced green fluorescent protein (IR-GFP) (FIG. 28A). IR-GFP was expressed in these homozygous cells at the same levels as WT IR and was functional, as cells expressing this fusion protein maintained insulin-induced phosphorylation of IR and insulin signaling proteins (FIGS. 28B-28C). A time course of insulin stimulation using live-cell imaging provided further evidence that insulin stimulation promotes an increase in IR-GFP signal in IR puncta (FIGS. 29A-29B), as well as an increase in the number of IR puncta in the nucleus and cytoplasm (FIG. 29C).

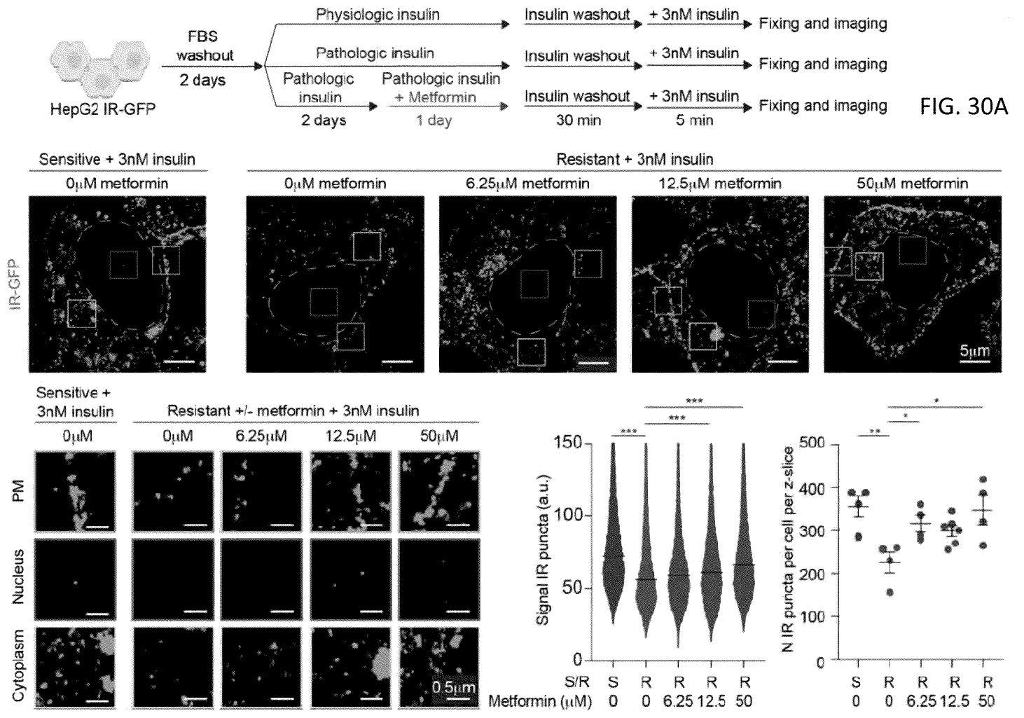

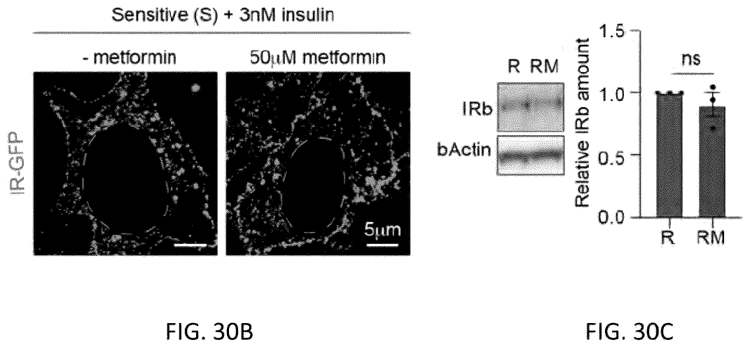

Given the observation that hepatocytes in liver tissue from metformin-treated T2D patients have IR puncta that resemble those in healthy donors, applicant investigated whether metformin could rescue the reduction in IR punctate signal seen in insulin-resistant HepG2 cells. Applicant again observed that IR-GFP HepG2 cells rendered insulin resistant showed reduced insulin-promoted incorporation of IR into puncta (FIGS. 16D-16E) and found that treatment of these insulin-resistant cells with metformin partially restored IR signal in these puncta (FIGS. 16D-16E and FIG. 30A). Treatment of insulin-sensitive HepG2 cells with metformin had little or no effect on IR puncta (FIG. 30B). The rescue of IR puncta phenotype in insulin-resistant cells was not due to changes in IR levels (FIG. 30C). IR condensate rescue was evident at 12.5 μM metformin (FIG. 30A), which approximates the concentration of metformin in the plasma of T2D patients⁵⁸⁻⁶⁰. These results indicate that the insulin-resistant state in these cells is associated with reduced IR incorporation in puncta, and that this dysfunction can be reversed to some extent by metformin, as observed in human liver tissue (compare FIG. 15A with FIG. 16D).

### Insulin Receptor Bodies in Primary Hepatocytes and Adipocytes

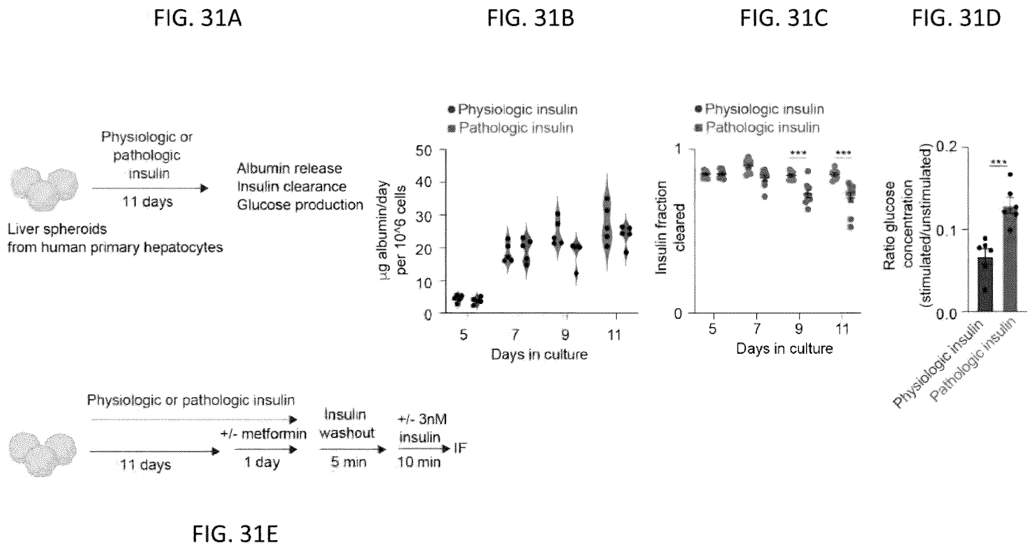

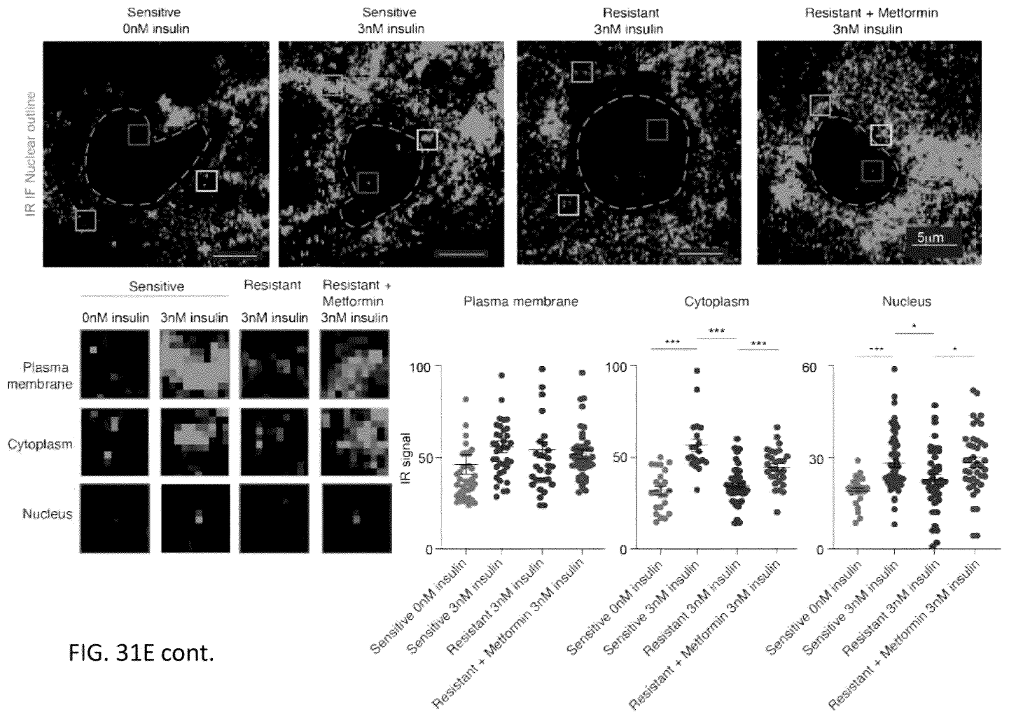

Applicant next investigated whether similar IR puncta occur in human primary hepatocytes, whether these are altered in insulin resistance, and studied the effects of metformin on such puncta. Primary human hepatocytes can form three-dimensional spheroids and can be cultured for days with physiological or pathological concentrations of insulin while maintaining their cell identity and function (FIGS. 31A-31B). These hepatocyte spheroids are insulin-sensitive if cultured with physiological concentrations of insulin and insulin-resistant if subjected to insulin levels characteristic of chronic hyperinsulinemia (FIGS. 31C-31D). In insulin-sensitive human liver spheroids, IR was found in puncta at the plasma membrane, cytoplasm and nucleus (FIG. 31E). As observed with HepG2 cells, insulin stimulation of hepatocyte spheroids produced an increase in IR signal intensity in cytoplasmic and nuclear puncta (FIG. 31E). In insulin-resistant spheroids, by contrast, IR incorporation into cytoplasmic and nuclear puncta was diminished (FIG. 31E), and metformin treatment partially rescued this attenuation of IR puncta signal (FIG. 31E). These results show that the phenotypes observed for IR puncta in insulin-sensitive and insulin-resistant HepG2 cells also occur in primary human hepatocyte spheroids.

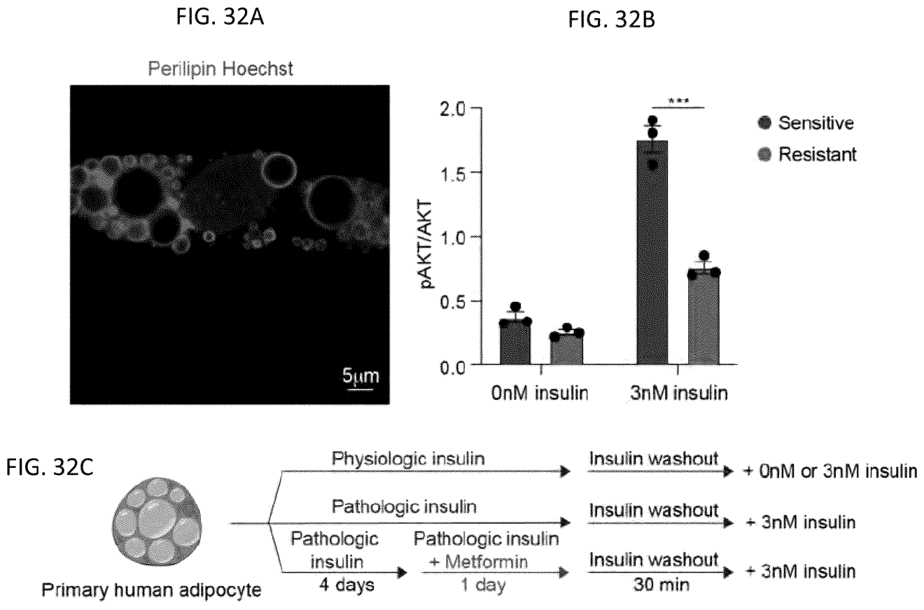

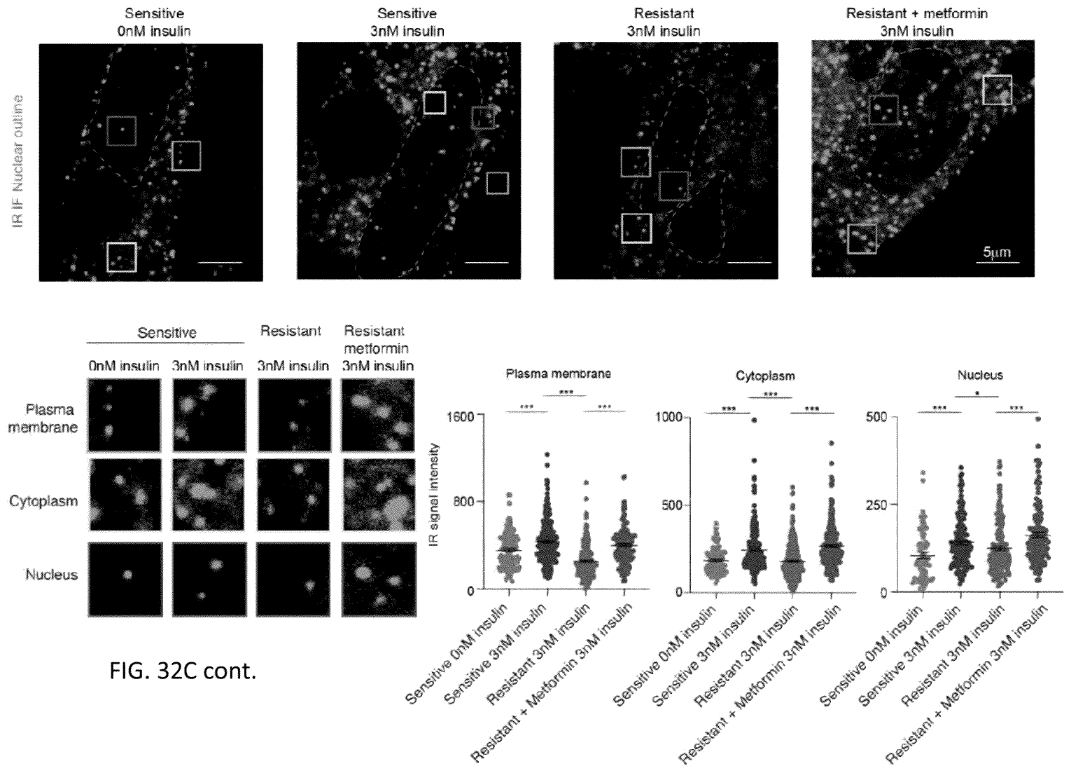

Adipocytes are among the cell types that exhibit insulin-resistant behavior, so applicant also investigated whether primary human adipocytes exhibit IR puncta phenotypes similar to those observed in hepatocytes (FIG. 32). Primary human pre-adipocytes were first differentiated into adipocytes (FIG. 32A) and then cultured for five days with either physiological concentrations of insulin or pathological concentration of insulin known to induce insulin-resistance in adipocytes⁶¹ (FIG. 32B). As observed with insulin-sensitive hepatocytes, IR-associated puncta were found at the plasma membrane, in the cytoplasm, and in nuclei of insulin-sensitive adipocytes, and insulin stimulation promoted further IR incorporation into these puncta (FIG. 32C). In the insulin-resistant adipocytes, insulin stimulation was less able to promote further IR incorporation into puncta and this reduction in signal was reversed by metformin (FIG. 32C). These results show that primary human adipocytes exhibit IR puncta phenotypes similar to those observed in hepatocytes.

### Insulin Receptor Bodies Exhibit Features of Liquid-Like Condensates

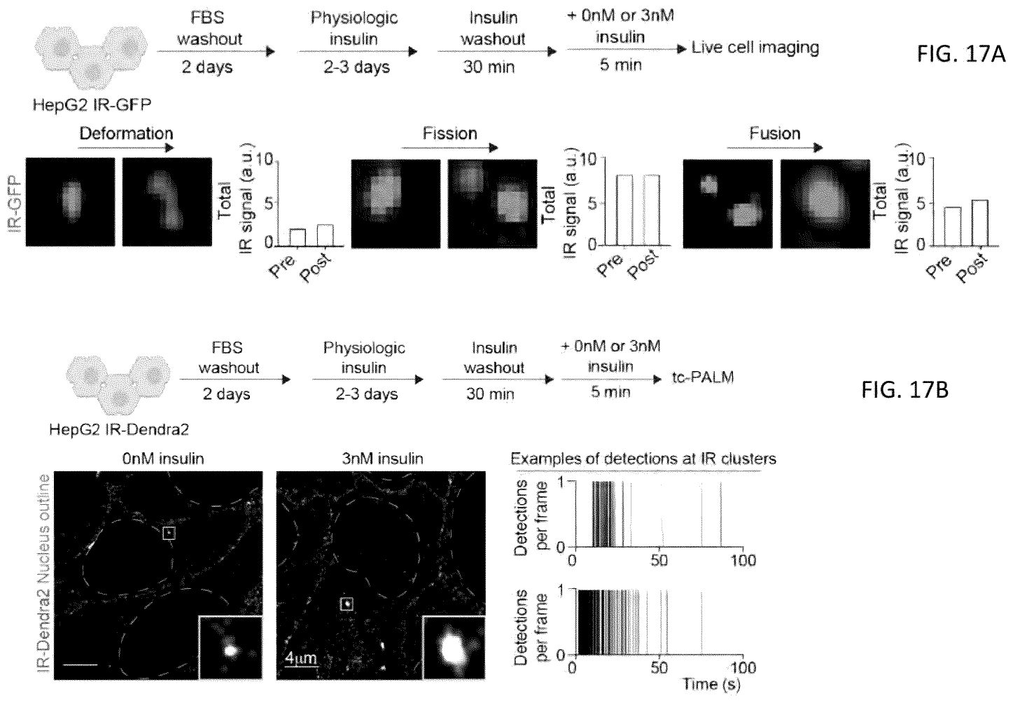

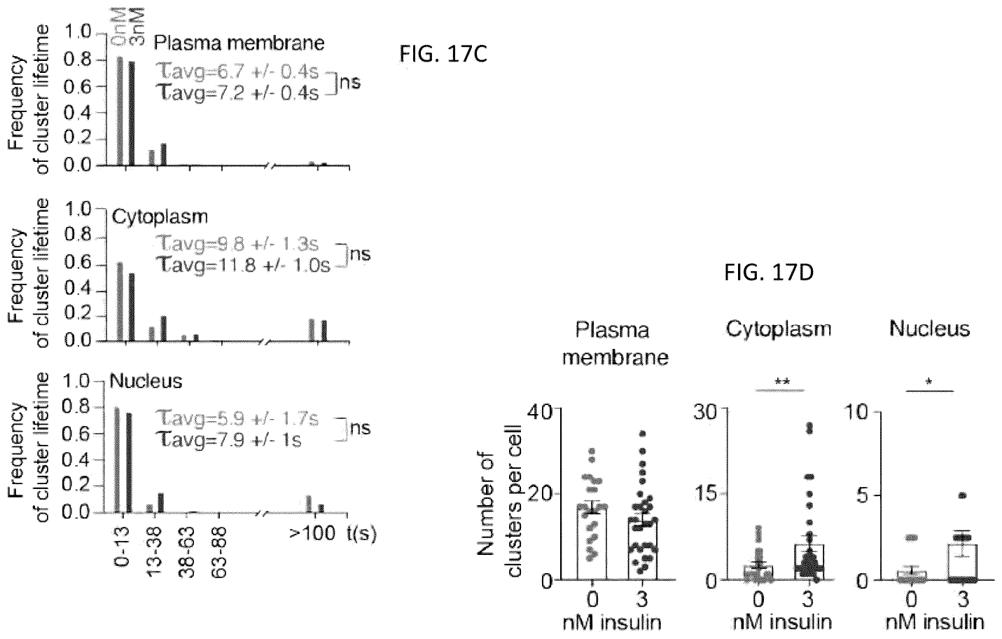

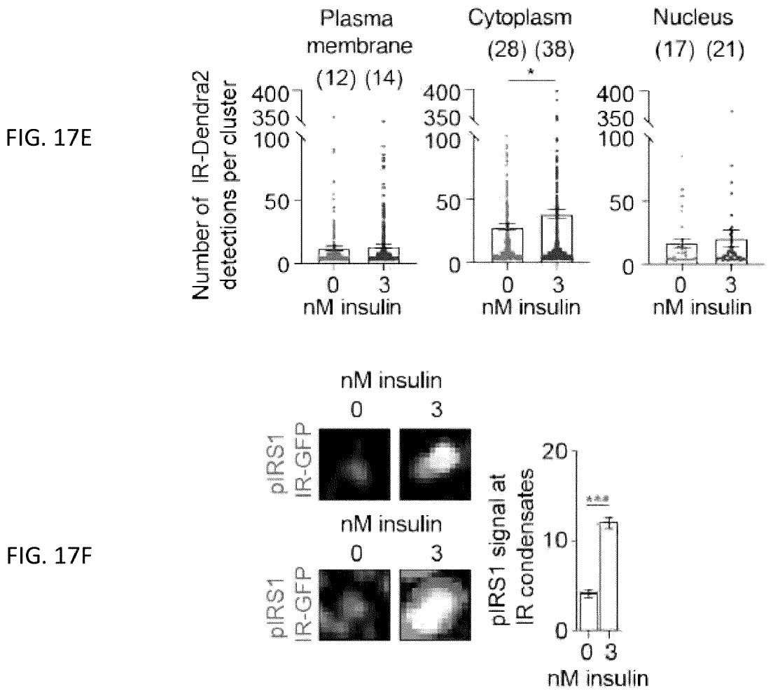

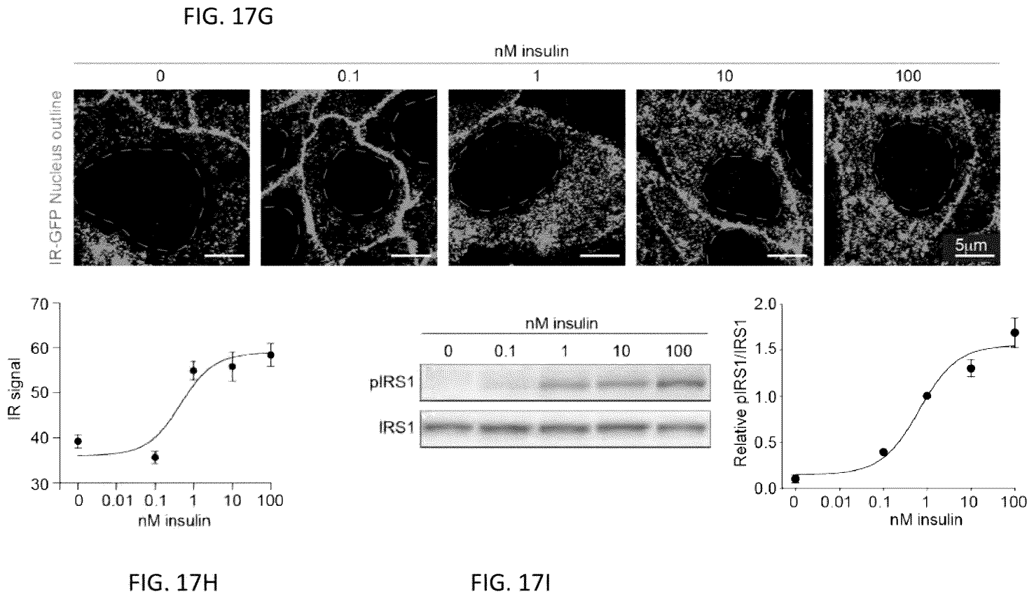

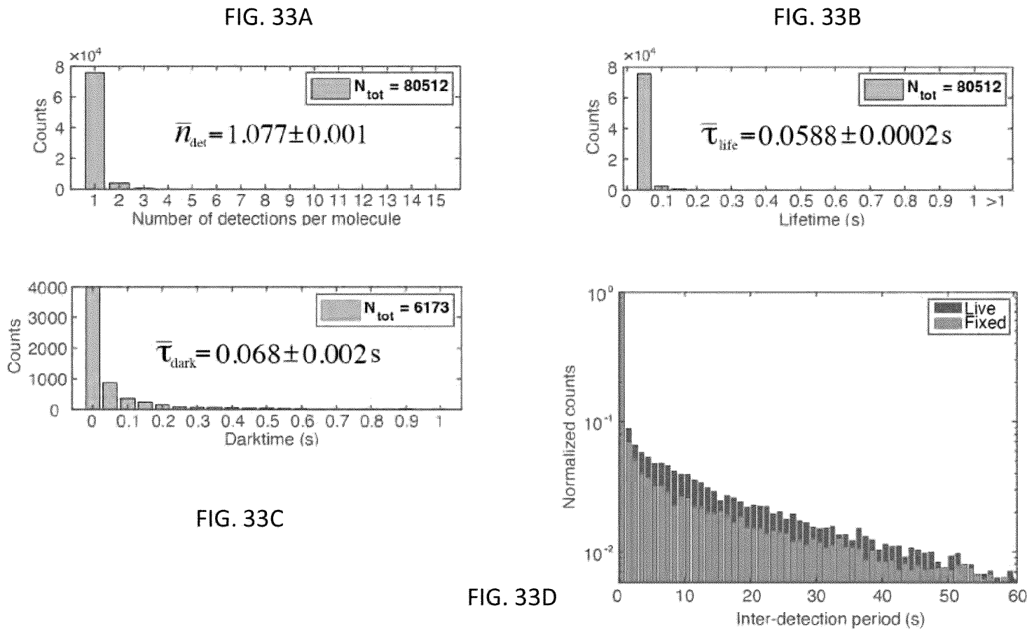

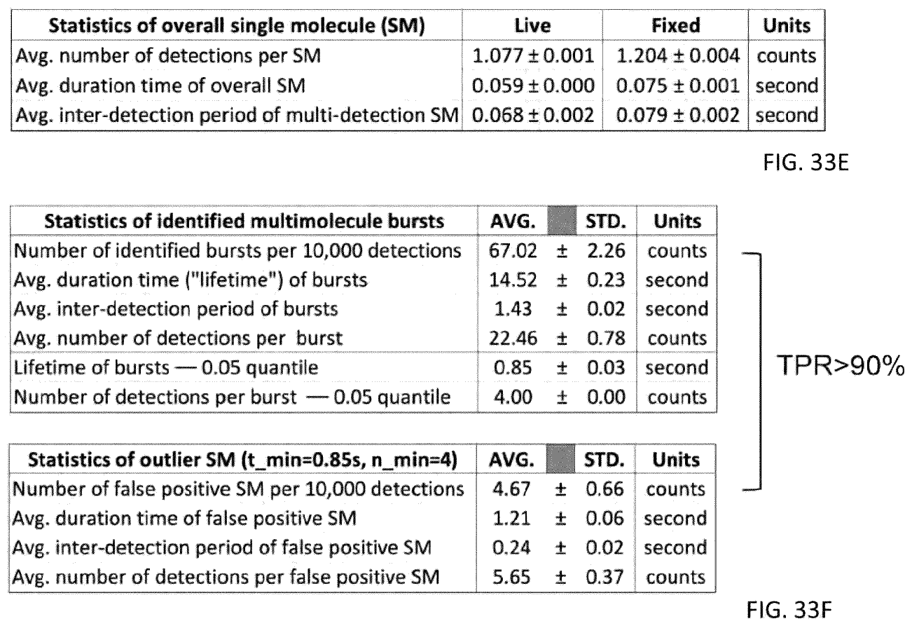

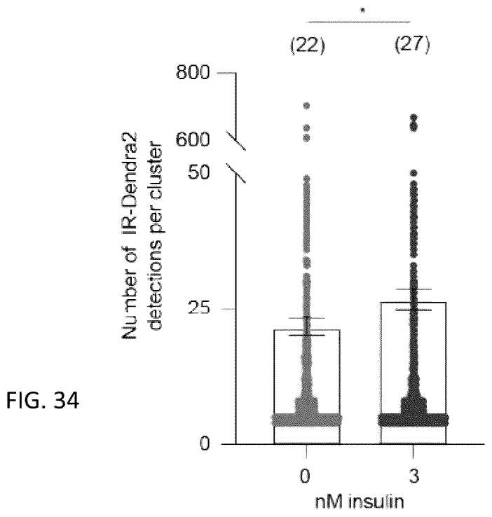

The appearance of proteins in punctate bodies might be due to their incorporation into biomolecular condensates^{34,62}, which are dynamic assemblies of molecules that can undergo deformation, fission, and fusion⁶³⁻⁶⁸. Super-resolution microscopy of HepG2 IR-GFP cells revealed IR-GFP puncta in the plasma membrane, cytoplasm and nucleus do indeed undergo deformation, fission and fusion (FIG. 17A). Another feature of liquid-like biomolecular condensates is that they form and dissolve in short (second-minute) time frames⁶³. To investigate this property, applicant used time-correlated photoactivation localization microscopy (tc-PALM)^{35,63,69} with a HepG2 cell line engineered to express endogenous IR as a fusion protein with Dendra2 (IR-Dendra2) (FIG. 28A). IR-Dendra2 was expressed at the same levels as WT IR and was functional, as this fusion protein maintained its kinase activity (FIGS. 28B-28C). IR-Dendra2 cells were subjected to tc-PALM and clusters of IR molecules were studied (FIG. 17B); several control analyses of the single molecule photochemistry were performed to validate the statistics of the molecular clusters examined here (FIG. 33). The results revealed that IR forms dynamic clusters at the plasma membrane, cytoplasm and nucleus that exhibit various lifetimes, consistent with dynamic condensate formation and dissolution (FIGS. 16B-16C). In cells with and without insulin stimulation, the majority of IR clusters (˜85%) were short-lived (lifetime <100 s) and had an average lifetime of 6-12 s (FIG. 17C), comparable to those measured for transient transcriptional condensates in various cell types^{35,63,69}. A smaller fraction of clusters (˜15%) were present for considerably longer lifetimes (>100 s) (FIG. 17C). Insulin stimulation resulted in an increase in the number of IR clusters in the cytoplasm and nucleus of these cells (FIG. 17D) and an increase in the number of IR detections in clusters in insulin-sensitive cells (FIG. 34). The average number of IR-Dendra2 detections per cluster was estimated to be 22 (range 4-609) in unstimulated cells, and 27 (range 4-539) in insulin-stimulated cells (FIG. 34). A similar trend was observed in IR clusters at the plasma membrane, in the cytoplasm and in the nucleus (FIG. 17E). These results suggest that multiple molecules of IR are incorporated into dynamic clusters, consistent with the behavior expected for biomolecular condensates, and that insulin stimulation leads to an increase in the number of IR-containing clusters and in the number of IR molecules per cluster in the cytoplasm and nucleus.

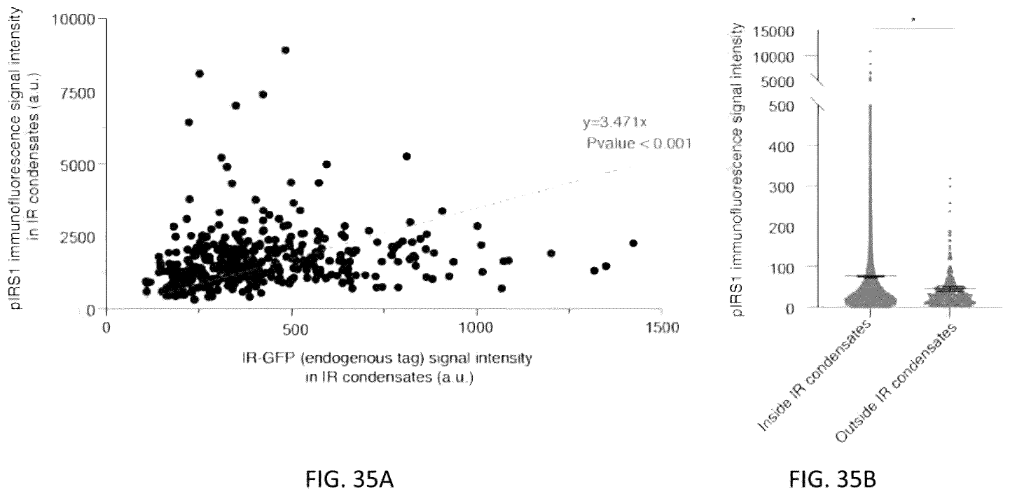

Applicant next sought evidence that IR molecules present in condensates are functionally active in those condensates. If IR kinase activity occurs in condensates, then applicant would expect that the level of phosphorylated IR substrate IRS1 would increase in these IR-associated condensates upon insulin stimulation. Indeed, immunofluorescence microscopy with an antibody specific for phosphorylated IRS1 (pIRS1) showed that insulin stimulation increased the intensity of pIRS1 at IR condensates (FIG. 17F). IR incorporation into condensates positively correlated with signal intensity of pIRS1 in these condensates (FIG. 35A) and pIRS1 was more concentrated inside IR condensates than outside (FIG. 35B). In addition, acute stimulation of insulin-sensitive cells with a range of insulin concentrations produced a non-linear transition in IR-incorporation into condensates (FIGS. 17G-17H), and the jump in IR condensate signal occurred coincident with insulin receptor activity and function measured by IRS1 phosphorylation (FIGS. 17G-17I). This non-linear association between IR incorporation in condensates and IR kinase activity is expected if the IR molecules incorporated into condensates are functional within condensates.

### Altered Insulin Receptor Dynamics in Insulin-Resistant Cells and Rescue by Metformin

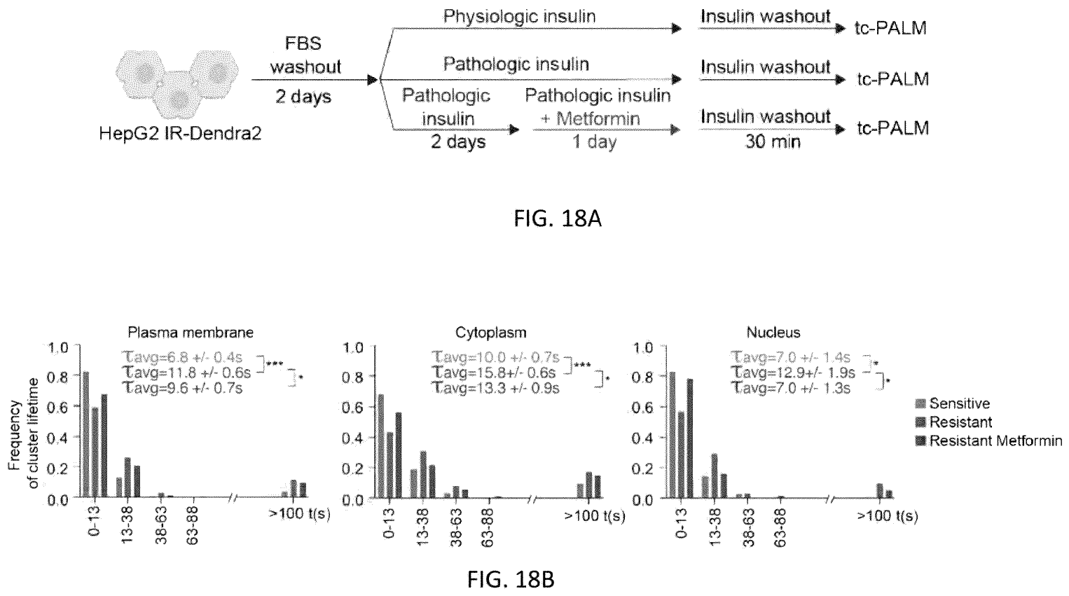

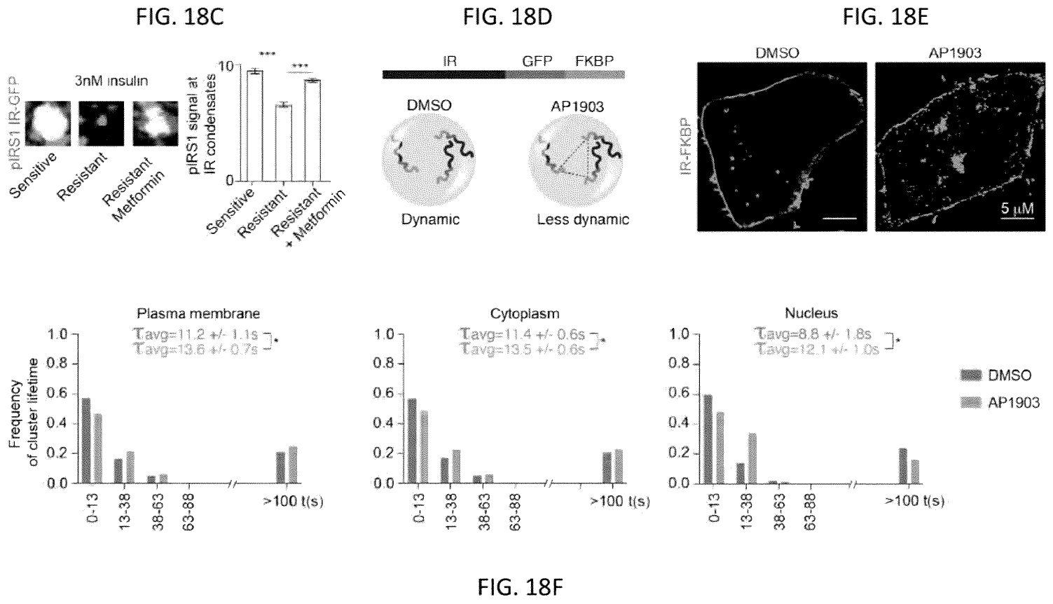

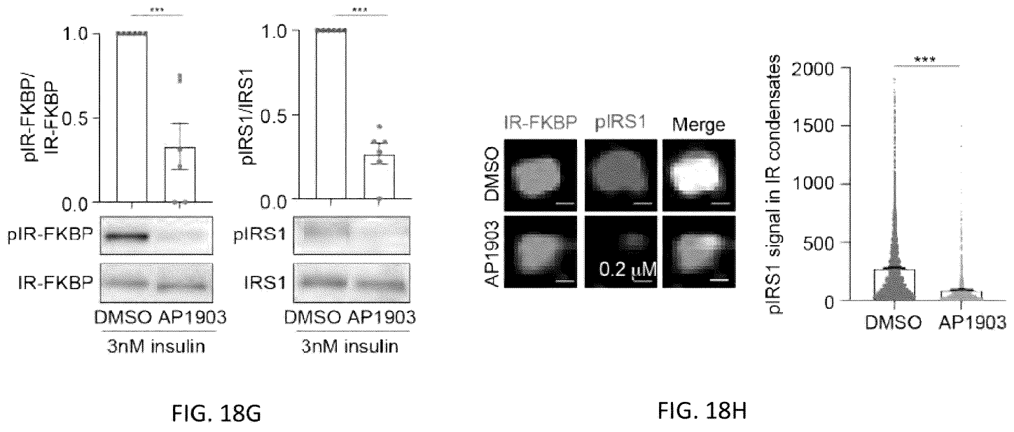

Chronic estrogen signaling was recently shown to reduce the dynamic properties of estrogen receptor condensates³⁶ so applicant investigated whether the dynamics of IR condensates are altered in insulin-resistant cells (FIG. 18). HepG2 cells expressing IR-Dendra2 were exposed to physiologic levels of insulin (0.1 nM) to maintain insulin sensitivity, or pathologic levels of insulin (3 nM)^{48-50,57} to promote insulin resistance (FIG. 18A). tc-PALM was used to measure IR condensate dynamics in the insulin-sensitive and resistant cells. The results showed that IR molecules remained in condensates for longer lifetimes in the cytoplasm and nucleus in insulin-resistant cells relative to insulin-sensitive cells (FIG. 18B). The average lifetime of short-lived IR condensates in sensitive versus insulin-resistant cells increased from 6.8 s to 11.8 s at the plasma membrane, from 10.0 s to 15.8 s in the cytoplasm and from 7.0 s to 12.9 s in the nucleus. The percentage of long-lived IR condensates also increased in the plasma membrane, cytoplasm and nucleus (FIG. 18B). These results suggest that the insulin-resistant state is associated with reduced IR condensate dynamics, reflected in the longer lifetime of these condensates, which may account for the attenuated responses observed during insulin stimulation.

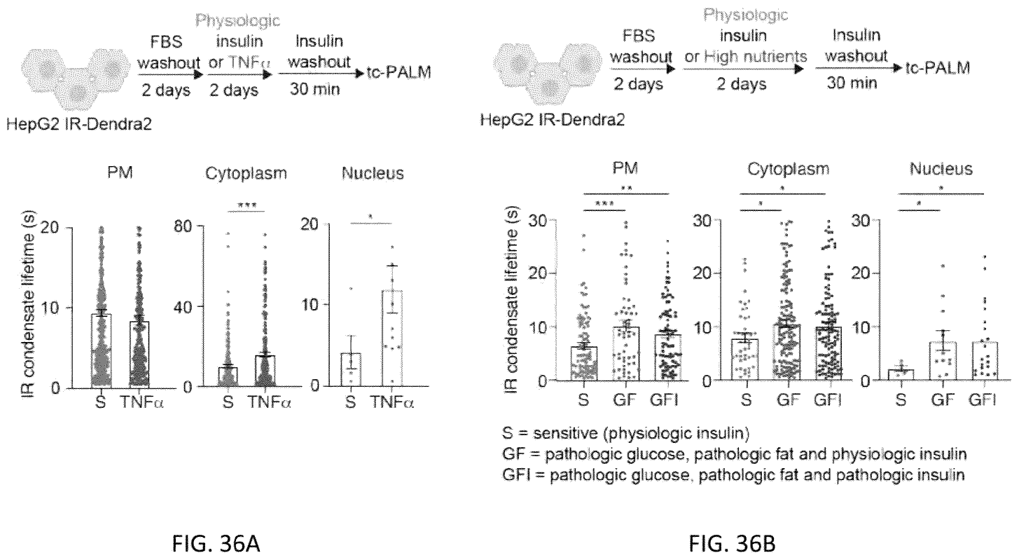

Applicant wondered whether IR condensate dynamics are also decreased in other models of insulin resistance. Treatment of cells with pathological concentrations of TNFα or with high nutrients decreased IR dynamics in condensates (FIGS. 36A-36B). These results indicate that IR condensate dysfunction, defined here with respect to accumulation and dynamics of molecules, may be a common feature of insulin resistance induced by diverse factors.

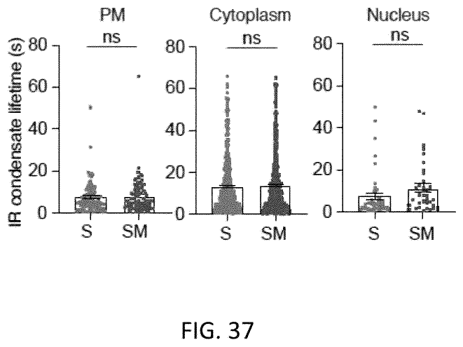

Applicant next examined the effect of metformin treatment on IR condensate dynamics. Metformin treatment of insulin-resistant cells rescued condensate lifetimes in the plasma membrane, cytoplasm and nucleus to times that were similar to those in insulin-sensitive cells (FIG. 18B). For example, while ˜40% of cytoplasmic IR condensates in insulin-resistant cells had a lifetime of 0-13 s, ˜60% of IR condensates in the cytoplasm of insulin-sensitive and metformin-treated resistant cells had a lifetime of 0-13 s (FIG. 18B). Similarly, the frequency of plasma membrane and nuclear IR condensates with 0-13 s lifetimes, which was reduced in the resistant cells relative to sensitive cells, was increased by the metformin treatment (FIG. 18B). In contrast, metformin did not decrease IR condensate lifetime in insulin-sensitive cells (FIG. 37). Thus, metformin treatment rescues the dynamic properties of IR-containing condensates that occur in insulin-resistant cells.

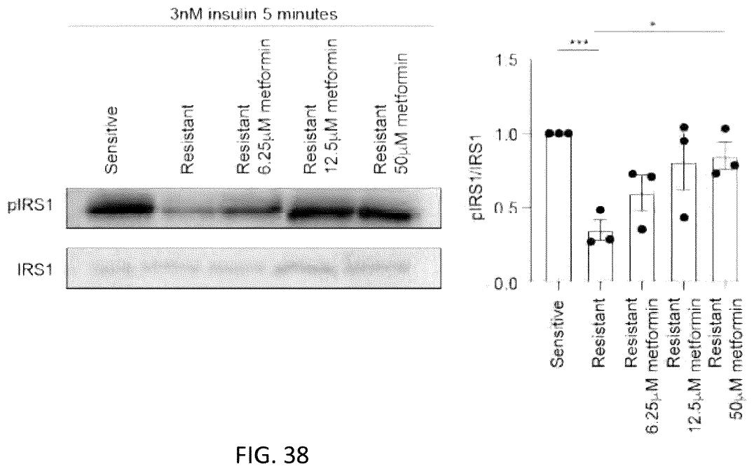

Applicant next investigated whether IR kinase activity differs in condensates in insulin-resistant cells and in these cells treated with metformin. Imaging experiments revealed reduced levels of phosphorylated IRS1 in IR-containing condensates in insulin-resistant cells as compared to insulin-sensitive cells (FIG. 18C). Metformin treatment partially rescued the levels of phosphorylated IRS1 in IR-containing condensates in insulin-resistant cells (FIG. 18C). These results were further supported by western blotting experiments that revealed a partial rescue of IRS1 phosphorylation in insulin-resistant cells by metformin treatment (FIG. 38). Taken together, these results suggest that IR kinase activity is reduced in condensates in insulin-resistant cells and that metformin treatment can partially reverse this effect.

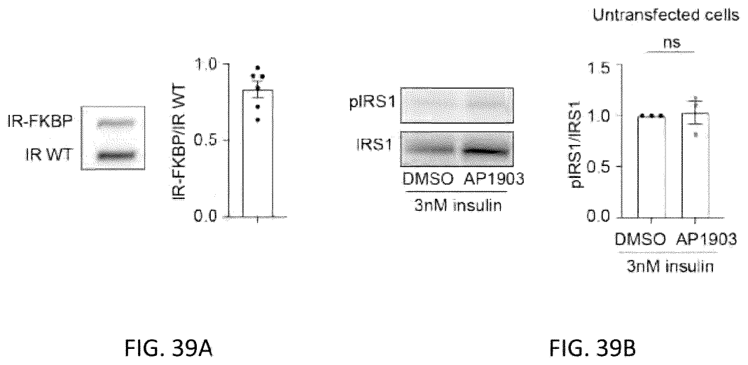

Applicant next explored whether changes in condensate dynamics might have a direct effect on IR kinase activity in the condensate. To artificially decrease IR molecule dynamics within condensates, applicant fused IR-GFP to 4 tandem repeats of FK506 binding protein (FKBP; IR-FKBP), which interact with each other only in the presence of the small molecule AP1903⁷⁰. IR-FKBP was expressed in HepG2 cells and these cells were treated with AP1903 or control DMSO (FIGS. 18D-18H). Treating HepG2 cells expressing IR-FKBP with AP1903 significantly increased the lifetime of IR condensates, consistent with a reduction in IR molecule dynamics in these condensates (FIG. 18F). Western blotting and imaging experiments revealed that IR was less functionally active in cells expressing IR-FKBP treated with AP1903 (FIGS. 18G-18H, FIG. 39). Taken together, these results indicate that a decrease in insulin receptor condensate dynamics can produce a decrease in IR activity.

### High ROS Levels Promote IR Condensate Dysregulation

Several observations led us to test the hypothesis that high levels of reactive oxygen species (ROS) contribute to dysregulated IR condensates in insulin-resistant cells. Insulin stimulation causes a transient increase in H₂O₂ levels⁷¹⁻⁷⁶. Many cell-extrinsic factors that promote insulin resistance, including hyperinsulinemia, TNFα and high nutrients lead to excessive production of ROS^{19,77-79} (FIG. 27C). Insulin-resistant cells and patients with T2D have been shown to have elevated levels of ROS^{43,44} and high ROS is a known cell-intrinsic factor that promotes insulin resistance^{19,77,80,81}. Metformin has been proposed to decrease ROS levels by multiple mechanisms, including inhibition of the mitochondrial complex I respiratory chain⁴⁵, inhibition of the redox shuttle enzyme mitochondrial glycerophosphate dehydrogenase⁴², and upregulation and activation of anti-oxidants⁸². Importantly, oxidative stress has previously been shown to affect condensate behaviors⁸³⁻⁸⁸.

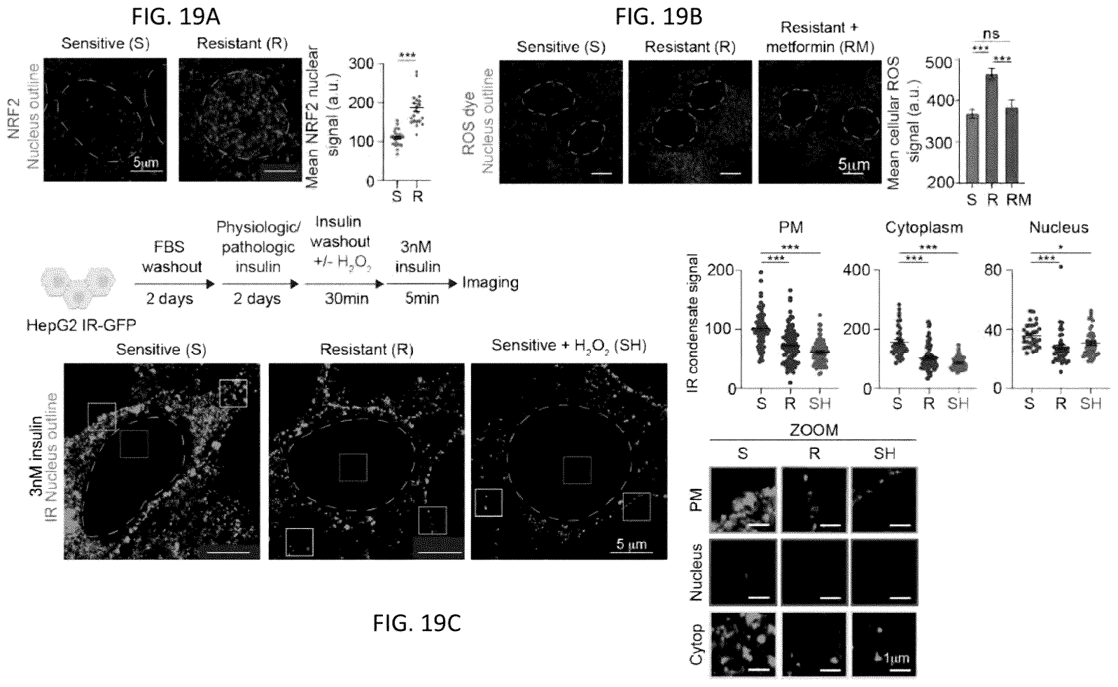

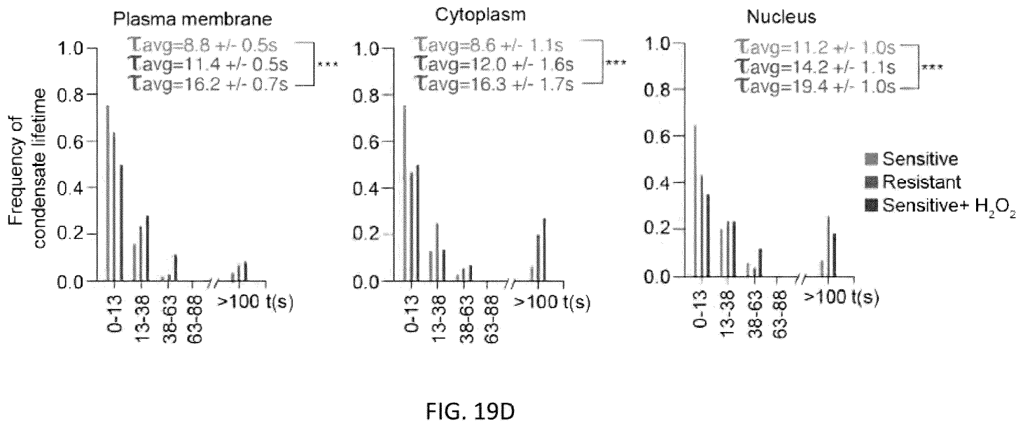

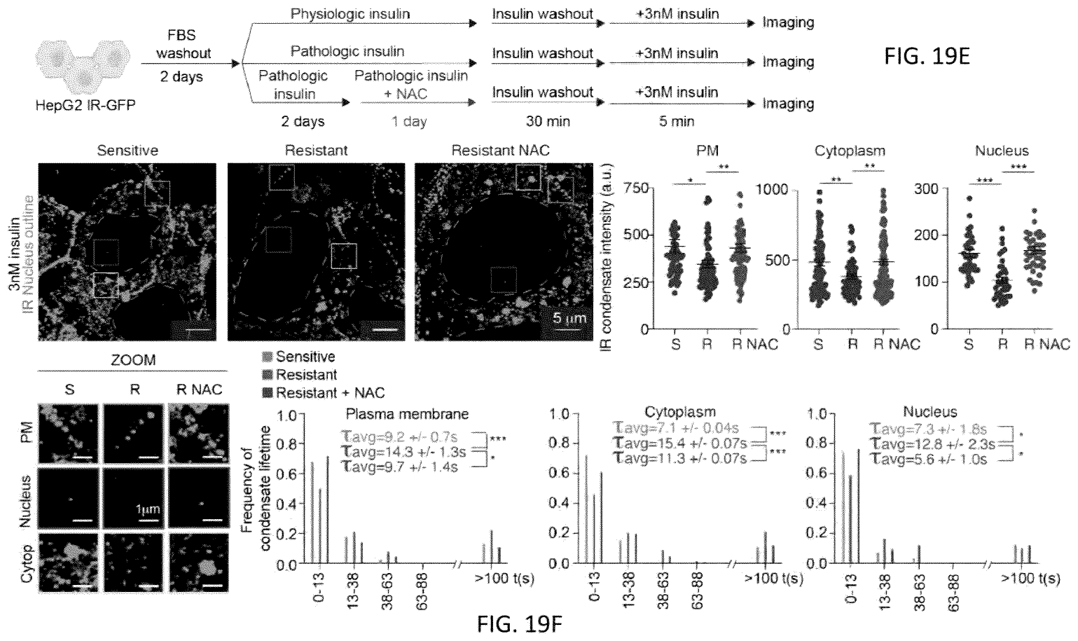

To test this idea, applicant first determined if insulin-resistant cells are subjected to higher levels of oxidative stress than insulin-sensitive cells. Imaging of NRF2, a marker of oxidative stress⁸⁹, revealed that insulin-resistant cells experienced higher levels of oxidative stress than insulin-sensitive cells (FIG. 19A). Quantification of ROS using a ROS-sensitive dye revealed that ROS levels were higher in insulin-resistant cells and, furthermore, that metformin treatment of these cells reduced ROS levels to those found in insulin-sensitive cells (FIG. 19B).

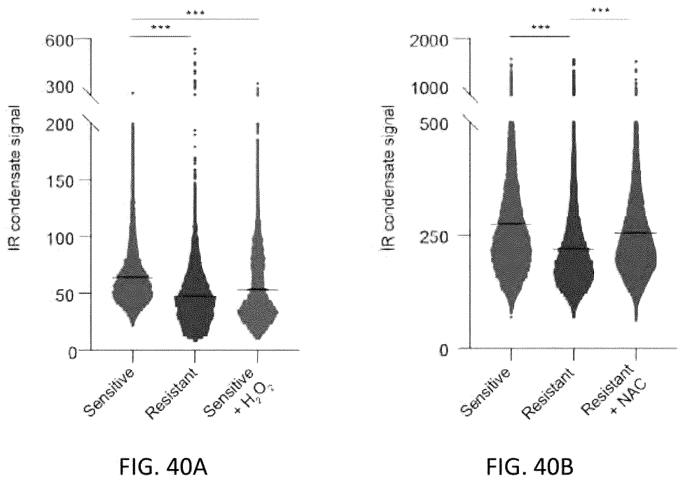

If oxidative stress causes condensate dysregulation, then treatment of insulin-sensitive cells with concentrations of an oxidizing agent known to cause oxidative stress might be expected to phenocopy the effects seen with insulin resistance. Similarly, if metformin acts by relieving the effects of oxidative stress, treatment of insulin-resistant cells with a reagent that reduces oxidative stress might phenocopy the effects of metformin. Indeed, applicant found that treating insulin-sensitive cells for 30 minutes with a concentration of H₂O₂ known to cause oxidative stress⁹⁰ caused a reduction in the incorporation of IR into condensates with insulin stimulation and altered IR condensate dynamics, phenocopying the condensate dysregulation seen in insulin-resistant cells (FIGS. 19C-19D, FIG. 40A). Furthermore, treatment of insulin-resistant cells with clinically relevant concentrations of N-acetyl cysteine (NAC)^{91,92} partially rescued IR condensates defects (FIGS. 19E-19F, FIG. 40B). Together, these results suggest that chronic hyperinsulinemia leads to excess levels of ROS in insulin-resistant HepG2 cells, that high levels of ROS alter IR incorporation into condensates, and that anti-oxidants can partially rescue the dysregulated IR-condensate behaviors as a consequence of reducing ROS levels.

### Discussion

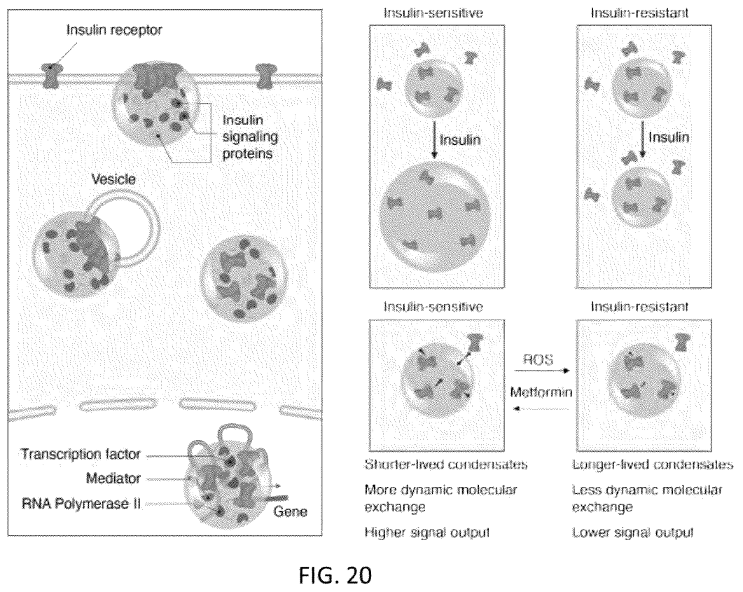

Applicant considered a condensate model for insulin receptor (IR) function in normal conditions and when dysregulated in insulin resistance (FIG. 20). The results reveal that IR is incorporated into dynamic condensates at the plasma membrane, in the cytoplasm and in the nucleus (FIG. 20A). Acute insulin stimulation promotes further incorporation of IR into condensates in insulin-sensitive cells, and this effect is attenuated in insulin-resistant cells (FIG. 20B). In insulin-resistant cells, IR-condensate dynamics are altered (FIG. 20C). This dysfunction is rescued with metformin treatment (FIG. 20C). In insulin-resistant cells, prolonged elevation of ROS levels appears to account for altered condensate dynamics because it can be phenocopied by H₂O₂ treatment of insulin-sensitive cells and rescued by NAC treatment of insulin-resistant cells (FIG. 20C). Metformin likely rescues condensate dynamics in insulin-resistant cells by reducing ROS levels.

Recent studies have shown that the components of diverse signaling pathways, including those involving receptor tyrosine kinases, T cell receptor, WNT, TGF-β, and JAK/STAT, involve the assembly of protein molecules into liquid-like biomolecular condensates at the plasma membrane, in the cytoplasm and nucleus²⁴⁻³³. The evidence indicates that this is also the case for the insulin receptor. There is also recent evidence that chronic nuclear receptor stimulation can lead to altered condensate dynamics; the estrogen receptor forms less dynamic condensates, and these are associated with decreased transcriptional output, when subjected to chronic high levels of estrogen³⁶. The evidence indicates that chronic hyperinsulinemia also alters IR condensate dynamics, and that this alteration is associated with reduced IR activity.

Applicants found that prolonged elevation of ROS levels in chronic hyperinsulinemia reduces the ability of insulin to promote further incorporation of IR molecules into condensates and extends the lifetime of IR molecules within the existing condensates. The known effects of ROS on proteins provides a mechanism to explain these findings. Transient insulin-induced H₂O₂ formation is essential for mediating insulin signaling⁷¹⁻⁷⁶, but ROS can cause protein oxidation, which can alter protein conformation and change the ability of proteins to be incorporated into condensates^{83,93}. It is possible that ROS-induced alteration of proteins in condensates may be a common mechanism in the pathogenesis of insulin resistance-associated diseases, including T2D, metabolic syndrome, non-alcoholic fatty liver disease (NAFLD), polycystic ovarian syndrome (PCOS), and Alzheimer's Disease. Indeed, metformin has been shown to decrease ROS production and improve patient outcomes in T2D and other diseases characterized by high ROS levels^{42,82,94-96}. The mitochondrial respiratory chain complex 1 is the primary target of metformin, but it may be the reduction in ROS levels and the consequent benefit to protein condensate dynamics that is key to normal condensate-associated IR signaling.

The proposal that insulin resistance is associated with IR condensate dysfunction is consistent with prior evidence that implicates defects of insulin signaling pathways in hepatocytes in vivo²⁰ and specific cellular stresses in both insulin resistance and condensate dysregulation. Some systemic and intracellular stresses that have been reported to induce insulin resistance, including oxidative stress and mitochondrial dysfunction, have independently been shown to influence the formation or behavior of cellular condensates^{93,97,98}. Further study of the molecular components of IR condensates and their oxidative modification should provide more detailed insights into the physicochemical properties that are altered in condensates by oxidative stress and mitochondrial dysfunction.

A condensate model for IR dysfunction has implications for development of novel therapeutics for T2D and other diseases that involve condensate dysregulation. For example, the condensate assays described here might be leveraged to develop new therapeutics that improve clinical outcomes for patients who cannot tolerate metformin or become resistant to the drug with prolonged use. Such therapeutics might also provide benefits to patients with other diseases where condensate dysregulation is also thought to play a role^{97,99,100}.

### REFERENCES

1. Batista, T. M., Haider, N. & Kahn, C. R. Defining the underlying defect in insulin action in type 2 diabetes. Diabetologia 64, 994-1006 (2021).
2. James, D. E., Stockli, J. & Birnbaum, M. J. The aetiology and molecular landscape of insulin resistance. Nat Rev Mol Cell Biol (2021).
3. Freychet, P., Roth, J. & Neville, D. M., Jr. Insulin receptors in the liver: specific binding of (125 I) insulin to the plasma membrane and its relation to insulin bioactivity. Proc Natl Acad Sci USA 68, 1833-1837 (1971).
4. Kasuga, M., Zick, Y., Blithe, D. L., Crettaz, M. & Kahn, C. R. Insulin stimulates tyrosine phosphorylation of the insulin receptor in a cell-free system. Nature 298, 667-669 (1982).
5. Yu, K. T. & Czech, M. P. Tyrosine phosphorylation of the insulin receptor beta subunit activates the receptor-associated tyrosine kinase activity. J Biol Chem 259, 5277-5286 (1984).
6. Yu, K. T., Pessin, J. E. & Czech, M. P. Regulation of insulin receptor kinase by multisite phosphorylation. Biochimie 67, 1081-1093 (1985).
7. White, M. F., Maron, R. & Kahn, C. R. Insulin rapidly stimulates tyrosine phosphorylation of a Mr-185,000 protein in intact cells. Nature 318, 183-186 (1985).
8. Pronk, G. J., McGlade, J., Pelicci, G., Pawson, T. & Bos, J. L. Insulin-induced phosphorylation of the 46- and 52-kDa She proteins. J Biol Chem 268, 5748-5753 (1993).
9. Furtado, L. M., Somwar, R., Sweeney, G., Niu, W. & Klip, A. Activation of the glucose transporter GLUT4 by insulin. Biochem Cell Biol 80, 569-578 (2002).
10. Goldfine, I. D. & Smith, G. J. Binding of insulin to isolated nuclei. Proc Natl Acad Sci USA 73, 1427-1431 (1976).
11. Hancock, M. L., et al. Insulin Receptor Associates with Promoters Genome-wide and Regulates Gene Expression. Cell 177, 722-736 e722 (2019).
12. Nelson, J. D., LeBoeuf, R. C. & Bomsztyk, K. Direct recruitment of insulin receptor and ERK signaling cascade to insulin-inducible gene loci. Diabetes 60, 127-137 (2011).
13. Fagerholm, S., Ortegren, U., Karlsson, M., Ruishalme, I. & Stralfors, P. Rapid insulin-dependent endocytosis of the insulin receptor by caveolae in primary adipocytes. PLoS One 4, e5985 (2009).
14. Hall, C., Yu, H. & Choi, E. Insulin receptor endocytosis in the pathophysiology of insulin resistance. Exp Mol Med 52, 911-920 (2020).
15. Iraburu, M. J., Garner, T. & Montiel-Duarte, C. Revising Endosomal Trafficking under Insulin Receptor Activation. Int J Mol Sci 22(2021).
16. Amaya, M. J., et al. The insulin receptor translocates to the nucleus to regulate cell proliferation in liver. Hepatology 59, 274-283 (2014).
17. Czech, M. P. Insulin action and resistance in obesity and type 2 diabetes. Nat Med 23, 804-814 (2017).
18. DeFronzo, R. A., et al. Type 2 diabetes mellitus. Nat Rev Dis Primers 1, 15019 (2015).
19. Petersen, M. C. & Shulman, G. I. Mechanisms of Insulin Action and Insulin Resistance. Physiol Rev 98, 2133-2223 (2018).
20. Ter Horst, K. W., et al. Hepatic Insulin Resistance Is Not Pathway Selective in Humans With Nonalcoholic Fatty Liver Disease. Diabetes Care 44, 489-498 (2021).
21. Furnica, R. M., Istasse, L. & Maiter, D. A severe but reversible reduction in insulin sensitivity is observed in patients with insulinoma. Ann Endocrinol (Paris) 79, 30-36 (2018).
22. Lemmer, I. L., Willemsen, N., Hilal, N. & Bartelt, A. A guide to understanding endoplasmic reticulum stress in metabolic disorders. Mol Metab 47, 101169 (2021).
23. Hurrle, S. & Hsu, W. H. The etiology of oxidative stress in insulin resistance. Biomed J 40, 257-262 (2017).
24. Su, X., et al. Phase separation of signaling molecules promotes T cell receptor signal transduction. Science 352, 595-599 (2016).
25. Ditlev, J. A., et al. A composition-dependent molecular clutch between T cell signaling condensates and actin. Elife 8(2019).
26. Zamudio, A. V., et al. Mediator Condensates Localize Signaling Factors to Key Cell Identity Genes. Mol Cell 76, 753-766 e756 (2019).
27. Jaqaman, K. & Ditlev, J. A. Biomolecular condensates in membrane receptor signaling. Curr Opin Cell Biol 69, 48-54 (2021).
28. Case, L. B., Ditlev, J. A. & Rosen, M. K. Regulation of Transmembrane Signaling by Phase Separation. Annu Rev Biophys 48, 465-494 (2019).
29. Zeng, M., et al. Reconstituted Postsynaptic Density as a Molecular Platform for Understanding Synapse Formation and Plasticity. Cell 174, 1172-1187 e1116 (2018).
30. Lin, C., et al. Receptor tyrosine kinases regulate signal transduction through a liquid-liquid phase separated state. bioRxiv (2019).
31. Huang, W. Y. C., et al. A molecular assembly phase transition and kinetic proofreading modulate Ras activation by SOS. Science 363, 1098-1103 (2019).
32. Zeng, M., et al. Phase Transition in Postsynaptic Densities Underlies Formation of Synaptic Complexes and Synaptic Plasticity. Cell 166, 1163-1175 eli 12 (2016).
33. Gao, X. K., et al. Phase separation of insulin receptor substrate 1 drives the formation of insulin/IGF-1 signalosomes. Cell Discov 8, 60 (2022).
34. Banani, S. F., Lee, H. O., Hyman, A. A. & Rosen, M. K. Biomolecular condensates: organizers of cellular biochemistry. Nat Rev Mol Cell Biol 18, 285-298 (2017).
35. Cho, W. K., et al. RNA Polymerase II cluster dynamics predict mRNA output in living cells. Elife 5(2016).
36. Nair, S. J., et al. Phase separation of ligand-activated enhancers licenses cooperative chromosomal enhancer assembly. Nat Struct Mol Biol 26, 193-203 (2019).
37. Li, W., et al. Biophysical properties of AKAP95 protein condensates regulate splicing and tumorigenesis. Nat Cell Biol 22, 960-972 (2020).
38. Niaki, A. G., et al. Loss of Dynamic RNA Interaction and Aberrant Phase Separation Induced by Two Distinct Types of ALS/FTD-Linked FUS Mutations. Mol Cell 77, 82-94 e84 (2020).
39. Boothe, T., et al. Inter-domain tagging implicates caveolin-1 in insulin receptor trafficking and Erk signaling bias in pancreatic beta-cells. Mol Metab 5, 366-378 (2016).
40. Gustavsson, J., et al. Localization of the insulin receptor in caveolae of adipocyte plasma membrane. FASEB J 13, 1961-1971 (1999).
41. S, K., P, T. & AD, A. Histopathology of Liver in Diabetes Mellitus—An Autopsy Study. International Journal of Scientific Study 4(2016).
42. Madiraju, A. K., et al. Metformin suppresses gluconeogenesis by inhibiting mitochondrial glycerophosphate dehydrogenase. Nature 510, 542-546 (2014).
43. Butkowski, E. G. & Jelinek, H. F. Hyperglycaemia, oxidative stress and inflammatory markers. Redox Rep 22, 257-264 (2017).
44. KAYAL R., BHATTACHARJEE D., BANERJEE S. k. & B., K. RELATIONSHIP BETWEEN PROTEIN CARBONYLATION AND TYPE-2 DIABETES MELLITUS IN KOLKATA, INDIA. International Journal of Advances in Science Engineering and Technology 6, 2321-9009 (2018).
45. Batandier, C., et al. The ROS production induced by a reverse-electron flux at respiratory-chain complex 1 is hampered by metformin. J Bioenerg Biomembr 38, 33-42 (2006).
46. Yadollah, S., Kazemipour, N., Bakhtiyari, S. & Nazifi, S. Palmitate-induced insulin resistance is attenuated by Pioglitazone and EGCG through reducing the gluconeogenic key enzymes expression in HepG2 cells. J Med Life 10, 244-249 (2017).
47. Sefried, S., Haring, H. U., Weigert, C. & Eckstein, S. S. Suitability of hepatocyte cell lines HepG2, AML12 and THLE-2 for investigation of insulin signalling and hepatokine gene expression. Open Biol 8(2018).
48. Song, S. H., et al. Direct measurement of pulsatile insulin secretion from the portal vein in human subjects. J Clin Endocrinol Metab 85, 4491-4499 (2000).
49. Storch, M., Rössle, M. & Kerp, L. Pulsatile Insulinsekretion in die Pfortader bei Leberzirrhose. Med Wochenschr 118, 134-138 (1993).
50. Porksen, N., et al. Human insulin release processes measured by intraportal sampling. Am J Physiol Endocrinol Metab 282, E695-702 (2002).
51. Watanabe, M., Hayasaki, H., Tamayama, T. & Shimada, M. Histologic distribution of insulin and glucagon receptors. Braz J Med Biol Res 31, 243-256 (1998).
52. Ramakrishnan, G., Arjuman, A., Suneja, S., Das, C. & Chandra, N. C. The association between insulin and low-density lipoprotein receptors. Diab Vase Dis Res 9, 196-204 (2012).
53. Wu, X., et al. Vesicle Tethering on the Surface of Phase-Separated Active Zone Condensates. Mol Cell 81, 13-24 e17 (2021).
54. Emperador-Melero, J., et al. PKC-phosphorylation of Liprin-alpha3 triggers phase separation and controls presynaptic active zone structure. Nat Commun 12, 3057 (2021).
55. Bergeron-Sandoval, L. P., et al. Endocytic proteins with prion-like domains form viscoelastic condensates that enable membrane remodeling. Proc Natl Acad Sci USA 118(2021).
56. Kozak, M. & Kaksonen, M. Condensation of Ede1 promotes the initiation of endocytosis. Elife 11(2022).
57. Saad, M. F., et al. Sequential changes in serum insulin concentration during development of non-insulin-dependent diabetes. Lancet 1, 1356-1359 (1989).
58. Kajbaf, F., De Broe, M. E. & Lalau, J. D. Therapeutic Concentrations of Metformin: A Systematic Review. Clin Pharmacokinet 55, 439-459 (2016).
59. Timmins, P., Donahue, S., Meeker, J. & Marathe, P. Steady-state pharmacokinetics of a novel extended-release metformin formulation. Clin Pharmacokinet 44, 721-729 (2005).
60. Frid, A., et al. Novel assay of metformin levels in patients with type 2 diabetes and varying levels of renal function: clinical recommendations. Diabetes Care 33, 1291-1293 (2010).
61. Friesen M., et al. Development of a physiological insulin resistance model in human stem cell-derived adipocytes. Science Advances 8, 1-9 (2022).
62. Forman-Kay, J. D., Ditlev, J. A., Nosella, M. L. & Lee, H. O. What are the distinguishing features and size requirements of biomolecular condensates and their implications for RNA-containing condensates?RNA 28, 36-47 (2022).
63. Cho, W. K., et al. Mediator and RNA polymerase II clusters associate in transcription-dependent condensates. Science 361, 412-415 (2018).
64. Brangwynne, C. P., et al. Germline P granules are liquid droplets that localize by controlled dissolution/condensation. Science 324, 1729-1732 (2009).
65. Hyman, A. A., Weber, C. A. & Julicher, F. Liquid-liquid phase separation in biology. Annu Rev Cell Dev Biol 30, 39-58 (2014).
66. Feric, M., et al. Coexisting Liquid Phases Underlie Nucleolar Subcompartments. Cell 165, 1686-1697 (2016).
67. Patel, A., et al. A Liquid-to-Solid Phase Transition of the ALS Protein FUS Accelerated by Disease Mutation. Cell 162, 1066-1077 (2015).
68. Brangwynne, C. P., Mitchison, T. J. & Hyman, A. A. Active liquid-like behavior of nucleoli determines their size and shape in Xenopus laevis oocytes. Proc Natl Acad Sci USA 108, 4334-4339 (2011).
69. Cisse, I I, et al. Real-time dynamics of RNA polymerase II clustering in live human cells. Science 341, 664-667 (2013).
70. Huoh, Y. S., et al. Dual functions of Aire CARD multimerization in the transcriptional regulation of T cell tolerance. Nat Commun 11, 1625 (2020).
71. Mukherjee, S. P., Lane, R. H. & Lynn, W. S. Endogenous hydrogen peroxide and peroxidative metabolism in adipocytes in response to insulin and sulfhydryl reagents. Biochem Pharmacol 27, 2589-2594 (1978).
72. May, J. M. & de Haen, C. Insulin-stimulated intracellular hydrogen peroxide production in rat epididymal fat cells. J Biol Chem 254, 2214-2220 (1979).
73. Krieger-Brauer, H. I. & Kather, H. Human fat cells possess a plasma membrane-bound H2O2-generating system that is activated by insulin via a mechanism bypassing the receptor kinase. J Clin Invest 89, 1006-1013 (1992).
74. Mahadev, K., et al. The NAD(P)H oxidase homolog Nox4 modulates insulin-stimulated generation of H₂O₂ and plays an integral role in insulin signal transduction. Mol Cell Biol 24, 1844-1854 (2004).
75. Krieger-Brauer, H. I., Medda, P. K. & Kather, H. Insulin-induced activation of NADPH-dependent H₂O₂ generation in human adipocyte plasma membranes is mediated by Galphai2. J Biol Chem 272, 10135-10143 (1997).
76. Iwakami, S., et al. Concentration-dependent dual effects of hydrogen peroxide on insulin signal transduction in H4IIEC hepatocytes. PLoS One 6, e27401 (2011).
77. Ge, X., Yu, Q., Qi, W., Shi, X. & Zhai, Q. Chronic insulin treatment causes insulin resistance in 3T3-L1 adipocytes through oxidative stress. Free Radic Res 42, 582-591 (2008).
78. Fischer, R. & Maier, O. Interrelation of oxidative stress and inflammation in neurodegenerative disease: role of TNF. Oxid Med Cell Longev 2015, 610813 (2015).
79. Yu, D., et al. High fat diet-induced oxidative stress blocks hepatocyte nuclear factor 4alpha and leads to hepatic steatosis in mice. J Cell Physiol 233, 4770-4782 (2018).
80. Rudich, A., Kozlovsky, N., Potashnik, R. & Bashan, N. Oxidant stress reduces insulin responsiveness in 3T3-L1 adipocytes. Am J Physiol 272, E935-940 (1997).
81. Boden, G., et al. Excessive caloric intake acutely causes oxidative stress, GLUT4 carbonylation, and insulin resistance in healthy men. Sci Transl Med 7, 304re307 (2015).
82. Hou, X., et al. Metformin reduces intracellular reactive oxygen species levels by upregulating expression of the antioxidant thioredoxin via the AMPK-FOXO3 pathway. Biochem Biophys Res Commun 396, 199-205 (2010).
83. Huang, X., et al. ROS regulated reversible protein phase separation synchronizes plant flowering. Nat Chem Biol 17, 549-557 (2021).
84. Guillen-Boixet, J., et al. RNA-Induced Conformational Switching and Clustering of G3BP Drive Stress Granule Assembly by Condensation. Cell 181, 346-361 e317 (2020).
85. Rayman, J. B., Karl, K. A. & Kandel, E. R. TIA-1 Self-Multimerization, Phase Separation, and Recruitment into Stress Granules Are Dynamically Regulated by Zn(2). Cell Rep 22, 59-71 (2018).
86. Chong, P. A. & Forman-Kay, J. D. Oxidative Inhibition of Pbp1 Phase Separation. Biochemistry 58, 3057-3059 (2019).
87. Kato, M., et al. Redox State Controls Phase Separation of the Yeast Ataxin-2 Protein via Reversible Oxidation of Its Methionine-Rich Low-Complexity Domain. Cell 177, 711-721 e718 (2019).
88. Yang, Y. S., et al. Yeast Ataxin-2 Forms an Intracellular Condensate Required for the Inhibition of TORC1 Signaling during Respiratory Growth. Cell 177, 697-710 e617 (2019).
89. Ahmed, S. M., Luo, L., Namani, A., Wang, X. J. & Tang, X. Nrf2 signaling pathway: Pivotal roles in inflammation. Biochim Biophys Acta Mol Basis Dis 1863, 585-597 (2017).
90. Peng, T. I., Lin, M. S. & Jou, M. J. Dual phases of respiration chain defect-augmented mROS-mediated mCa 2+ stress during oxidative insult in normal and rho 0 RBA1 astrocytes. Oxid Med Cell Longev 2013, 159567 (2013).
91. Rehman, T., Fought, J. & Solomon, R. N-acetylcysteine effect on serum creatinine and cystatin C levels in CKD patients. Clin J Am Soc Nephrol 3, 1610-1614 (2008).
92. Radtke, K. K., et al. Interaction of N-acetylcysteine and cysteine in human plasma. J Pharm Sci 101, 4653-4659 (2012).
93. La Cunza, N., et al. Mitochondria-dependent phase separation of disease-relevant proteins drives pathological features of age-related macular degeneration. JCI Insight 6(2021).
94. Soberanes, S., et al. Metformin Targets Mitochondrial Electron Transport to Reduce Air-Pollution-Induced Thrombosis. Cell Metab 29, 503 (2019).
95. Ashabi, G., Khalaj, L., Khodagholi, F., Goudarzvand, M. & Sarkaki, A. Pre-treatment with metformin activates Nrf2 antioxidant pathways and inhibits inflammatory responses through induction of AMPK after transient global cerebral ischemia. Metab Brain Dis 30, 747-754 (2015).
96. Prasad, S., et al. Role of Nrf2 and protective effects of Metformin against tobacco smoke-induced cerebrovascular toxicity. Redox Biol 12, 58-69 (2017).
97. Alberti, S. & Hyman, A. A. Biomolecular condensates at the nexus of cellular stress, protein aggregation disease and ageing. Nat Rev Mol Cell Biol 22, 196-213 (2021).
98. Shin, Y. & Brangwynne, C. P. Liquid phase condensation in cell physiology and disease. Science 357(2017).
99. Tsang, B., Pritisanac, I., Scherer, S. W., Moses, A. M. & Forman-Kay, J. D. Phase Separation as a Missing Mechanism for Interpretation of Disease Mutations. Cell 183, 1742-1756 (2020).
100. Hayashi, Y., Ford, L. K., Fioriti, L., McGurk, L. & Zhang, M. Liquid-Liquid Phase Separation in Physiology and Pathophysiology of the Nervous System. J Neurosci 41, 834-844 (2021).

### Methods

### Human Liver Donor Samples

Samples of human livers were purchased from BioIVT or shared by collaborators at MGH (Hannah K Drescher and Lea M Bartsch). Informed consent was obtained by BioIVT or MGH from all human research participants. Sample ID numbers and donor information are obtained from either BioIVT or MGH and reported in FIG. 41. Frozen samples were embedded in OCT compound (Tissue-Tek, 4583), re-frozen on dry ice and stored at −80° C. Embedded samples were sectioned using the cryostat at the W.M. Keck Microscopy Facility, MIT. Sectioning was performed at −21° C. to generate 10 μm-thick slices that were then placed on a Superfrost Plus VWR Micro Slides (VWR, 48311-703) and stored at −20° C. Images of hematoxylin and eosin (H&E) stained liver tissue were obtained from BioIVT.

IRB 1999P004983 and IRB 2019P001245, COUHES E3272 and COUHES E3665.

### Cell Culture

HepG2 cells (ATCC HB-8065™) were used because of their demonstrated utility in the study of insulin signaling and resistance, and because they are amenable to genetic modification^{2,8,9}. HepG2 cells were cultured in EMEM (ATCC 30-2003) supplemented with 10% FBS (Sigma Aldrich, F4135) at 37° C. with 5% CO2 in a humidified incubator. For passaging, cells were washed in PBS (Gibco, 10010-023) and TrypLE Express Enzyme (Life Technologies, 12604021) was used to detach cells from plates and dissociate cell clumps. To ensure proper cell dissociation, cells were incubated with TrypLE at 37° C. with 5% CO₂ in a humidified incubator for 5 minutes; they were then mechanically dissociated by pipetting them up and down 8 times using a 5 mL serological pipette attached to an unfiltered 200 μl pipette tip. The 5 minutes incubation and mechanical dissociation were repeated one more time. TrypLE was quenched with EMEM supplemented with 10% FBS and cells were plated in new tissue culture-grade plates.

HEK293T cells (ATCC, CRL-3216) were used for the production of purified IRb protein. HEK293T cells were cultured in DMEM (GIBCO, 11995-073) supplemented with 10% FBS (Sigma Aldrich, F4135), 2 mM L-glutamine (Gibco, 25030) and 100 U/mL penicillin-streptomycin (Gibco, 15140), at 37° C. with 5% CO₂ in a humidified incubator.

Primary pre-adipocytes from (ATCC, PCS-210-010) were cultured in in Fibroblast Growth Kit-Low Serum (ATCC PCS-201-041), as per manufacturer's instructions. Cells for experiments were dissociated and plated at 18,000 cells/cm2. Two days later pre-adipocytes were differentiated in adipocyte differentiation media (ATCC PCS-500-050), as per manufacturer's instructions. Briefly, cells were washed and medium was replaced with adipocyte differentiation initiation medium. After 48 hours, half the medium was replaced. At day 4, medium was changed to adipocyte differentiation maintenance medium, and replaced every three days. At day 12, cells were rinsed and incubated in DMEM (Thermo Fisher, 11885084) with 0.1 or 3 nM of insulin for 5 days, replacing medium every other day. In the last 24 hours, 12.5 μM metformin was added. On day 17, cells were prepared for the assays by rinsing, and 30 minutes of washing. Afterwards, for imaging, cells were exposed to 3 nM insulin for 5 minutes, rinsed with PBS and fixed in 4% PFA for 15 minutes at room temperature. For pAKT ELISAs cells were exposed to 3 nM insulin for 15 minutes and harvested in cell lysis buffer (Cell Signaling Technology, #9803) with phosphatase inhibitor (Thermo Fisher Scientific, 78442).

For human liver spheroids, primary human hepatocytes from a 50-year-old male donor (BioIVT; lot #SMC) were used. Cells were thawed in Cryopreserved Hepatocyte Recovery Media (CHRM, ThermoFisher), spun down at 100×g for 8 minutes, and resuspended in seeding medium (William's E with 5.5 mM glucose, 2 mM GlutaMax, 15 mM HEPES, 5% FBS, 1% Pen/Strep, 100 nM hydrocortisone, and insulin 200 pM or 800 pM corresponding to the proper experimental group). Spheroids were formed using custom alginate microwells. In brief, 120,000 cells were seeded per well and spun at 50×g for 2 minutes to seed microwells, and cultured in a volume of 300 PL seeding medium. After 24 hours, cells were switched to maintenance media for the remainder of the experiment. This maintenance media was composed of William's E plus 6.25 μg/ml transferrin, 6.25 ng/ml selenium, 0.125% fatty acid-free BSA, 20 μM linoleic acid, 5.5 mM glucose, 2 mM GlutaMax, 15 mM HEPES, 0.5% Pen/Strep, and 100 nM hydrocortisone. Insulin was supplemented with concentrations adjusted to mimic healthy and disease-inducing states, either 200 pM for physiological or 800 pM for pathologic insulin levels. Media was exchanged every 48 hours throughout the experiment.

### Endogenously-Tagged Cell Line Generation

A CRISPR/Cas9 system was used to generate genetically modified HepG2 cell lines. Target sequences were cloned into a plasmid containing sgRNA backbone, a codon-optimized version of Cas9 and mCherry. For IR targeting, two Cas9 gRNAs were used. For the generation of the IR-mEGFP, IR-Dendra2, and IR-Dendra2-FKBP endogenously tagged lines, homology directed repair templates were cloned into pUC19 using NEBuilder HiFi DNA Assembly Master Mix (NEB, E2621S). For IR-mEGFP and IR-Dendra2 cell lines, the homology repair template consisted of mEGFP or Dendra2 cDNA sequence flanked on either side by 800 bp homology arms amplified from HepG2 genomic DNA using PCR (FIG. 27A). For the IR-Dendra2-FKBP cell line, the homology repair template consisted of Dendra2 cDNA sequence followed by four FK506 binding protein (FKBP) binding domains¹⁰ flanked on either side by 800 bp homology arms amplified from HepG2 genomic DNA using PCR. The following sgRNA sequences with PAM sequence in parentheses were used for CRISPR/Cas9 targeting:

sgRNA_IR_C-term_1:
(SEQ ID NO: 13)
CACGGTAGGCACTGTTAGGA(AGG)
sgRNA_IR_C-term_2:
(SEQ ID NO: 14)
TAGGCACTGTTAGGAAGGAT(TGG)

To generate genetically modified cell lines, 2×10⁶ cells were transfected with 500 ng of Cas9 plasmid 1, 500 ng of Cas9 plasmid 2, and 1,000 ng of non-linearized homology repair template using Lipofectamine 3000 (Invitrogen, L3000). Cells were sorted 48 hours after transfection for the presence of the mCherry fluorescent protein encoded on the Cas9 plasmid to enrich for transfected cells. This population of cells was allowed to expand for 1.5 to 2 weeks before sorting a second time for the presence of mEGFP or Dendra2 and single cells were plated into individual wells of a 96-well plate. The single cells were cultured in conditioned EMEM media (described below) for 1-1.5 months. 20-30 colonies were screened for successful targeting using PCR genotyping to confirm insertion. PCR genotyping was performed using Phusion polymerase (Thermo Scientific, F531S). Using the following primers, PCR products were amplified according to manufacturer specifications:

IR fwd:
(SEQ ID NO: 15)
GGAGAATGTGCCCCTGGAC
IR_rev:
(SEQ ID NO: 16)
TTGGTAACCAAACGAGTCCACCT

To make conditioned media, applicant cultured HepG2 cells in fresh EMEM media (ATCC, 30-2003) supplemented with 10% FBS (Sigma Aldrich, F4135) for 3 days and saved the media (old EMEM media). The composition of conditioned EMEM media is as follows: 50% fresh EMEM media and 50% old EMEM media. The conditioned media was filter sterilized prior to use.

HepG2 cells expressing IR-mEGFP were used for super-resolution microscopy with LSM880 or LSM980 with Airyscan detector.

HepG2 cells expressing IR-Dendra2 were used for single-molecule super-resolution microscopy, Dendra2 is a green-to-red photo-switchable protein that allows for single-molecule imaging.

### Constructs

For experiments that forced reduction in IR condensate dynamics, the vector used in this assay was modified from pJH135_pb_MCPx2_mCherry_rTTA vector¹¹. IR-mEGFP-FKBP, which consists of the insulin receptor cDNA, flexible linker 1, mEGFP, flexible linker 2, and four copies of FKBP, was cloned into PmeI and NheI digested pJH135_pb_MCPx2_mCherry_rTTA using Gibson cloning by following the manufacturer's instructions (NEB, E2621S). This vector is called JP204 pb_TetON_INSR_2A_GFP_Dmbr4.

### Cell Treatments

For insulin sensitivity and resistance experiments in HepG2 cells, cells were washed once with EMEM alone, without any supplements (ATCC, 30-2003) and cultured in EMEM for two days. Cells were then treated for two days with either physiological (0.1 nM) or pathological (3 nM) levels of insulin (Sigma Aldrich, I9278-5ML) in EMEM supplemented with 1.25% fatty acid-free bovine serum albumin (BSA; Sigma Aldrich, A8806-5G). Media was replenished every 12 hours. To wash out insulin, cells were washed with EMEM seven times, including: three quick washes, three 5-minute washes and a long 20 minute-wash in EMEM at 37° C. In order to investigate insulin response, cells were acutely treated for 5 minutes with insulin diluted in EMEM supplemented with 1.25% fatty acid-free BSA at 37° C. with 5% CO₂ in a humidified incubator. Concentration of insulin used varied and is reported in the figures.

For TNFα treatment, cells were cultured in EMEM BSA containing 10 pg/ml¹² of Human TNF-alpha Recombinant Protein (Thermo Fisher Scientific, PHC3016) for 2 days. Media was replenished every 12 hours. Insulin washout and insulin stimulation was performed as above.

For high nutrient condition, cells were cultured for 2 days in EMEM containing either: 1) 10 mM glucose, 45 μM oleic acid (CAYMAN CHEMICAL, 29557), 30 PM palmitic acid (CAYMAN CHEMICAL, 29558) and 3 nM insulin (called in the text “pathologic glucose, pathologic fat, and pathologic insulin (GFI)”) or 2) 10 mM glucose, 45 μM oleic acid, 30 μM palmitic acid and 0.1 nM insulin (called in the text “pathologic glucose, pathologic fat, and physiologic insulin (GF)”). Control cells were cultured for 2 days with EMEM containing BSA control (CAYMAN CHEMICAL, 29556). Media was replaced every 12 hours. Insulin washout and insulin stimulation was performed as above.

For metformin treatment, metformin (Sigma Aldrich, D150959-5G) was resuspended in sterile water to a concentration of 1M and diluted in cell media to the reported concentrations. Insulin-resistant cells were treated with pathological concentrations of insulin and metformin at various concentration reported in the figures. Media was replenished every 12 hours. Insulin washout and insulin stimulation was performed as above.

For N-acetyl cysteine treatment, insulin-resistant cells were treated with pathological concentrations of insulin and 1 mM N-acetyl cysteine (Sigma Aldrich, A9165-25G) for 24 hours. Media was replenished every 12 hours. Insulin washout and insulin stimulation was performed as above.

For oxidative stress, insulin sensitive cells were treated with 20 mM H₂O₂(Sigma Aldrich, H1009) for 30 minutes. Insulin stimulation was performed as above.

For adipocytes, following differentiation, cells were cultured with EMEM for 2 days and with EMEM containing either physiological (0.1 nM) or pathological (3 nM) concentrations of insulin for 2 days. Cells were then cultured with EMEM containing either physiological (0.1 nM) or pathological (3 nM) of insulin or with pathological (3 nM) concentrations of insulin and 12.5 μM of metformin for 1 day. Cells were then washed with EMEM and acutely stimulated with or without 3 nM insulin for 5 minutes prior to cell collection for immunofluorescence or ELISA.

For experiments that forced reduction in IR condensate dynamics, 1×10⁵ cells/cm² HepG2 cells were transfected with 0.07 μg/cm² pJP204_pb_TetON_INSR_2A_GFP_Dmbr4 using Lipofectamine 3000 (Invitrogen, L3000). On day 2, the cells were treated with 100 ng/ml doxycycline (Sigma, D9891-5G). On day 3, the cells were treated with EMEM, 100 ng/ml doxycycline containing either 5 μM AP1903 (MedChemExpress, NC1416062) or 5 μM DMSO (Sigma, D2650-100ML) for 16 hours and then harvested for imaging and western blot.

### Cell Viability

Cells were detached from plates and dissociated from clumps using TrypLE as described above. TrypLE was quenched with EMEM supplemented with 10% FBS. Dead cells were stained with trypan blue (Life Technologies, T10282) and the percentage of cell viability was then measured using the Countess II FL (Applied Biosystems, A27977) according to the manufacturer's specifications.

### Insulin Clearance

Insulin sensitive HepG2 cells were cultured in EMEM for 30 minutes and then in 3 nM insulin for 0, 5 or 24 hours. Culture media was collected and insulin concentration was measured at all timepoints using Human/Canine/Porcine Insulin DuoSet ELISA kit (R&D Systems, DY8056-05) according to the manufacturer's specifications. Clearance fraction was calculated by dividing the measured insulin concentration in cultured media by the measured insulin concentration in the cell-free control wells.

Insulin clearance in human liver spheroids was evaluated by collecting media after 48 hours in culture media was removed and insulin concentration was measured using Human/Canine/Porcine Insulin DuoSet ELISA kit (R&D Systems, DY8056-05) according to the manufacturer's specifications. Clearance fraction was calculated by dividing the measured insulin concentration in cultured media by the measured insulin concentration in the cell-free control wells.

### Glucose Production

Insulin sensitive and resistant cells were cultured in EMEM for 30 minutes and then cells were treated with 0, 0.1, 1 and 10 nM insulin in glucose production media, containing DMEM (Thermo Fisher Scientific, A1443001), 15 mM HEPES (Gibco, 15630-080), 1 mM pyruvate (Sigma Aldrich, P5280), 20 mM lactate (Sigma Aldrich, L7022-5G) for 4-5 hours. Media was removed and glucose production was measured using Amplex™ Red Glucose/Glucose oxidase assay kit (Thermo Fisher Scientific, A22189) according to the manufacturer's specifications.

Measurement of glucose production in human liver spheroids was performed as follows. At Day 10 in culture, spheroids were washed 5 times with glucose free William's E media (Thermo Fisher, ME18082L1), followed by culture for 24 hours in glucose free William's E maintenance media, supplemented 1 mM pyruvate, 20 mM lactate, and between 0 to 10 nM insulin stimulation. After 24 hours, media was collected and glucose quantified with the Amplex Red Glucose Assay Kit (Thermo Fisher, A22189) according to manufacturer instructions.

### Albumin Quantification

To assess hepatocyte spheroid function, media was collected during every media exchange and albumin secretion was assayed via ELISA kit (Bethyl Laboratories, E80-129) following the manufacturer's instructions.

siRNA Experiments

HepG2 cells were reverse transfected using Lipofectamine™ RNAiMAX Transfection reagent (Thermo Fisher Scientific, 13778100) following the manufacturer's instructions. Cells were dissociated using TrypLE as previously described then seeded in 6-multiwells in 1 ml EMEM supplemented with 10% FBS and the transfection reagent. Cells were cultured with the transfection reagent for 2-3 days prior to collection for Western blot and immunofluorescence.

The INSR siRNA pool (Dharmacon Inc, L-003014-00-0005) and the ON-TARGETplus Non-targeting Control Pool (Horizon Discovery, D-001810-01-05) were used.

### Western Blot

Cells were washed with ice-cold PBS (Life Technologies, AM9625) and lysed in Cell Lytic M (Sigma Aldrich C2978) supplemented with protease and phosphatase inhibitors (Sigma Aldrich, 11873580001 and 4906837001) directly on the wells. Lysates were placed into a 1.5 ml tube and mixed at 4° C. for 20 minutes, sonicated and then centrifuged at 12,000×g for 15 minutes. Super natant was collected and protein concentration was determined using a BCA Protein Assay Kit (Life Technologies, 23250) according to the manufacturer's instructions. Equal amounts of protein (5-50 μg per sample) were separated on 10% or 12% Bis-Tris gels in 5% XT MOPS running buffer (Bio-Rad Laboratories, 1610788) at 100V until dye front reached the end of the gel. Protein was then transferred to a 0.45 μM PVDF membrane (Millipore, IPVH00010) in ice cold transfer buffer (25 mM Tris, 192 mM glycine, 20% methanol) at 300 mA for 1 hour or 250 mA for 2 hours at 4° C. After the transfer, membranes were blocked in either 5% non-fat milk (LabScientific, M0842) dissolved in TBST (2% Tris HCl pH 8.0, 1.3% 5M NaCl, 0.05% Tween 20) or 5% BSA (VWR, 102643-516) in 1×TBST for 15 minutes to 1 hour at room temperature with shaking. Membranes were then incubated overnight at 4° C. in 1:1000 primary antibody (specific antibodies listed below) in 5% non-fat milk in TBST or 5% BSA in TBST. BSA was used for immunoblotting phosphorylated proteins, otherwise milk was used. Membranes were then washed three times for 5 minutes in TBST shaking at room temperature prior to incubation in 1:10,000 secondary antibody (specific antibodies listed below) in 5% non-fat milk in TBST for 1 hour at room temperature. This was followed by three 10-minute washes in TBST. Membranes were developed with ECL substrate (Thermo Scientific, 34080) and imaged using a CCD camera (BIO RAD, 1708265). Immunoblot quantification was performed using the “analyze gel” tool on Fiji/ImageJ v2.1.0/153c.

The following primary antibodies were used for WB: anti-phosphorylated insulin receptor (Abcam, ab60946; Cell Signaling, 3026), anti-insulin receptor beta (Cell Signaling, 23413; Bethyl, A303-712A; Cell Signaling, 3025), anti-insulin receptor alpha (Cell Signaling, 74118), anti-phosphorylated IRS1 (Cell Signaling, 3070), anti-IRS1 (Cell Signaling, 2382), anti-phosphorylated AKT (Cell Signaling, 4056), anti-AKT (Cell Signaling, 9272), anti-phosphorylated ERK (Cell Signaling, 4377), anti-ERK (Cell Signaling, 9102), anti-pGSK α,β (Cell Signaling, 8566), anti-GSK α,β (Cell Signaling, 4337 and 12456), anti-beta Actin (Sigma Aldrich, A5441), and anti-GAPDH (Abcam, ab8245). The following secondary antibodies were used: donkey anti-rabbit IgG (Cytiva Life Sciences, NA934-1ML) and sheep anti-mouse IgG (Sigma Aldrich, NXA931V).

For quantitative western blot analysis, equal numbers of cells were cultured in each well of on a 6-well plate. To estimate the number of cells per well, cells in two wells were dissociated with TrypLE (Life Technologies, 12604021) and counted using the Countess II (Applied Biosystems, A27977). Cells from another well were lysed on the plate as described above. A dilution series of purified IRb-mCherry and HepG2 cellular lysate was separated on 10% Bis-Tris gels in 5% XT MOPS running buffer. Immunoblotting was performed as above. Bands were quantitated using Fiji/ImageJ v2.1.0/153c, from which applicant calculated the estimated number of molecules of IRb per HepG2 cell.

### Proteolytic Surface Shaving Experiment

To compare IR amounts in whole cells and at the plasma membrane, proteolytic surface shaving experiment was performed. Equal numbers of cells were cultured in each well of on a 6-well plate and cultured with either 0.1 nM insulin or 3 nM insulin for 2 days. To estimate the relative IR amounts in the whole cell, cells were washed in PBS and lysed in ice-cold Cell Lytic M supplemented with protease and phosphatase inhibitors as previously described. To estimate the relative IR amounts at the plasma membrane (labeled in the figure as “Digested”), cells were digested with TrypLE for 10 minutes at 37° C. and quenched with EMEM 10% FBS. Cells were spun down at 300 g for 5 min, washed in PBS and ice-cold Cell Lytic M supplemented with protease and phosphatase inhibitors. Samples were then processed for Western blot as previously described and immunoblotted for insulin receptor alpha and beta actin.

### Metabolomics

Metabolite isolation from liver tissue: Flash frozen tissues were pulverized with a mortar and pestle in a liquid nitrogen bath. Tissue powder was transferred into Eppendorf tubes and re-suspended in 800 uL ice-cold LC-MS grade 60:40 methanol:water (ThermoFisher). Samples were vortexed for 10 minutes at 4° C. Then, 500 μL of ice-cold LC-MS grade chloroform (provided by the Metabolomics core) was added to the lysate and samples were vortexed for an additional 10 minutes at 4° C. Samples were centrifuged at 16,000 g for 10 minutes at 4° C., creating three layers: the top layer containing polar metabolites, the bottom layer containing non-polar metabolites, and the middle layer containing protein. The top layer was transferred to a new tube, dried down in a speedvac, and subsequently stored at −80° C. until they were analyzed by LC-MS.

Stable isotope tracing for lipogenesis in HepG2 cells: Cells were cultured with 0.1 nM or 3 nM insulin (Sigma Aldrich, I9278-5ML) for 2 days as detailed above. To wash out insulin, cells were washed with EMEM (ATCC 30-2003) seven times, including: three quick washes, three 5-minute washes and a long 20 minute-wash in EMEM at 37° C. Cells were then cultured in EMEM supplemented with 1 mM Sodium acetate-¹³C₂(Sigma Aldrich, 282014) and with either 0 nM insulin or 1 nM insulin for 36 hours. Media was replenished after 24 hours. Cells were then processed for metabolite isolation (see below).

Stable isotope tracing for gluconeogenesis in HepG2 cells: Cells were cultured with 0.1 nM or 3 nM insulin (Sigma Aldrich, I9278-5ML) for 2 days as detailed above. To wash out insulin, cells were washed with EMEM (ATCC 30-2003) seven times, including: three quick washes, three 5-minute washes and a long 20 minute-wash in EMEM at 37° C. Cells were washed with glucose-free RPMI (Gibco, 11879-020) then cultured in glucose-free RPMI for 3 hours. Cells were then cultured in glucose-free RPMI supplemented with 5 mM sodium pyruvate-¹³C₃(Cambridge Isotope Laboratories, NC1345852) and 5 mM Sodium L-lactate (Sigma Aldrich, L7022) and with either 0 nM, 1 nM, 10 nM or 100 nM insulin (Sigma Aldrich, I9278-5ML) for 16 hours. Cells were then processed for metabolite isolation (see below).

Metabolite isolation from HepG2 cells: cells were washed in ice-cold PBS (Life Technologies, AM9625), 500 μl of cold 80% MeOH (shared by the Metabolite Profiling Core Facility) was added per well of a 6-well plate and the plate was placed at −80° C. for at least 15 minutes. Following the −80 incubation, the plate was scraped on dry ice and the solution was transferred to a 1.5 ml tube and then vortexed for 5 minutes. To remove cellular debris, the samples were centrifuged at maximum speed for 10 minutes at 4° C. and the supernatant was transferred to a new 1.5 mL tube on dry ice. To remove solvents, the samples were lyophilized using Refrigerated CentriVap Benchtop Vacuum Concentrator connected to a CentriVap-105 Cold Trap (Labconco). Metabolite pellets were re-suspended in LC-MS grade water (ThermoFisher) and vortexed for 10 minutes at 4° C. Samples were centrifuged at 16,000 g for 10 minutes at 4° C. and supernatant was moved into LC-MS vials. Liquid Chromatography and Mass Spectrometry was performed by the Whitehead metabolomics core.

### Immunofluorescence

HepG2 cells, human liver spheroids, human primary adipocytes and human tissue liver sections were fixed in 4% PFA (VWR, BT140770-10X10) in PBS (Life Technologies, AM9625) for 10 minutes at room temperature. Cells were washed three times for 5 minutes in PBS, permeabilized with 0.5% TritonX100 (Sigma Aldrich, X100) in PBS, washed three times for 5 minutes in PBS, and then blocked with 4% IgG-free BSA (VWR, 102643-516) for 15-60 minutes at room temperature. Afterwards, the cells were incubated with 1:500 or 1:1000 primary antibody (specific antibodies listed below) in 4% IgG-free BSA in PBS at 4° C. overnight. The next day, cells were washed three times with PBS and incubated with 1:500 or 1:1000 secondary antibodies (specific antibodies listed below) in 4% IgG-free BSA at room temperature for 1 hr covered in foil. Cells were washed three times with PBS for 5 minutes. DNA was stained using 1:5000 Hoechst (Thermo Fischer Scientific, 3258) in PBS for 5 minutes at RT. Cells were washed three times with PBS for 5 minutes, stored at 4° C. until imaging. For tissue sections, samples were mounted using Vectashield mounting media (Vector Laboratories, Inc, H-1000). LSM880 or LSM980 microscope with Airyscan detector (ZEISS) was used for image acquisition. Images were then processed using Fiji/ImageJ v2.1.0/153c.

Primary antibodies used were anti-insulin receptor beta (Cell Signaling, 23413), anti-NRF2 antibody (Abcam, ab62352, 1:500 dilution), anti-cytokeratin 18 (CK18) (Abcam, ab668, ab62352, 1:500 dilution), anti-PI3K (Abcam, ab135253, ab62352, 1:500 dilution), anti-AKT (Cell Signaling, 2920, ab62352, 1:500 dilution), anti-clathrin (Abcam, ab24578, ab62352, 1:500 dilution), anti-LAMP1 (abeam, ab25630, ab62352, 1:500 dilution), and anti-EEA1 (Abcam, ab70521, ab62352, 1:500 dilution), anti-pIRS1 (Abcam, ab4873, ab62352, 1:1000 dilution), anti-perilipin (Sigma, P1873, 1:500 dilution). Secondary antibodies used were Alexa Fluor 488 goat anti-rabbit IgG (Thermo Fischer Scientific, A1 1008), Alexa Fluor 647 goat anti-rabbit IgG (Thermo Fischer Scientific, A21244), Alexa Fluor 568 goat anti-mouse IgG (Thermo Fischer Scientific, A11031).

Images were acquired at LSM880 or LSM980 Microscope with Airyscan detector with 63× objective using Zen Black software (ZEISS) at the W.M. Keck Microscopy Facility, MIT. Images were then processed using Fiji/ImageJ v2.1.0/153c. Scale bars were determined using Fiji/ImageJ v2.1.0/153c and, when scale bars were obscured by fluorescence intensity, a black background was added to improve visibility.

### Live-Cell Imaging

Cells expressing endogenous IR tagged with GFP were grown on 35 mm glass bottom dishes (MatTek Corporation, P35G-1.5-20-C). Cells were imaged at 37° C. using the LSM880 or LSM980 Microscope with Airyscan detector with 63× objective and Zen Black software (ZEISS) at the W.M. Keck Microscopy Facility, MIT. Images were then processed using Fiji/ImageJ v2.1.0/153c.

### ROS Staining and Live-Cell Imaging

After culturing the HepG2 cells with physiological insulin concentrations or pathological insulin concentrations or with TNFα or high nutrients for 2 days, media was removed and cells were cultured with ROS Deep Red Stock Solution (Abcam, ab186029) diluted to 1× in Dulbecco's PBS (Gibco, 14040-133). Cells were incubated at 37° C. with 5% CO₂ in a humidified incubator for 30 minutes. Cells were imaged at 37° C. using the LSM880 Microscope with Airyscan detector with 63× objective and Zen Black software (ZEISS) at the W.M. Keck Microscopy Facility, MIT. Images were then processed using Fiji/ImageJ v2.1.0/153c.

### RNA Fish

Pipettes and laboratory bench were treated with RNaseZap (Life Technologies, AM9780). Cells were fixed with 4% PFA (VWR, BT140770-10X10) in PBS (Life Technologies, AM9625) for 10 minutes at RT. Cells were washed three times with PBS for 5 minutes. Cells were permeabilized with 0.5% TritonX100 (Sigma Aldrich, X100) in 1×RNasefree PBS (Invitrogen, AM9625) for 10 minutes at room temperature. Cells were washed three times with RNase-free PBS for 5 minutes. Cells were washed once with 20% Stellaris RNA FISH Wash Buffer A (Biosearch Technologies, Inc., SMF-WA1-60), 10% Deionized Formamide (EMD Millipore, S4117) in RNase-free water (Life Technologies, AM9932) for 5 minutes at room temperature. Cells were then hybridized with 90% Stellaris RNA FISH Hybridization Buffer (Biosearch Technologies, SMF-HB1-10), 10% Deionized Formamide, 12.5 μM Stellaris RNA FISH probes designed to hybridize intronic regions of each transcript (FASN, SREBF1, and TIMM22; probes listed below). Hybridization was performed overnight at 37° C. Cells were then washed twice with Wash Buffer A for 30 minutes at 37° C. and once with Stellaris RNA FISH Wash Buffer B (Biosearch Technologies, SMF-WB1-20) for 5 minutes at room temperature. Images were acquired at LSM880 or LSM980 Microscope with Airyscan detector with 63× objective using Zen Black software (ZEISS) at the W.M. Keck Microscopy Facility, MIT.

Stellaris® FISH Probes, Custom Assay with TAMRA Dye (LGC Bioserch, SMF-1001-5).

FASN RNA FISH probe sequence:
(SEQ ID NO: 17)
cgagagcggaggatgaggag
(SEQ ID NO: 18)
aaggggcacgaacaccgaga
(SEQ ID NO: 19)
gaaatggggatagcctatgc
(SEQ ID NO: 20)
cattcagttcagggtattgg
(SEQ ID NO: 21)
acaaaggtggagatggagct
(SEQ ID NO: 22)
gtgcaatgtccaggaaggag
(SEQ ID NO: 23)
ctccgtaacaagcagatggg
(SEQ ID NO: 24)
gagcacatctggtatgcaac
(SEQ ID NO: 25)
tagagctgctctgtggaaga
(SEQ ID NO: 26)
tctaccatgctgactcacag
(SEQ ID NO: 27)
gactggtacgaccagatctg
(SEQ ID NO: 28)
caaacaatggagcaggctcc
(SEQ ID NO: 29)
aactgaggactctctgctat
(SEQ ID NO: 30)
gacaggcgggtgtttaaatc
(SEQ ID NO: 31)
tggggtgcagttgggaaact
(SEQ ID NO: 32)
atcagaagcgaggagactgg
(SEQ ID NO: 33)
ctcaagaacctcctgctttg
(SEQ ID NO: 34)
tggcgggagagggttgaaat
(SEQ ID NO: 35)
aggtcaaatgccagtcagag
(SEQ ID NO: 36)
gagcacctggatcttaagaa
(SEQ ID NO: 37)
gagcataaccttagtcttgg
(SEQ ID NO: 38)
aggagacaaaggccaagtgt
(SEQ ID NO: 39)
agaacaggccgtgataagga
(SEQ ID NO: 40)
aacaggaaccaggtcacaga
(SEQ ID NO: 41)
tgccacaaacagtggtcaag
(SEQ ID NO: 42)
cacactcacctgcaaggaag
(SEQ ID NO: 43)
tagtgggtcaggaagctgta
(SEQ ID NO: 44)
tgtgcctggtctctctaaag
(SEQ ID NO: 45)
gtgagtgatcatgggtgttg
(SEQ ID NO: 46)
ctgtggcctgaacactaagg
(SEQ ID NO: 47)
ctaattctgccatggcacag
(SEQ ID NO: 48)
caaggctctgcctagcaaag
(SEQ ID NO: 49)
gataggaaagaagcccctat
(SEQ ID NO: 50)
ggaacaggacactcaggttg
(SEQ ID NO: 51)
tctgagggaaccctgatgac
(SEQ ID NO: 52)
agagatgggtgccaacagag
(SEQ ID NO: 53)
gtggaggtgtcattacagag
(SEQ ID NO: 54)
gagccttcatgagaaaggtt
(SEQ ID NO: 55)
acaagtgtggtaatggcagc
(SEQ ID NO: 56)
aagggacctgaggacaaacc
(SEQ ID NO: 57)
caagggaaccgagagggaat
(SEQ ID NO: 58)
tattagtccacctggacatc
(SEQ ID NO: 59)
ctacggagaagggaggcatg
(SEQ ID NO: 60)
tgtggggagggaagagtctg
(SEQ ID NO: 61)
gggagagttggagatcagag
(SEQ ID NO: 62)
tggaaagggaggtgcggaag
(SEQ ID NO: 63)
agaagcaagtctggggtcaa
(SEQ ID NO: 64)
actgaggaacagtgaccagg
SREBFI RNA FISH probe sequence:
(SEQ ID NO: 65)
ttggctgtaagctgtgtgtc
(SEQ ID NO: 66)
ctaaataaacgaggctggcc
(SEQ ID NO: 67)
acctttattgaagaggcctg
(SEQ ID NO: 68)
gatgcagacagcagggtctg
(SEQ ID NO: 69)
cgggcatagggttagaatgt
(SEQ ID NO: 70)
tggagctcaataaagccagg
(SEQ ID NO: 71)
aaggctagagaagaggccag
(SEQ ID NO: 72)
gactagagctgaatgcaggg
(SEQ ID NO: 73)
ctttcaggaccagaggtaac
(SEQ ID NO: 74)
ctttgatggggctgggttag
(SEQ ID NO: 75)
ggactactgtagctcctaaa
(SEQ ID NO: 76)
aaaggctggatatgtgaccc
(SEQ ID NO: 77)
cgaaaggaacagagccagga
(SEQ ID NO: 78)
atgaggctcagaggatatgg
(SEQ ID NO: 79)
taggatctgttagggtcttc
(SEQ ID NO: 80)
cttacctatggacagaggga
(SEQ ID NO: 81)
agcaggaacaagggttgaca
(SEQ ID NO: 82)
gaggacgggacagattcatg
(SEQ ID NO: 83)
cagtcctacctgtggatgag
(SEQ ID NO: 84)
gagagagctgcagggataag
(SEQ ID NO: 85)
taagagagcacctgtagggg
(SEQ ID NO: 86)
ctgcatgtgcttctgaaagc
(SEQ ID NO: 87)
tcacttctccaattagccat
(SEQ ID NO: 88)
gagaagatgccattgttggc
(SEQ ID NO: 89)
gcttccaaaggaaaaccgac
(SEQ ID NO: 90)
ggtggacataactatcacca
(SEQ ID NO: 91)
ctgccaaggacaggggaaag
(SEQ ID NO: 92)
gagggcacaacgacacttac
(SEQ ID NO: 93)
gagctattctcagaatcccg
(SEQ ID NO: 94)
gaggaatgaagcgtgcatgg
(SEQ ID NO: 95)
ctgtcggaacagatggcagg
(SEQ ID NO: 96)
tcacctgtggaaggagagag
(SEQ ID NO: 97)
tgagaagggagccaggacag
(SEQ ID NO: 98)
aacaaaggctgagtgaggca
(SEQ ID NO: 99)
ccctgaggaaaaaaggtggt
(SEQ ID NO: 100)
cagaagagtgccagtcagac
(SEQ ID NO: 101)
ccagggaatggaaagctgaa
(SEQ ID NO: 102)
gaagccttagccaaaaagca
(SEQ ID NO: 103)
gcaatgcaacagcaatgcac
(SEQ ID NO: 104)
tgctgagcagacagcacatc
(SEQ ID NO: 105)
ttggtatcacatcccatgtg
(SEQ ID NO: 106)
ccactgattccttgtgaaag
(SEQ ID NO: 107)
gtatcccacaaatgacagtc
(SEQ ID NO: 108)
cacagactgagtcacgcacg
(SEQ ID NO: 109)
gtctcagcccacacacaaag
(SEQ ID NO: 110)
tgtacctggcacacaggtac
(SEQ ID NO: 111)
acgaggatgtgtcagggatg
TIMM22 RNA FISH probe sequence:
(SEQ ID NO: 112)
tggtcttcteggcagagatc
(SEQ ID NO: 113)
agggtgtggtcaaggtcaag
(SEQ ID NO: 114)
acgcccgattcacgaacgag
(SEQ ID NO: 115)
attttcatcaggaaagccgg
(SEQ ID NO: 116)
cacagcaccgattctctaac
(SEQ ID NO: 117)
atgtaataactttcaggccc
(SEQ ID NO: 118)
tcacctggcacgtgatattg
(SEQ ID NO: 119)
tgaagaccaggctcttgtct
(SEQ ID NO: 120)
acatagtataatacaggccc
(SEQ ID NO: 121)
ttcctgctcaacattcttct
(SEQ ID NO: 122)
tgcatgtgcatttccatatg
(SEQ ID NO: 123)
aacaactgctgccctagagt
(SEQ ID NO: 124)
tctttcagtttttcaaacct
(SEQ ID NO: 125)
cggggctgtctagtcacaat
(SEQ ID NO: 126)
acaagtgcagacttcgtctc
(SEQ ID NO: 127)
ataaaatcatgggcacctcc
(SEQ ID NO: 128)
acctcatggtcaatatgagt
(SEQ ID NO: 129)
actgagcacatgccagtatg
(SEQ ID NO: 130)
gtgacattcacagtaatgct
(SEQ ID NO: 131)
ttgcaaatcactctcttggc
(SEQ ID NO: 132)
ccctcaacttcagctatcaa
(SEQ ID NO: 133)
attctctctacattctctca
(SEQ ID NO: 134)
aagattccatctttaaccct
(SEQ ID NO: 135)
ctgtcattccctaacatttc
(SEQ ID NO: 136)
ctccgtctctaagatttett
(SEQ ID NO: 137)
gggtctatgttactgacatc
(SEQ ID NO: 138)
ttgcctggagacttgcaatc
(SEQ ID NO: 139)
ctcttgccactcaaactctc
(SEQ ID NO: 140)
atgcttttggatgaccaccg
(SEQ ID NO: 141)
tgggatcatcctggaaggga
(SEQ ID NO: 142)
catgttttctgcattactct
(SEQ ID NO: 143)
acagagatgaaggtgtcttt
(SEQ ID NO: 144)
agacacttacctagaagcaa
(SEQ ID NO: 145)
gcaaggatttcttagaaggc
(SEQ ID NO: 146)
aagtaatgaaatggtggccc
(SEQ ID NO: 147)
tgcttctgatttgctttcta
(SEQ ID NO: 148)
ctctccaataagtctcgttt
(SEQ ID NO: 149)
ccagcatttggaatgtaatc
(SEQ ID NO: 150)
caccagagtgctgaaaccaa
(SEQ ID NO: 151)
ttcatagatgcttcctgcag

### Temperature Experiment

After culturing HepG2 cells with 0.1 nM insulin for 2 days, insulin was washed out as previously described. Cells were cultured at 37° C. or on ice at 4° C. in EMEM for 30 minutes and then treated with the reported concentrations of insulin diluted in EMEM containing BSA (media was warmed at 37° C. for the 37° C. samples or was cooled to 4° C. for the 4° C. samples before being added to the cells). After 5 minutes, cells were wither fixed with 4% PFA in PBS

### Imaging Analyses

Fiji/ImageJ v2.1.0/153c was used to quantify IR fluorescence intensity per cell for the IR antibody validation experiment. With the polygon selection tool, a polygon was drawn around a cell outline. The average fluorescence intensity in the polygon (=in the cell) was determined using the measure tool on Fiji/ImageJ v2.1.0/153c. Background was then subtracted by a threshold determined by averaging the background intensity in a rectangular region outside of the cells.

To manually quantify IR fluorescent signal in puncta and condensates, Fiji/ImageJ v2.1.0/153c was used. A circle or an oval was drawn around IR puncta using the oval selection tool and the average fluorescence intensity in the circle or oval (=in the puncta) was determined using the measure tool on Fiji. Background was then subtracted as previously described. To quantify IR fluorescent signal in puncta and condensates in various cellular compartments, applicant identified the location of the plasma membrane, cytoplasm, and nucleus as follows. The plasma membrane location was identified based on IR immunofluorescence signal, IR-GFP fluorescent signal or CK18 immunofluorescence signal or cell edge. The nucleus was determined by the Hoechst stain for immunofluorescence and tc-PALM experiments. For IR-GFP experiments, Hoechst dye could not be used, because of bleed-through of the Hoechst fluorescence into the GFP channel confounded the identification of IR-GFP puncta. In these cases, the nuclear outline was inferred based on the very clear IR signal difference between the nucleus and the cytoplasm.

To computationally measure IR fluorescent signal in puncta and condensates, Airyscan images from all conditions were maximally-projected in the z-plane and background subtracted by a threshold determined by averaging the background intensity in a rectangular region outside of the cells. For segmenting IR puncta, the images were first subtracted by a median-filtered image (10 px) and then subjected to a Laplace of Gaussian filter (sigma=1). Filtered images were then thresholded on signal intensity (intensity>mean image intensity+2*standard deviation of image intensity). Thresholded binary images were then subjected to a morphological opening operation with a 3×3 filled structuring element to remove small objects. The mean intensity of the background-subtracted raw image was then measured for each segmented puncta (c-in), and background intensity (c-out) was calculated from the mean intensity of an inverted mask of the called puncta.

To quantify pIRS1 fluorescent signal in IR puncta/condensates manually, Airyscan images were opened on Fiji as composite images. A circle or an oval was drawn around IR puncta using the oval selection tool and the average fluorescence intensity of pIRS1 in the circle or oval (=in the puncta/condensates) was determined using the measure tool on Fiji.

To quantify pIRS1 fluorescent signal in IR puncta/condensates computationally using Fiji, 3D object counter tool was used. Briefly, images were thresholded on signal intensity and IR puncta/condensates were identified. 3D object counter tool then determined the intensity pIRS1 channel in the identified IR puncta. pIRS1 signal intensity was then background subtracted by a threshold determined by averaging the background intensity in a rectangular region outside of the cells.

To estimate the number of IR puncta at the plasma membrane, cytoplasm, nucleus in an entire cell, IR puncta were initially counted at various cellular locations in a cell slice using Fiji as described above. The number obtained from the cell slice was then multiplied based on the estimated surface area of the plasma membrane, volume of the cytoplasm, volume of the nucleus or volume of the entire cell, which were obtained considering the length and width of the cell under investigation and the estimated height (˜5 μm).

Quantification of the ROS dye fluorescence intensity per cell was performed using Fiji. Using the polygon selection tool on Fiji/ImageJ v2.1.0/153c, a polygon was drawn around a cell outline, which was identified by looking at the IR-GFP channel. The average ROS dye fluorescence intensity in the polygon (=in the cell) was determined using the measure tool on Fiji/ImageJ v2.1.0/153c.

Fusion, fission, or deformation events were identified in time-lapse images of the endogenously-tagged IR-GFP HepG2 line. To confirm bona fide deformation, fusion or fission events, applicant quantified the total IR intensity before and after the event as a product of IR fluorescence intensity and area of the IR puncta. The total intensity was conserved in bona fide deformation, fusion and fission events.

### ELISA

PathScan® Total Insulin Receptor β Sandwich ELISA kit (Cell Signaling, 7069) was used to quantify insulin receptor levels, PathScan Phospho-Akt2 (Ser474) and Total Akt2 Sandwich ELISA kits were used to quantify AKT2 levels (Cell Signaling Technology, #7048 and #7046, respectively) as per the manufacturer's instructions, by colorimetric reading at 450 nm on a Thermo Fisher Multiskan Go plate reader.

### Chromatin Immunoprecipitation-Sequencing (ChIP-Seq)

ChIP-seq experiments were performed by the Center for Functional Cancer Epigenetics (CFCE) at the Dana-Farber Cancer Institute. For ChIP-seq analysis, cells were cross-linked with 2 mM DSG (VWR, PI20593) for 45 minutes at room temperature followed by fixation for 10 minutes with 1% formaldehyde (Tousimis Research Corporation, 1008A) at room temperature on a shaker at 850 rpm. Crosslinked nuclei were quenched with 0.125M glycine (Sigma Aldrich, G7126) for 5 minutes at room temperature and washed with PBS (Life Technologies, AM9625) that contained protease inhibitor (Roche, 11836170001) and HDAC inhibitor Sodium Butyrate. After fixation, pellets were resuspended in 200 μl of 1% SDS, 50 mM Tris-HCl pH 8, 10 mM EDTA and sonicated in 1 ml AFA fiber millitubes (Covaris, 520135) for 25 minutes using a Covaris E220 instrument (setting: 140 peak incident power, 5% duty factor and 200 cycles per burst) 600 seconds per sample. Chromatin was diluted 5 times with ChIP Dilution buffer (1% Triton X-100, 2 mM EDTA pH 8, 150 mM NaCl, 20 mM Tris-HCl pH 8) and was immunoprecipitated with 10 μg of primary antibody against IR (Bethyl, A303-712A) and Dynabeads® Protein A/G (Thermo Fisher, 10015D). ChIP-seq libraries were constructed using NEBNext Ultra™ II kit (NEB, E7645S) according to the manufacturer's specifications. 75-bp paired-end reads were sequenced on a NextSeq instrument. 75-bp single-end reads were sequenced on an Illumina NextSeq instrument.
MED1 ChIP-seq was used from GEO: GSM2040029
RPB1 ChIP-seq was used from GEO: GSM2864931 (14)

### ChIP-Seg Analysis

ChIP-seq bioinformatics analysis for insulin receptor was performed on the Whitehead High-Performance Computing Facility using the nf-core ChIP-seq pipeline v1.2.1¹³ with Nextflow v20.04.1. Quality control of .fastq files was performed with FastQC v0.11.9. Trim Galore!v0.6.4_dev was used to trim low quality reads. Alignment was performed against the hg19 genome assembly using BWA v0.7.17-r1188¹⁴. Peak calling was performed using MACS2 v2.2.7.1¹⁵. Preseq v2.0.3¹⁶ and MultiQC v1.9 were used for quality control. Browser tracks were prepared to represent reads per million per basepair (rpm/bp).

### RT-qPCR and RNA-Sequencing

RNA was extracted using TRIzol™ reagent (Thermo Fisher Scientific, 15596026) following the manufacturer's instructions. cDNA synthesis was performed using qScript cDNA Supermix (QuantaBio, 95048-500) according to the manufacturer's instructions, using 1000 ng RNA as starting material.

qPCR was performed on a Thermo Fisher Scientific QuantStudio 6 machine using Fast SYBR™ Green Master Mix (Thermo Fisher, 4385618) and primers (listed below) according to the manufacturer's instructions. Expression data is presented after calculating the relative expression compared with the housekeeping gene RPLP0, using the equation Relative Quantification (RQ)=100/(2\hat{ }(Target Gene Ct—RPLP0 Ct). When data is reported relative to a sample condition, the condition of reference was set as 1 and the data of the other conditions were reported as a ratio (condition/condition of reference).

RNA sequencing was performed by the Whitehead Institute Genome Technology Core. Libraries were prepared using the KAPA HyperPrep stranded RNA kit (Roche, KK8540) following manufacturer's instructions. Samples were sequenced on a HiSeq2500 in High-Output mode generating 50 bases, single-end reads.

RT-qPCR primers
RPLP0_qF
(SEQ ID NO: 152)
GCAGCATCTACAACCCTGAAG
RPLP0_qR
(SEQ ID NO: 153)
GCAGACAGACACTGGCAACA
FASN_qF
(SEQ ID NO: 154)
CCGAGACACTCGTGGGCTA
FASN_qR
(SEQ ID NO: 155)
CTTCAGCAGGACATTGATGCC
PCK1_qF
(SEQ ID NO: 156)
GCTGGTGTCCCTCTAGTCTATG
PCK1_qR
(SEQ ID NO: 157)
GGTATTTGCCGAAGTTGTAG

### RNA-Sequencing Analysis

RNA-sequencing (RNA-seq) bioinformatics analysis was performed on the Whitehead High-Performance Computing Facility using the nf-core RNA-seq pipeline v1.4.2¹³ with Nextflow v20.04.1. Quality control of .fastq files was performed with FastQC v0.11.8. The reads were single-end and the strandedness was set to reverse. Low quality sequences were trimmed using Trim Galore!v0.6.4. Alignment was performed against the hg19 genome assembly using STAR v2.6.1d¹⁷ and duplicates were marked using Picard MarkDuplicates v2.21.1. Quantification of transcripts was performed using featureCounts v1.6.4¹⁸. Differential expression analysis was performed using edgeR v3.26.5¹⁹. deepTools v3.3.1²⁰, dupRadar v1.14.0²¹, Qualimap v.2.2.2-dev²², and MultiQC v1.7 were used for quality control.

### Functional Profiling of RNA-Sequencing

For functional profiling of RNA-sequencing, differentially expressed genes (based on adjusted p-value <0.05 and no log 2FC cut-off) from the RNA-seq experiment were uploaded to the online version of g:Profiler23. Over-representation analysis was performed using g:GOSt selecting Homo sapiens (Human) as organism and treating the query set as unordered. The selected statistical domain scope was “Only annotated genes” and the significance threshold (g:SCS) was set to 0.05. Significant KEGG pathways were selected for visual representation.

### Time-Correlated Photoactivation Localization Microscopy (Tc-PALM)

Widefield, live-cell, super-resolution imaging was performed in a photo-activation localization microscopy (PALM) approach using a Nikon Eclipse Ti microscope with a 100× oil immersion objective. The 405 nm and 561 nm laser beams were combined in an external platform with customized power densities to image Dendra2-tagged molecules as previously reported²⁴. Cells were cultured on imaging dishes (MatTek, P35G-1.5-20-C) and then imaged while maintaining both the temperature at 37° C. with a temperature-controlled platform and the level of CO₂ at 5% with Leibovitz's L-15 Medium with no phenol red (Thermo Fisher, 21083027). During each imaging cycle, a 2400-frame video stream including a (256 pixel)² region of interest (ROI) was recorded in 20 Hz acquisition rate with the EM-gain setting as 1000 on an Andor iXon Ultra 897 EMCCD. Each pixel conjugates with a (160 nm)² area on the sample side. After PALM imaging, the Hoechst-stained nuclei of the same ROI were imaged using a stronger 405 nm excitation through DAPI filter. For insulin stimulation, cells were first imaged in 1.5 ml insulin-free L-15 medium for 15-20 minutes. Afterwards, the cells were stimulated with insulin by adding 1.5 ml of prewarmed and freshly-made L-15 medium, 6 nM insulin to the same dish containing the original 1.5 ml of insulin-free L-15 medium. Following a 5-minute wait, the cells were imaged for 15-20 minutes. For the insulin-unstimulated condition, cells were imaged in 1.5 ml insulin-free L-15 Medium for 15-20 minutes. For the insulin-treated condition, 1.5 ml fresh-made, prewarmed L-15 medium containing 2× insulin (6 nM) was directly added to the same dish while it was still on the platform, followed by a 5-minute wait, then cells were imaged for 15-20 minutes.

### Tc-PALM Analysis

Detection localization: For each frame of a raw image, Gaussian particles were identified by pixelwise test of hypotheses, whose peak positions were individually fitted at subpixel resolution by maximum-likelihood regression with Gauss-Newton method²⁵. An additional deflation loop was performed to avoid missing dimmer particles when they were overshadowed by neighboring brighter ones. This multi-particle detection localization procedure has been integrated in a published, open-source MATLAB software called MTT²⁵.

Spatial clustering. DBSCAN and “manual selection” hybridized approach was applied to group spatially clustered detections via the qSR software²⁶. Firstly, DBSCAN was performed to generalize a proposal map of spatially clustered detections. Given that IR condensates can be tiny and transient, a “loose” parameter setting was used when performing the DBSCAN (length-scale=120 nm, N_min=4). This parameter combination was determined by comparing the rendered, super-resolved reconstructions with the color-coded cluster maps until the clustering results visually make sense for most ROIs. Second, individual clusters were manually selected based on the clustering proposal map from previous step. Custom MATLAB code was used to reconstruct the IR distribution of each ROI superposed with the corresponding nuclei image, which was further cross-compared with the corresponding cluster map to determine which region each cluster belongs to (i.e., plasma-membrane, cytoplasm, or nucleus).

Temporal clustering: For each spatial cluster, time-correlated PALM (tc-PALM) analysis was performed along the time axis to extract the truly colocalized, time-correlated multi-molecule bursting events. The lifetime of a burst is simply defined as the timespan from the first to the last detections. More details about the quantitative validations and statistics of tc-PALM analysis can be found in FIG. 30.

### Cryo-Immuno Electron Microscopy (EM)²⁷

The cells were fixed mildly Using PLP (paraformaldehyde/lysine/sodium periodate) fixative for 4 hours. Cells were pelleted. Infused with a cryo-protectant for at least one hour (PVP/sucrose). Blocks were mounted onto cryo-pins, and snap frozen in liquid nitrogen cooled ethane. Ultrathin sections were cut at −140 degrees C. with a Leica UC7 equipped with a FC7 cryo-stage using a glass knife, and immunolabeled, stained and embedded using the Tokuyasu technique. The material was examined using a Hitach 7800.

Antibody used were anti-insulin receptor beta antibody (Cell Signaling, 23413) for WT HepG2 cells or anti-GFP (Abcam, ab6556) for HepG2 cells expressing endogenous IR tagged with GFP).

### Protein Purification

Human cDNA encoding the beta subunit of the insulin receptor (IRb; residues 763-1382) was cloned into a mammalian expression vector. The base vector was engineered to include sequences encoding an N-terminal FLAG tag followed by mCherry and a 14 amino acid linker sequence “GAPGSAGSAAGGSG (SEQ ID NO: 158).” cDNA sequences were inserted in-frame following the linker sequence using NEBuilder HiFi DNA Assembly Master Mix (NEB, E2621S). The expression construct was subjected to Sanger sequencing to confirm the sequence.

For protein expression, IRb-mcherry plasmid was transfected into HEK293T cells (ATCC, CRL-3216) using Polyethylenimine (Fisher Scientific, NC1014320). Cells were cultured for 72 hours, scraped off the plate, and washed with ice-cold PBS (Life Technologies, AM9625). Cells were centrifuged at 500×g for 5 minutes and the cell pellet was stored at −80° C.

The cell pellet was resuspended in 35 ml Lysis Buffer (20 mM HEPES pH7.4, 150 mM NaCl, 1 mM EDTA, 0.5% NP40, with fresh inhibitors and 1 mM DTT). Cell lysate was rocked for 30 minutes at 4° C. and spun down at 12,000×g 15 minutes. 35 ml of supernatant was removed to a fresh tube and centrifuged again if cloudy. 300 μl of washed Anti-Flag M2 magnetic beads (Sigma Aldrich, M8823) was added to the lysate, which was then rotated overnight at 4° C. The next day beads were pelleted at 500 rpm for 5 minutes, washed with 35 ml BD Buffer (10 mM HEPES, 450 mM NaCl, 5% glycerol with fresh inhibitors), transferred to Eppendorf tube. Tubes were then placed in a magnetic rack to pellet beads and washed 3-5 times with BD Buffer, with resuspension of the pellet for each wash. Elution was performed overnight with 500 μl Dialysis Buffer (50 mM HEPES, 150 mM NaCl, 5 mM MgCl2, 5% glycerol) plus 50 μl Flag peptide (5 mg/ml stock solution). The next day the sample was eluted with the magnetic rack and washed with 250 μl Dialysis buffer with no peptide. The sample was dialyzed with 500 ml buffer, which was changed 1 to 2 times at 4° C.

### Insulin Binding Assay

Insulin-sensitive and insulin-resistant cells were washed in EMEM for 30 minutes as previously described, incubated on ice at 4° C. for 30 minutes and treated with 3 nM insulin for 60 minutes on ice at 4° C. with gentle agitation. Cells were washed five times with ice-cold PBS. TrypLE was added to the wells and cells were gently detached from the wells and added to a 1.5 ml tube. Cells were incubated at 37° C. for 10 minutes with gentle shaking. Cells were then pelleted and supernatant was collected into new 1.5 ml tube and incubated O/N at 37° C. with gentle shaking. Samples were then used for proteomics.

### Proteomics

SDB-RPS extraction of tryptic peptides. Tryptic digests were extracted by using custom-made SDB-RPS tips (CDS Analytical, Oxford, PA, USA) following the descriptions by Rappsilber et al. (2007)²⁸. Peptides were eluted from SBD-RPS filters with 50% (v/v) ACN and 5% (v/v) ammonium hydroxide, dried in a lyophilizer and taken up in 20 μL 0.2% (v/v) formic acid. Particles were removed from extracts by centrifugation for 10 min at 20,000 g at 4° C. before LC-MS analysis.

nanoLC-MS and data analysis. The LC-MS/MS analysis was performed using an Easy-nLC 1200 system connected to an Orbitrap Exploris 480 mass spectrometer (Thermo Fisher Scientific, Waltham, MA, USA) equipped with an Easy Spray ESI source for the ionization of eluting fractions. Peptide separation, collection of MS1 and MS2 profiles, and statistical data analyses were carried out as described by Schulte et al. (2019)²⁹.

### Statistical Analysis

Statistical analysis was performed using Prism (GraphPad, La Jolla, CA). Data was analyzed using t-test or t-test with Welch's correction two-tailed. One-tailed t-test was only used for FIG. 18F. Data is represented as individual values and mean or as individual values and mean±SEM. In all figures, * p<0.05, ** p<0.01, ***p<0.001.

### Schematics

BioRender was used to make the graphics reported in the figures (BioRender.com).

### Photochemistry of Single Dendra2 Molecules

Given that there could be an ambiguous mapping from the number of detections to the number of Dendra2 molecules, several control analyses of the single molecule photochemistry have to be done to validate the statistics of the real clusters (which ideally consist of colocalized, time-correlated, multimolecule bursts). Imaging of IR in either fixed or live IR-Dendra2 cells was performed in L-15 medium using the same laser setups as described above. After the same ROI was imaged for a long time, most Dendra2 molecules were photo-converted and bleached, whereupon the rest of intact single molecules were sparsely photo-converted and recorded, and the consequent colocalized detections from the same molecule can be well spatiotemporally isolated and grouped. The statistics of live-cell Dendra2 single molecules are shown in FIGS. 30A-30C. The comparisons of Dendra2 single molecules in live and fixed samples are shown in FIG. 30E. 94% of the single molecules only generate one detection (FIG. 30A), which results in the average number of detections per molecule being close to one (n _{det}≈1.077). The average lifetime of single molecules is 0.059 s, and only 1% of them has a lifetime longer than 0.25 s (FIG. 30B). Among those multiple-detection molecules, 65% of them result in the same emitting event occupying two adjacent frames (FIG. 30C), and the real average dark-time between blinking events is around 0.2 s.

### Validation of the Existence of Dynamic Clustering in Live Cells

Applicant identified pseudo-transient clusters in fixed cells with the exact procedures and criteria as for searching transient clusters in live cells. For spatially clustered structures, significantly larger dark times in live cells, compared to fixed cells under identical condition, is a sign of the bursting dynamics in live cells³⁰. This is exactly what was observed (FIG. 30D), and such larger dark times of clusters in live cells cannot be explained by longer intrinsic inter-detection period of Dendra2 single molecules in live-cell samples (FIG. 30E). Furthermore, applicant normalized the number of tc-PALM identified bursts by the total number of detections of the same ROI, thus are able to estimate the number of identified bursts per 10,000 detections as 67.02 (FIG. 30F). Meanwhile, among the tc-PALM identified bursts, applicant obtained the number of detections and lifetime of the 0.05 quantile at the lower-bound side as 4 and 0.85 s, respectively (FIG. 30F). If applicant uses these two numbers as the cut-off for the set of Dendra2 single molecules applicant measured in live samples, only 4.67 molecules among 10,000 detections can pass the threshold. This indicates that the true positive rate (TPR) can easily go beyond 90%: 67.02÷(67.02+4.67)=93.5%; even in the worst case (all the bursts below the 0.05 quantile were single molecules), the corresponding TPR is 93.5% x95%≈89%. Even for the outlier single molecules that pass the cut-off, their statistics (including duration time, inter-detection period, and number of detections) are still quite different from the of tc-PALM identified bursts (FIG. 30F). In another extreme test, applicant applied several additional high cut-offs to the tc-PALM identified bursts (in some cases, the TPR was pushed to 98%), whereupon applicants are still able to recapitulate all the significant trends of lifetime-shifting in cytoplasm and nuclei after different perturbations (data not shown). This observation is reasonable: given that IR molecules are much less abundant in the cytoplasm and nuclei, un-clustered background of randomly bound IR molecules can be safely ignored. Therefore, any time-correlated, multi-detection events inside in the cytoplasm or nuclei are very likely to result from real clusters, which are insensitive to the FPR cut-off. Gathering all these evidences together, applicant was able to validate the existence of multi-molecule dynamical clustering of IR molecules in live cells, which yields transient bursting dynamics with distinct properties than single molecules and are robustly, physiologically responsive.

### SUPPLEMENTARY REFERENCES

1. Ramakrishnan, G., Arjuman, A., Suneja, S., Das, C. & Chandra, N. C. The association between insulin and low-density lipoprotein receptors. Diab Vase Dis Res 9, 196-204 (2012).
2. Hancock, M. L., et al. Insulin Receptor Associates with Promoters Genome-wide and Regulates Gene Expression. Cell 177, 722-736 e722 (2019).
3. Foretz, M., Guichard, C., Ferre, P. & Foufelle, F. Sterol regulatory element binding protein-1c is a major mediator of insulin action on the hepatic expression of glucokinase and lipogenesis-related genes. Proc Natl Acad Sci USA 96, 12737-12742 (1999).
4. Hillgartner, F. B., Salati, L. M. & Goodridge, A. G. Physiological and molecular mechanisms involved in nutritional regulation of fatty acid synthesis. Physiol Rev 75, 47-76 (1995).
5. Fortez M, et al. ADD1/SREBP-1c is required in the activation of hepatic lipogenic gene expression by glucose. Mol Cell Biol 19, 3760-3768 (1999).
6. Claycombe, K. J., et al. Insulin increases fatty acid synthase gene transcription in human adipocytes. Am J Physiol 274, R1253-1259 (1998).
7. Dif, N., et al. Insulin activates human sterol-regulatory-element-binding protein-1c (SREBP-1c) promoter through SRE motifs. Biochem J 400, 179-188 (2006).
8. Yadollah, S., Kazemipour, N., Bakhtiyari, S. & Nazifi, S. Palmitate-induced insulin resistance is attenuated by Pioglitazone and EGCG through reducing the gluconeogenic key enzymes expression in HepG2 cells. J Med Life 10, 244-249 (2017).
9. Sefried, S., Haring, H. U., Weigert, C. & Eckstein, S. S. Suitability of hepatocyte cell lines HepG2, AML12 and THLE-2 for investigation of insulin signalling and hepatokine gene expression. Open Biol 8(2018).
10. Huoh, Y. S., et al. Dual functions of Aire CARD multimerization in the transcriptional regulation of T cell tolerance. Nat Commun 11, 1625 (2020).
11. Henninger, J. E., et al. RNA-Mediated Feedback Control of Transcriptional Condensates. Cell 184, 207-225 e224 (2021).
12. Alzamil, H. Elevated Serum TNF-alpha Is Related to Obesity in Type 2 Diabetes Mellitus and Is Associated with Glycemic Control and Insulin Resistance. J Obes 2020, U.S. Pat. No. 5,076,858 (2020).
13. Ewels, P. A., et al. The nf-core framework for community-curated bioinformatics pipelines. Nat Biotechnol 38, 276-278 (2020).
14. Li, H. & Durbin, R. Fast and accurate short read alignment with Burrows-Wheeler transform. Bioinformatics 25, 1754-1760 (2009).
15. Zhang, Y., et al. Model-based analysis of ChIP-Seq (MACS). Genome Biol 9, R137 (2008).
16. Daley, T. & Smith, A. D. Predicting the molecular complexity of sequencing libraries. Nat Methods 10, 325-327 (2013).
17. Dobin, A., et al. STAR: ultrafast universal RNA-seq aligner. Bioinformatics 29, 15-21 (2013).
18. Liao, Y., Smyth, G. K. & Shi, W. featureCounts: an efficient general purpose program for assigning sequence reads to genomic features. Bioinformatics 30, 923-930 (2014).
19. Robinson, M. D., McCarthy, D. J. & Smyth, G. K. edgeR: a Bioconductor package for differential expression analysis of digital gene expression data. Bioinformatics 26, 139-140 (2010).
20. Ramirez, F., Dundar, F., Diehl, S., Gruning, B. A. & Manke, T. deepTools: a flexible platform for exploring deep-sequencing data. Nucleic Acids Res 42, W187-191 (2014).
21. Sayols, S., Scherzinger, D. & Klein, H. dupRadar: a Bioconductor package for the assessment of PCR artifacts in RNA-Seq data. BMC Bioinformatics 17, 428 (2016).
22. Okonechnikov, K., Conesa, A. & Garcia-Alcalde, F. Qualimap 2: advanced multi-sample quality control for high-throughput sequencing data. Bioinformatics 32, 292-294 (2016).
23. Reimand, J., Kull, M., Peterson, H., Hansen, J. & Vilo, J. g:Profiler—a web-based toolset for functional profiling of gene lists from large-scale experiments. Nucleic Acids Res 35, W193-200 (2007).
24. Cho, W. K., et al. Mediator and RNA polymerase II clusters associate in transcription-dependent condensates. Science 361, 412-415 (2018).
25. Serge, A., Bertaux, N., Rigneault, H. & Marguet, D. Dynamic multiple-target tracing to probe spatiotemporal cartography of cell membranes. Nat Methods 5, 687-694 (2008).
26. Andrews, J. O., et al. qSR: a quantitative super-resolution analysis tool reveals the cell-cycle dependent organization of RNA Polymerase I in live human cells. Sci Rep 8, 7424 (2018).
27. Tokuyasu, K. T. Immunochemistry on ultrathin frozen sections. Histochem J 12, 381-403 (1980).
28. Rappsilber, J., Mann, M. & Ishihama, Y. Protocol for micro-purification, enrichment, pre-fractionation and storage of peptides for proteomics using StageTips. Nat Protoc 2, 1896-1906 (2007).
29. Schulte, F., Hasturk, H. & Hardt, M. Mapping Relative Differences in Human Salivary Gland Secretions by Dried Saliva Spot Sampling and nanoLC-MS/MS. Proteomics 19, e1900023 (2019).
30. Cisse, I I, et al. Real-time dynamics of RNA polymerase II clustering in live human cells. Science 341, 664-667 (2013).

1. A method of decreasing insulin resistance in an insulin resistant cell, comprising contacting the cell with an agent that decreases a level of reactive oxygen species (ROS) in the cell, wherein the agent is not metformin.

2. The method of claim 1, wherein contact of the cell with the agent and insulin increases incorporation of insulin receptor (IR) into cytoplasmic and/or nuclear condensates in the cell.

3. The method of claim 1, wherein contact of the cell with the agent and insulin increases incorporation of insulin receptor (IR) into transcriptional condensates.

4. The method of claim 3, wherein the transcriptional condensates comprise transcriptional condensates modulating the expression of one or more insulin responsive genes.

5. The method of claim 1, wherein the cell is a mammalian fat, liver, brain, kidney, muscle, or pancreatic islet cell.

6. The method of claim 1, wherein the agent is contacted with the cell in vivo in a subject in need thereof.

7.-11. (canceled)

12. A method of screening for a candidate agent to treat insulin resistance comprising contacting a test agent with a condensate and insulin receptor (IR) in an elevated reactive oxygen species (ROS) environment, wherein if the test agent increases flux of IR incorporation into the condensate as compared to a control then the test agent is identified as a candidate agent to treat insulin resistance.

13.-18. (canceled)

19. The method of claim 12, wherein the condensate is a synthetic in vitro condensate or an ex vivo condensate isolated from a cell.

20. The method of claim 19, wherein the ex vivo condensate is isolated from an insulin resistant cell.

21. The method of claim 20, wherein the insulin resistant cell is a mammalian fat, liver, brain, kidney, muscle, or pancreatic islet cell.

22. (canceled)

23. (canceled)

24. The method of claim 12, wherein the condensate(s) is/are in a cell.

25. The method of claim 24 wherein the cell is a mammalian fat, liver, brain, kidney, muscle or pancreatic islet cell.

26. The method of claim 24, wherein the cell is an insulin resistant cell.

27. The method of claim 26, wherein the cell is an insulin resistant and metformin resistant cell.

28. A method of characterizing an agent, comprising (i) contacting the agent with a cell comprising the insulin receptor, and (ii) measuring ability of the agent to modulate one or more of the following: (a) flux of IR incorporation into condensates within the cell in response to insulin; (b) average number of IR molecules in IR condensates in the cell; (c) average lifetime of IR condensates in the cell; (d) percentage of long-lived IR condensates in the cell.

29. The method of claim 28 wherein the cell is a mammalian fat, liver, brain, kidney, muscle or pancreatic islet cell.

30. The method of claim 29, wherein the cell is an insulin resistant cell.

31. The method of claim 29, wherein the cell is an insulin resistant and metformin resistant cell.

32. The method of claim 28, wherein the cell is contacted with insulin prior to step (i).

33. The method of claim 28, wherein the cell is contacted with insulin prior to step (ii).


SEQUENCE LISTING (158 sequences)

SEQ ID NO: 1 - RNA, 23 nt, synthetic construct
  source 1..23: mol_type = other RNA
cacggtaggcactgttaggaagg

SEQ ID NO: 2 - RNA, 23 nt, synthetic construct
  source 1..23: mol_type = other RNA
taggcactgttaggaaggattgg

SEQ ID NO: 3 - DNA, 19 bp, synthetic construct
  source 1..19: mol_type = other DNA
ggagaatgtgcccctggac

SEQ ID NO: 4 - DNA, 23 bp, synthetic construct
  source 1..23: mol_type = other DNA
ttggtaaccaaacgagtccacct

SEQ ID NO: 5 - DNA, 960 bp, synthetic construct
  source 1..960: mol_type = other DNA
acacttcacacaccaaagcagacatgtgctgcaaaatcctgggaacaattacagctcctaaccagtgagtttctctaagtcaacatgaaatgcgtcccatatgccaaatgcccaaaactcgcaggtaccccacaaaaaagcaagctcagttcctgaagagaacaataccctcagtctcagaacatcctgagcgtgttattctttcaaaagctgccacttcgagctcaaactttgaagcctctcgctacaaagaggacagcgccaataaagttcccttttgttacaaaactctctgccctggaaaaggaggaggtttgggcaggaaaacagcacacagggcctgcaatgctggaattccaaaaaaatgtcccattttcccaacaatctcctggaagcaagtggattgtctatctgtacgtcctttcaaaagacacccatccatgacacatccatgtccaagtgatagcttccaacacatgccaaaatgaaagcccccagacggaggctagaagatgagtctgatactgactcaaggacgcttatttttgaaggctctcctcggggtgcattttcagattatgcaatgtttcctaagctactcccagttcatgatcacaatttcagtgaaaccatgagctgttcactccacaatggcattagcatgctacagtgtcaccaaatacaagtctacgcagctacacaactgctttgagagtgctgttttagtgtgcgagcaagtcaagtgtctaccatcatcaggcttgctcaggtaacacacagaaaccagagggaactttcctgaacttgggcaaggacatcatacagtatcctttcacgaggtgaaaatagcccggagaggatcttggtgtgttaaccactaccaacaggtttaggtagagtttctctctctagcagatactgagttggcttcgtaagaagcgtgaaagttatcaccctgtt

SEQ ID NO: 6 - DNA, 21 bp, synthetic construct
  source 1..21: mol_type = other DNA
gcagcatctacaaccctgaag

SEQ ID NO: 7 - DNA, 20 bp, synthetic construct
  source 1..20: mol_type = other DNA
gcagacagacactggcaaca

SEQ ID NO: 8 - DNA, 20 bp, synthetic construct
  source 1..20: mol_type = other DNA
gggctttcagattcccaact

SEQ ID NO: 9 - DNA, 21 bp, synthetic construct
  source 1..21: mol_type = other DNA
ctcgtcttctcctcccagtat

SEQ ID NO: 10 - protein, 14 aa, synthetic construct
GAPGSAGSAAGGSG

SEQ ID NO: 11 - protein, 21 aa, synthetic construct
GIVEQCCTSICSLYQLENYCN

SEQ ID NO: 12 - protein, 7 aa, synthetic construct
GFFYTPK

SEQ ID NO: 13 - RNA, 23 nt, synthetic construct
  source 1..23: mol_type = other RNA
cacggtaggcactgttaggaagg

SEQ ID NO: 14 - RNA, 23 nt, synthetic construct
  source 1..23: mol_type = other RNA
taggcactgttaggaaggattgg

SEQ ID NO: 15 - DNA, 19 bp, synthetic construct
  source 1..19: mol_type = other DNA
ggagaatgtgcccctggac

SEQ ID NO: 16 - DNA, 23 bp, synthetic construct
  source 1..23: mol_type = other DNA
ttggtaaccaaacgagtccacct

SEQ ID NO: 17 - DNA, 20 bp, synthetic construct
  source 1..20: mol_type = other DNA
cgagagcggaggatgaggag

SEQ ID NO: 18 - DNA, 20 bp, synthetic construct
  source 1..20: mol_type = other DNA
aaggggcacgaacaccgaga

SEQ ID NO: 19 - DNA, 20 bp, synthetic construct
  source 1..20: mol_type = other DNA
gaaatggggatagcctatgc

SEQ ID NO: 20 - DNA, 20 bp, synthetic construct
  source 1..20: mol_type = other DNA
cattcagttcagggtattgg

SEQ ID NO: 21 - DNA, 20 bp, synthetic construct
  source 1..20: mol_type = other DNA
acaaaggtggagatggagct

SEQ ID NO: 22 - DNA, 20 bp, synthetic construct
  source 1..20: mol_type = other DNA
gtgcaatgtccaggaaggag

SEQ ID NO: 23 - DNA, 20 bp, synthetic construct
  source 1..20: mol_type = other DNA
ctccgtaacaagcagatggg

SEQ ID NO: 24 - DNA, 20 bp, synthetic construct
  source 1..20: mol_type = other DNA
gagcacatctggtatgcaac

SEQ ID NO: 25 - DNA, 20 bp, synthetic construct
  source 1..20: mol_type = other DNA
tagagctgctctgtggaaga

SEQ ID NO: 26 - DNA, 20 bp, synthetic construct
  source 1..20: mol_type = other DNA
tctaccatgctgactcacag

SEQ ID NO: 27 - DNA, 20 bp, synthetic construct
  source 1..20: mol_type = other DNA
gactggtacgaccagatctg

SEQ ID NO: 28 - DNA, 20 bp, synthetic construct
  source 1..20: mol_type = other DNA
caaacaatggagcaggctcc

SEQ ID NO: 29 - DNA, 20 bp, synthetic construct
  source 1..20: mol_type = other DNA
aactgaggactctctgctat

SEQ ID NO: 30 - DNA, 20 bp, synthetic construct
  source 1..20: mol_type = other DNA
gacaggcgggtgtttaaatc

SEQ ID NO: 31 - DNA, 20 bp, synthetic construct
  source 1..20: mol_type = other DNA
tggggtgcagttgggaaact

SEQ ID NO: 32 - DNA, 20 bp, synthetic construct
  source 1..20: mol_type = other DNA
atcagaagcgaggagactgg

SEQ ID NO: 33 - DNA, 20 bp, synthetic construct
  source 1..20: mol_type = other DNA
ctcaagaacctcctgctttg

SEQ ID NO: 34 - DNA, 20 bp, synthetic construct
  source 1..20: mol_type = other DNA
tggcgggagagggttgaaat

SEQ ID NO: 35 - DNA, 20 bp, synthetic construct
  source 1..20: mol_type = other DNA
aggtcaaatgccagtcagag

SEQ ID NO: 36 - DNA, 20 bp, synthetic construct
  source 1..20: mol_type = other DNA
gagcacctggatcttaagaa

SEQ ID NO: 37 - DNA, 20 bp, synthetic construct
  source 1..20: mol_type = other DNA
gagcataaccttagtcttgg

SEQ ID NO: 38 - DNA, 20 bp, synthetic construct
  source 1..20: mol_type = other DNA
aggagacaaaggccaagtgt

SEQ ID NO: 39 - DNA, 20 bp, synthetic construct
  source 1..20: mol_type = other DNA
agaacaggccgtgataagga

SEQ ID NO: 40 - DNA, 20 bp, synthetic construct
  source 1..20: mol_type = other DNA
aacaggaaccaggtcacaga

SEQ ID NO: 41 - DNA, 20 bp, synthetic construct
  source 1..20: mol_type = other DNA
tgccacaaacagtggtcaag

SEQ ID NO: 42 - DNA, 20 bp, synthetic construct
  source 1..20: mol_type = other DNA
cacactcacctgcaaggaag

SEQ ID NO: 43 - DNA, 20 bp, synthetic construct
  source 1..20: mol_type = other DNA
tagtgggtcaggaagctgta

SEQ ID NO: 44 - DNA, 20 bp, synthetic construct
  source 1..20: mol_type = other DNA
tgtgcctggtctctctaaag

SEQ ID NO: 45 - DNA, 20 bp, synthetic construct
  source 1..20: mol_type = other DNA
gtgagtgatcatgggtgttg

SEQ ID NO: 46 - DNA, 20 bp, synthetic construct
  source 1..20: mol_type = other DNA
ctgtggcctgaacactaagg

SEQ ID NO: 47 - DNA, 20 bp, synthetic construct
  source 1..20: mol_type = other DNA
ctaattctgccatggcacag

SEQ ID NO: 48 - DNA, 20 bp, synthetic construct
  source 1..20: mol_type = other DNA
caaggctctgcctagcaaag

SEQ ID NO: 49 - DNA, 20 bp, synthetic construct
  source 1..20: mol_type = other DNA
gataggaaagaagcccctat

SEQ ID NO: 50 - DNA, 20 bp, synthetic construct
  source 1..20: mol_type = other DNA
ggaacaggacactcaggttg

SEQ ID NO: 51 - DNA, 20 bp, synthetic construct
  source 1..20: mol_type = other DNA
tctgagggaaccctgatgac

SEQ ID NO: 52 - DNA, 20 bp, synthetic construct
  source 1..20: mol_type = other DNA
agagatgggtgccaacagag

SEQ ID NO: 53 - DNA, 20 bp, synthetic construct
  source 1..20: mol_type = other DNA
gtggaggtgtcattacagag

SEQ ID NO: 54 - DNA, 20 bp, synthetic construct
  source 1..20: mol_type = other DNA
gagccttcatgagaaaggtt

SEQ ID NO: 55 - DNA, 20 bp, synthetic construct
  source 1..20: mol_type = other DNA
acaagtgtggtaatggcagc

SEQ ID NO: 56 - DNA, 20 bp, synthetic construct
  source 1..20: mol_type = other DNA
aagggacctgaggacaaacc

SEQ ID NO: 57 - DNA, 20 bp, synthetic construct
  source 1..20: mol_type = other DNA
caagggaaccgagagggaat

SEQ ID NO: 58 - DNA, 20 bp, synthetic construct
  source 1..20: mol_type = other DNA
tattagtccacctggacatc

SEQ ID NO: 59 - DNA, 20 bp, synthetic construct
  source 1..20: mol_type = other DNA
ctacggagaagggaggcatg

SEQ ID NO: 60 - DNA, 20 bp, synthetic construct
  source 1..20: mol_type = other DNA
tgtggggagggaagagtctg

SEQ ID NO: 61 - DNA, 20 bp, synthetic construct
  source 1..20: mol_type = other DNA
gggagagttggagatcagag

SEQ ID NO: 62 - DNA, 20 bp, synthetic construct
  source 1..20: mol_type = other DNA
tggaaagggaggtgcggaag

SEQ ID NO: 63 - DNA, 20 bp, synthetic construct
  source 1..20: mol_type = other DNA
agaagcaagtctggggtcaa

SEQ ID NO: 64 - DNA, 20 bp, synthetic construct
  source 1..20: mol_type = other DNA
actgaggaacagtgaccagg

SEQ ID NO: 65 - DNA, 20 bp, synthetic construct
  source 1..20: mol_type = other DNA
ttggctgtaagctgtgtgtc

SEQ ID NO: 66 - DNA, 20 bp, synthetic construct
  source 1..20: mol_type = other DNA
ctaaataaacgaggctggcc

SEQ ID NO: 67 - DNA, 20 bp, synthetic construct
  source 1..20: mol_type = other DNA
acctttattgaagaggcctg

SEQ ID NO: 68 - DNA, 20 bp, synthetic construct
  source 1..20: mol_type = other DNA
gatgcagacagcagggtctg

SEQ ID NO: 69 - DNA, 20 bp, synthetic construct
  source 1..20: mol_type = other DNA
cgggcatagggttagaatgt

SEQ ID NO: 70 - DNA, 20 bp, synthetic construct
  source 1..20: mol_type = other DNA
tggagctcaataaagccagg

SEQ ID NO: 71 - DNA, 20 bp, synthetic construct
  source 1..20: mol_type = other DNA
aaggctagagaagaggccag

SEQ ID NO: 72 - DNA, 20 bp, synthetic construct
  source 1..20: mol_type = other DNA
gactagagctgaatgcaggg

SEQ ID NO: 73 - DNA, 20 bp, synthetic construct
  source 1..20: mol_type = other DNA
ctttcaggaccagaggtaac

SEQ ID NO: 74 - DNA, 20 bp, synthetic construct
  source 1..20: mol_type = other DNA
ctttgatggggctgggttag

SEQ ID NO: 75 - DNA, 20 bp, synthetic construct
  source 1..20: mol_type = other DNA
ggactactgtagctcctaaa

SEQ ID NO: 76 - DNA, 20 bp, synthetic construct
  source 1..20: mol_type = other DNA
aaaggctggatatgtgaccc

SEQ ID NO: 77 - DNA, 20 bp, synthetic construct
  source 1..20: mol_type = other DNA
cgaaaggaacagagccagga

SEQ ID NO: 78 - DNA, 20 bp, synthetic construct
  source 1..20: mol_type = other DNA
atgaggctcagaggatatgg

SEQ ID NO: 79 - DNA, 20 bp, synthetic construct
  source 1..20: mol_type = other DNA
taggatctgttagggtcttc

SEQ ID NO: 80 - DNA, 20 bp, synthetic construct
  source 1..20: mol_type = other DNA
cttacctatggacagaggga

SEQ ID NO: 81 - DNA, 20 bp, synthetic construct
  source 1..20: mol_type = other DNA
agcaggaacaagggttgaca

SEQ ID NO: 82 - DNA, 20 bp, synthetic construct
  source 1..20: mol_type = other DNA
gaggacgggacagattcatg

SEQ ID NO: 83 - DNA, 20 bp, synthetic construct
  source 1..20: mol_type = other DNA
cagtcctacctgtggatgag

SEQ ID NO: 84 - DNA, 20 bp, synthetic construct
  source 1..20: mol_type = other DNA
gagagagctgcagggataag

SEQ ID NO: 85 - DNA, 20 bp, synthetic construct
  source 1..20: mol_type = other DNA
taagagagcacctgtagggg

SEQ ID NO: 86 - DNA, 20 bp, synthetic construct
  source 1..20: mol_type = other DNA
ctgcatgtgcttctgaaagc

SEQ ID NO: 87 - DNA, 20 bp, synthetic construct
  source 1..20: mol_type = other DNA
tcacttctccaattagccat

SEQ ID NO: 88 - DNA, 20 bp, synthetic construct
  source 1..20: mol_type = other DNA
gagaagatgccattgttggc

SEQ ID NO: 89 - DNA, 20 bp, synthetic construct
  source 1..20: mol_type = other DNA
gcttccaaaggaaaaccgac

SEQ ID NO: 90 - DNA, 20 bp, synthetic construct
  source 1..20: mol_type = other DNA
ggtggacataactatcacca

SEQ ID NO: 91 - DNA, 20 bp, synthetic construct
  source 1..20: mol_type = other DNA
ctgccaaggacaggggaaag

SEQ ID NO: 92 - DNA, 20 bp, synthetic construct
  source 1..20: mol_type = other DNA
gagggcacaacgacacttac

SEQ ID NO: 93 - DNA, 20 bp, synthetic construct
  source 1..20: mol_type = other DNA
gagctattctcagaatcccg

SEQ ID NO: 94 - DNA, 20 bp, synthetic construct
  source 1..20: mol_type = other DNA
gaggaatgaagcgtgcatgg

SEQ ID NO: 95 - DNA, 20 bp, synthetic construct
  source 1..20: mol_type = other DNA
ctgtcggaacagatggcagg

SEQ ID NO: 96 - DNA, 20 bp, synthetic construct
  source 1..20: mol_type = other DNA
tcacctgtggaaggagagag

SEQ ID NO: 97 - DNA, 20 bp, synthetic construct
  source 1..20: mol_type = other DNA
tgagaagggagccaggacag

SEQ ID NO: 98 - DNA, 20 bp, synthetic construct
  source 1..20: mol_type = other DNA
aacaaaggctgagtgaggca

SEQ ID NO: 99 - DNA, 20 bp, synthetic construct
  source 1..20: mol_type = other DNA
ccctgaggaaaaaaggtggt

SEQ ID NO: 100 - DNA, 20 bp, synthetic construct
  source 1..20: mol_type = other DNA
cagaagagtgccagtcagac

SEQ ID NO: 101 - DNA, 20 bp, synthetic construct
  source 1..20: mol_type = other DNA
ccagggaatggaaagctgaa

SEQ ID NO: 102 - DNA, 20 bp, synthetic construct
  source 1..20: mol_type = other DNA
gaagccttagccaaaaagca

SEQ ID NO: 103 - DNA, 20 bp, synthetic construct
  source 1..20: mol_type = other DNA
gcaatgcaacagcaatgcac

SEQ ID NO: 104 - DNA, 20 bp, synthetic construct
  source 1..20: mol_type = other DNA
tgctgagcagacagcacatc

SEQ ID NO: 105 - DNA, 20 bp, synthetic construct
  source 1..20: mol_type = other DNA
ttggtatcacatcccatgtg

SEQ ID NO: 106 - DNA, 20 bp, synthetic construct
  source 1..20: mol_type = other DNA
ccactgattccttgtgaaag

SEQ ID NO: 107 - DNA, 20 bp, synthetic construct
  source 1..20: mol_type = other DNA
gtatcccacaaatgacagtc

SEQ ID NO: 108 - DNA, 20 bp, synthetic construct
  source 1..20: mol_type = other DNA
cacagactgagtcacgcacg

SEQ ID NO: 109 - DNA, 20 bp, synthetic construct
  source 1..20: mol_type = other DNA
gtctcagcccacacacaaag

SEQ ID NO: 110 - DNA, 20 bp, synthetic construct
  source 1..20: mol_type = other DNA
tgtacctggcacacaggtac

SEQ ID NO: 111 - DNA, 20 bp, synthetic construct
  source 1..20: mol_type = other DNA
acgaggatgtgtcagggatg

SEQ ID NO: 112 - DNA, 20 bp, synthetic construct
  source 1..20: mol_type = other DNA
tggtcttctcggcagagatc

SEQ ID NO: 113 - DNA, 20 bp, synthetic construct
  source 1..20: mol_type = other DNA
agggtgtggtcaaggtcaag

SEQ ID NO: 114 - DNA, 20 bp, synthetic construct
  source 1..20: mol_type = other DNA
acgcccgattcacgaacgag

SEQ ID NO: 115 - DNA, 20 bp, synthetic construct
  source 1..20: mol_type = other DNA
attttcatcaggaaagccgg

SEQ ID NO: 116 - DNA, 20 bp, synthetic construct
  source 1..20: mol_type = other DNA
cacagcaccgattctctaac

SEQ ID NO: 117 - DNA, 20 bp, synthetic construct
  source 1..20: mol_type = other DNA
atgtaataactttcaggccc

SEQ ID NO: 118 - DNA, 20 bp, synthetic construct
  source 1..20: mol_type = other DNA
tcacctggcacgtgatattg

SEQ ID NO: 119 - DNA, 20 bp, synthetic construct
  source 1..20: mol_type = other DNA
tgaagaccaggctcttgtct

SEQ ID NO: 120 - DNA, 20 bp, synthetic construct
  source 1..20: mol_type = other DNA
acatagtataatacaggccc

SEQ ID NO: 121 - DNA, 20 bp, synthetic construct
  source 1..20: mol_type = other DNA
ttcctgctcaacattcttct

SEQ ID NO: 122 - DNA, 20 bp, synthetic construct
  source 1..20: mol_type = other DNA
tgcatgtgcatttccatatg

SEQ ID NO: 123 - DNA, 20 bp, synthetic construct
  source 1..20: mol_type = other DNA
aacaactgctgccctagagt

SEQ ID NO: 124 - DNA, 20 bp, synthetic construct
  source 1..20: mol_type = other DNA
tctttcagtttttcaaacct

SEQ ID NO: 125 - DNA, 20 bp, synthetic construct
  source 1..20: mol_type = other DNA
cggggctgtctagtcacaat

SEQ ID NO: 126 - DNA, 20 bp, synthetic construct
  source 1..20: mol_type = other DNA
acaagtgcagacttcgtctc

SEQ ID NO: 127 - DNA, 20 bp, synthetic construct
  source 1..20: mol_type = other DNA
ataaaatcatgggcacctcc

SEQ ID NO: 128 - DNA, 20 bp, synthetic construct
  source 1..20: mol_type = other DNA
acctcatggtcaatatgagt

SEQ ID NO: 129 - DNA, 20 bp, synthetic construct
  source 1..20: mol_type = other DNA
actgagcacatgccagtatg

SEQ ID NO: 130 - DNA, 20 bp, synthetic construct
  source 1..20: mol_type = other DNA
gtgacattcacagtaatgct

SEQ ID NO: 131 - DNA, 20 bp, synthetic construct
  source 1..20: mol_type = other DNA
ttgcaaatcactctcttggc

SEQ ID NO: 132 - DNA, 20 bp, synthetic construct
  source 1..20: mol_type = other DNA
ccctcaacttcagctatcaa

SEQ ID NO: 133 - DNA, 20 bp, synthetic construct
  source 1..20: mol_type = other DNA
attctctctacattctctca

SEQ ID NO: 134 - DNA, 20 bp, synthetic construct
  source 1..20: mol_type = other DNA
aagattccatctttaaccct

SEQ ID NO: 135 - DNA, 20 bp, synthetic construct
  source 1..20: mol_type = other DNA
ctgtcattccctaacatttc

SEQ ID NO: 136 - DNA, 20 bp, synthetic construct
  source 1..20: mol_type = other DNA
ctccgtctctaagatttctt

SEQ ID NO: 137 - DNA, 20 bp, synthetic construct
  source 1..20: mol_type = other DNA
gggtctatgttactgacatc

SEQ ID NO: 138 - DNA, 20 bp, synthetic construct
  source 1..20: mol_type = other DNA
ttgcctggagacttgcaatc

SEQ ID NO: 139 - DNA, 20 bp, synthetic construct
  source 1..20: mol_type = other DNA
ctcttgccactcaaactctc

SEQ ID NO: 140 - DNA, 20 bp, synthetic construct
  source 1..20: mol_type = other DNA
atgcttttggatgaccaccg

SEQ ID NO: 141 - DNA, 20 bp, synthetic construct
  source 1..20: mol_type = other DNA
tgggatcatcctggaaggga

SEQ ID NO: 142 - DNA, 20 bp, synthetic construct
  source 1..20: mol_type = other DNA
catgttttctgcattactct

SEQ ID NO: 143 - DNA, 20 bp, synthetic construct
  source 1..20: mol_type = other DNA
acagagatgaaggtgtcttt

SEQ ID NO: 144 - DNA, 20 bp, synthetic construct
  source 1..20: mol_type = other DNA
agacacttacctagaagcaa

SEQ ID NO: 145 - DNA, 20 bp, synthetic construct
  source 1..20: mol_type = other DNA
gcaaggatttcttagaaggc

SEQ ID NO: 146 - DNA, 20 bp, synthetic construct
  source 1..20: mol_type = other DNA
aagtaatgaaatggtggccc

SEQ ID NO: 147 - DNA, 20 bp, synthetic construct
  source 1..20: mol_type = other DNA
tgcttctgatttgctttcta

SEQ ID NO: 148 - DNA, 20 bp, synthetic construct
  source 1..20: mol_type = other DNA
ctctccaataagtctcgttt

SEQ ID NO: 149 - DNA, 20 bp, synthetic construct
  source 1..20: mol_type = other DNA
ccagcatttggaatgtaatc

SEQ ID NO: 150 - DNA, 20 bp, synthetic construct
  source 1..20: mol_type = other DNA
caccagagtgctgaaaccaa

SEQ ID NO: 151 - DNA, 20 bp, synthetic construct
  source 1..20: mol_type = other DNA
ttcatagatgcttcctgcag

SEQ ID NO: 152 - DNA, 21 bp, synthetic construct
  source 1..21: mol_type = other DNA
gcagcatctacaaccctgaag

SEQ ID NO: 153 - DNA, 20 bp, synthetic construct
  source 1..20: mol_type = other DNA
gcagacagacactggcaaca

SEQ ID NO: 154 - DNA, 19 bp, synthetic construct
  source 1..19: mol_type = other DNA
ccgagacactcgtgggcta

SEQ ID NO: 155 - DNA, 21 bp, synthetic construct
  source 1..21: mol_type = other DNA
cttcagcaggacattgatgcc

SEQ ID NO: 156 - DNA, 22 bp, synthetic construct
  source 1..22: mol_type = other DNA
gctggtgtccctctagtctatg

SEQ ID NO: 157 - DNA, 20 bp, synthetic construct
  source 1..20: mol_type = other DNA
ggtatttgccgaagttgtag

SEQ ID NO: 158 - protein, 14 aa, synthetic construct
GAPGSAGSAAGGSG